(19) United States
(12) Patent Application Publication
(10) Pub. No.: US 2014/0208449 A1
(43) Pub. Date: Jul. 24, 2014

(54) GENETICS OF GENDER DISCRIMINATION IN DATE PALM

(71) Applicant: Joel A. Malek, Beverly, MA (US)

(72) Inventor: Joel A. Malek, Beverly, MA (US)

(73) Assignee: CORNELL UNIVERSITY, Ithaca, NY (US)

(21) Appl. No.: 14/008,012

(22) PCT Filed: Mar. 29, 2012
(86) PCT No.: PCT/US2012/031166

§ 371 Date: Dec. 2, 2013

Related U.S. Application Data
(60) Provisional application No. 61/469,032, filed on Mar. 29, 2011.

(51) Int. Cl. C12Q 1/68; A01G 1/00

(52) U.S. Cl. CPC C12Q 1/6895; A01G 1/001
USPC 800/260; 435/6.12; 800/298; 536/24.33; 536/24.3; 47/58.1 FV

(57) ABSTRACT

This invention relates to the genetics of gender discrimination in the dioecious date palm. Methods of the present invention involve analyzing DNA or RNA from a date palm plant, tissue, germplasm, or seed for the presence of (i) a nucleic acid sequence or genotype that identifies the sex of the plant, tissue, germplasm, or seed or (ii) a molecular marker in linkage disequilibrium with the nucleic acid sequence or genotype. Also disclosed are kits for selecting male and female date palm plants prior to flowering, methods of breeding a date palm plant, and a method of planting a date palm seed of a known sex.

This application claims the benefit of U.S. Provisional Patent Application Ser. No. 61/469,032, filed Mar. 29, 2011, which is hereby incorporated by reference in its entirety.

### FIELD OF THE INVENTION

This invention relates to the genetics of gender discrimination in the dioecious date palm.

### BACKGROUND OF THE INVENTION

Date palm (Phoenix dactylifera), a member of the Palm family in the Arecales order (see FIG. 1), is one of the oldest cultivated trees in the world, with evidence of domestication dating back over 5,000 years (Zohary et al., “Beginnings of Fruit Growing in the Old World,” Science 187:319-327 (1975)). Dates have been found in the tombs of Pharaohs and in neolithic sites dating 7,000 to 8,000 years ago (Kwaasi, DATE PALMS, Elsevier Science Ltd. 2003), demonstrating their historical significance in human nutrition. Date palm trees grow in hot, arid environments and are critical to the agriculture in these regions. For many countries in the Arabian Gulf, date production is the most important agricultural product. Total global production of dates in 2007 reached 6.9 million tons (http://faostat.fao.org).

Date palm biotechnology faces multiple challenges, including long plant generation times, the inability to simply distinguish between the many varieties of date palm, and the inability to distinguish female from male trees at an early stage. There are more than 2,000 date varieties with differences in fruit color, flavor, shape, size, and ripening time (Al-Farsi et al., “Nutritional and Functional Properties of Dates: A Review,” Critical Reviews in Food Science and Nutrition 48:877-887 (2008)). Likewise, the genetic component of gender determination is not well understood (Ainsworth et al., “Sex Determination in Plants,” Current Topics in Developmental Biology 38:167-223 (1997)). Specifically, date palms take 5-8 years after planting before they flower, at which point male and female trees can be distinguished. Date palm orchards can be rapidly ravished by disease, so the ability to quickly replant orchards from seeds known to be female would be of great benefit.

There are no easily distinguishable sex chromosomes in date palm, though there is some cytological evidence that they exist (Siljak-Yakovlev et al., “Chromosomal Sex Determination and Heterochromatin Structure in Date Palm,” Sexual Plant Reproduction 9:127-132 (1996)). Biochemical studies have yielded little plant gender-distinguishing power (Qacif et al., “Biochemical Investigations on Peroxidase Contents of Male and Female Inflorescences of Date Palm (Phoenix dactylifera L.),” Scientia Horticulturae 114:298-301 (2007)). A search for DNA sequences or sequence polymorphisms that are gender-specific could provide access to tools for efficient determination of date palm gender. Given the long generation time of date palm, it is not surprising that few genetic resources exist. However, a backcrossing program for date palm was initiated in California in the 1940's (Barrett, “Date Breeding and Improvement in North America,” Fruit Varieties Journal 27:50-55 (1973)). This program provided a unique genetic resource that required over 30 years to generate and is still maintained.

There is no publicly available physical or genetic map for the genome of any date palm, and recently only ˜100 kbp of nuclear date palm DNA sequences were found in GenBank (http://www.ncbi.nlm.nih.gov, which is hereby incorporated by reference in its entirety). Hence, date palm researchers need additional resources before comprehensive efforts to study or improve this important crop can begin.

The present invention is directed to overcoming these and other deficiencies in the art.

### SUMMARY OF THE INVENTION

One aspect of the present invention relates to a method of identifying the sex of a date palm plant. This method involves analyzing DNA or RNA from a date palm plant, tissue, germplasm, or seed for the presence of (i) a nucleic acid sequence that identifies the sex of the plant, tissue, germplasm, or seed or (ii) a molecular marker in linkage disequilibrium with the nucleic acid sequence. The sex of the plant, tissue, germplasm, or seed is identified based on whether or not the plant, tissue, germplasm, or seed contains the nucleic acid sequence or the molecular marker.

Another aspect of the present invention relates to a method of identifying the sex of a date palm plant. This method involves analyzing DNA or RNA from a date palm plant, tissue, germplasm, or seed for the presence of (i) a genotype that identifies the sex of the plant, tissue, germplasm, or seed, or (ii) a molecular marker linked to the genotype. The sex of the plant, tissue, germplasm, or seed is identified based on whether or not the plant, tissue, germplasm, or seed contains the genotype or the molecular marker.

A further aspect of the present invention relates to a method of selecting a male or female date palm plant prior to flowering. This method involves detecting in a date palm plant, tissue, germplasm, or seed (i) a genotype that identifies the plant, tissue, germplasm, or seed as male or female, or (ii) a molecular marker in linkage disequilibrium with the genotype. The plant, tissue, germplasm, or seed possessing the genotype or the molecular marker is selected.

Yet another aspect of the present invention relates to a kit for selecting a male or female date palm plant prior to flowering. The kit includes primers or probes for detecting in a date palm plant, tissue, germplasm, or seed (i) a genotype that identifies the plant, tissue, germplasm, or seed as male or female, or (ii) a molecular marker in linkage disequilibrium with the genotype. The kit also includes instructions for using the primers or probes for detecting the genotype or the molecular marker.

Yet a further aspect of the present invention relates to a method of selecting a male or female date palm plant prior to flowering. This method involves detecting in a date palm plant, tissue, germplasm, or seed (i) a nucleic acid sequence that identifies the plant, tissue, germplasm, or seed as male or female or (ii) a molecular marker in linkage disequilibrium with the nucleic acid sequence. The plant, tissue, germplasm, or seed possessing the nucleic acid sequence or the molecular marker is selected.

Still another aspect of the present invention relates to a kit for selecting a male or female date palm plant prior to flowering. The kit includes primers or probes for detecting in a date palm plant, tissue, germplasm, or seed (i) a nucleic acid sequence that identifies the plant, tissue, germplasm, or seed as male or female or (ii) a molecular marker in linkage disequilibrium with the nucleic acid sequence. The kit also includes instructions for using the primers or probes for detecting the nucleic acid sequence or the molecular marker.

Still a further aspect of the present invention relates to a method of breeding a date palm plant. This method involves providing a date palm plant having a sex determined by detecting in the plant or a seed, tissue, or germplasm from which it was derived (i) a genotype that identifies the plant as either male or female, or (ii) a molecular marker in linkage disequilibrium with the genotype. The date palm plant is bred with a plant of the opposite sex.

Yet another aspect of the present invention relates to a method of breeding a date palm plant. This method involves providing a date palm plant having a sex determined by detecting in the plant or a seed, tissue, or germplasm from which it was derived (i) a nucleic acid sequence that identifies the plant as male or female or (ii) a molecular marker in linkage disequilibrium with the nucleic acid sequence. The date palm plant is bred with a plant of the opposite sex.

Yet a further aspect of the present invention relates to a method of planting a date palm seed of a known sex. This method involves providing a seed having a known male or female sex and planting the seed.

The ability of the date palm plant to withstand extremely harsh conditions, while producing highly nutritious fruit with relatively minimal care, makes it a good candidate for improving arid land agriculture. Challenges such as generation times of approximately 5-8 years and dioecy (separate male and female trees) have hindered genetic studies of the date palm. To provide the foundation for date palm genetic studies, the genome of a ‘Khalas’ variety female date palm was shotgun sequenced using massively parallel sequencing. A de novo assembly of ˜380 Mbp, spanning mainly gene-rich regions, was generated using only the shotgun reads and over 25,000 gene models were predicted. To help energize date palm biotechnology, 8 additional genomes were sequenced, including those of the economically important Deglet Noor and Medjool variety females, together with their backcrossed males. Over 3.5 million polymorphic sites were identified, including >10,000 genic copy number variations. A small subset of polymorphisms capable of distinguishing multiple varieties was discovered. For the first time, a region of the genome linked to gender was identified, and evidence is presented herein that date palm employs an XY system of gender-inheritance.

### BRIEF DESCRIPTION OF THE DRAWINGS

FIG. 1 is a taxonomic tree of sequenced plant genomes. Date palm is the first available monocot (Liliopsida) draft sequence in the Arecales order. Other sequenced monocot genomes are mainly grasses.

FIGS. 2A-AK are tables setting forth 972 polymorphic sites for gender discrimination in date palm. Each DNA sequence (SEQ ID NOs:1-972) is identified by scaffold name and single nucleotide polymorphism (“SNP”) ID. Each DNA sequence is 100 nucleotides in length with the nucleotide at position 51 being the sex-determining nucleotide. The male allele (MA) at position 51 is identified for each sequence.

FIGS. 3A-D are graphs illustrating date palm SNP analysis. SNPs were analyzed between parental alleles of the Khalas reference genome and between different varieties. FIG. 3A shows that the distance between parental allele SNPs in Khalas is not normally distributed. The skewed distribution of adjacent SNP distances demonstrates the occurrence of high and low polymorphism islands in the genome. About 49% of SNPs occur within 50 bp of another SNP. This trend was maintained even after removing SNPs likely to be in repetitive regions (KhlsFilter). FIG. 3B shows that backcrossed varieties of date palm, on average, show high levels of similarity to their recurrent parent with numbers of generations (ranging from Backcross 1 to 5 generations) of backcrossing having little effect on similarity levels (error bars are quite small). Inter-variety comparisons show significantly more sites with different genotypes. FIG. 3C is a graph showing Principal Component Analysis (“PCA”) of sequenced genomes based on 3.5 million polymorphic sites. Khalas and backcrossed variants are essentially on top of each other. In FIG. 3D, PCA of sequenced genomes based on 32 Decision Tree selected polymorphic sites reveals little loss of discrimination quality with much reduced genotyping required. DN is Deglet Noor, Dy is Dayri, Mj is Medjool, BC is Backcross.

FIG. 4 is a graph showing Imbalanced Sequence Count Regions (“ISCR”) analysis among date palm genomes. The vertical axis represents the number of unique ISCRs remaining in each genome after comparison with other genomes. Only non-backcrossed genomes were considered to avoid bias from inbreeding. Approximately 7% of ISCRs were unique to any single genome while the majority were observed in at least one other genome.

FIG. 5 is a graph showing enrichment of Gene Ontology categories for genes covered by ISCRs. Gene Ontology categories from genes covered by ISCRs in at least 2 genomes were analyzed for enrichment. Gene counts in each category were normalized to total gene counts in either the genome or ISCRs. A false discovery rate of 0.2 was applied and only categories showing at least 2-fold enrichment in the ISCRs are reported.

FIGS. 6A-B are illustrations showing pedigree and genotype information for gender-discriminating regions. Date palms of known genealogy were genotyped at multiple gender-discriminating regions. FIG. 6A shows a section of the full pedigree used for linkage analysis showing the complex relationship of the trees. DN is Deglet Noor, Dy is Dayri, Mj is Medjool, BC is Backcross, and DnPr represents the initial donor parents. Grey boxes indicate an unknown but theoretically determined genotype. The genotype in each individual is the genotype found at the first gender-discriminating SNP that was genotyped. Segregation of heterozygosity with the male phenotype is clear. FIG. 6B shows genotypes from 4 scaffolds (scales with exons annotated as ticks and repeats as rectangles) with the largest number of male-specific SNPs (MS-SNPs). Genotypes from selected regions are presented with their scaffold base pair location above each genotype. The number observed (both empirically and theoretically) for each gender in each genotype is included.

FIG. 7 is an illustration showing the workflow of SNP finding. Assembly was conducted using paired-end sequences from small inserts that cannot span medium sized repeats (grey box in gene). Scaffolding utilized longer mate pairs of 2-5 kb to join adjacent contigs by spanning repeats. During “Annotation,” genes were predicted along the scaffolds. SNP finding was conducted by matching sequences to the reference and detecting regions where multiple high quality sequence reads disagreed with the reference sequence. Copy Number Variation (“CNV”) finding required matching sequence reads from a reference and test genome and detecting regions where the test genome had significantly deviated numbers of reads compared to the reference genome. For detecting regions associated with gender (M/F comparison), the genome was scanned for scaffolds containing high levels of gender segregating SNPs. In this case, scaffolds with large numbers of SNPs that were only heterozygous in males were found.

FIG. 8 is an illustration showing the comparison of seven fosmids to the assembly. The horizontal axis shows coordinates in fosmids. Regular genes in fosmids are shown as bars above (forward chain) and below (backward chain) the axes. TE genes are also shown. White bars in the black windows show matched regions between the indicated fosmid and assembly.

FIG. 9 is a chart showing clustering of 13 genomes genotyped at 32 variety discriminating SNPs. Even closely related genomes like the backcrossed males and their recurrent parents separate well using this subset of the 3.5 million original polymorphisms. “1” represents homozygous match to the reference Khalas genome, “2” represents a heterozygous position, and “3” indicates a homozygous mismatch to the reference Khalas genome. Column titles include Scaffold ID followed by base pair location of the SNP.

FIG. 10 is a graph showing coverage at ˜1.2 million locations in the genome by the test set of ˜210 million reads.

FIG. 11 is a graph showing Kmer coverage in a test set of ˜210 million reads.

FIGS. 12A-B show results of the PCR-RFLP assay based on BclI digestion. FIG. 12A shows the scaffold ID (GenBank accession numbers) followed by the primer IDs lead ([4675] (SEQ ID NO:976; [4820]-[4830] (SEQ ID NO:977); [5090] (SEQ ID NO:978)). PCR primer sites are in bold and underlined with an arrow showing the direction of amplification. DNA sequences are flanked by base pair coordinates in the scaffold. The restriction sites are in bold with the female/male allele in square brackets where they differ. In FIG. 12B, PCR-RFLP results are shown for each date palm variety assayed using the appropriate variety number from Table 10. PCR product size is 405 bp and the BclI site is at by 143. Expected product sizes from digestion are 143 bp and 262 bp. In this assay, the female allele does not contain the restriction site and is not digested while the male allele is.

FIGS. 13A-B show PCR-RFLP based on HpaII digestion. FIG. 13A shows the scaffold ID (GenBank accession numbers) followed by the primer IDs lead ([8110] (SEQ ID NO:979); [8290148300] (SEQ ID NO:980); [8475]-[8485] (SEQ ID NO:981); [8570] (SEQ ID NO:982)). PCR primer sites are in bold and underlined with an arrow showing the direction of amplification. DNA sequences are flanked by base pair coordinates in the scaffold. The restriction sites are in bold with the female/male allele in square brackets where they differ. In FIG. 13B, PCR-RFLP results are shown for each date palm variety assayed using the appropriate variety number from Table 10. PCR product size is 452 bp and HapaII sites are at hp 180, 369, and 393, although only the site at by 180 is specific to the female allele. Digestion of the female allele results in products of size 24 bp, 59 hp, 180 bp, and 189 bp, Digestion of the male allele results in products of size 24 bp, 59 bp, and 369 bp.

FIGS. 14A-B show PCR-RFLP based on RsaI digestion. FIG. 14A shows the scaffold ID (GenBank accession numbers) followed by the primer IDs lead ([41360] (SEQ ID NO:983; [41650]-[41660] (SEQ ID NO:984); [41870] (SEQ ID NO:985)). PCR primer sites are in bold and underlined with an arrow showing the direction of amplification. DNA sequences are flanked by base pair coordinates in the scaffold. The restriction sites are in bold with the female/male allele in square brackets where they differ. In FIG. 14B, PCR-RFLP results are shown for each date palm variety assayed using the appropriate variety number from Table 10. PCR product size is 493 bp and RsaI sites are at by 5 and 288. Expected product sizes from digestion of the female allele are 5 bp, 205 bp, and 283 bp. Two males (1M and 8M) did not contain the male-specific allele, resulting in digestion and suggesting that allele is not as widespread in the population.

FIGS. 15A-C are results of a PCR-only-based assay. FIG. 15A shows the scaffold ID (GenBank accession numbers) followed by the primer IDs lead ([4630Fem] (SEQ ID NO:986; [4630Mal] (SEQ ID NO:987); [5075Fem] (SEQ ID NO:988); [5075Mal] (SEQ ID NO:989)). Primers designed to match the female allele are highlighted while mismatched bases in the same strand of the male allele are shown. Arrows indicate direction of the primers. In FIG. 15B, primers designed to match the male allele are shown ([4650Fem] (SEQ ID NO:990; [4650Mal] (SEQ ID NO:991); [4980Fem] (SEQ ID NO:992); [4980Mal] (SEQ ID NO:993)). A polymorphism detected only in Deglet Noor males and not in other male sequences is identified. FIG. 15C shows PCR-only-based assay results on seven males and seven females. Abbreviations are as in Table 11. While all tests show primer dimers, female samples show the expected single band with the male samples showing the expected two bands.

### DETAILED DESCRIPTION OF THE INVENTION

The present invention pertains to date palm plants, which are dioecious plants of the species Phoenix dactylifera. According to one aspect, the present invention relates to a method of identifying the sex of a date palm plant. This method involves analyzing DNA or RNA from a date palm plant, tissue, germplasm, or seed for the presence of (i) a nucleic acid sequence that identifies the sex of the plant, tissue, germplasm, or seed or (ii) a molecular marker in linkage disequilibrium with the nucleic acid sequence. The sex of the plant, tissue, germplasm, or seed is identified based on whether or not the plant, tissue, germplasm, or seed contains the nucleic acid sequence or the molecular marker.

The terms plant, issue, germplasm, and seed refer to any of whole plants, plant parts, plant components or organs (e.g., leaves, stems, roots, floral structures, etc.), plant tissue, seeds, plant cells, and/or progeny of the same. A plant cell is a cell of a plant, taken from a plant, or derived through culture from a cell taken from a plant.

Analyzing DNA or RNA from a date palm plant, tissue, germplasm, or seed pursuant to the present invention can be carried out by methods well-known in the art. Such methods include, e.g., DNA sequencing, hybridization assays, PCR-based assays, detection of markers (e.g., SNPs, simple sequence repeats (“SSRs”), restriction fragment length polymorphisms (“RFLPs”), amplified fragment length polymorphisms (“AFLPs”), and isozyme markers). Well-established methods are also known for the detection of expressed sequence tags (“ESTs”) and SSR markers derived from EST sequences and randomly amplified polymorphic DNA.

According to one embodiment of the present invention, analyzing DNA or RNA from a date palm plant involves detecting, in a hybridization assay, whether a nucleic acid sequence that identifies the sex of the date palm plant, tissue, germplasm, or seed hybridizes to an oligonucleotide probe. Alternatively, analyzing involves detecting, in a PCR-based assay, whether oligonucleotide primers amplify a nucleic acid sequence indicative of the gender of the date palm plant, tissue, germplasm, or seed being analyzed.

In one embodiment of the present invention, the presence of a nucleic acid sequence that identifies the sex of a date palm is detected using a direct sequencing technique. Specifically, DNA samples are first isolated from a date palm plant using any suitable method. The region of interest is cloned into a suitable vector and amplified by growth in a host cell (e.g., bacteria). Alternatively, DNA in the region of interest is amplified using PCR.

Following amplification, DNA in the region of interest (e.g., the region containing the gender indicative SNP or marker) is sequenced using any suitable method including, but not limited to, manual sequencing using radioactive marker nucleotides and automated sequencing. The results of the sequencing are displayed using any suitable method. The sequence is examined and the presence or absence of a given SNP or marker is determined.

Alternatively, a PCR-based assay is used, which employs oligonucleotide primers that hybridize only to a gender indicative SNP or allele. Primers are used to amplify a sample of DNA. For example, primers can be constructed pursuant to well-known methods in the art to amplify, e.g., only nucleotide sequences possessing a male allele. If the primers result in a PCR product, then the plant has the male allele and the plant is identified as male.

In a hybridization assay, the presence or absence of a given SNP (e.g., a gender indicative allele) or marker is determined based on the ability of the DNA from the sample to hybridize to a complementary DNA molecule (e.g., an oligonucleotide probe). A variety of hybridization assays using a variety of technologies for hybridization and detection are available and include, without limitation, direct detection of hybridization, detection of hybridization using DNA chip assays, and enzymatic detection of hybridization.

In direct detection of hybridization, hybridization of a probe to the sequence of interest (e.g., a gender indicative SNP or marker) is detected directly by visualizing a bound probe (e.g., a Northern or Southern assay). In these assays, genomic DNA (Southern) or RNA (Northern) is isolated from a plant. The DNA or RNA is then cleaved with a series of restriction enzymes that cleave infrequently in the genome and not near any of the markers being assayed. The DNA or RNA is then separated (e.g., on an agarose gel) and transferred to a membrane. A labeled (e.g., by incorporating a radionucleotide) probe or probes specific for the gender indicative SNP or marker being detected is allowed to contact the membrane under low, medium, or high stringency conditions. Unbound probe is removed and the presence of binding is detected by visualizing the labeled probe.

In detection of hybridization using DNA chip assays, a series of oligonucleotide probes are affixed to a solid support. The oligonucleotide probes are designed to be unique to a given SNP or marker. The DNA sample of interest is contacted with the DNA chip and hybridization is detected. In some embodiments, the DNA chip assay is a GeneChip (Affymetrix, Santa Clara, Calif.; see, e.g., U.S. Pat. Nos. 6,045,996; 5,925,525; and 5,858,659 which are hereby incorporated by reference in their entirety) assay. The GeneChip technology uses miniaturized, high-density arrays of oligonucleotide probes affixed to a chip. Probe arrays are manufactured, e.g., by Affymetrix's light-directed chemical synthesis process, which combines solid-phase chemical synthesis with photolithographic fabrication techniques employed in the semiconductor industry. Using a series of photolithographic masks to define chip exposure sites, followed by specific chemical synthesis steps, the process constructs high-density arrays of oligonucleotides, with each probe in a predefined position in the array. Multiple probe arrays are synthesized simultaneously on a large glass wafer. The wafers are then diced, and individual probe arrays are packaged in injection-molded plastic cartridges, which protect them from the environment and serve as chambers for hybridization.

The nucleic acid to be analyzed is isolated, amplified by PCR, and labeled with a fluorescent reporter group. The labeled nucleic acid is then incubated with the array using a fluidics station. The array is then inserted into the scanner, where patterns of hybridization are detected. The hybridization data are collected as light emitted from the fluorescent reporter groups are incorporated into the target, which is bound to the probe array. Probes that perfectly match the target generally produce stronger signals than those that have mismatches. Since the sequence and position of each probe on the array are known, by complementarity, the identity of the target nucleic acid applied to the probe array can be determined.

In other embodiments, a DNA microchip containing electronically captured probes (Nanogen, San Diego, Calif.) is utilized (see, e.g., U.S. Pat. Nos. 6,017,696; 6,068,818; and 6,051,380; which are hereby incorporated by reference in their entirety). Through the use of microelectronics, Nanogen's technology enables the active movement and concentration of charged molecules to and from designated test sites on its semiconductor microchip. DNA capture probes unique to a given SNP or marker are electronically placed at, or “addressed” to, specific sites on the microchip. Since DNA has a strong negative charge, it can be electronically moved to an area of positive charge.

First, a test site or a row of test sites on the microchip is electronically activated with a positive charge. Next, a solution containing the DNA probes is introduced onto the microchip. The negatively charged probes rapidly move to the positively charged sites, where they concentrate and are chemically bound to a site on the microchip. The microchip is then washed and another solution of distinct DNA probes is added until the array of specifically bound DNA probes is complete.

A test sample is then analyzed for the presence of target DNA molecules by determining which of the DNA capture probes hybridize, with complementary DNA in the test sample (e.g., a PCR amplified gene of interest). An electronic charge is also used to move and concentrate target molecules to one or more test sites on the microchip. The electronic concentration of sample DNA at each test site promotes rapid hybridization of sample DNA with complementary capture probes (hybridization may occur in minutes). To remove any unbound or nonspecifically bound DNA from each site the polarity or charge of the site is reversed to negative, thereby forcing any unbound or nonspecifically bound DNA back into solution away from the capture probes. A laser-based fluorescence scanner is used to detect binding.

In still further embodiments, an array technology based upon the segregation of fluids on a flat surface (chip) by differences in surface tension (ProtoGene, Palo Alto, Calif.) is utilized (see, e.g., U.S. Pat. Nos. 6,001,311; 5,985,551; and 5,474,796; which are hereby incorporated by reference in their entirety). Protogene's technology is based on the fact that fluids can be segregated on a flat surface by differences in surface tension that have been imparted by chemical coatings. Once so segregated, oligonucleotide probes are synthesized directly on the chip by ink-jet printing of reagents. The array, with its reaction sites defined by surface tension, is mounted on an X/Y translation stage under a set of four piezoelectric nozzles, one for each of the four standard DNA bases. The translation stage moves along each of the rows of the array and the appropriate reagent is delivered to each of the reaction sites. For example, the A amidite is delivered only to the sites where amidite A is to be coupled during that synthesis step and so on. Common reagents and washes are delivered by flooding the entire surface and then removing them by spinning.

DNA probes unique for the SNP or marker of interest are affixed to the chip using Protogene's technology. The chip is then contacted with the PCR-amplified genetic region of interest. Following hybridization, unbound DNA is removed and hybridization is detected using any suitable method (e.g., by fluorescence de-quenching of an incorporated fluorescent group).

In yet other embodiments, a “bead array” is used for the detection of polymorphisms (Illumina, San Diego, Calif.; see, e.g., WO 99/67641 and WO 00/39587, which are hereby incorporated by reference in their entirety). Illumina uses a BEAD ARRAY technology that combines fiber optic bundles and beads that self-assemble into an array. Each fiber optic bundle contains thousands to millions of individual fibers depending on the diameter of the bundle. The beads are coated with an oligonucleotide specific for the detection of a given SNP or marker. Batches of beads are combined to form a pool specific to the array. To perform an assay, the BEAD ARRAY is contacted with a prepared subject sample (e.g. DNA). Hybridization is detected using any suitable method.

In enzymatic detection of hybridization, hybridization of a bound probe is detected using a TaqMan assay (PE Biosystems, Foster City, Calif.; see, e.g., U.S. Pat. Nos. 5,962,233 and 5,538,848, which are hereby incorporated by reference in their entirety). The assay is performed during a PCR reaction. The TaqMan assay exploits the 5′-3′ exonuclease activity of DNA polymerases such as AMPLITAQ DNA polymerase. A probe, specific for a given SNP or marker, is included in the PCR reaction. The probe consists of an oligonucleotide with a 5′-reporter dye (e.g. a fluorescent dye) and a 3′-quencher dye. During PCR, if the probe is bound to its target, the 5′-3′ nucleolytic activity of the AMPLITAQ polymerase cleaves the probe between the reporter and the quencher dye. The separation of the reporter dye from the quencher dye results in an increase of fluorescence. The signal accumulates with each cycle of PCR and can be monitored with a fluorimeter.

In still further embodiments, polymorphisms are detected using the SNP-IT primer extension assay (Orchid Biosciences, Princeton, N.J.; see, e.g., U.S. Pat. Nos. 5,952,174 and 5,919,626, which are hereby incorporated by reference in their entirety). In this assay, SNPs are identified by using a specially synthesized DNA primer and a DNA polymerase to selectively extend the DNA chain by one base at the suspected SNP location. DNA in the region of interest is amplified and denatured. Polymerase reactions are then performed using miniaturized systems called microfluidics. Detection is accomplished by adding a label to the nucleotide suspected of being at the SNP or marker location. Incorporation of the label into the DNA can be detected by any suitable method (e.g., if the nucleotide contains a biotin label, detection is via a fluorescently labeled antibody specific for biotin). Numerous other assays are known in the art.

Additional detection assays that are suitable for use in the present invention include, but are not limited to, enzyme mismatch cleavage methods (e.g., Variagenics, U.S. Pat. Nos. 6,110,684; 5,958,692; and 5,851,770, which are hereby incorporated by reference in their entirety); polymerase chain reaction; branched hybridization methods (e.g., Chiron, U.S. Pat. Nos. 5,849,481; 5,710.264; 5,124,246; and 5,624,802; which are hereby incorporated by reference in their entirety); rolling circle replication (e.g., U.S. Pat. Nos. 6,210,884 and 6,183.960, which are hereby incorporated by reference in their entirety); NASBA (e.g., U.S. Pat. No. 5,409,818, which is hereby incorporated by reference in its entirety); molecular beacon technology (e.g., U.S. Pat. No. 6,150,097, which is hereby incorporated by reference in its entirety); E-sensor technology (Motorola, U.S. Pat. Nos. 6,248,229; 6,221,583; 6,013,170; and 6,063,573; which are hereby incorporated by reference in their entirety): INVADER assay (Third Wave Technologies; see, e.g, U.S. Pat. Nos. 5,846,717; 6,090,543; 6,001,567; 5,985,557; and 5,994,069; which are hereby incorporated by reference in their entirety); cycling probe technology (e.g., U.S. Pat. Nos. 5,403,711; 5,011,769; and 5,660,988: which are hereby incorporated by reference in their entirety); Dade Behring signal amplification methods (e.g., U.S. Pat. Nos. 6,121,001; 6,110,677; 5,914,230; 5,882,867; and 5,792,614; which are hereby incorporated by reference in their entirety); ligase chain reaction (Bamay, Proc. Natl. Acad. Sci USA 88:189-93 (1991), which is hereby incorporated by reference in its entirety); and sandwich hybridization methods (e.g., U.S. Pat. No. 5,288,609, which is hereby incorporated by reference in its entirety).

In some embodiments, a MassARRAY system (Sequenom, San Diego, Calif.) is used to detect variant sequences (see, e.g., U.S. Pat. Nos. 6,043,031; 5,777,324; and 5,605,798; which are hereby incorporated by reference in their entirety). DNA is isolated from cell samples using standard procedures. Next, specific DNA regions containing the SNP or marker of interest, about 200 base pairs in length, are amplified by PCR. The amplified fragments are then attached by one strand to a solid surface and the non-immobilized strands are removed by standard denaturation and washing. The remaining immobilized single strand then serves as a template for automated enzymatic reactions that produce genotype specific diagnostic products.

Very small quantities of the enzymatic products, typically five to ten nanoliters, are then transferred to a SpectroCHIP array for subsequent automated analysis with the SpectroREADER mass spectrometer. Each spot is preloaded with light absorbing crystals that form a matrix with the dispensed diagnostic product. The MassARRAY system uses MALDI-TOF (Matrix Assisted Laser Desorption Ionization Time of Flight) mass spectrometry. In a process known as desorption, the matrix is hit with a pulse from a laser beam. Energy from the laser beam is transferred to the matrix and it is vaporized resulting in a small amount of the diagnostic product being expelled into a flight tube. As the diagnostic product is charged when an electrical field pulse is subsequently applied to the tube they are launched down the flight tube towards a detector. The time between application of the electrical field pulse and collision of the diagnostic product with the detector is referred to as the time of flight. This is a very precise measure of the product's molecular weight, as a molecule's mass correlates directly with time of flight with smaller molecules flying faster than larger molecules. The entire assay is completed in less than one thousandth of a second, enabling samples to be analyzed in a total of 3-5 seconds, including repetitive data collection. The SpectroTYPER software then calculates, records, compares, and reports the genotypes at the rate of three seconds per sample.

The methods of the present invention may involve an automated system for detecting nucleic acid sequences and/or markers. For example, an automated system may include a set of marker probes or primers configured to detect at least one gender indicative SNP or marker as described herein.

A typical system may include a detector that is configured to detect one or more signal outputs from the set of marker probes or primers, or amplicon thereof, thereby identifying the presence or absence of an allele. A wide variety of signal detection apparatus are available, including photo multiplier tubes, spectrophotometers, CCD arrays, arrays and array scanners, scanning detectors, phototubes and photodiodes, microscope stations, galvo-scans, microfluidic nucleic acid amplification detection appliances, and the like. The precise configuration of the detector will depend, in part, on the type of label used to detect the marker allele, as well as the instrumentation that is most conveniently obtained for the user. Detectors that detect fluorescence, phosphorescence, radioactivity, pH, charge, absorbance, luminescence, temperature, magnetism, or the like can be used. Typical detector examples include light (e.g., fluorescence) detectors or radioactivity detectors. For example, detection of a light emission (e.g., a fluorescence emission) or other probe label is indicative of the presence or absence of an allele. Fluorescent detection is generally used for detection of amplified nucleic acids (however, upstream and/or downstream operations can also be performed on amplicons, which can involve other detection methods). In general, the detector detects one or more label (e.g., light) emission from a probe label, which is indicative of the presence or absence of a marker.

The detector(s) optionally monitors one or a plurality of signals from an amplification reaction. For example, the detector can monitor optical signals which correspond to “real time” amplification assay results.

System instructions that correlate the presence or absence of the gender indicative SNP or marker with the predicted tolerance are also contemplated by the present invention. For example, the instructions can include at least one look-up table that includes a correlation between the presence or absence of an allele and the predicted sex of the plant. The precise form of the instructions can vary depending on the components of the system, e.g., they can be present as system software in one or more integrated units of the system (e.g., a microprocessor, computer, or computer readable medium), or can be present in one or more units (e.g., computers or computer readable media) operably coupled to the detector. In one typical example, the system instructions may include at least one look-up table that includes a correlation between the presence or absence of the allele(s) and predicted tolerance or improved tolerance. The instructions also typically include instructions providing a user interface with the system, e.g., to permit a user to view results of a sample analysis and to input parameters into the system.

A system may typically include components for storing or transmitting computer readable data representing or designating the allele(s) detected by the methods of the present invention, e.g., in an automated system. The computer readable media can include, for example, cache, main, and storage memory and/or other electronic data storage components (hard drives, floppy drives, storage drives, etc.) for storage of computer code. Data representing alleles detected by the methods of the present invention can also be electronically, optically, or magnetically transmitted in a computer data signal embodied in a transmission medium over a network such as an intranet or internet or combinations thereof. The system can also, or alternatively, transmit data via wireless, or other available transmission alternatives.

During operation, the system may typically comprise a sample that is to be analyzed, such as a plant tissue, or material isolated from the tissue such as genomic DNA, amplified genomic DNA, cDNA, amplified cDNA, RNA, amplified RNA, or the like.

Automated systems for detecting nucleic acid sequences and/or markers and/or correlating the nucleic acid sequences and/or markers with a male or female phenotype may involve data entering a computer which corresponds to physical objects or processes external to the computer, e.g., a marker allele, and a process that, within a computer, causes a physical transformation of the input signals to different output signals. In other words, the input data, e.g., amplification of a particular marker allele, is transformed to output data, e.g, the identification of the allelic form of a chromosome segment. The process within the computer is a set of instructions, or program, by which positive amplification or hybridization signals are recognized by the integrated system and attributed to individual samples as a genotype. Additional programs correlate the identity of individual samples with a sex-related phenotype or marker alleles, e.g. statistical methods. In addition, there are numerous, e.g., C/C++ programs for computing, Delphi and/or Java programs for GUI interfaces, and productivity tools (e.g., Microsoft Excel and/or SigmaPlot) for charting or creating look up tables of relevant allele-trait correlations. Other useful software tools in the context of the integrated systems of the invention include statistical packages such as SAS, Genstat, Matlab, Mathematica, and S-Plus and genetic modeling packages such as QU-GENE. Furthermore, additional programming languages such as visual basic are also suitably employed in the integrated systems.

By way of example, sex identifying marker alleles assigned to a population are recorded in a computer readable medium. Data regarding genotype for one or more molecular markers, e.g. SSR, RFLP, AFLP, SNP, isozyme markers or other markers as described herein, are similarly recorded in a computer accessible database. Optionally, marker data is obtained using an integrated system that automates one or more aspects of the assay (or assays) used to determine marker genotype. In such a system, input data corresponding to genotypes for molecular markers are relayed from a detector, e.g., an array, a scanner, a CCD, or other detection device directly to files in a computer readable medium accessible to the central processing unit. A set of system instructions (typically embodied in one or more programs) encoding the correlations between tolerance and the alleles of the invention is then executed by the computational device to identify correlations between marker alleles and predicted trait phenotypes.

Typically, the system also includes a user input device, such as a keyboard, a mouse, a touchscreen, or the like, for, e.g., selecting files, retrieving data, reviewing tables of maker information, etc. and an output device (e.g., a monitor, a printer, etc.) for viewing or recovering the product of the statistical analysis.

Integrated systems comprising a computer or computer readable medium comprising set of files and/or a database with at least one data set that corresponds to the marker alleles herein are provided. The system optionally also includes a user interface allowing a user to selectively view one or more of these databases. In addition, standard text manipulation software such as word processing software (e.g., Microsoft Word™ or Corel Wordperfect™) and database or spreadsheet software (e.g., spreadsheet software such as Microsoft Excel™, Corel Quattro Pro™, or database programs such as Microsoft Access™ or Paradox™) can be used in conjunction with a user interface (e.g., a GUI in a standard operating system such as a Windows, Macintosh, Unix or Linux system) to manipulate strings of characters corresponding to the alleles or other features of the database.

The system may optionally include components for sample manipulation, e.g., incorporating robotic devices. For example, a robotic liquid control armature for transferring solutions (e.g., plant cell extracts) from a source to a destination, e.g., from a microtiter plate to an array substrate, is optionally operably linked to the digital computer (or to an additional computer in the integrated system). An input device for entering data to the digital computer to control high throughput liquid transfer by the robotic liquid control armature and, optionally, to control transfer by the armature to the solid support is commonly a feature of the integrated system. Many such automated robotic fluid handling systems are commercially available. For example, a variety of automated systems are available from Caliper Technologies (Hopkinton, Mass.), which utilize various Zymate systems, which typically include, e.g., robotics and fluid handling modules. Similarly, the common ORCA® robot, which is used in a variety of laboratory systems, e.g., for microtiter tray manipulation, is also commercially available, e.g., from Beckman Coulter, Inc. (Fullerton, Calif.). As an alternative to conventional robotics, microfluidic systems for performing fluid handling and detection are now widely available, e.g., from Caliper Technologies Corp. (Hopkinton, Mass.) and Agilent technologies (Palo Alto, Calif.).

Systems for molecular marker analysis can include a digital computer with one or more of high-throughput liquid control software, image analysis software for analyzing data from marker labels, data interpretation software, a robotic liquid control armature for transferring solutions from a source to a destination operably linked to the digital computer, an input device (e.g., a computer keyboard) for entering data to the digital computer to control high throughput liquid transfer by the robotic liquid control armature and, optionally, an image scanner for digitizing label signals from labeled probes hybridized, e.g., to markers on a solid support operably linked to the digital computer. The image scanner interfaces with the image analysis software to provide a measurement of, e.g., nucleic acid probe label intensity upon hybridization to an arrayed sample nucleic acid population (e.g., comprising one or more markers), where the probe label intensity measurement is interpreted by the data interpretation software to show whether, and to what degree, the labeled probe hybridizes to a marker nucleic acid (e.g., an amplified marker allele). The data so derived is then correlated with sample identity, to determine the gender of a date palm plant.

Optical images, e.g., hybridization patterns viewed (and, optionally, recorded) by a camera or other recording device (e.g., a photodiode and data storage device) are optionally further processed in any of the embodiments herein, e.g., by digitizing the image and/or storing and analyzing the image on a computer. A variety of commercially available peripheral equipment and software is available for digitizing, storing, and analyzing a digitized video or digitized optical image, e.g., using PC (Intel x86 or pentium chip-compatible DOS™, OS2™, WINDOWS™, WINDOWS NT™ or WINDOWS95™. based machines), MACINTOSH™, LINUX, or UNIX based (e.g. SUN™ work station) computers.

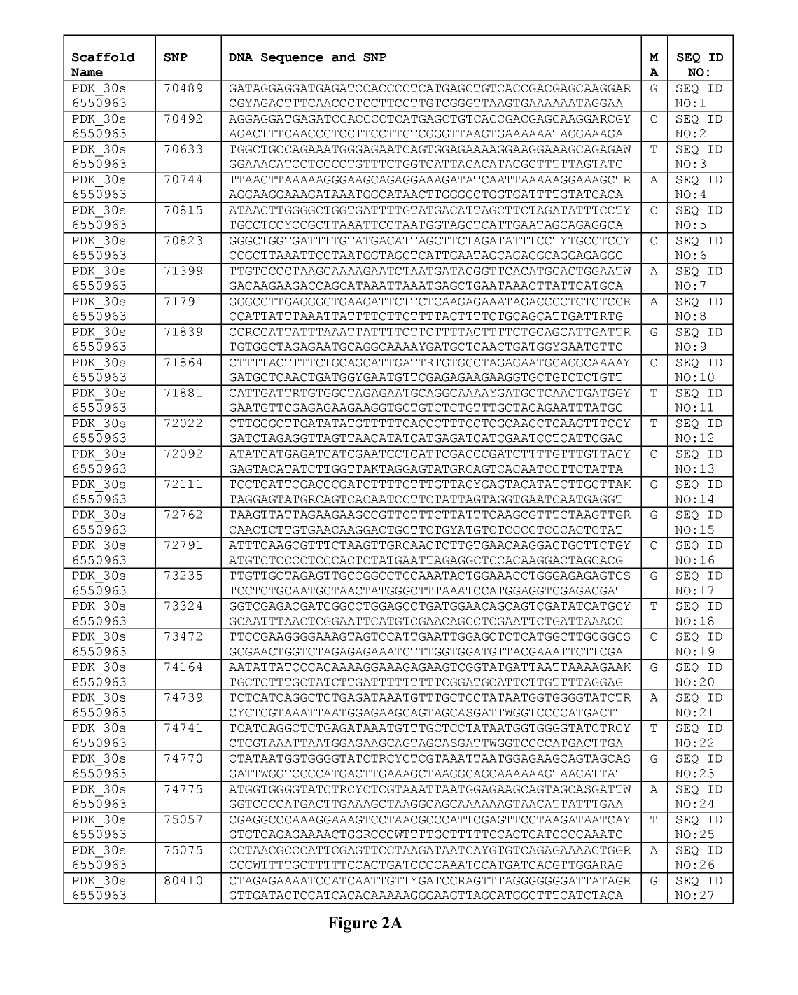

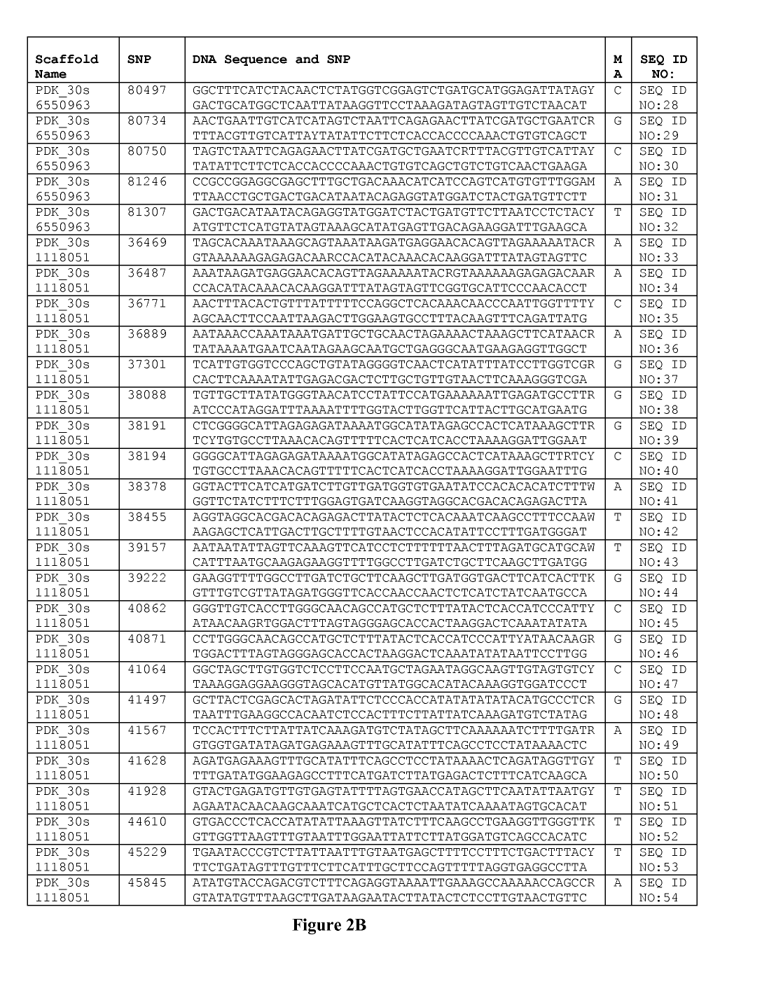

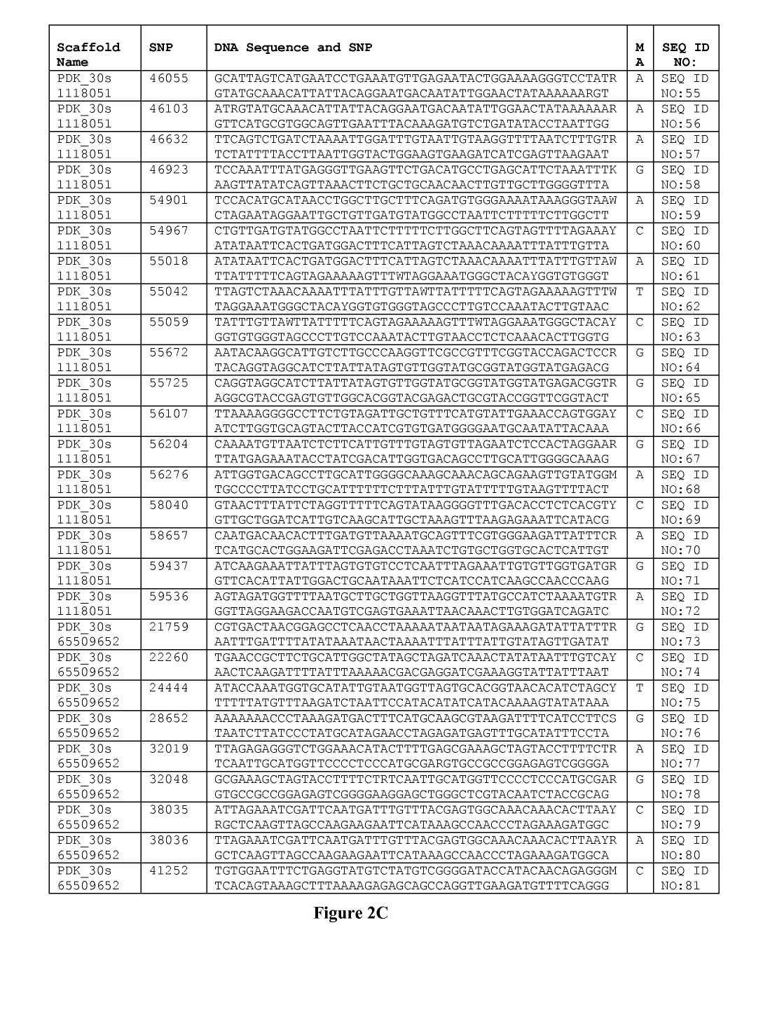

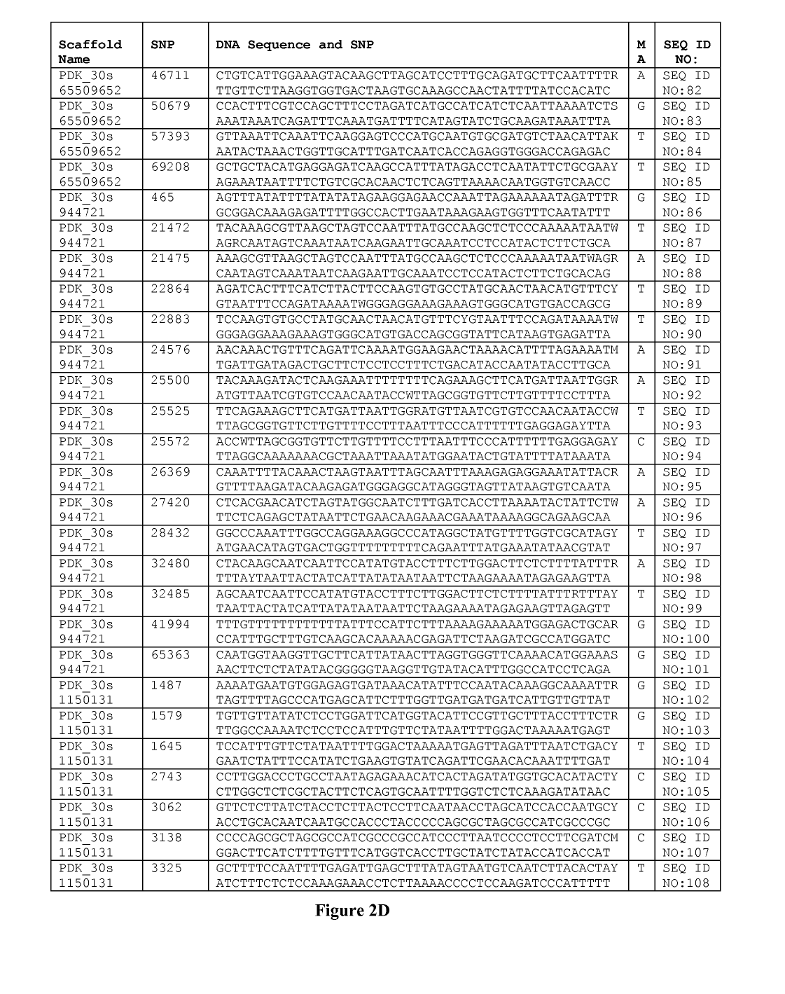

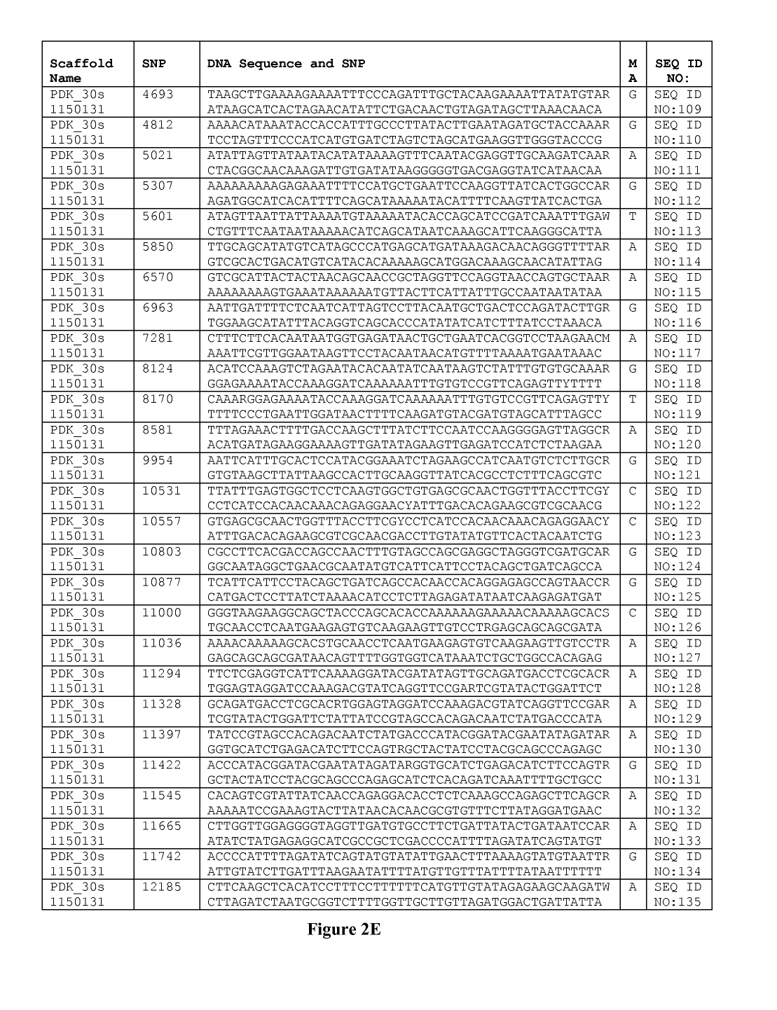

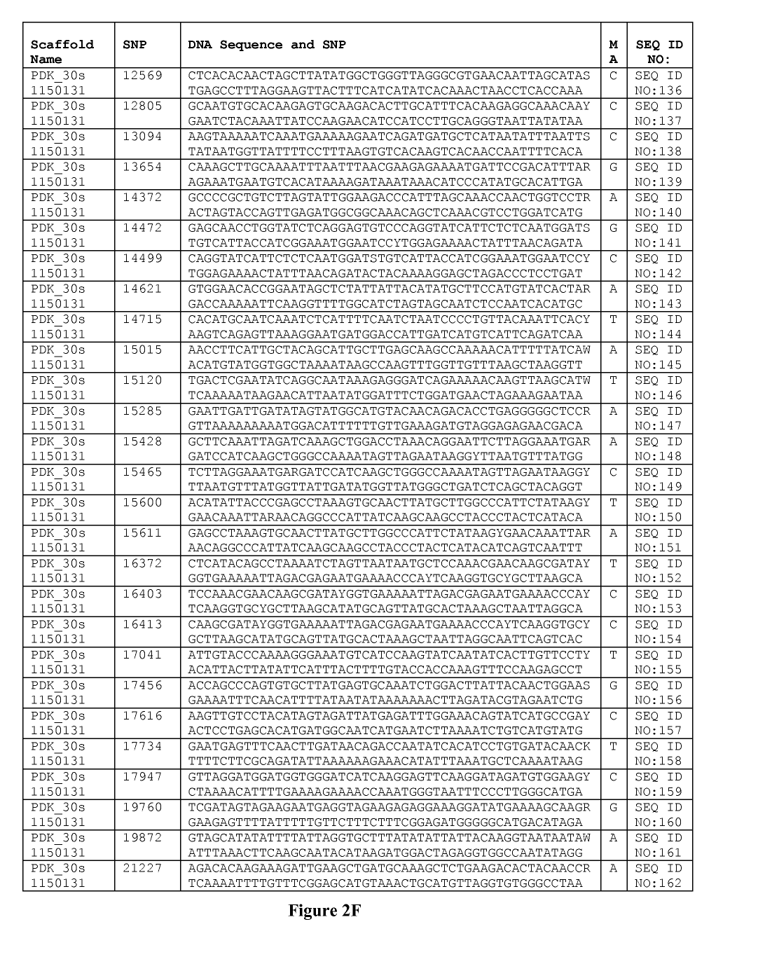

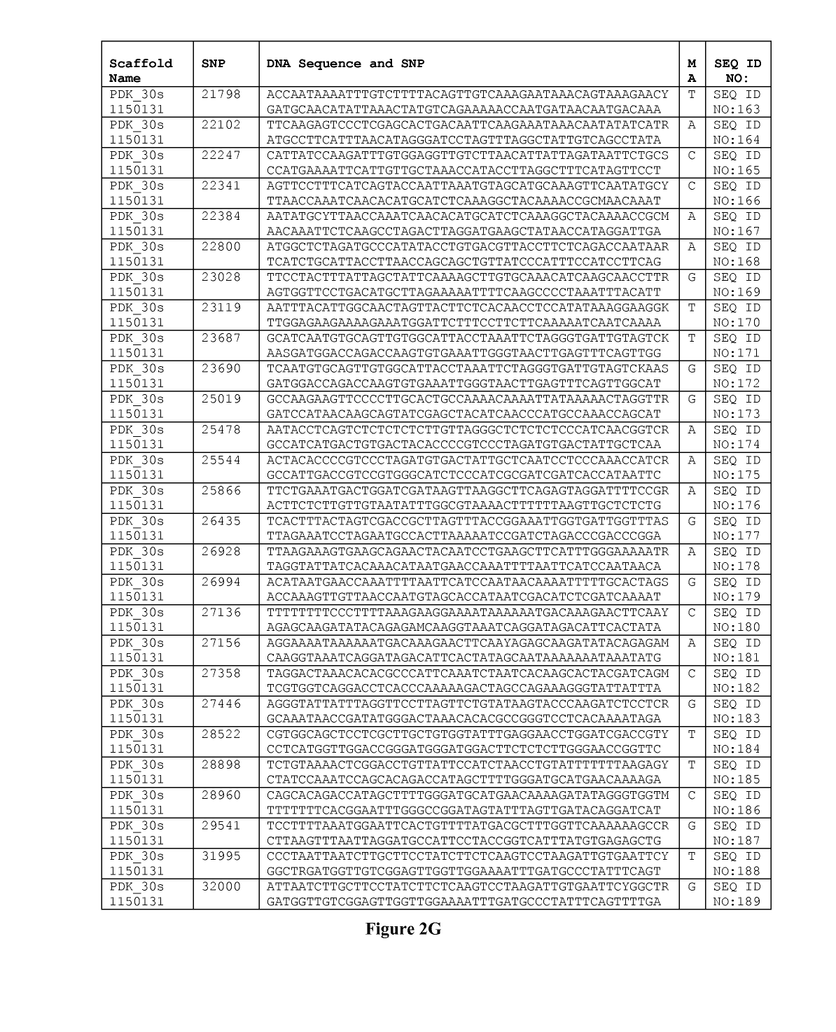

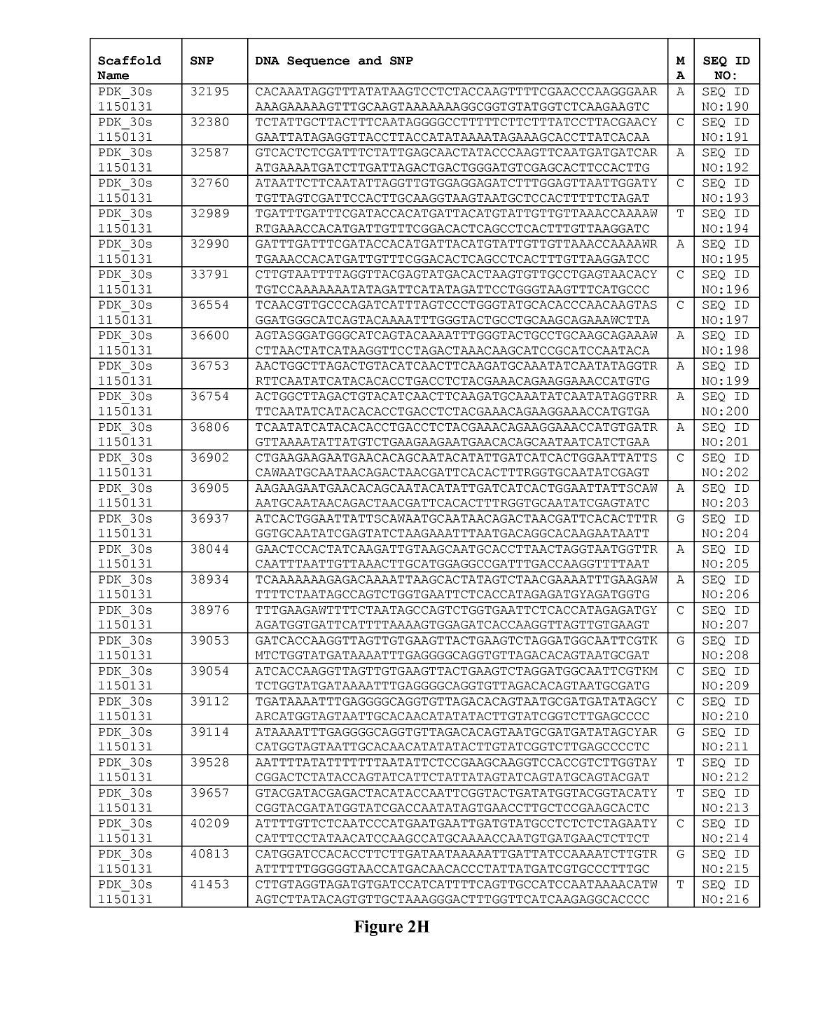

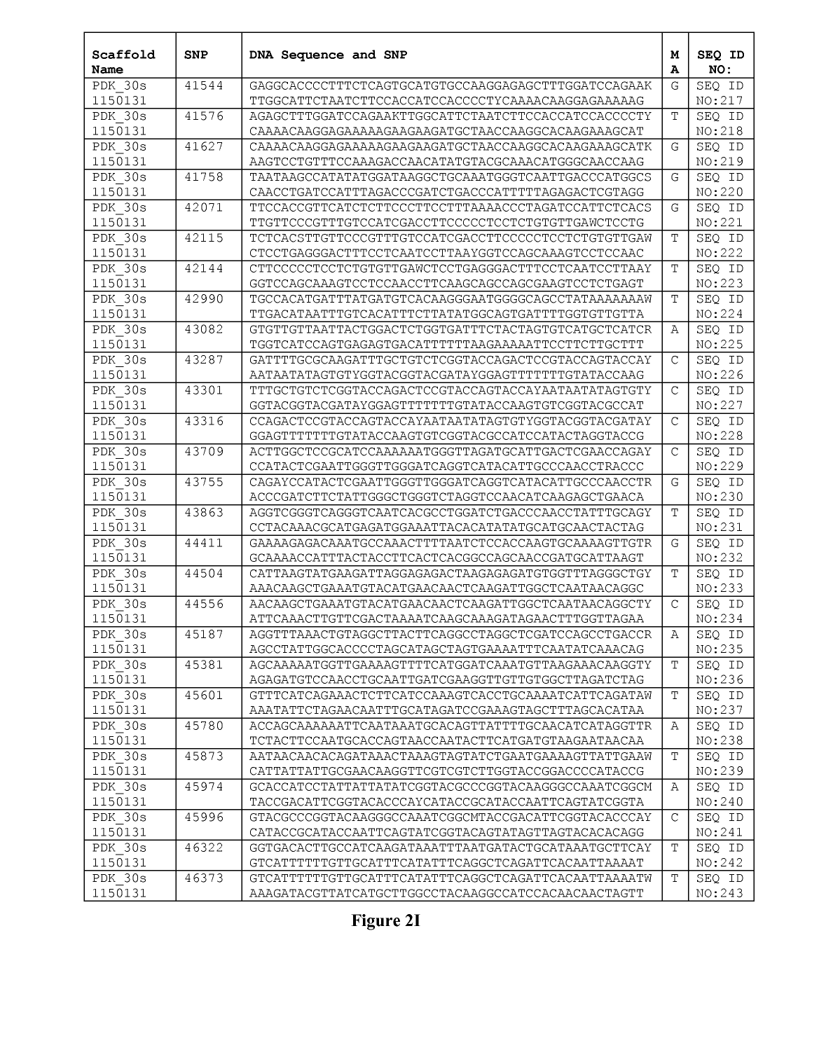

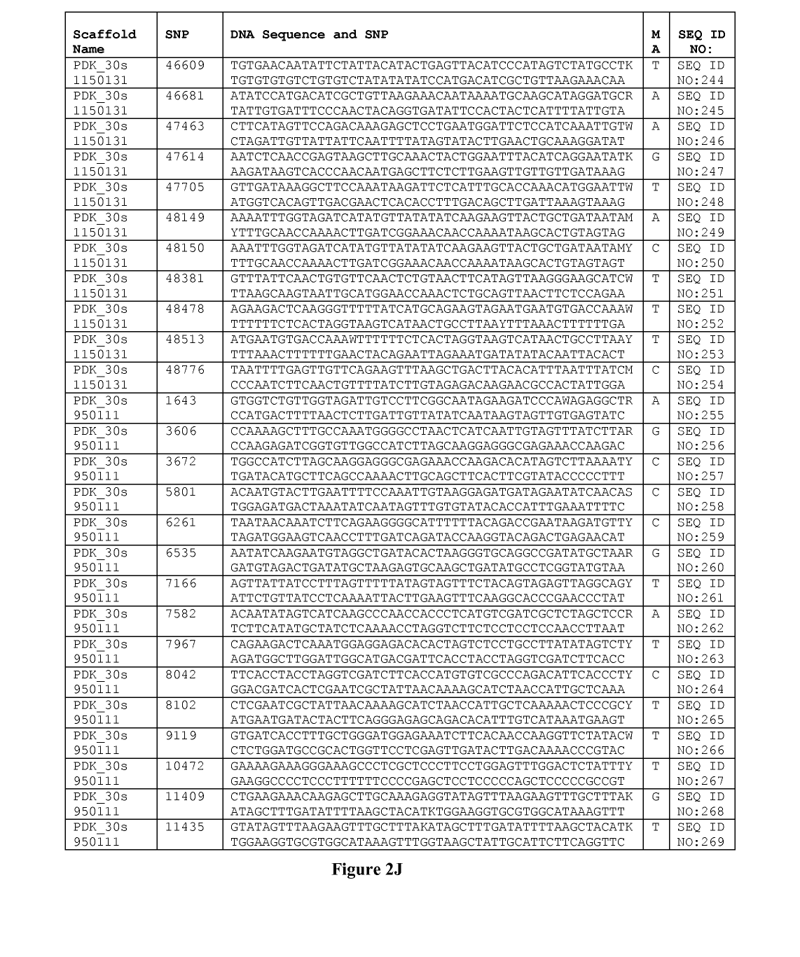

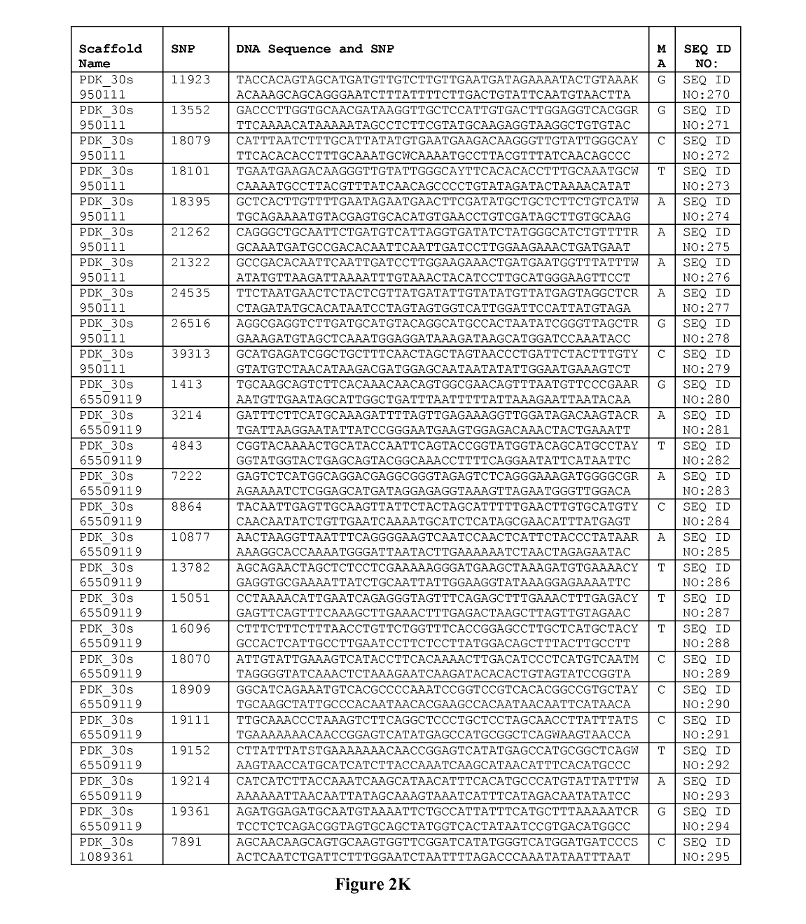

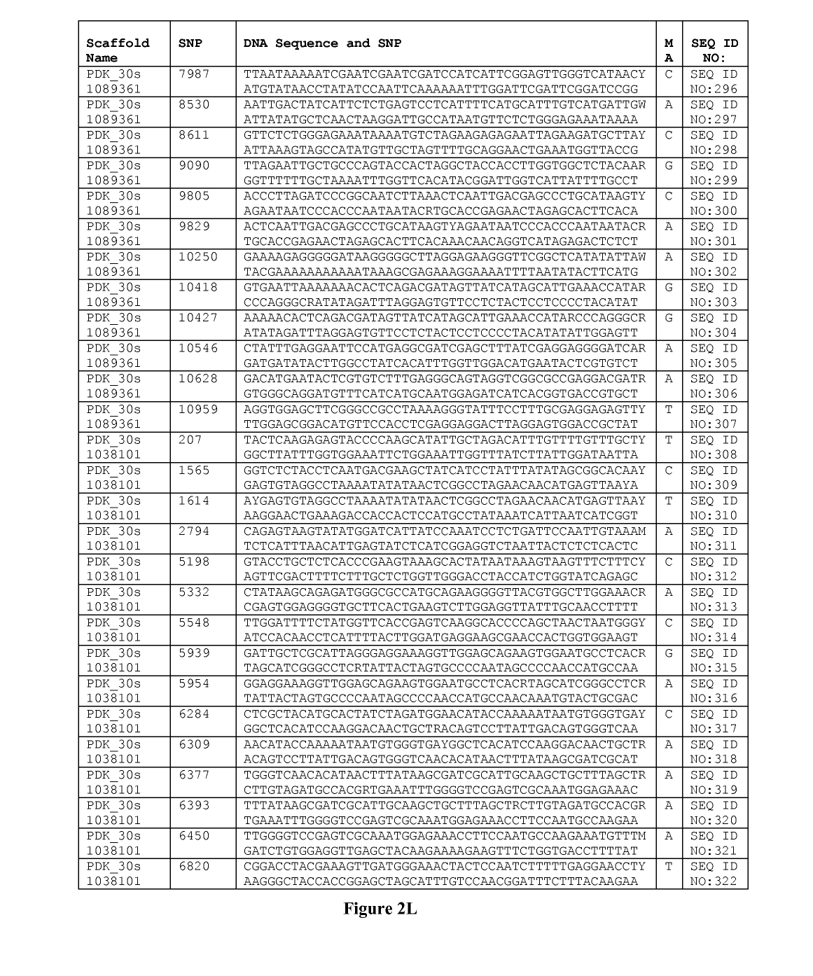

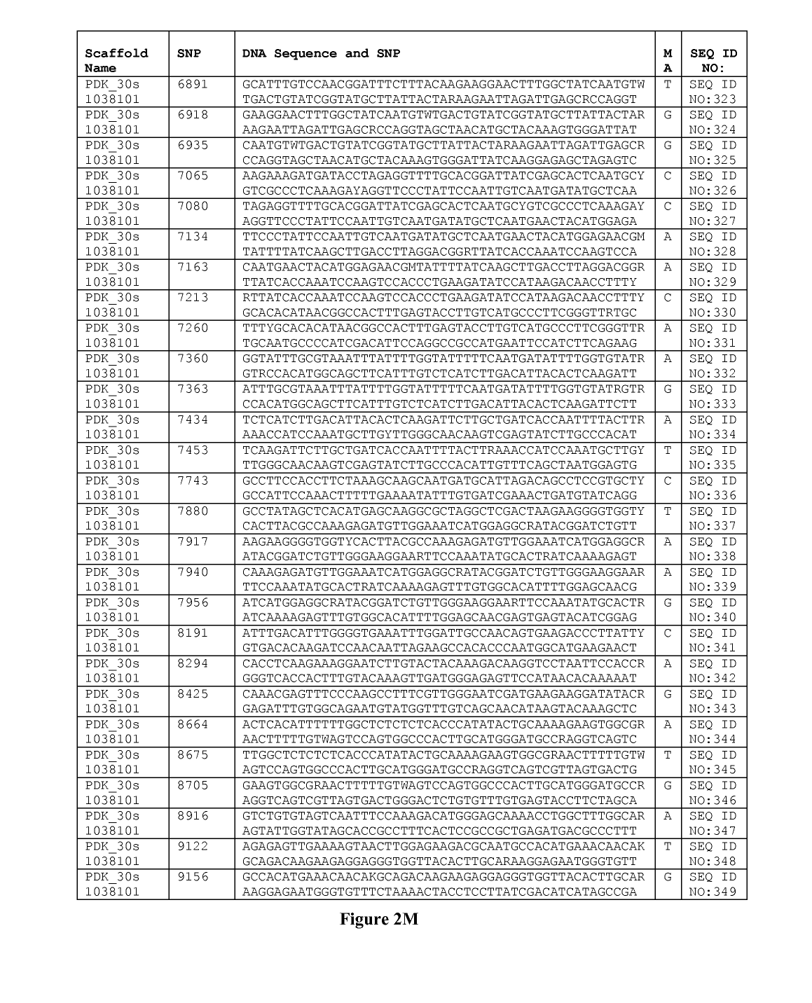

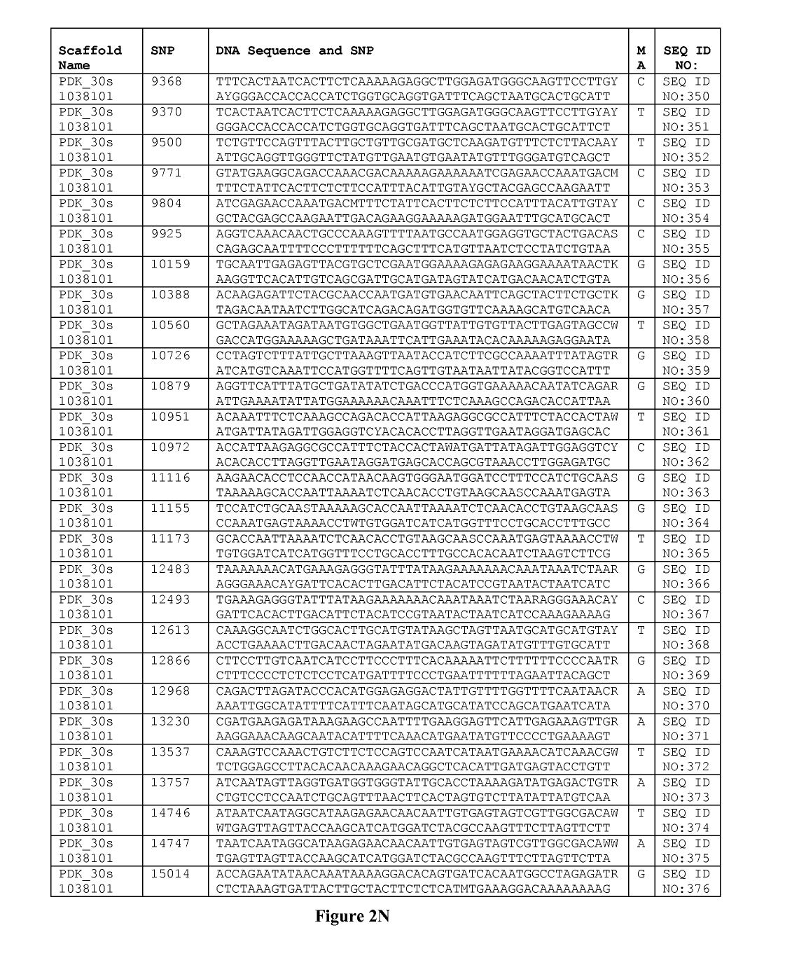

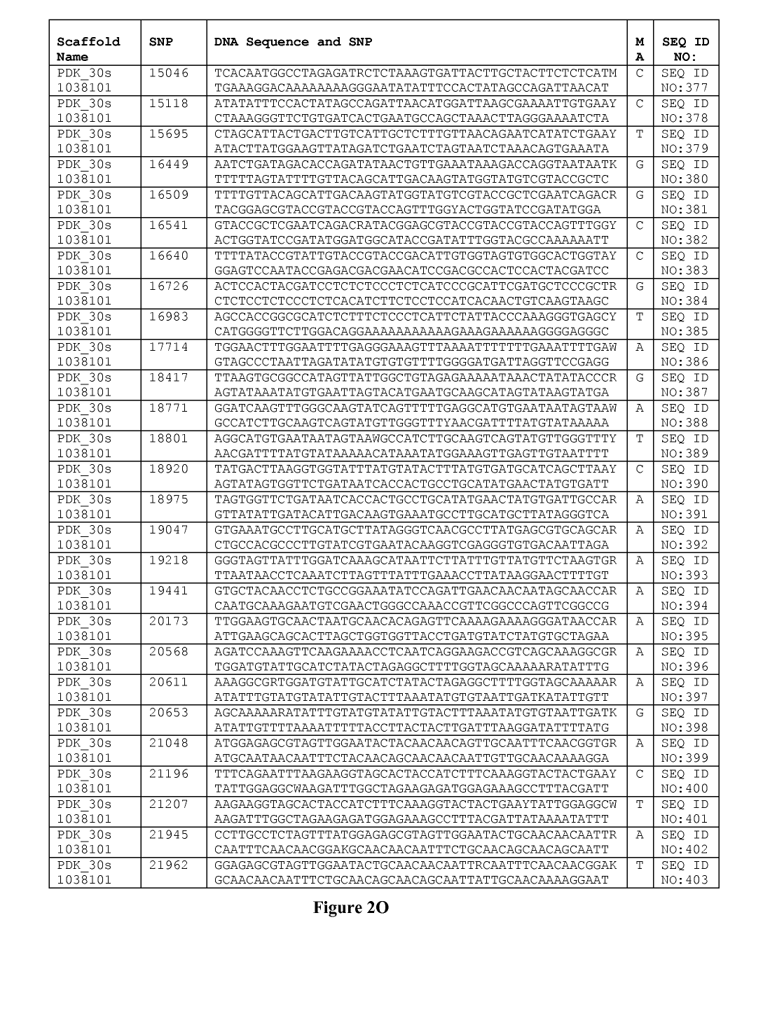

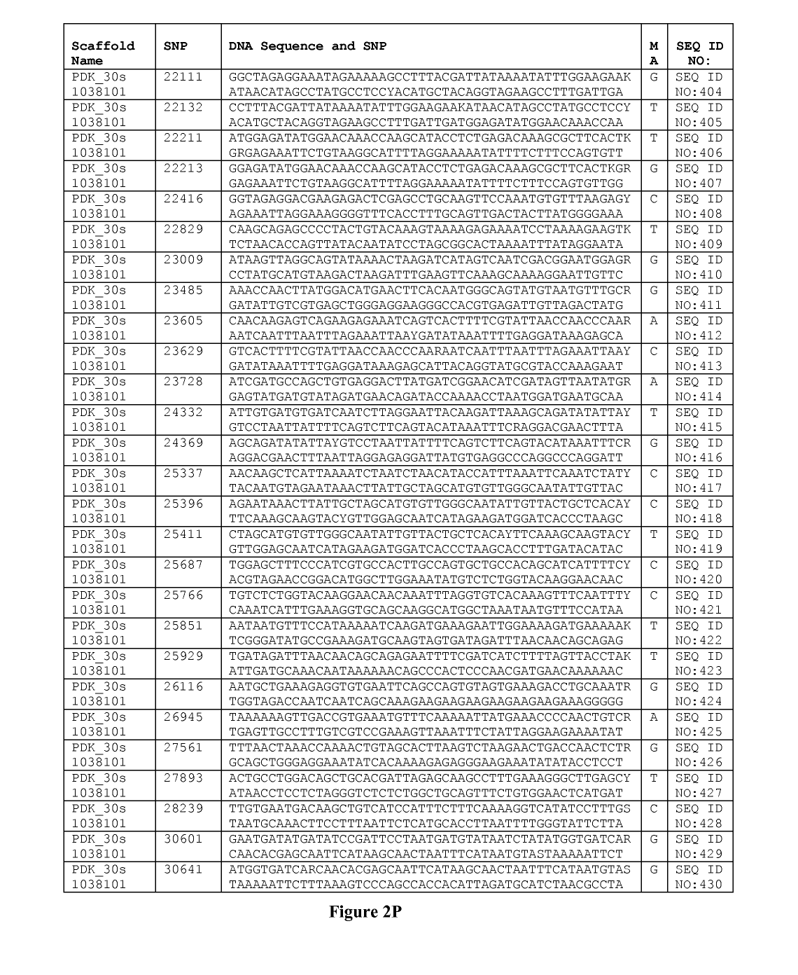

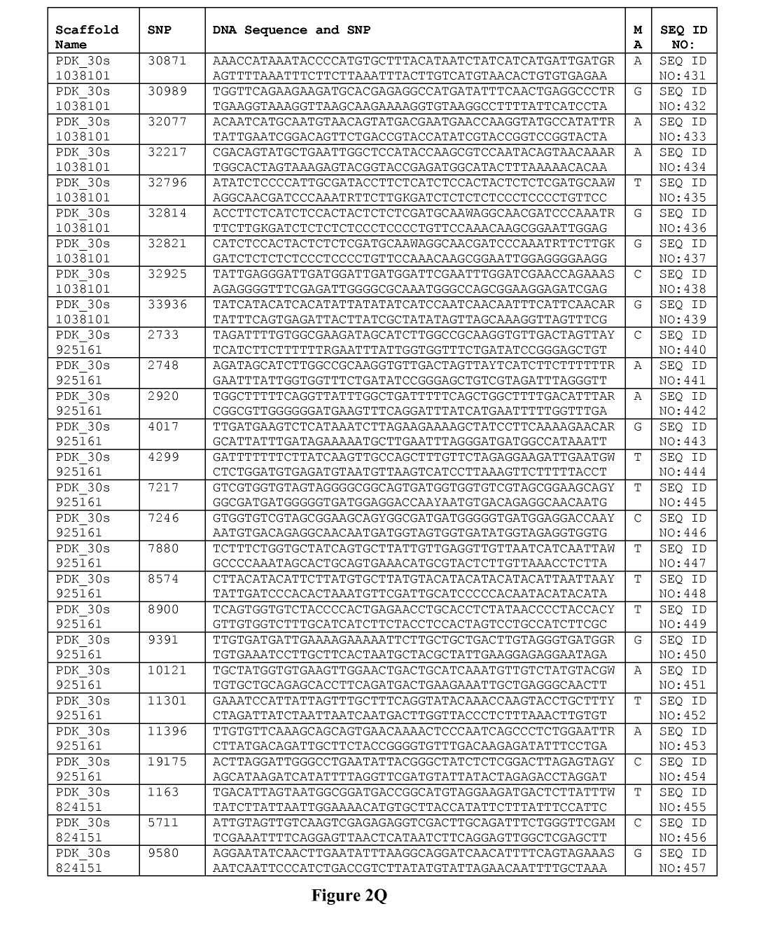

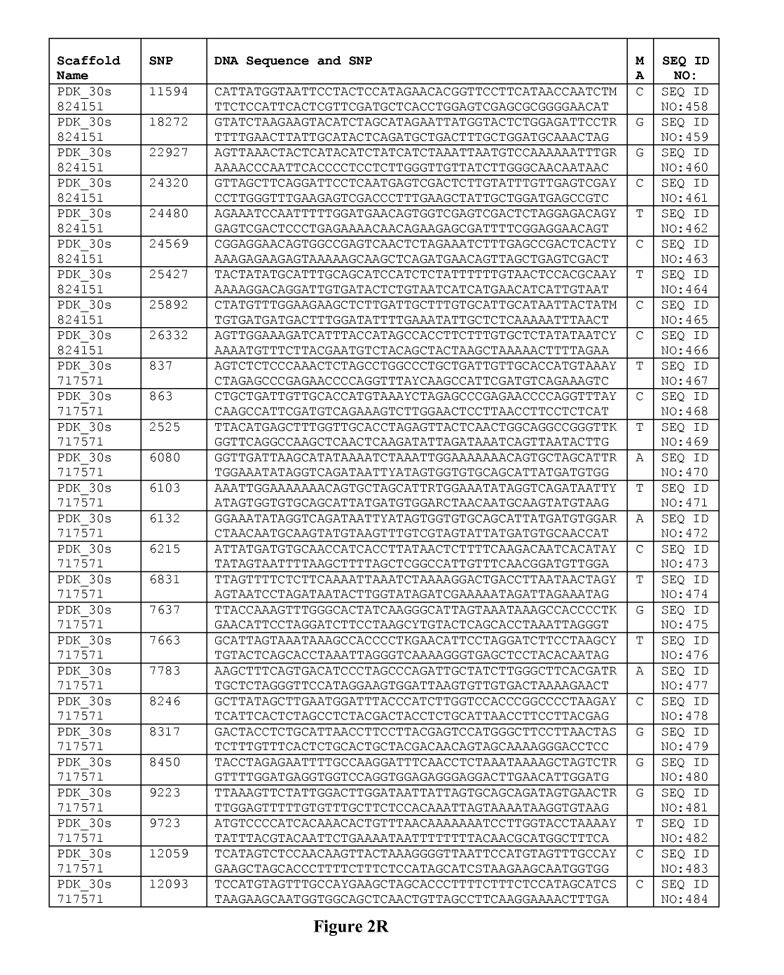

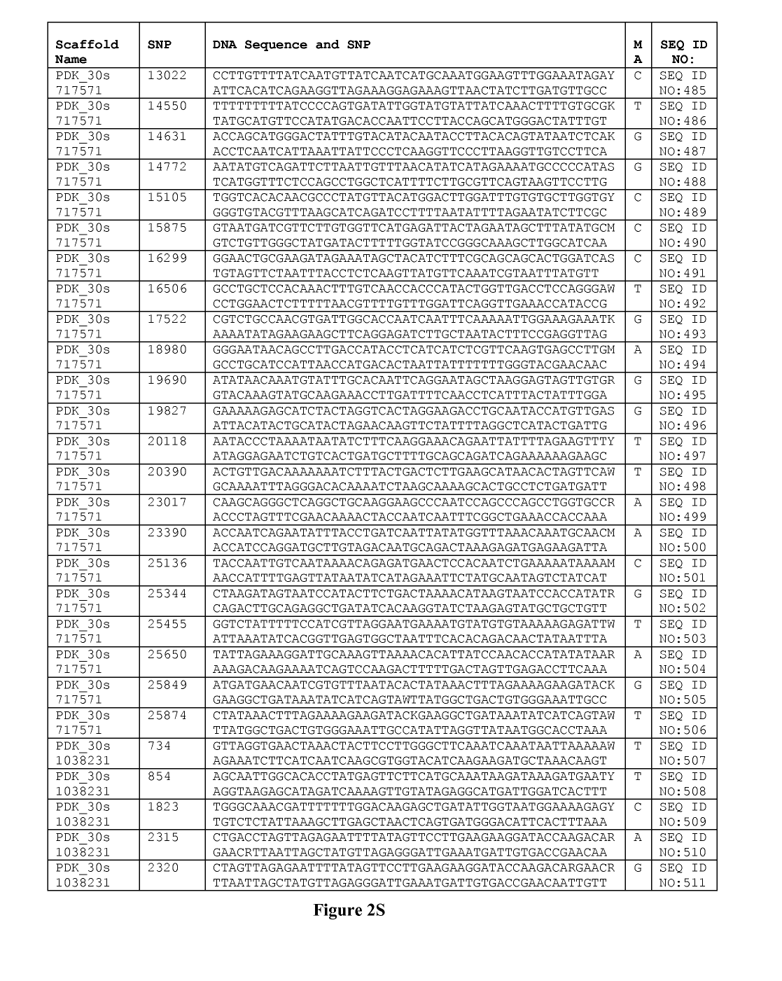

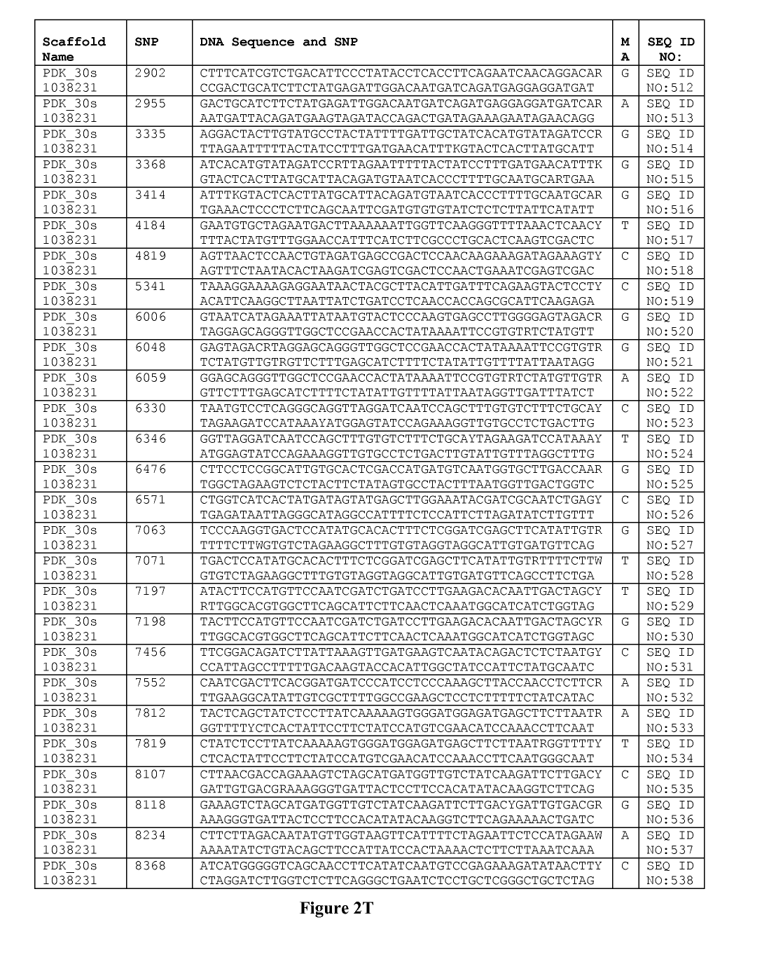

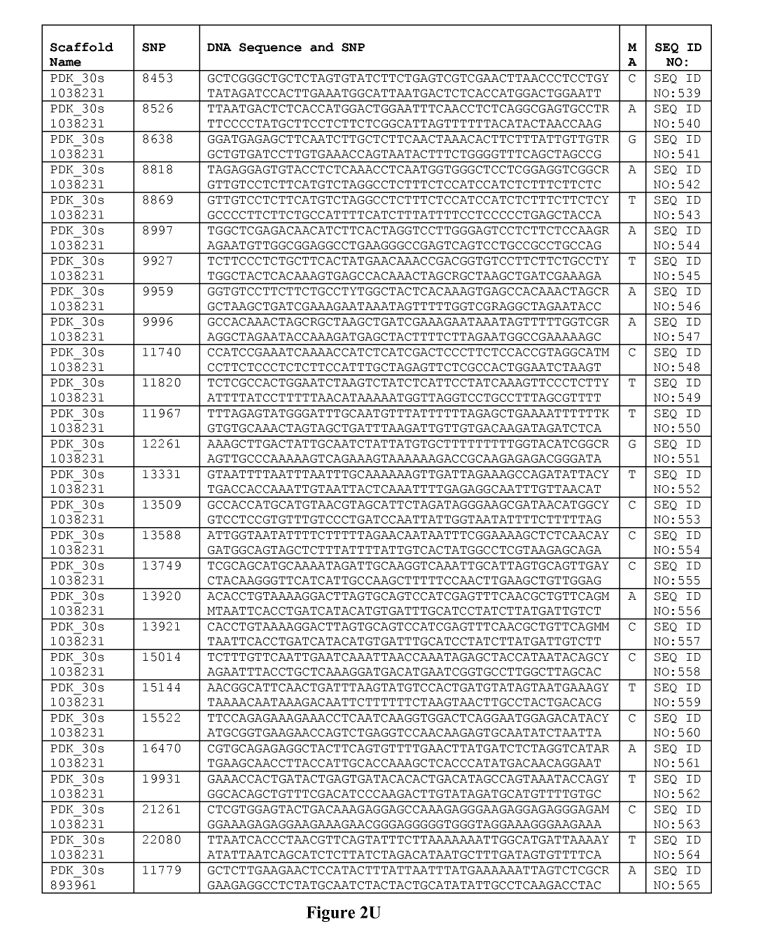

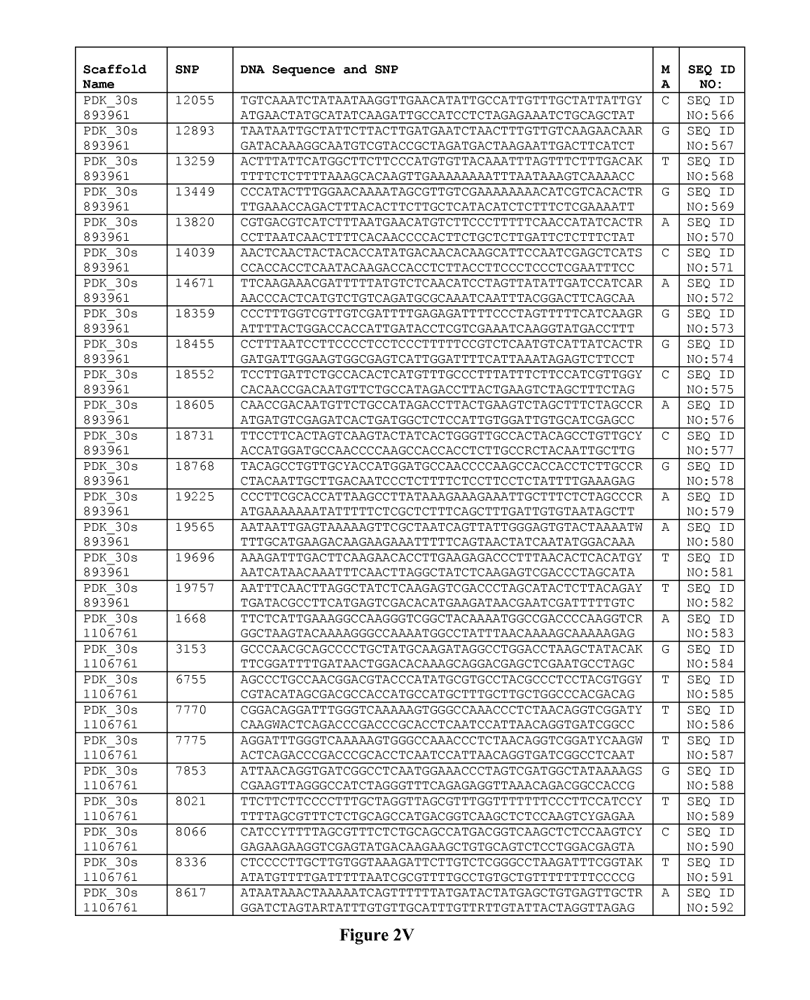

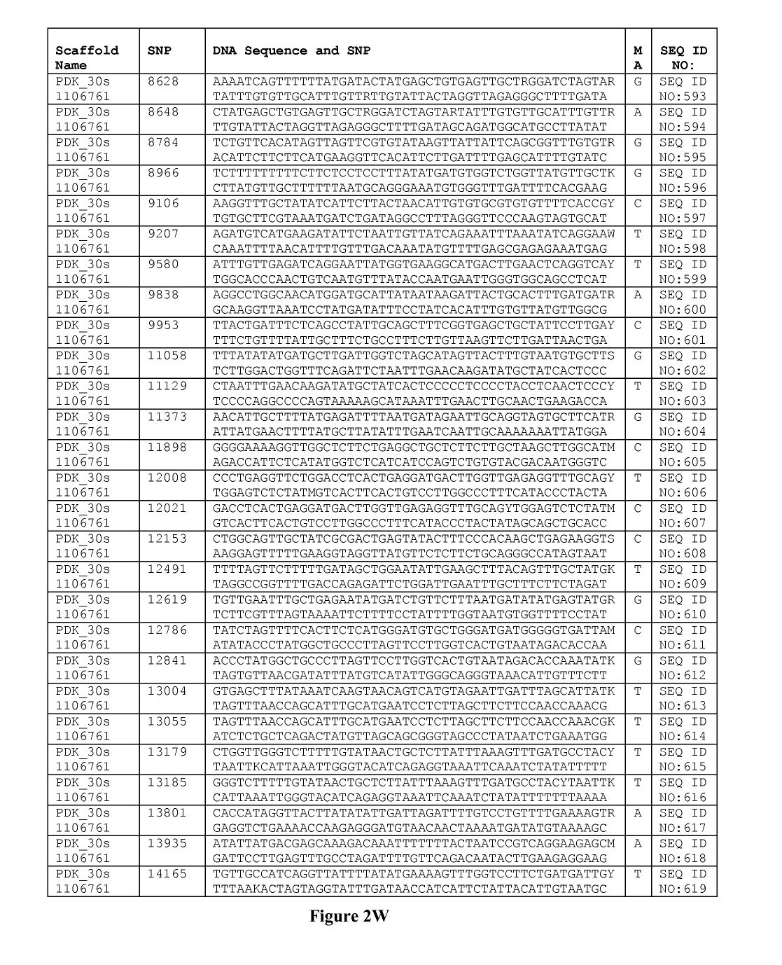

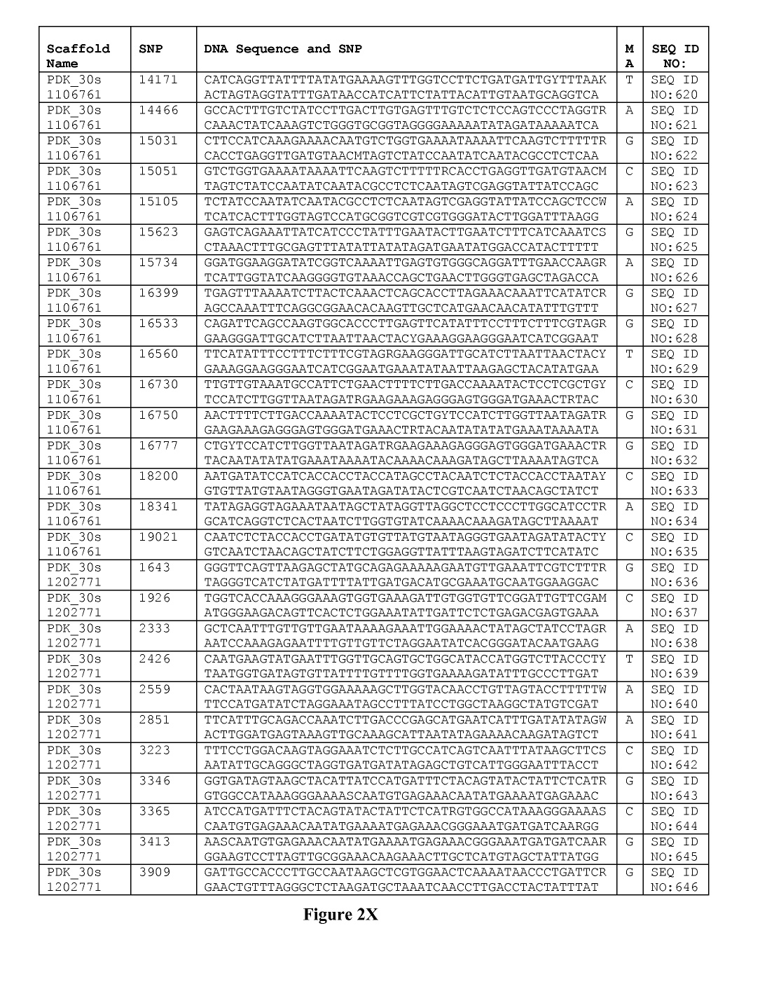

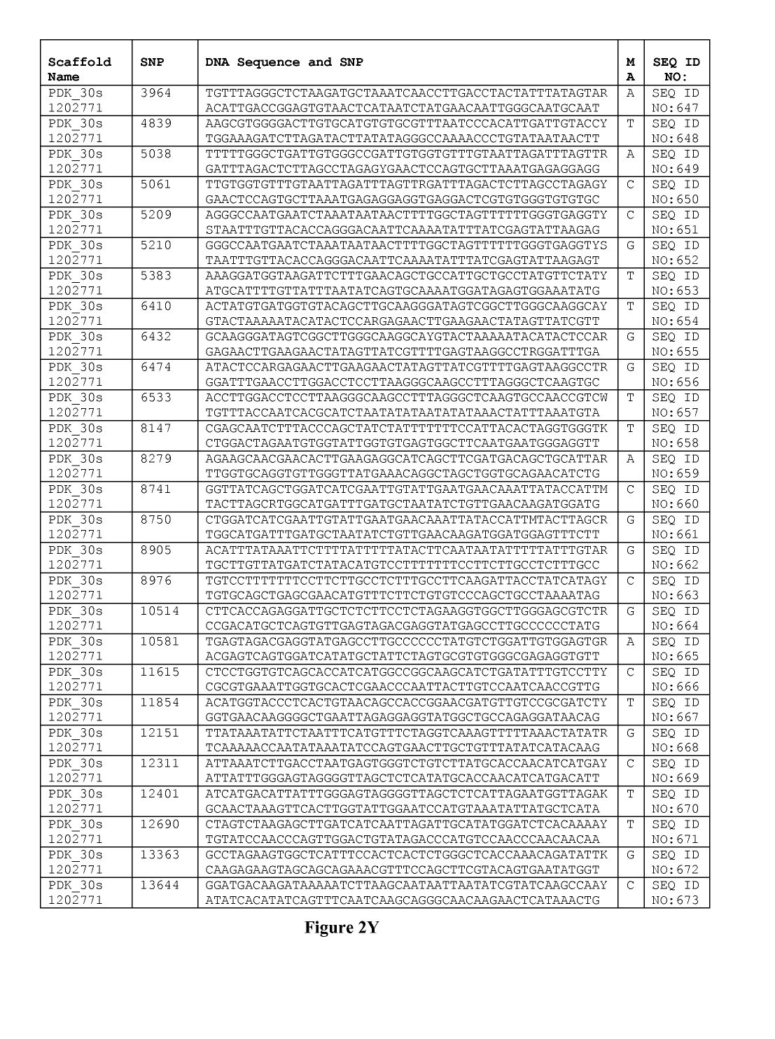

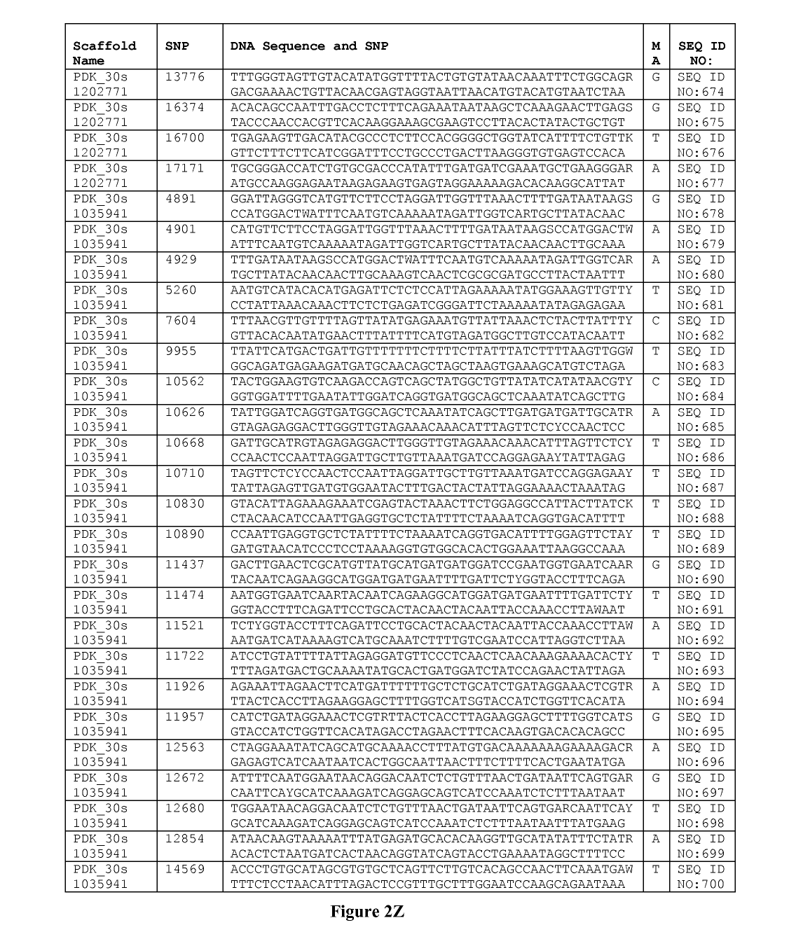

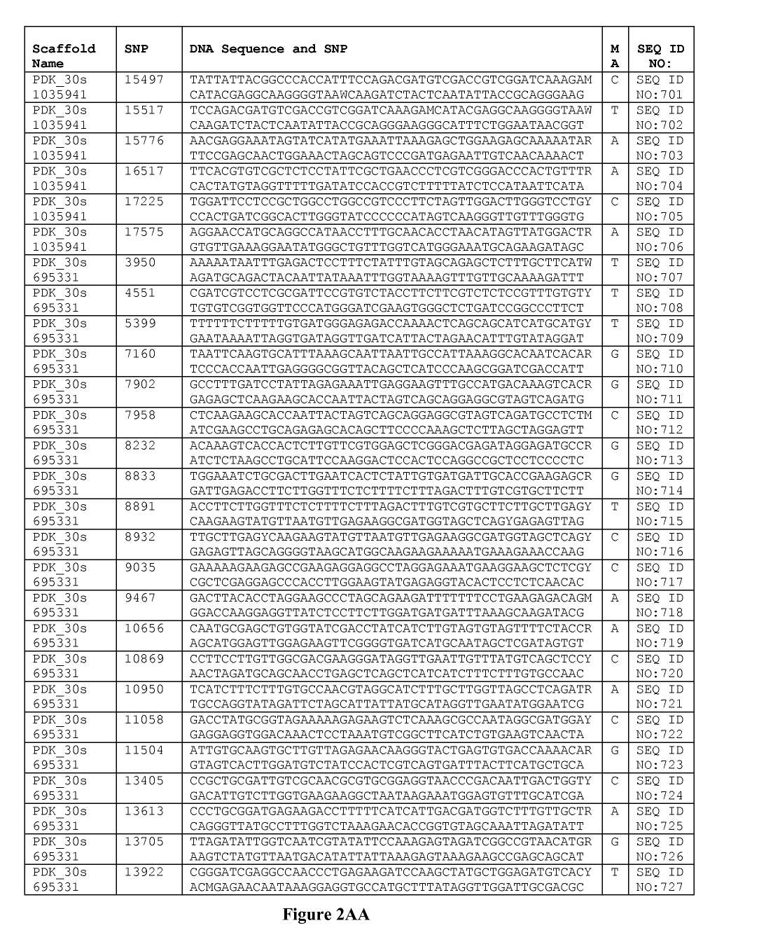

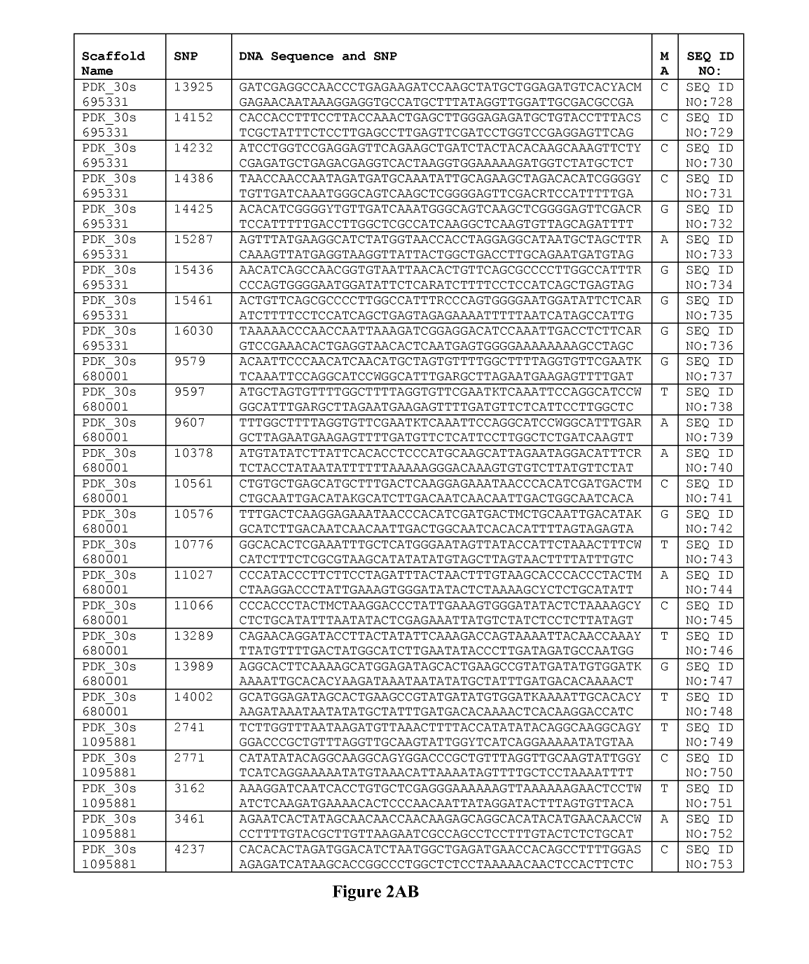

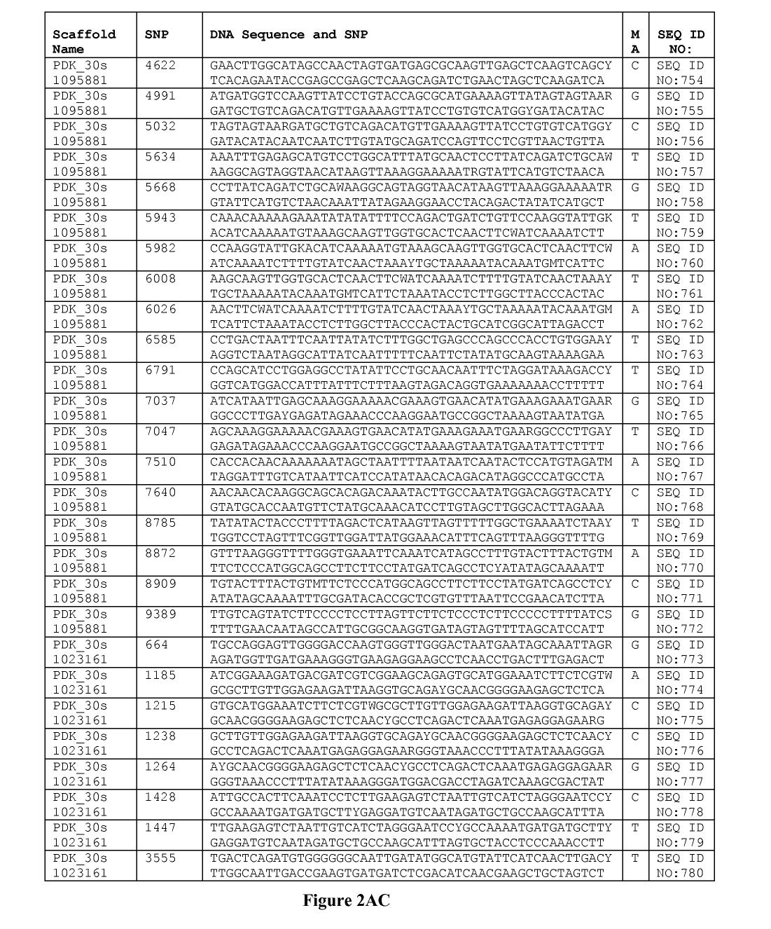

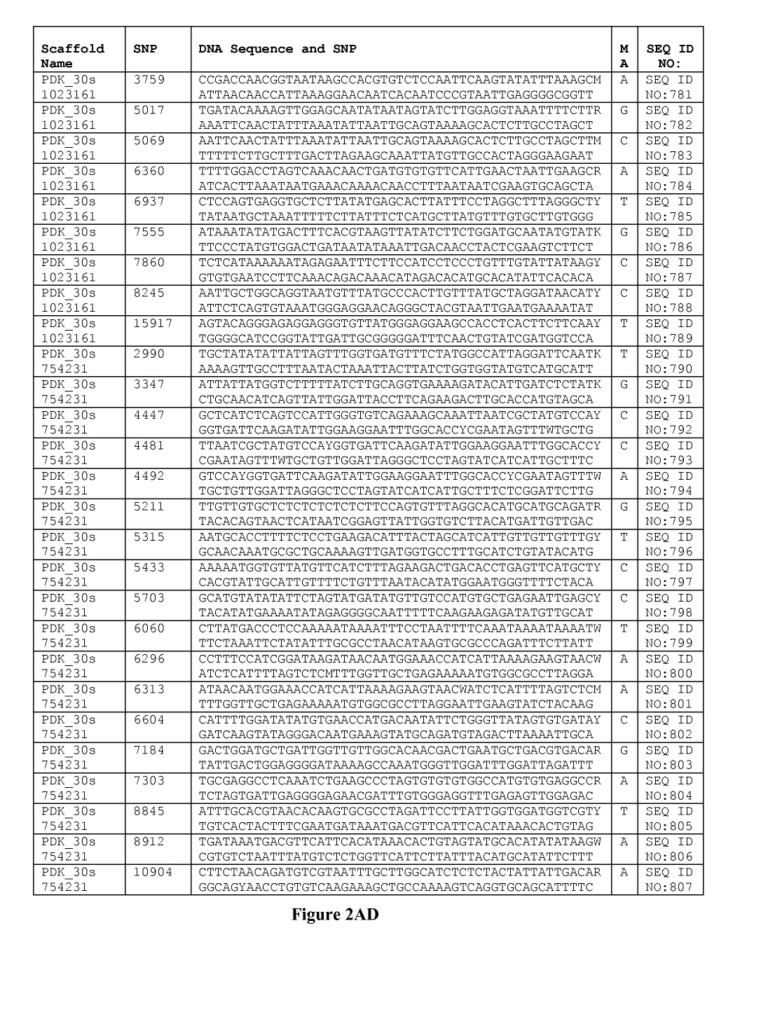

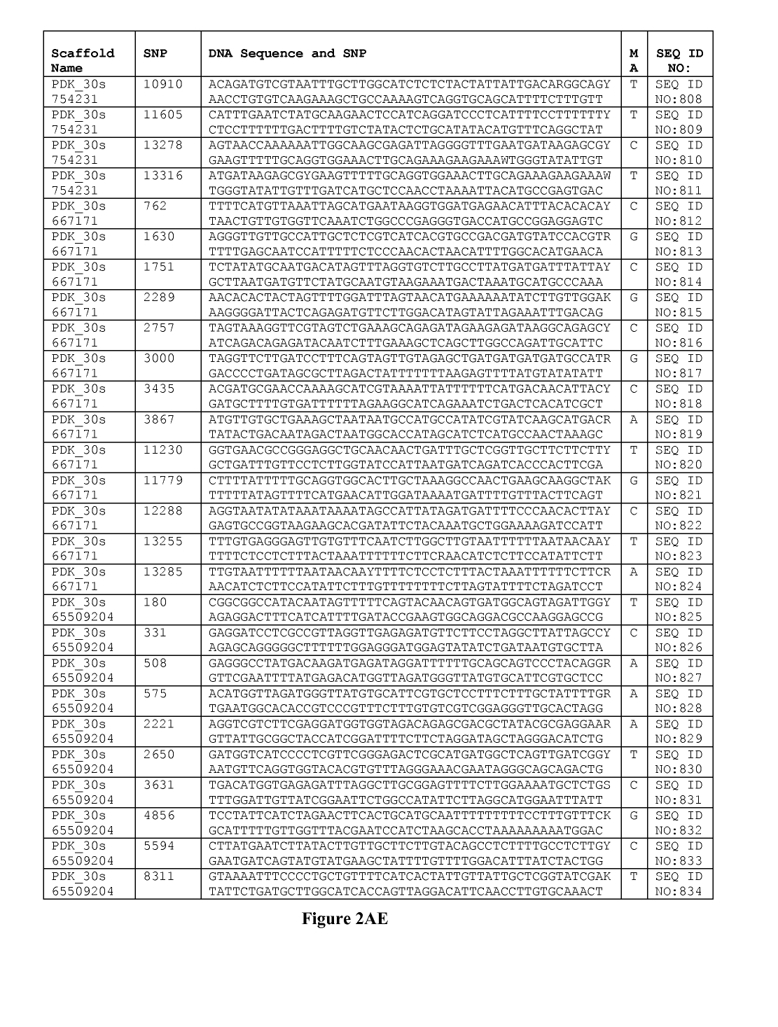

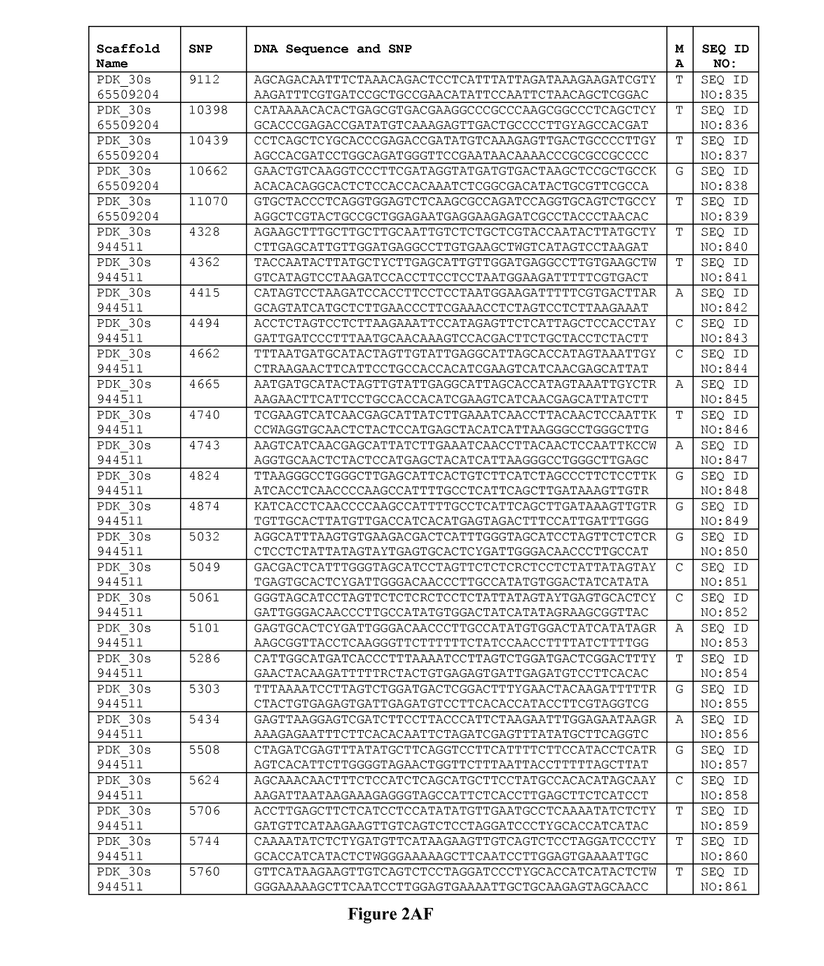

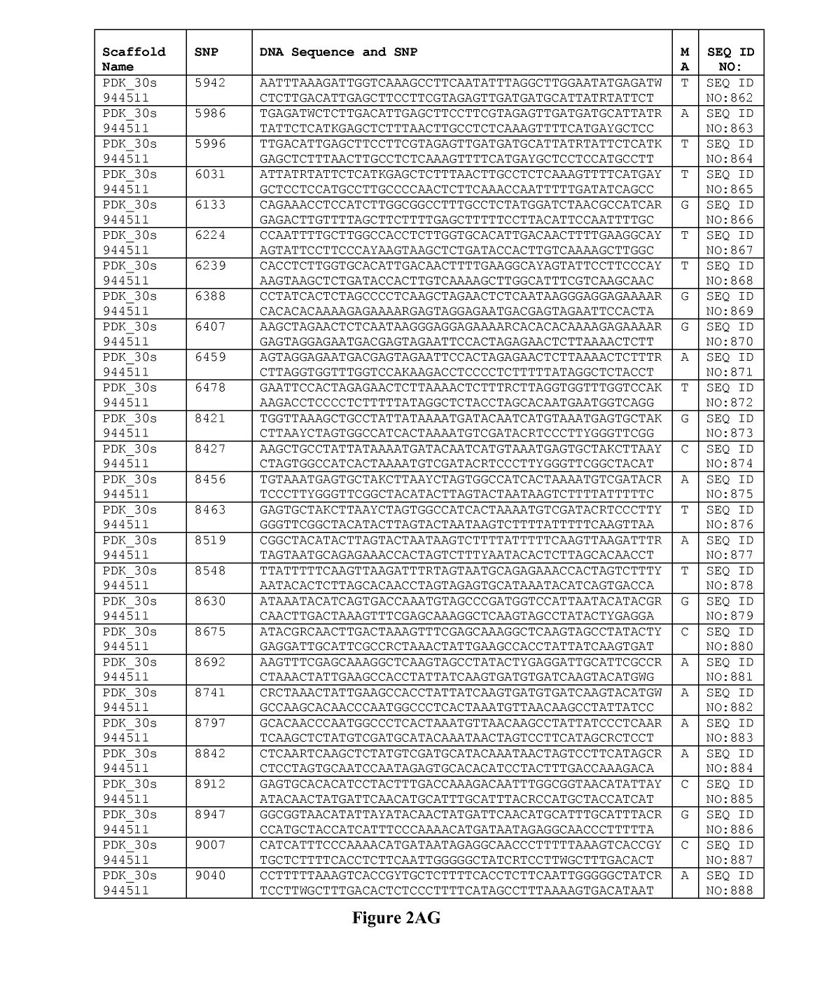

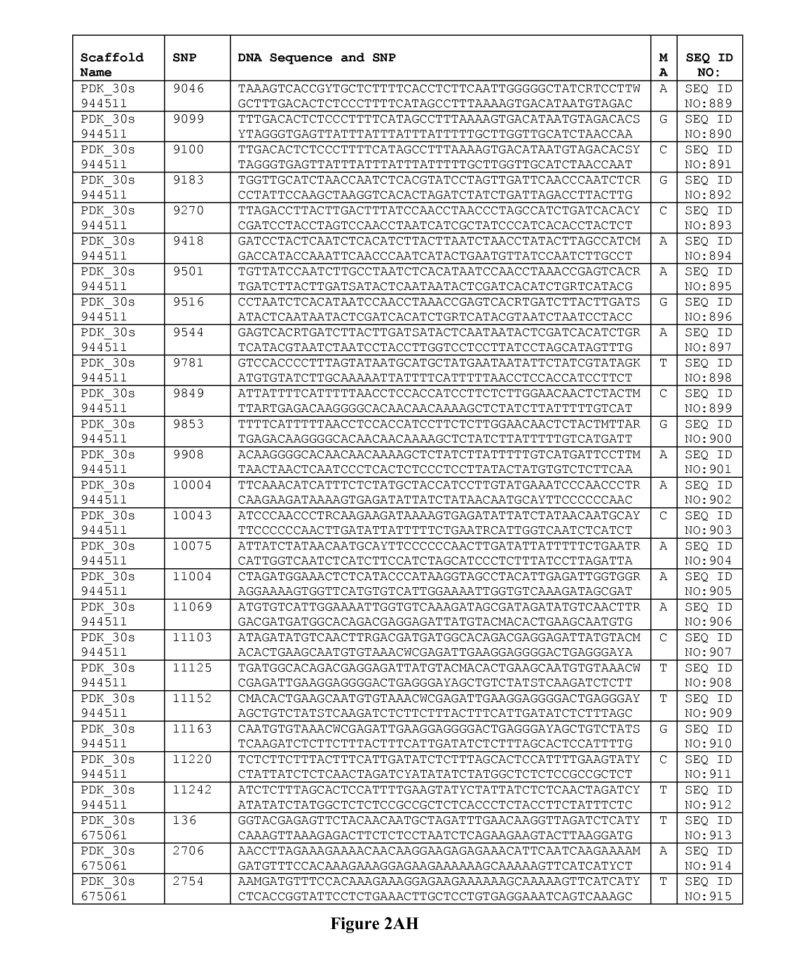

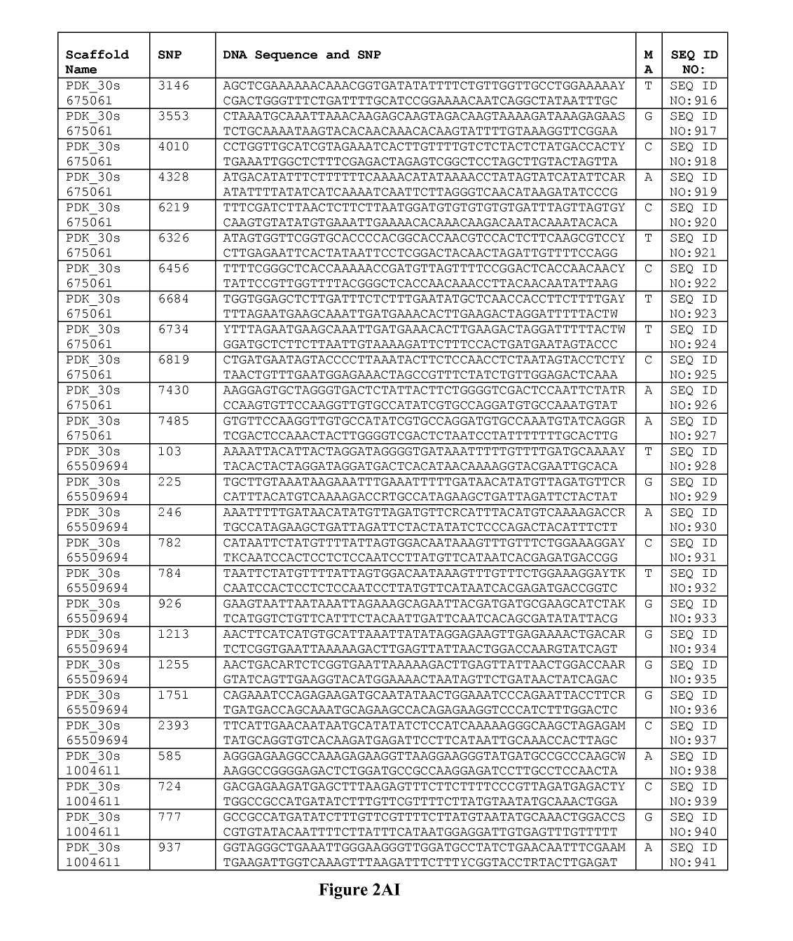

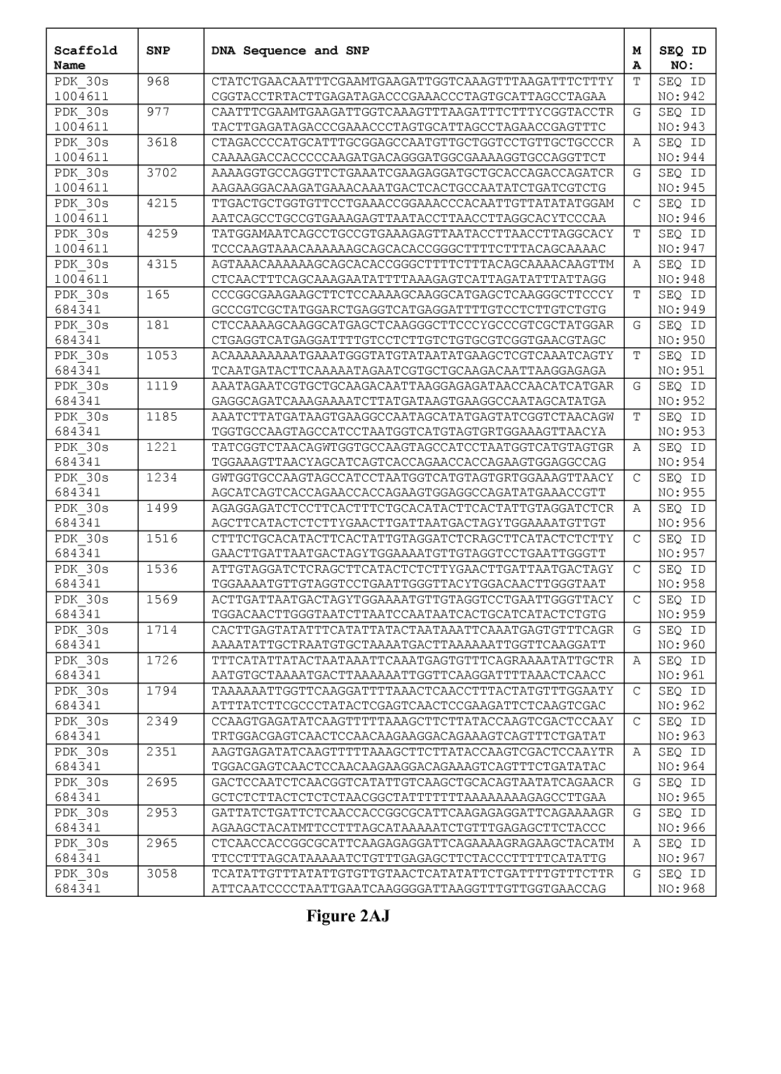

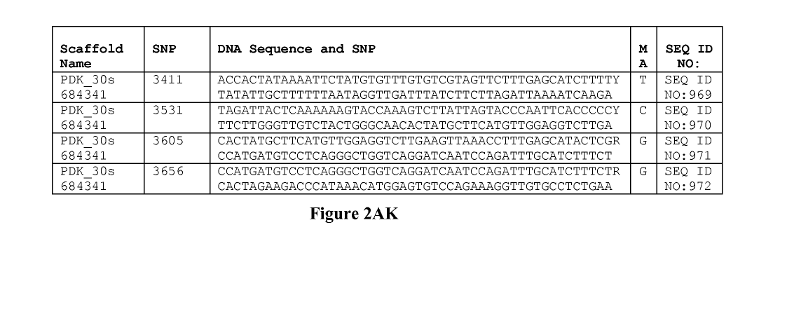

Pursuant to the methods of the present invention, nucleic acid sequences that identify the sex of a date palm plant include the nucleotide sequences of SEQ ID NOs:1-972 of FIGS. 2A-AK. Thus, in one embodiment, analyzing is carried out to determine the presence of a male allele at the nucleotide corresponding to position 51 of any one of SEQ ID NOs:1-972, as set forth in FIGS. 2A-AK. In an alternative embodiment, the plant, tissue, germplasm, or seed does not contain the male allele of SEQ ID NOs:1-972, as set forth in FIGS. 2A-AK, and the plant, tissue, germplasm, or seed is identified as a female plant.

In an alternative embodiment, DNA or RNA from a date palm plant, tissue, germplasm, or seed is analyzed for the presence of a molecular marker in linkage disequilibrium with the nucleic acid sequence that identifies the sex of the date palm plant. According to this embodiment, the molecular marker is present in SEQ ID NOs:1-972, as set forth in FIGS. 2A-AK, or a corresponding RNA molecule. Molecular markers now known, or to be discovered, that are in linkage disequilibrium with a nucleic acid molecule that identifies the sex of a date palm plant are contemplated by the present invention.

As used herein, a marker is a nucleotide sequence or encoded product thereof (e.g., a protein) used as a point of reference. For markers to be useful at detecting recombinations, they need to detect differences, or polymorphisms, within the population being monitored. For molecular markers, this means differences at the DNA level due to polynucleotide sequence differences (e.g., SNP, SSR, RFLP, AFLP). As used herein, markers define a specific locus on the date palm genome. Each marker is therefore an indicator of a specific segment of DNA, having a unique nucleotide sequence.

When a trait is stated to be linked to a given marker it will be understood that the actual DNA segment whose sequence affects or indicates the trait generally co-segregates with the marker. More precise and definite localization of a trait may be obtained if markers are identified on both sides of the trait. By measuring the appearance of the marker(s) in progeny of crosses, the existence of the trait can be detected by relatively simple molecular tests without actually evaluating the trait itself, which can be difficult and time-consuming because the actual evaluation of the trait requires growing plants to a stage where the trait can be expressed.

The genomic variability of a marker can be of any origin, for example, insertions, deletions, duplications, repetitive elements, point mutations, recombination events, or the presence and sequence of transposable elements (“TE”). Molecular markers can be derived from genomic or expressed nucleic acids (e.g., ESTs) and can also refer to nucleic acids used as probes or primer pairs capable of amplifying sequence fragments via the use of PCR-based methods.

In the context of the present invention, DNA or RNA is analyzed for the presence of a molecular marker in linkage disequilibrium with a nucleic acid sequence that identifies the sex of the plant. By “linkage disequilibrium,” it is meant that the nucleic acid and the trait are found together in progeny plants more often than if the nucleic acid and phenotype segregated separately.

Recombination frequency measures the extent to which a molecular marker is linked with a particular allele. Lower recombination frequencies, typically measured in centiMorgans (“cM”), indicate greater linkage between the allele and the molecular marker. The extent to which two features are linked is often referred to as the genetic distance. The genetic distance is also typically related to the physical distance between the marker and the allele. However, certain biological phenomenon (including recombinational “hot spots”) can affect the relationship between physical distance and genetic distance. Generally, the usefulness of a molecular marker is determined by the genetic and physical distance between the marker and the selectable trait of interest. The linkage relationship between a molecular marker and a phenotype is given as a “probability” or “adjusted probability.” Linkage can be expressed as a desired limit or range. For example, in some embodiments, any marker is linked (genetically and physically) to any other marker when the markers are separated by less than 50, 40, 30, 25, 20, or 15 map units (or cM). In some aspects, it is advantageous to define a bracketed range of linkage, for example, between 10 and 20 cM, between 10 and 30 cM, or between 10 and 40 cM. The more closely a marker is linked to a second locus, the better an indicator for the second locus that marker becomes. Thus, “closely linked loci” such as a marker locus and a second locus display an inter-locus recombination frequency of 10% or less, preferably about 9% or less, still more preferably about 8% or less, yet more preferably about 7% or less, still more preferably about 6% or less, yet more preferably about 5% or less, still more preferably about 4% or less, yet more preferably about 3% or less, and still more preferably about 2% or less. In highly preferred embodiments, the relevant loci display a recombination frequency of about 1% or less, e.g., about 0.75% or less, more preferably about 0.5% or less, or yet more preferably about 0.25% or less. Two loci that are localized to the same chromosome, and at such a distance that recombination between the two loci occurs at a frequency of less than 10% (e.g., about 9%, 8%, 7%, 6%, 5%, 4%, 3%, 2%, 1%, 0.75%, 0.5%, 0.25%, or less) are also said to be “proximal to” each other. Since one cM is the distance between two markers that show a 1% recombination frequency, any marker is closely linked (genetically and physically) to any other marker that is in close proximity, e.g., at or less than 10 cM distant. Two closely linked markers on the same chromosome can be positioned 9, 8, 7, 6, 5, 4, 3, 2, 1, 0.75, 0.5 or 0.25 cM or less from each other.

Data provided herein set forth a “logarithm of odds (LOD) value” or “LOD score” (Risch, “Genetic Linkage: Interpreting LOD Scores,” Science 255:803-804 (1992), which is hereby incorporated by reference in its entirety). This is used in interval mapping to describe the degree of linkage between two marker loci. A LOD score of three 0 between two markers indicates that linkage is 1000 times more likely than no linkage, while a LOD score of two (2.0) indicates that linkage is 100 times more likely than no linkage. LOD scores greater than or equal to two (2.0) may be used to detect linkage.

In addition to the markers identified herein, other markers linked to the markers described herein can be used to predict the sex of a date palm plant and are therefore also useful in carrying out the methods of the present invention. This includes any marker within, e.g., 50 cM of the markers associated with sex identification in date palm at a p-level ≦0.01 in the association analysis. The closer a marker is to a gene controlling a trait of interest, the more effective and advantageous that marker is as an indicator for the desired trait. Closely linked loci display an inter-locus cross-over frequency of about 10% or less, preferably about 9% or less, still more preferably about 8% or less, yet more preferably about 7% or less, still more preferably about 6% or less, yet more preferably about 5% or less, still more preferably about 4% or less, yet more preferably about 3% or less, and still more preferably about 2% or less. In highly preferred embodiments, the relevant loci (e.g., a marker locus and a target locus) display a recombination frequency of about 1% or less, e.g., about 0.75% or less, more preferably about 0.5% or less, or yet more preferably about 0.25% or less. Thus, the loci are about 10 cM, 9 cM, 8 cM, 7 cM, 6 cM, 5 cM, 4 cM, 3 cM. 2 cM, 1 cM. 0.75 cM, 0.5 cM or 0.25 cM or less apart.

Methods of the present invention are carried out to determine the sex of a date palm plant in a population, group, variety, or other classification of date palms where sex determination by genetic analysis is not otherwise known. For example, the methods of the present invention may be carried out to determine the sex of a plant of the variety Khalas, Deglet Noor, or Medjool. Other varieties of date palm are known and cultivated and are suited to the methods of the present invention.

Having identified the sex of a date palm plant, tissue, seed, or germplasm, the plant, tissue, seed, or germplasm may then be planted or transplanted in a location suitable for the identified sex. For example, it may be desirable in a date palm orchard (also referred to as a “garden”) to maximize the number of fruit-bearing (i.e., female) plants. Thus, it may be desirable to have more female plants than male plants in a particular geographical location. The ideal number of male to female plants may depend on several factors, including the size of the orchard, the fecundity of the male or female plants, the climate, etc. In addition, male plants, which spread pollin to fertilize the female flowers of the female plants, may be planted at locations in the orchard most likely to result in an ideal amount of pollination. The present invention permits the identification of plants of a particular male or female sex, which then permits a grower to determine the number and location of plants of a particular sex type to be planted in a given location. This can all be accomplished several years before the plant has reached a maturity sufficient to determine sex type based on floral structure.

The methods of the present invention involve growing a fruit-bearing plant from a plant, tissue, germplasm, or seed identified as a female plant pursuant to the methods of the present invention. The fruit is then harvested from the fruit-bearing plant.

The methods of the present invention also involve breeding a plant, after the sex of the plant has been determined pursuant to the methods of the present invention.

The methods of the present invention also involve marking a plant, tissue, seed, or germplasm based on its identified sex. For example, it may be desirable to analyze DNA or RNA from a date palm seed to identify the sex of the date palm seed. Upon identifying the sex of the seed, it is marked or segregated according to its sex. According to this embodiment, a grower can then select a seed based on its sex and plant the seed at a desirable location.

Another aspect of the present invention relates to a method of identifying the sex of a date palm plant. This method involves analyzing DNA or RNA from a date palm plant, tissue, germplasm, or seed for the presence of (i) a genotype that identifies the sex of the plant, tissue, germplasm, or seed, or (ii) a molecular marker linked to the genotype and identifying the sex of the plant, tissue, germplasm, or seed based on whether or not the plant, tissue, germplasm, or seed contains the genotype or the molecular marker.

Genotypes of the present invention include three possible alleles (AA, AB, BB). Thus, in one embodiment of the present invention, the sex of a date palm plant can be determined by detecting a genotype at position 51 of any of SEQ ID NOs:1-972, as set forth in FIGS. 2A-AK. In particular, the homozygous female allele (A/A) is associated with a female plant. The heterozygous (A/B) and homozygous male allele (B/B) are associated with a male plant.

The present invention also relates to methods of selecting a male or female date palm plant prior to flowering. In one aspect, the method involves detecting in a date palm plant, tissue, germplasm, or seed (i) a genotype that identifies the plant, tissue, germplasm, or seed as male or female, or (ii) a molecular marker in linkage disequilibrium with the genotype and selecting the plant, tissue, germplasm, or seed possessing the genotype or the molecular marker. In another aspect, the method involves detecting in a date palm plant, tissue, germplasm, or seed (i) a nucleic acid sequence that identifies the plant, tissue, germplasm, or seed as male or female or (ii) a molecular marker in linkage disequilibrium with the nucleic acid sequence and selecting the plant, tissue, germplasm, or seed possessing the nucleic acid sequence or the molecular marker.

The materials and methods described supra can be used to carry out the aspects of the present invention set forth in the preceding paragraph.

The present invention also relates to kits for selecting a male or female date palm plant prior to flowering. In one aspect, the kit includes primers or probes for detecting in a date palm plant, tissue, germplasm, or seed (i) a genotype that identifies the plant, tissue, germplasm, or seed as female, or (ii) a molecular marker in linkage disequilibrium with the genotype and instructions for using the primers or probes for detecting the genotype or the molecular marker. In another aspect, the kit includes primers or probes for detecting in a date palm plant, tissue, germplasm, or seed (i) a nucleic acid sequence that identifies the plant, tissue, germplasm, or seed as male or female or (ii) a molecular marker in linkage disequilibrium with the nucleic acid sequence and instructions for using the primers or probes for detecting the nucleic acid sequence or the molecular marker.

The materials and methods described supra can be used to carry out the aspects of the present invention set forth in the preceding paragraph.

Kits of the present invention may contain reagents specific for the detection of mRNA or cDNA (e.g., oligonucleotide probes or primers). The kits of the present invention may contain all of the components necessary to perform a detection assay, including all controls, directions for performing assays, and any necessary software for analysis and presentation of results. In some embodiments, individual probes and reagents for detection of nucleic acid sequences that identify the sex of a date palm plant or are provided as analyte specific reagents are included in the kit. In other embodiments, the kits are provided as in vitro diagnostics.

The present invention also relates to methods of breeding a date palm plant. In one aspect, the method involves providing a date palm plant having a sex determined by detecting in the plant or a seed, tissue, or germplasm from which it was derived (i) a genotype that identifies the plant as either male or female, or (ii) a molecular marker in linkage disequilibrium with the genotype and breeding the date palm plant with a plant of the opposite sex. In another aspect, the method involves providing a date palm plant having a sex determined by detecting in the plant or a seed, tissue, or germplasm from which it was derived (i) a nucleic acid sequence that identifies the plant as male or female or (ii) a molecular marker in linkage disequilibrium with the nucleic acid sequence and breeding the date palm plant with a plant of the opposite sex.

The materials and methods described supra can be used to carry out the aspects of the present invention set forth in the preceding paragraph.

Yet a further aspect of the present invention relates to a method of planting a date palm seed of a known sex. This method involves providing a seed having a known male or female sex and planting the seed.

The materials and methods described supra can be used to carry out the aspect of the present invention set forth in the preceding paragraph.

### EXAMPLES

The following examples are provided to illustrate embodiments of the present invention but are by no means intended to limit its scope.

### Example 1

### Materials and Methods

Date palm genomic DNA was extracted from leaves obtained from farmed trees in the Doha, Qatar area and at the USDA collection in Riverside, Calif. The Khalas female had been grown from well-documented plant tissue culture. The Alrijal female and Khalt male were seed grown but otherwise of unknown descent. Genomic libraries of various sizes were constructed. Paired-end sequencing on the Illumina Genome Analyzer II (Illumina, San Diego, Calif.) was carried out according to the manufacturer's protocols. The genome was assembled and scaffolded using SOAPdenovo v1.4 (Li et al., “De novo Assembly of Human Genomes with Massively Parallel Short Read Sequencing,” Genome Research 20:265-72 (2010), which is hereby incorporated by reference in its entirety) with a kmer of 31. Scaffolding using type III restriction libraries was conducted in BAMBUS (Pop et al., “Hierarchical Scaffolding with Bambus,” Genome Research 14:149-159 (2004), which is hereby incorporated by reference in its entirety) using 60 N's to designate a scaffold gap. Functional annotation was carried out using a local implementation of the BLAST2GO (Conesa et al “Blast2GO: A Comprehensive Suite for Functional Analysis in Plant Genomics,” Int. J. Plant Genomics Pub. ID No. 619832 (2008), which is hereby incorporated by reference in its entirety) software. All predicted genes were searched using BLASTP (e-value cutoff of 10⁻⁵) against the NR database at NCBI (http://www.ncbi.nlm.nih.gov, which is hereby incorporated by reference in its entirety) and also searched using the INTERPRO database at EBI. Functional assignments, Gene Ontology, and Enzyme Commission numbers were assigned whenever possible. For polymorphism calling, sequences where matched to the genome using BWA and SNPs called using the SAMTOOLS package (Li et al., “The Sequence Alignment/Map Format and SAMtools,” Bioinformatics 25:2078-9 (2009), which is hereby incorporated by reference in its entirety) with default parameters and requiring a minimum of 5 and no more than 70 sequences to call a SNP. CNVs were detected using CNV-SEQ (Xie et al., “CNV-seq, a New Method to Detect Copy Number Variation Using High-throughput Sequencing,” BMC Bioinformiatics 10:80 (2009), which is hereby incorporated by reference in its entirety).

Validation Sequencing

Approximately 4900 bp fragments of Khalas DNA were cloned into a pBR322-based low-copy-number vector using Khalas female DNA randomly sheared by nebulization. Three clones were selected for repeated paired-end sequence analysis. Extracted DNA was sequenced on a 3730XL DNA Analyzer (Applied Biosystems, Foster City, Calif.) using the manufacturer's recommended protocol. Multiple sequences from the same clones were assembled and aligned to the date palm reference sequence using the STADEN Package (Staden, “The Staden Sequence Analysis Package,” Mol. Biotechnol. 5:233-241 (1996), which is hereby incorporated by reference in its entirety).

TE-Related Genes Annotation

Protein and intron sequences of the 19,414 annotated genes were compared with the database of known TE proteins and significant matches were verified manually by further comparing them with NCBI NR database (http://www.ncbi.nlm.nih.gov, which is hereby incorporated by reference in its entirety).

Inference of Backcross Genotypes

When a pedigree includes a heterozygote male (e.g. A/G) progeny from a backcross with a homozygous recurrent parent female (e.g. A/A), the male parent must have been the donor of the ‘G’ allele. In the case of the pedigree used here, many donor parents are progeny of backcrosses themselves, Any progeny of a cross between a homozygous (A/A) parent and a heterozygous or homozygous B parent (A/G or G/G) w result in all progeny being either A/A or A/G. Because the ‘G’ allele was maintained through multiple backcross generations, all donor parents that are themselves progeny of a cross to the recurrent parent must have been the A/G genotype. One can therefore infer all donor parents (males) to have been heterozygous (A/G) up to the F1 generation.

Quantitative PCR

qPCR primers were designed on the Khalas genome in 5 regions: 3 male-amplified regions and 2 male-deleted regions. Amplifications were preferred as these are less likely to produce false positive results caused by polymorphism within the PCR primers. QuantiFast SYBR Green PCR mix (QIAGEN) was used in a 20 reaction. Samples were run on the Applied Biosystems 7500 real-time PCR machine a minimum of 4 times to produce an average. Delta-delta Ct was calculated against results from the Khalas genome using a region shown to be unamplified in all genomes as a baseline. A second region with no ISCRs called was used as negative control.

Genotyping Gender-Linked Regions

Regions with suspected linkage to gender based on genome polymorphism data were selected for further genotyping using PCR and sequencing. Primers were designed to create ˜400 bp PCR products. Regions were amplified with AmpliTaq Gold (Applied Biosystems, Foster City, Calif.) according to the manufacturer's protocol. PCR and cycle sequenced products were cleaned with Ampure XL and CleanSeq (Beckman Coulter, Beverly, Mass.). Cycle sequencing was conducted with BigDye v3.1 (Applied Biosystems, Foster City, Calif.). Samples were loaded on a 3130XL DNA Analyzer and sequence traces were visually inspected at all genotyped locations to determine homozygous or heterozygous changes.

### Example 2

### Genomic Libraries and Sequencing

DNA was extracted from the fresh leaves of date palm trees using the Wizard Genomic DNA preparation kit (Promega, Madison, Wis.). Leaves used for preparation of DNA employed in generating the Deglet Noor fosmid library were derived from the seedling of a single germinated seed.

Library construction for the short-paired libraries was conducted according to the manufacturer's protocol (Illumina, San Diego, Calif.). Two paired libraries of average insert size 172 bp and 370 bp were utilized. Longer mate-pair libraries were constructed using a linker sequence-modified version of the Type restriction enzyme EcoP15I library method as used by McKernan et al., “Sequence and Structural Variation in a Human Genome Uncovered by Short-read, Massively Parallel Ligation Sequencing Using Two-base Encoding,” Genome Research 19:1527-41 (2009) (which is hereby incorporated by reference in its entirety), producing 25-27 bp from either end of a DNA molecule. Fosmid library construction in vector pCC1FOS (Epicentre, Madison, Wis.) was as previously described (Pontaroli et al., “Gene Content and Distribution in the Nuclear Genome of Fragaria vesca,” The Plant Genome 2:93-101 (2009), which is hereby incorporated by reference in its entirety).

### Example 3

### Annotation

A repeat masked version of the genome was utilized for gene prediction. Ten million random short reads were assembled to create an initial repetitive region database to screen against the sequence data using REPEATMASKER. Previously trained monocot gene prediction parameters were used with the FGENESH++ pipeline, and the entire Plant section of REFSEQ was employed as input for homology searches. For the fosmid sequences, predicted Open Reading Frames (“ORFs”) were searched against the GenBank nonredundant nt and EST databases using BLASTN and against the nr database using BLASTX. A cutoff value of e⁻¹⁰ was used as the significance similarity threshold for the comparison.

### Example 4

### Transposable Element (TE) Identification

TE identification and quantification were by a series of complementary approaches. Small non-coding TEs such as MITEs were found by MITE-Hunter (Han et al., “MITE-Hunter: A Program for Discovering Miniature Inverted-repeat Transposable Elements from Genomic Sequences.” Nucleic Acids Research (2010).doi:10.1093/nar/gkq862, which is hereby incorporated by reference in its entirety) and RepeatModeler (http://www.repeatmasker.org/RepeatModeler.html). Protein-coding TEs were mainly identified by homology to TE-encoded proteins using BLASTX and required Expect value of 10⁻⁵ between predicted peptides. Intact LTR retrotransposons were found using LTR_FINDER (Xu et al., “LTR_FINDER: An Efficient Tool for the Prediction of Full-length LTR Retrotransposons.” Nucleic Acids Research 35:W265-8 (2007), which is hereby incorporated by reference in its entirety) and LTR_STRUC (McCarthy et al., “LTR_STRUC: A Novel Search and Identification Program for LTR Retrotransposons,” Bioinformatics 19:362-367 (2003), which is hereby incorporated by reference in its entirety). Once TEs were identified, their multiple copies were found by homology in the full genome assembly and in the shotgun reads.

### Example 5

### Polymorphism Detection

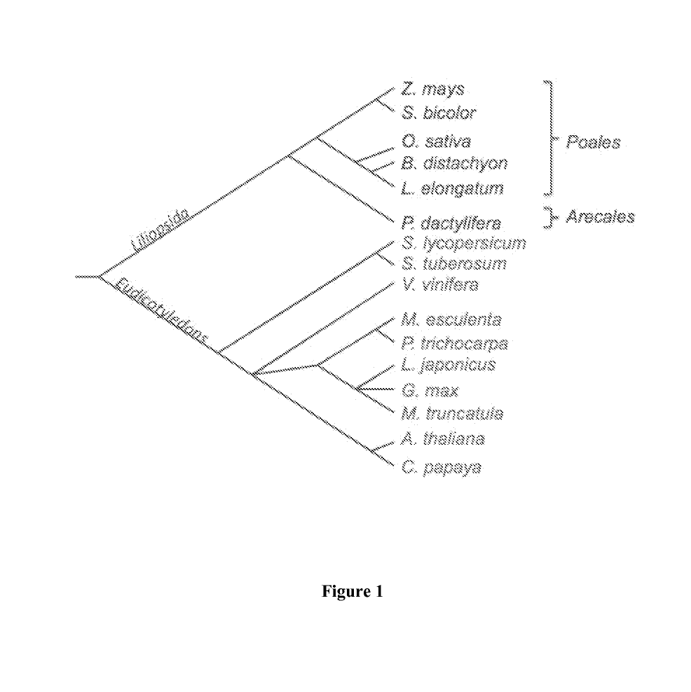

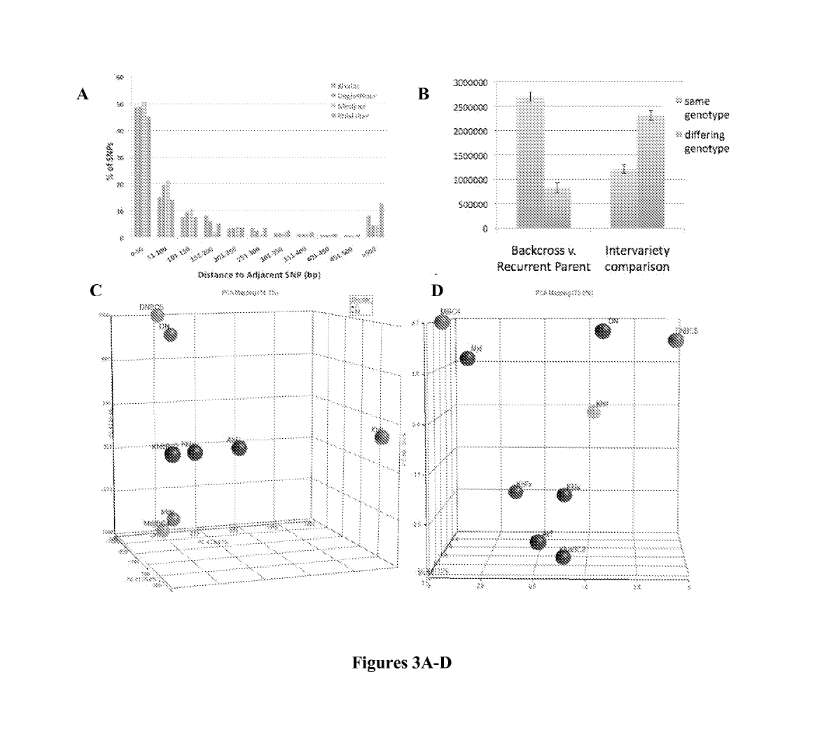

SNPs were called by matching the original shotgun sequences to the de novo assembly reference sequence and documenting regions where it was apparent that the reads represented two alleles (FIG. 1). For SNP calling in Khalas, only the longest paired-end sequences were used, resulting in 29.3× (of a total 53.4× used in assembly) coverage of 84 bp sequences. To avoid calling SNPs due to low quality sequence or collapsed repetitive sequence, a SNP was required to have at least 5-fold, and not more than 70-fold, coverage. For filtered SNP analysis in the measure of inter-SNP distance (FIG. 3A), 500 bp from either end of a contig and regions with greater than 38× or less than 20× (1.3× and 0.7× the mean sequence coverage) were removed.

Using CNV-SEQ, the window size for a detectable ISCR with an absolute log 2 value of 0.6 or greater ranged in size from 800 bp to 1000 bp, depending on depth of sequence coverage for the test genome. To be conservative, a universal window size of 1600 bp was set to call an ISCR. This was >1.5× larger than the window size required for statistically significant ISCR calling. At least 3 adjacent windows were required before annotating the region as an ISCR. Global normalization was used to take into account the lack of chromosome sized contigs.

ISCRs were annotated by documenting all locations of an ISCR in each sequenced genome. If the regions between any two genomes overlapped, this was collapsed and considered one ISCR region. All genomes were then documented for their level of sequence variation in these ISCR regions. Only those ISCRs that overlapped a coding region were documented.

Polymorphisms linked to gender were detected by scanning the genotypes of all genomes at the 3.5 million documented polymorphic sites. Scaffolds were identified that had more than 10 gender-segregating SNPs.

### Example 6

### Statistical Analysis

LOD scores were calculated as described (Lathrop et al., “Easy Calculations of LOD Scores and Genetic Risks on Small Computers,” American Journal of Human Genetics 36:460-5 (1984), which is hereby incorporated by reference in its entirety). Gene Ontology enrichment was calculated using the GOSSIP algorithm within the BLAST2GO package (Conesa et al., “Blast2GO: A Comprehensive Suite for Functional Analysis in Plant Genomics,” Int. J. Plant Genomics Art. ID No. 619832 (2008), which is hereby incorporated by reference in its entirety), which provides False Discovery Rates. Chi-Square Analysis was conducted using expected numbers of heterozygote SNPs based on the entire genome in a contingency table with heterozygous or homozygous as the two categories. Of all recorded genotyped positions in the genome, males were on average heterozygous in 25% while females were heterozygous in 36% of positions. In the suspected gender-linked scaffolds, all genotyped polymorphic sites in Deglet Noor and Medjool females and their backcrossed males were documented for homozygous or heterozygous changes and these used for the observed numbers.

Principal component analysis of the cultivar genotypes was carried out using the Partek Genomics Suite (Partek, St. Louis, Mo.). Genotypes were transformed to numeric genotypes with 1 representing homozygous matching the Khalas reference, 2 representing heterozygous, and 3 representing homozygous difference to the Khalas reference. The Decision Tree algorithm within the Willows package (Zhang et al., “Willows: A Memory Efficient Tree and Forest Construction Package,” BMC Bioinformatics 10:130 (2009), which is hereby incorporated by reference in its entirety) was utilized to find the best cultivar-discriminating SNPs. The top 1,000 m informative SNPs were selected based on a showing of all 3 possible alleles (AA, AB. BB) in the 9 sequenced genomes. From this set, the Decision Tree algorithm was used to select the fewest number of SNPs that could distinguish the 9 sequenced varieties. Though only 5 SNPs were enough to separate all 9 genomes, the backcrossed genomes did not always cluster with their recurrent parents accurately. SNPs with the most distinguishing power in the decision tree (32 SNPs) were chosen to provide a set from which a future subset can be selected once testing in a much larger and more diverse population is completed.

### Example 7

### Genome Sequencing and Assembly

The date palm genome contains 18 pairs of chromosomes (Siljak-Yakovlev et al. “Chromosomal Sex Determination and Heterochromatin Structure in Date Palm,” Sexual Plant Reproduction 9:127-132 (1996), which is hereby incorporated by reference in its entirety) and has been predicted to have a genome size of approximately 658 Mbp. A flow cytometric analysis of the date palm genome in cultivar Deglet Noor indicated a genome size of ˜680 Mbp (compared to a 382 Mbp rice genome standard). Moreover, comparison of the date palm draft genome to fully sequenced fosmids revealed that draft genome scaffolds spanned approximately 60% of the fosmids. This suggests that the draft genome of 381 Mbp is missing approximately 40% of the total genome; primarily in repetitive regions. This leads to a calculated genome size of ˜633 Mbp using this approach. Averaging the results of the two methods a genome size of approximately 658 Mb is predicted.

A de novo next-generation sequencing of the date palm genome was undertaken with the expectation that intragenic regions would have few large repeats, as is true in the similarly small genomes of rice (Yu et al., “A Draft Sequence of the Rice Genome (Oryza saliva L. ssp. indica),” Science 296:79-92 (2002), which is hereby incorporated by reference in its entirety) and sorghum (Ware et al., “Mehboob-ur-Rahman the Sorghum Bicolor Genome and the Diversification of Grasses,” Nature 457:551-556 (2009), which is hereby incorporated by reference in its entirety). If this is the case in date palm, then most genic regions should assemble uninterrupted by repeats, thus allowing a relatively unbiased view of the gene space. To this end, sequences ranging from 36 to 84 bp in length from fragments of ˜170 bp or ˜370 bp were generated on the Genome Analyzer IIx (Illumina, San Diego, Calif.). Assembly was conducted using the SOAPdenovo genome assembler (Li et al., “De Novo Assembly of Human Genomes with Massively Parallel Short Read Sequencing,” Genome Research 20:265-72 (2010), which is hereby incorporated by reference in its entirety) employed on other large genomes (Li et al., “The Sequence and De Novo Assembly of the Giant Panda Genome,” Nature 463:311-7 (2010), which is hereby incorporated by reference in its entirety) and which can utilize paired-end information for resolving repeats. Sequence reads were corrected prior to assembly using the SOAP Correction Tool and gaps were closed where possible with the SOAP GapCloser.

The assembly stage used 526,443,374 sequences as input and yielded an N50 contiguous sequence (contig), the contig size above which half of the genome assembly length is contained, of 6,441 bp and a scaffold N50 size of 9,339 bp when scaffolds less than 500 bp were excluded. SOAPdenovo scaffolds were further joined into larger scaffolds with 28.6× physical coverage from Type III restriction enzyme libraries (2,000-5,000 bp) (McKernan et al., “Sequence and Structural Variation in a Human Genome Uncovered by Short-read, Massively Parallel Ligation Sequencing Using Two-base Encoding,” Genome Research 19:1527-41 (2009), which is hereby incorporated by reference in its entirety) using the BAMBUS software (Pop et al., “Hierarchical Scaffolding with Rambus,” Genome Research 14:149-159 (2004), which is hereby incorporated by reference in its entirely) and requiring at least 3 longer mate-pair links to join contigs to scaffolds. This resulted in 57,277 scaffolds with an N50 size of 30,480 bp spanning 381 Mb of sequence. Post-assembly matching of sequences revealed sequence redundancy of 53.4× from reads of average length 64 bp. This coverage is greater than the theoretically determined minimum for a high quality assembly using reads of this length (Li et al., “De novo Assembly of Human Genomes with Massively Parallel Short Read Sequencing,” Genome Research 20:265-72 (2010), which is hereby incorporated by reference in its entirety). With a heterozygous genome, it is possible for the assembler to have split alleles and assemble them separately. This would result in contigs with half the sequence coverage of the genome average. However, distribution of coverage on the assembly showed no secondary peak at half the mean coverage (FIG. 10), indicating that assembly of separate haplotypes is most likely localized to short areas. With this short read strategy, contigs broken by short repeats are joined to a scaffold by paired end information. Large repetitive regions are expected to be intractable with this approach and are not included in the assembly.

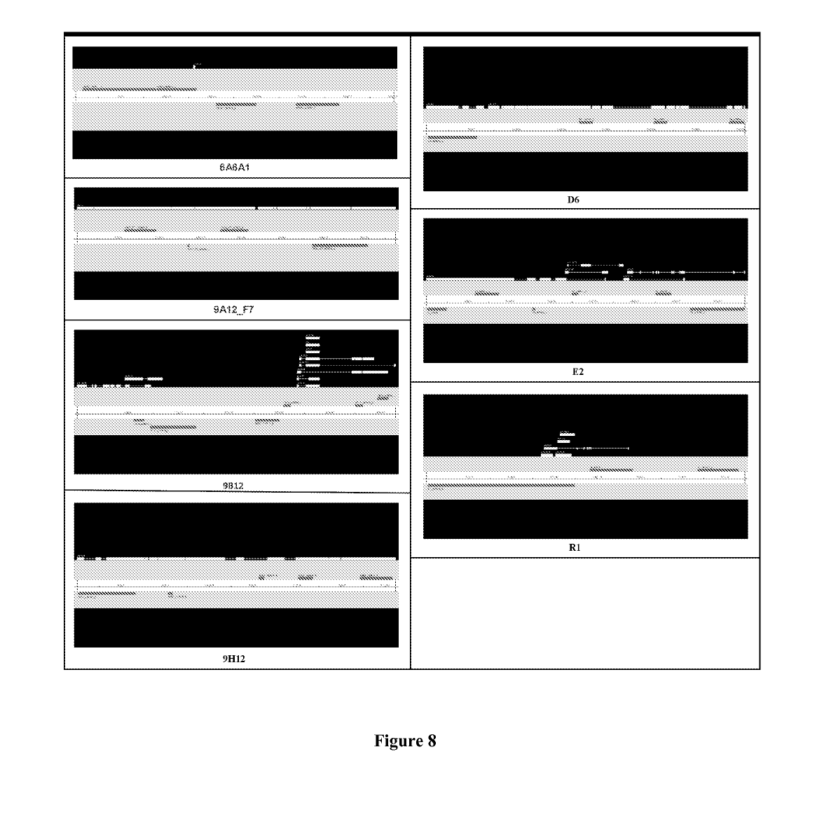

To investigate accuracy and completeness of the full genome assembly, comparisons were made to fully sequenced genomic DNA regions from both Khalas and Deglet Noor cultivars (Table 1; FIG. 8). Six fosmids containing Deglet Noor inserts were fully sequenced using Sanger technology. Scaffolds from the assembly aligned to 60% of the mainly gene-rich total fosmid sequence giving an indication of the completeness of the draft genome sequence. Analysis of the fosmid sequence not captured in the fall genome assembly revealed that the majority of these regions are highly repetitive (Table 2). This investigation indicates that gene-rich regions were better reconstructed than TE-rich regions, and genes were much better recovered than repeat sequences.

TABLE 1
Results of Comparison of the Assembly to 6 Fosmids.

| | | | | Perc. |
|---|---|---|---|---|
| Subject | Category | Number | Reconstructed | (%) |
| Fosmid | Only Contain TE Proteins | 1 | 0 | 0 |
| | Mix of TE and non-TE | 2 | 0 | 0 |
| | proteins | | | |
| | Only Contain non-TE | 3 | 3 | 100 |
| | proteins | | | |
| Gene in | TE gene | 10 | 4 | 40 |
| fosmid | Hypothetical gene | 5 | 4 | 80 |
| | Non-TE gene | 13 | 11 | 85 |

(1) Reconstructed means 80% of the subject matches one sub-region of a contig

TABLE 2
Comparison of Fully Sequenced Fosmid Bases Covered or Missed by the
WGS Assembly

| | Bases covered by | Bases missed by |
|---|---|---|
| | WGS scaffolds | WGS scaffolds |
| Median Coverage | 29X | 3219X |
| % bases >60X | 13.02 | 93.56 |
| % bases >90X | 10.95 | 92.35 |

It is clear that most missed bases in the WGS assembly are within very frequent repeats.

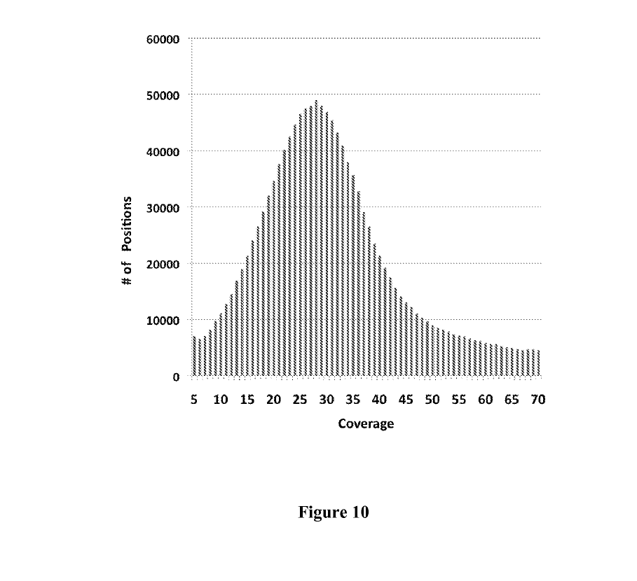

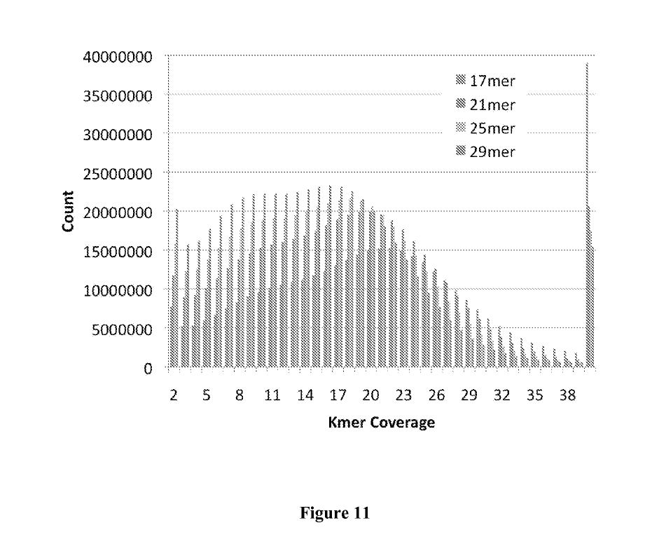

A test set of ˜210 million reads from the full set of ˜526 million (˜50% of high quality bases) was used for this analysis. All possible k-mers were documented in the reads using JELLYFISH (Marcais et al., “A Fast Lock-free Approach for Efficient Parallel Counting of Occurrences of K-mers,” Bioinfimatics (Oxford, England) btr 011 (2011), which is hereby incorporated by reference in its entirety) and plotted (FIG. 11). It is clear that as the k-mer length increases from 17 to 29, the bimodal distribution becomes more pronounced. The primary maximum represents coverage of homozygous regions of the genome and is approximately twice the coverage of the secondary maximum. The secondary maximum is likely the result of heterozygous regions where the two alleles result in different k-mers. It is important to note that, given this test set contains half the number of high quality bases, k-mer coverage of both alleles is likely high enough (18-20× Kmer coverage per allele) in the full read set to assemble both alleles independently.

Independent assembly of alleles seems to have occurred relatively infrequently as coverage of the genome by the same test set of reads has a single maximum (FIG. 10). If alleles had been assembled separately on a large scale, this would have been expected to be bimodal with split alleles obtaining half the level of coverage from the main maximum. This was not observed. Furthermore, the less stringent gap closing stage of the assembly likely fills gaps created by heterozygous regions.

It is possible that some regions of the genome are absent from the assembly because the two alleles were too different to join either at the graph stage or the more liberal gap filling stage. These would result in gaps rather than separately assembled alleles. From the fully sequenced fosmid data it is predicted that the regions of the genome absent due to high heterozygosity amount to less than 7% of the missing sequence. The remaining 93% of missing sequence is likely due to high frequency repeats that could not be correctly assembled. This is based on matching the test set of reads to the fully sequenced fosmids (which are homozygous). No restriction was placed on how frequently reads could match the fosmids. Coordinates of where scaffolds from the whole genome shotgun assembly (WGS) matched the fosmids were documented and sequence coverage from the test set of reads was inspected both within and outside these assembled regions (Table 2). Median coverage in regions where the WGS scaffolds matched the fosmids was similar to that obtained for the rest of the genome. In contrast, regions not captured by WGS scaffolds had extremely high coverage indicating they contain frequently repeated sequence (Table 2). ˜7% of the fosmids bases that were not matched by the WGS scaffolds had coverage consistent with the non-repetitive portion of the genome. These regions were short regions interspersed among the repeats. Their length was such that they would not have made the 500 bp cutoff required for a scaffold to be included in the assembly.

The genome assembly was further compared to 109,244 contigs of assembled date palm ESTs. Using BLAT (Kent, “BLAT—the BLAST-like Alignment Tool” Genome Research 12:656-64 (2002), which is hereby incorporated by reference in its entirety), 72% of EST contigs matched at least 90% of their length, while 86% of high quality EST bases could be aligned to the reference sequence with a minimum of 98% sequence identity. Furthermore, using the CEGMA pipeline (Parra et al., “Assessing the Gene Space in Draft Genomes,” Nucleic Acids Research 37:289-297 (2009), which is hereby incorporated by reference in its entirely), which checks for full-length models of core genes, 94% of core eukaryotic genes were found in the assembly, and 71% of these were recovered as full-length gene models. Taken together, the data suggest that ˜90% of date palm genes and ˜60% of the full date palm genome sequence are described in this assembly. The uncaptured regions of the genome are likely to be highly repetitive and thereby intractable to the assembly approach.

### Example 8

### Genome Annotation

Repeat masked scaffolds were passed to the FGENESH++ pipeline for both de novo and homology-based gene prediction (Solovyev et al., “Automatic Annotation of Eukaryotic Genes, Pseudogenes and Promoters,” Genome Biology 7 (Suppl 1):S10.1-12 (2006), which is hereby incorporated by reference in its entirely). A total of 28,890 gene models were predicted. Of these, 25,059 predicted protein-encoding genes had significant BLAST similarity to proteins from other organisms in the NR database at NCBI (http://www.ncbi.nlm.nih.gov. which is hereby incorporated by reference in its entirety). Gene ontology information was assigned using BLAST2GO (Conesa et al., “Blast2GO: A Comprehensive Suite for Functional Analysis in Plant Genomics,” Int. J. Plant Genomics Pub. ID No. 619832 (2008), which is hereby incorporated by reference in its entirety). GC content within coding DNA sequence was 47.6%, while the entire assembled genome has a GC content of 38.5%.

The top BLAST hits for 9.022 of the date palm predicted proteins matched predicted proteins from Vitis vinifera, a eudicot, followed by 5,094 top matches to predicted proteins from the monocot Oryza sativa. This higher protein sequence similarity between the two less phylogenetically-related plants (the monocot date palm and the eudicot grapevine) has been observed by others in gene families from oil palm (Adam et al., “MADS Box Genes in Oil Palm (Elaeis guineensis): Patterns in the Evolution of the SQUAMOSA, DEFICIENS, GLOBOSA, AGAMOUS, and SEPALLATA Subfamilies,” Journal of Molecular Evolution 62:15-31 (2006), which is hereby incorporated by reference in its entirety) and for oil palm ESTs (Jouannic et al. “Analysis of Expressed Sequence Tags from Oil Palm (Elaeis guineensis),” FEBS Letters 579:2709-14 (2005), which is hereby incorporated by reference in its entirely). Initial suggestions are that the grasses are a more diverged monocot group.

A total of 2,949 (10%) gene models with high homology to TE genes were found. Among them, the protein coding regions of 2,097 models matched TE proteins (BLASTP, E-value=10-5). The other 852 models matched predicted TE genes in their intron regions. These TEs within genes are likely to be low copy number in the genome, or they would not have been assembled. Overall, 55,855 sequences were identified in the full genome assembly that had the characteristics of TEs. Some of these, including a few long terminal repeat (“LTR”) retrotransposons (45 families) and the tiny TEs called MITEs (35 families) were found by structural criteria (Xu et al., “LTR_FINDER: An Efficient Tool for the Prediction of Full-length LTR Retrotransposons,” Nucleic Acids Research 35:W265-8 (2007); McCarthy et al., “LTR_STRUC: A Novel Search and Identification Program for LTR Retrotransposons,” Bioinformatics 19:362-367 (2003); Han et al., “MITE-Hunter: A Program for Discovering Miniature Inverted-repeat Transposable Elements from Genomic Sequences,” Nucleic Acids Research doi:10.1093/nar/gkq862 (2010), which are hereby incorporated by reference in their entirety).

One intact LTR retrotransposon of the Copia superfamily was found on sequenced fosmid R1 by both LTR_FINDER (Xu et al., “LTR_FINDER: An Efficient Tool for the Prediction of Full-length LTR Retrotransposons,” Nucleic Acids Res. 35:W265-8 (2007), which is hereby incorporated by reference in its entirety) and LTR_STRUC (McCarthy et al., “LTR_STRUC: A Novel Search and Identification Program for LTR Retrotransposons,” Bioinformatics 19:362-367 (2003), which is hereby incorporated by reference in its entirety). This new LTR retrotransposon was given the name “vose.” Table 3 below represents its characteristics, and its sequence is provided. This element constitutes 0.4% of the assembly and 2.3% of the 1× random reads set. No intact LTR elements were detected in the other 6 fosmids that were sequenced.

TABLE 3
MITE and LTR Families.

| | |
|---|---|
| Overall size (bp) | 10411 |
| Location in fosmid (bp range) | 21946-32356 |
| Length of 5′ LTR (bp) | 1693 |
| Length of 3′ LTR (bp) | 1692 |
| LTR pair homology | 99.8% |
| Ends dinucleotides | TG/CA |
| TSD | GGGGA/GGGGA |

These MITE and LTR families identified by structural search accounted for 2% and 4% of the assembly, respectively.

However, most MITE and TEs were found by homology to known TE proteins. The TEs found in the full genome assembly were compared with the raw genomic sequence data. As expected, because of the inability of short reads to resolve long repeats, many more TE-related sequences were identified in the raw shotgun data than in the assemblies (Table 4). The most abundant TEs identified in date palm, LTR retrotransposons of the Copia (˜3.1% of reads) and Gypsy (˜1.4% of reads) superfamilies, were found to be a respective 50-fold and 25-fold lower in assembled reads than in shotgun reads. The most abundant DNA TEs were found to be the CACTA elements (0.03% of shotgun reads) (Table 4). Because only predicted protein homologies were used to identify TEs and because all TEs contain extensive non-coding DNA, it is expected that the vast majority of the TE-related DNA in the date palm genome assembly was missed by this approach.

TABLE 4
Date Palm Assembly TE Analysis.

| | | | Number in reads |
|---|---|---|---|
| | | Copy number | (random 1X |
| Elements | | in assembly | sequences) |
| Non-coding TE | MITE | 38822 | 42620 |
| Protein homology | DNA/CACTA | 280 | 2839 |
| in TE (e-value = | DNA/PIF- | 69 | 143 |
| 10⁻⁵) | Harbinger | | |
| | DNA/Helitron | 190 | 320 |
| | DNA/MULE | 300 | 541 |
| | DNA/hAT | 980 | 2360 |
| | DNA/Other | 5 | 9 |
| | LINE | 4009 | 832 |
| | LTR/Copia | 6083 | 307405 |
| | LTR/Gypsy | 5271 | 132056 |
| | LTR/Other | 126 | 116 |
| Overall | | 55855 | 486393 |

### Example 9

### Polymorphism and Comparative Genomics

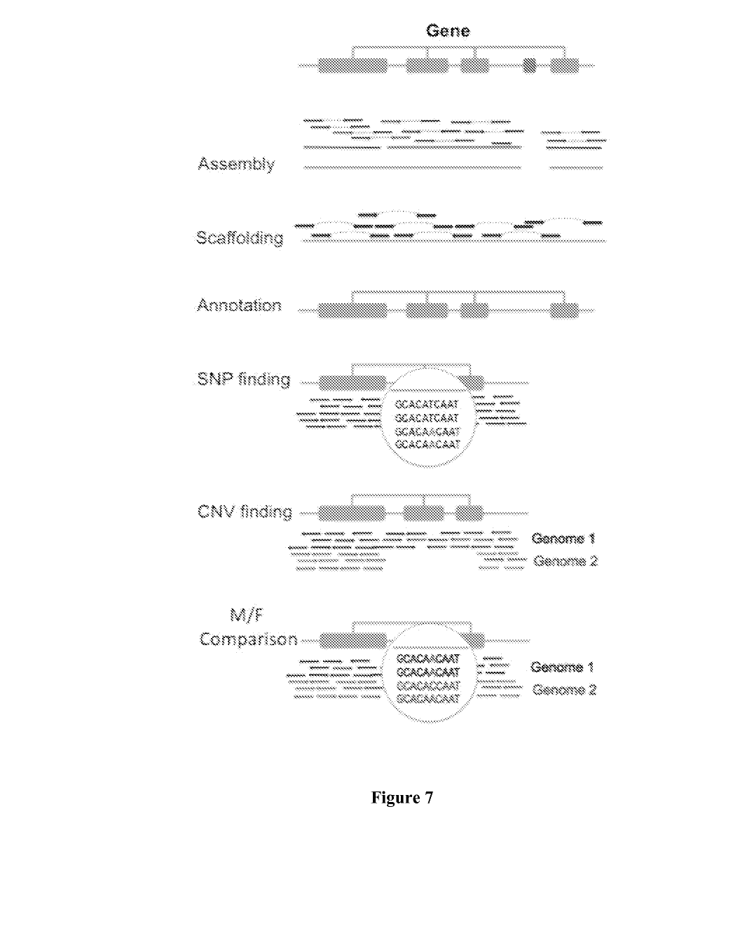

Using massively parallel sequencing on a cultivar of date palm with no documented in-breeding allowed detection of a large number of parental allelic differences (FIG. 7) using BWA (Li et al., “Fast and Accurate Short Read Alignment with Burrows-Wheeler Transform,” Bioinformatics 25:1754-60 (2009), which is hereby incorporated by reference in its entirely) and SAMTOOLS software (Li et al., “The Sequence Alignment/Map Format and SAMtools,” Bioinformatics 25:2078-9 (2009), which is hereby incorporated by reference in its entirety). 1,748,109 SNPs were called in 381 Mb of sequence, yielding a heterozygosity rate of 0.46% or 1 SNP/217 bp. However, the distribution was significantly skewed, with 49% of SNPs being found within 50 bp of another SNP (FIG. 3A). These results were observed even when suspected repetitive regions, including ends of contigs and high sequence coverage regions were excluded from the analysis. These results suggest that there are islands of higher polymorphism within the genome, and this observation is important to subsequent large polymorphism analysis. A total of 100.019 of the parental SNPs occurred within a predicted gene-encoding sequence, and 53,890 of these would lead to an amino acid change. This yields a nonsynonomous-to-synonomous SNP ratio of 1.17; a ratio similar to that of 1.2 reported in rice (McNally et al., “Genomewide SNP Variation Reveals Relationships Among Landraces and Modern Varieties of Rice,” PNAS 06: 2273 12278 (2009), which is hereby incorporated by reference in its entirety).

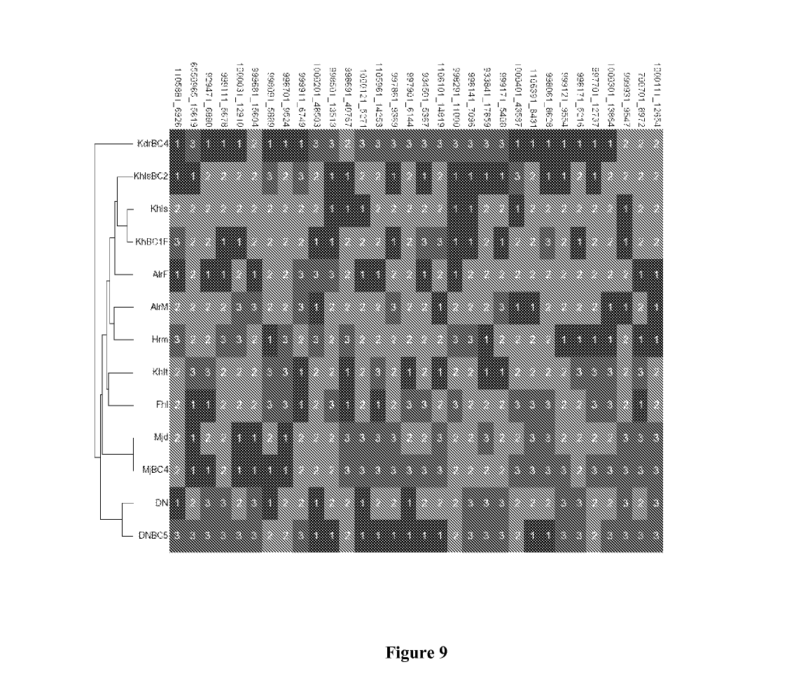

To better characterize polymorphism in date palm from a biotechnology perspective, genomes from top commercial varieties Deglet Noor and Medjool, and one non-commercial female (AlrijalF), were sequenced to varying levels of coverage (Table 5). Additionally, to characterize possible gender differences, two backcrossed males, two backcrossed females, and one non-backcrossed male (Table 5) were also sequenced. A total of 3,518,029 SNPs were identified in 381 Mb that were polymorphic in at least one of the sequenced genomes. The genotypes of all sequenced genomes were documented at these sites. Genotypes were much more conserved across the backcrossed genomes and their recurrent parents than between different varieties (FIG. 3B). Indeed, clustering of the genomes by the genotypes at 3.5 million locations revealed the close relationship of the backcrossed trees and their recurrent parents (FIG. 3C). Moreover, the genome of a Khalas variety collected in Qatar clustered very close with trees backcrossed to a California Khalas believed to have been imported from Arabia almost 100 years ago (Hodel et al., Dates, Imported and American Varieties of Dates in the United States (ANR Publications 2007), which is hereby incorporated by reference in its entirety). Using a decision tree algorithm (Zhang et al., “Willows: A Memory Efficient Tree and Forest Construction Package,” BMC Bioinformatics 10:130 (2009), which is hereby incorporated by reference in its entirety), the fewest number of SNPs capable of separating the nine sequenced genomes were identified. Selecting for the most variety informative SNPs revealed a minimum of 5 SNPs could be used to separate the 9 sequenced varieties (Table 6). A total of 32 SNPs (Table 6) were highly informative in discriminating the varieties. Using just these 32 SNPs to separate the varieties provided little loss of discrimination power, with the top 3 principal components decreasing from 74% to 71% when comparing results with 3.5 million versus the core 32 SNPs (FIG. 3D). An additional 4 genomes were genotyped using these 32 SNPs and clustering again showed strong separation power (FIG. 9). This set of SNPs will provide a resource for future development of a variety of specific DNA markers.

TABLE 5
Date Palm Genomes Sequenced for this Study.

| Date Palm | Presumed | Collected | | Sequence |
|---|---|---|---|---|
| Cultivar | Origin | in | Gender | coverage |
| Khalas | Arabia | Qatar | Female | 53.4X |
| Khalas x Khalas | California | California | Female | 19.3X |
| F1 | | | | |
| Khalas BC2 | California | California | Female | 10.1X |
| Deglet Noor | North | California | Female | 19.8X |
| | Africa | | | |
| Deglet Noor BC5 | California | California | Male | 19.6X |
| Medjool | North | California | Female | 14.9X |
| | Africa | | | |
| Medjool BC4 | California | California | Male | 13.1X |
| Khalt | Qatar | Qatar | Male | 11.2X |
| AlrijalF | Qatar | Qatar | Female | 10.7X |

Three commercially important female genomes together with various backcrosses and uncultivated varieties were sequenced to various levels of sequence redundancy to permit SNP identfication.

TABLE 6
SNPs with High Discriminating Power among Date Palm Varieties.

| Scaffold | Location |
|---|---|
| PDK_30s1000031 | 12910 |
| PDK_30s1000111 | 12654 |
| PDK_30s1000301 | 13864 |
| PDK_30s1000121 | 5271 |
| PDK_30s1000401 | 43587 |
| PDK_30s999911 | 6749 |
| PDK_30s999931 | 9547 |
| PDK_30s999681 | 15604 |
| PDK_30s1000201 | 48503 |
| PDK_30s999171 | 5408 |
| PDK_30s999121 | 9554 |
| PDK_30s999111 | 5678 |
| PDK_30s998701 | 9524 |
| PDK_30s998691 | 40767 |
| PDK_30s998501 | 13513 |
| PDK_30s998291 | 11000 |
| PDK_30s998171 | 5216 |
| PDK_30s998141 | 7096 |
| PDK_30s998091 | 5889 |
| PDK_30s998061 | 8628 |
| PDK_30s997901 | 6144 |
| PDK_30s997861 | 9369 |
| PDK_30s997701 | 12737 |
| PDK_30s1106391 | 8431 |
| PDK_30s1106101 | 14819 |
| PDK_30s1105961 | 14253 |
| PDK_30s1105881 | 6926 |
| PDK_30s700701 | 8972 |
| PDK_30s6550965 | 15619 |
| PDK_30s934501 | 5367 |
| PDK_30s933841 | 17859 |
| PDK_30s929471 | 6880 |

32 SNPs with the highest power to discriminate date palm varieties are presented. 5 SNPs (in bold font) could distinguish 9 sequenced genomes. The combination of 32 SNPs showed very little loss of discrimination power with respect to the total set of 3.5 million SNPs.

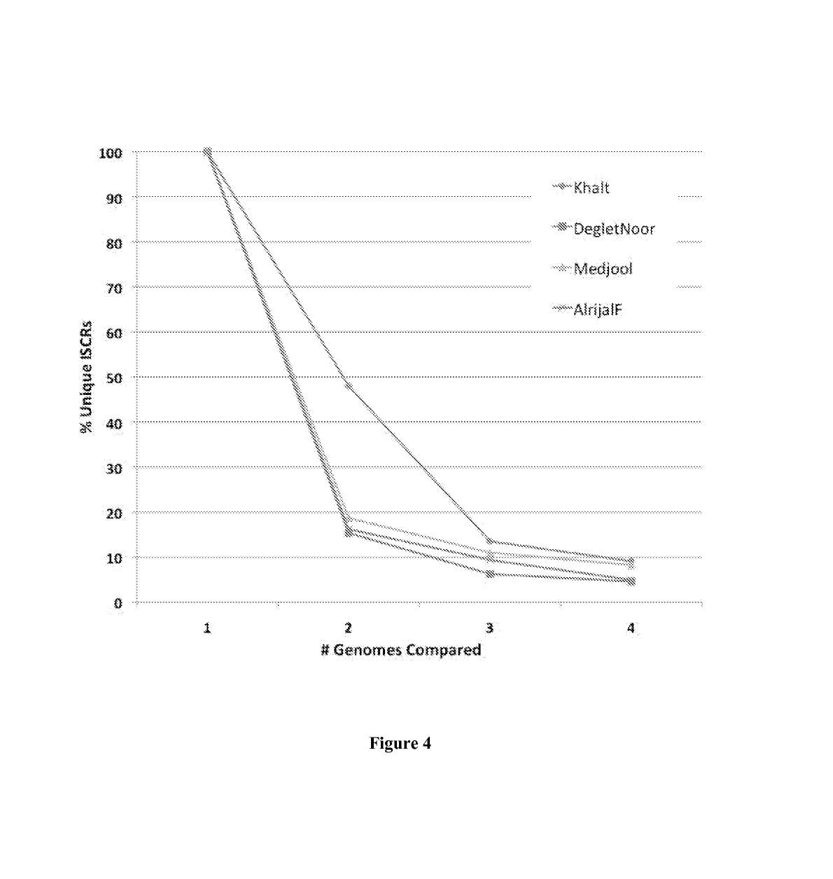

Large scale polymorphisms, including CNVs can be detected from sequence data by identifying regions where the observed number of matching sequences from a genome significantly deviate (either up or down) from the expected numbers (FIG. 7). By matching sequences from each genome to the Khalas reference, gene-sized regions with significantly imbalanced counts of sequences were detected using the CNV-SEQ software (Xie et al., “CNV-seq, a New Method to Detect Copy Number Variation Using High-throughput Sequencing,” BMC Bioinformatics 10:80 (2009), which is hereby incorporated by reference in its entirety). These are termed ISCRs to distinguish them from more rigorously proven CNVs. The analysis was restricted to only non-backcrossed genomes to avoid duplication of results from inbreeding. As with the SNP data, extensive conservation of ISCRs was observed between backcrossed genomes and their recurrent parent. A total of 10,388 ISCRs were detected that both overlap a predicted gene-coding region and occur in at least two genomes. At most, 10% of ISCRs were unique to a given genome (FIG. 4).

While uneven distribution of polymorphism with high sequence conservation in gene regions (Ma et al., “Rapid Recent Growth and Divergence of Rice Nuclear Genomes,” PNAS 101:12404-10 (2004); Yu et al., “The Genomes of Oryza sativa: A History of Duplications,” PLoS Biology 3:e38 (2005), which are hereby incorporated by reference in their entirety) may lead to false ISCR detection, modeling suggests that most of these ISCRs are real.

Plant cultivars are known to exhibit high levels of polymorphism across the genome, punctuated by regions of low polymorphism in gene regions (Ma et al., “Rapid Recent Growth and Divergence of Rice Nuclear Genomes,” PNAS 101:12404-10 (2004); Yu et al., “The Genomes of Oryza sativa: A History of Duplications,” PLoS Biology 3:e38 (2005), which are hereby incorporated by reference in their entirety). The date palm genome appears similar as uneven distribution of parental allele SNPs were observed in the Khalas female (FIG. 3A). The method used to detect ISCRs is based on sequence alignment and thus requires reasonably high sequence similarity between compared genomes. If the genomes were too divergent, sequences from the test genome would not match in polymorphic regions, causing deletions to be called. Matching sequences would be distributed only to regions of higher similarity, resulting in these regions being called amplifications. However, there was not a correlation between SNP density and propensity to call ISCRs in genes. Moreover, modeling of ISCR calling on the genome with in silico created polymorphisms, punctuated by regions of low polymorphism, resulted in 56 false ISCRs compared to the 10,000 detected here. These results suggest that most ISCRs are either true CNVs or interesting regions of extremely high polymorphism.

If sequence-level polymorphism is indeed responsible for a large portion of the ISCRs, then deletion ISCRs should be more likely to occur in genes that have high numbers of SNPs. This was checked with empirical SNP data. In fact, polymorphism rates were slightly higher in amplification ISCRs (0.74% heterozygosity) than in deletion ISCRs (0.66% heterozygosity). Correlation of the frequency of SNPs in a gene, detected from the Khalas parental alleles, was also checked with the likelihood that the same gene was involved in a deletion ISCR in any number of cultivars. A positive correlation would only be observed if gene regions dense in polymorphisms between the parental alleles of the Khalas reference genome were also more likely to be polymorphic in other cultivars of date palm. Genes were ranked by how many times they were observed in a deletion ISCR in the multiple cultivars, and further ranked by how many SNPs per 1000 bp were observed in the parental alleles of the Khalas strain. All 1,911 genes that contained at least 1 SNP in the Khalas parental alleles and were in at least 1 ISCR among 4 genomes compared were utilized. The Spearman's Rank Correlation Coefficient between the two ranked groups was 0.095 and −0.011 (uncorrected/corrected) with a p-value of 0, showing a lack of correlation between levels of SNPs in a gene in Khalas and the propensity to call a deletion ISCR.

To further understand the level of ISCRs detected due to sequence dissimilarity between the genomes, ISCR was modeled, calling on in silico mutated versions of the Khalas reference strain. The goal was to observe the frequency of ISCRs called on in silico mutated sequence and from this to estimate the number of ISCRs in the dataset that are most likely due to sequence dissimilarity, rather than true large scale amplifications or deletions. An in silico mutated genome that was a patchwork of polymorphism rates was created. A SNP rate of 1.5% and an indel rate of 0.3%, punctuated by simulated gene regions, presumably more highly conserved, with 0.6% SNP and 0.12% indel rates were used. Gene regions were 4-6 kbp in length, summing to a total of 86 Mbp, which is the predicted amount of genic DNA in the Khalas genome. The modeled SNP rates are higher in intragenic regions than empirically observed date palm rates in order to exaggerate possible aggravation of ISCR detection. Using this model, 56 ISCRs were reported with 49 being called as deletions and 7 as amplifications. Across all modeled genic regions with the lower polymorphism rate there was an average log 2 increase of 0.167 (S.D. 0.027), which is well outside the log 2 of 0.6 required to call an ISCR as significant. While detection of 56 ISCRs is significant, most of the genomes studied here had on the order of 10,000s ISCRs. The results of this modeling suggest that a large number of the regions detected as variable between the genomes are most likely either copy number variations or regions of extremely high polymorphism. Importantly, this modeling shows that standard polymorphism rates among cultivars alone, even in the presence of variable polymorphism rates in genes, should not cause detection of high numbers of false CNVs.

Furthermore, quantitative PCR (“qPCR”) of 5 ISCRs on the 4 test genomes (20 different tests), gave 16 results consistent with expectation (amplified or deleted). Visual inspection of the sequence alignment in the 4 ISCR regions that failed to validate revealed that, in some cases, sequence coverage variability is due to very high sequence polymorphism rather than absolute loss of sequence.

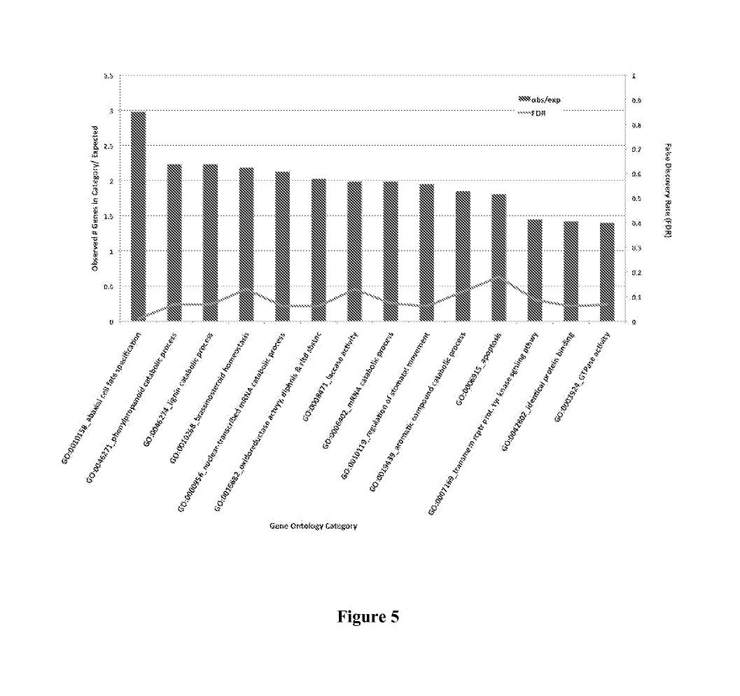

Genes exhibiting ISCRs in at least 2 genomes were analyzed for Gene Ontology enrichment using the GOSSIP package within BLAST2GO (Conesa et al., “Blast2GO: A Comprehensive Suite for Functional Analysis in Plant Genomics,” Int. J. Plant Genomics Pub. ID. No. 619832 (2008), which is hereby incorporated by reference in its entirety), and enrichment was found in certain functional categories (FIG. 5). Interestingly, the categories for lignin, laccase, and phenylpropanoid metabolism were overrepresented in ISCR regions. Genes in these processes are important in fruit flavor and ripening (Singh et al. “Phenylpropanoid Metabolism in Ripening Fruits,” Comprehensive Reviews in Food Science and Food Safety 9:398-416 (2010), which is hereby incorporated by reference in its entirety), one of the most distinguishable differences between date palm varieties, and thus offer a resource for studying date palm fruit properties. The large number of ISCRs between the genomes analyzed here are not entirely unexpected. It has been shown that a significant amount of variation among genomes is related to indels (Britten et al., “Majority of Divergence Between Closely Related DNA Samples is Due to Indels,” PNAS 100:4661-4665 (2003), which is hereby incorporated by reference in its entirety) and, in some plants, this amounts to 10-20% of genome variation between cultivars (Ding et al., “Highly Asymmetric Rice Genomes,” BMC Genomics 8:154 (2007); Huang et al., “Genome-wide Analysis of Transposon Insertion Polymorphisms Reveals Intraspecific Variation in Cultivated Rice,” Plant Physiology 148:25-40 (2008), which are hereby incorporated by reference in their entirety).

No ISCRs were found to segregate with gender. Recognizing that comparing all genomes to the Khalas female genome could only identify female-specific sequences, an attempt was made to assemble male-specific sequences. Reads from the male Deglet Noor BC5 genome were assembled. Very short contigs were expected, because sequence redundancy (20×) was not high, but this served as a first check for male-specific sequences. Sequences from the Medjool, Khalas, and Deglet Noor female genomes were matched to the Deglet Noor BC5 male contigs. No contigs were observed to be absent in all 6 female genomes. Annotation of the short contigs revealed high frequencies of LTR retrotransposons, but no distinguishable male-specific genes.

### Example 10

### Identification of Gender-Linked Scaffolds

The 3.5 million SNP genotypes were scanned in the male and female genomes to identify polymorphisms segregating with gender (FIG. 7). The observed results best fit an XY sex determination model with males being the heterogamete. Applying a heterogamete male model, 1,605 SNPs were observed that segregated with gender. Of these, 934 (58%) localized to 344 kb within 24 scaffolds spanning 602 kb (Table 7). Specifically, all male genomes shared mainly the same heterozygous genotypes while female genomes shared mainly the same homozygous genotypes in these scaffolds. The scaffolds are broken by gaps that probably contain significant amounts of repetitive DNA. Analyzing two scaffolds with the most gender-segregating SNPs, an approximate 3-fold difference was observed in divergence from the reference sequence between male and female haplotypes. Furthermore, a near 30-fold difference was observed between the number of male and female heterozygous SNPs within these regions. The genotypes of polymorphic sites were recorded for all genomes in these regions, as set forth in FIGS. 2A-AK.

| # of | | | | | region | contig | | |
|---|---|---|---|---|---|---|---|---|
| SNPs | Contig | Start | Stop | bp | size | Length | Genes within region | Annotation |
| 154 | PDK_30s1150131 | 1487 | 48776 | 307 | 47289 | 50308 | PDK_30s1150131L001 | cell differentiation |
| | | | | | | | | protein |
| | | | | | | | PDK_30s115013L002 | rab geranylgeranyl |
| | | | | | | | | transferase like |
| | | | | | | | | protein |
| 133 | PDK_30s1038101 | 207 | 33936 | 253 | 33729 | 34146 | PDK_30s1038101L001 | partial gene, no |
| | | | | | | | | protein identified |
| | | | | | | | PDK_30s1038101L002 | hypothetical protein |
| | | | | | | | PDK_30s1038101L003 | transcriptional |
| | | | | | | | | regulators of-like |
| 73 | PDK_30s944511 | 4328 | 11242 | 94 | 6914 | 11556 | Contains Retrotransposon | |
| 58 | PDK_30s1038231 | 734 | 22080 | 368 | 21346 | 23119 | PDK_30s1038231L001 | predicted protein |
| 53 | PDK_30s1106761 | 1668 | 19021 | 327 | 17353 | 20200 | PDK_30s1106761L001 | 60s acidic ribosomal |
| | | | | | | | | protein p0 |
| 50 | PDK_30s1118051 | 36469 | 59536 | 461 | 23067 | 77159 | PDK_30s1118051L004 | partial gene, no |
| | | | | | | | | protein identified |
| | | | | | | | PDK_30s1118051L005 | partial gene, no |
| | | | | | | | | protein identified |
| 46 | PDK_30s6550963 | 70489 | 81307 | 235 | 10818 | 95115 | PDK_30s6550963L005 | Myb Superfamily |
| 43 | PDK_30s1202771 | 1643 | 17171 | 361 | 15528 | 20074 | PDK_30s1202771L001 | translation initiation |
| | | | | | | | | factor if-2 |
| | | | | | | | PDK_30s1202771L002 | fug1 (fu-gaeri1) |
| | | | | | | | | translation initiation |
| | | | | | | | | factor |
| 40 | PDK_30s717571 | 837 | 25874 | 625 | 25037 | 26372 | PDK_30s717571L001 | SAPS superfamily - |
| | | | | | | | | interacts for G1 |
| | | | | | | | | cyclin transcription |
| 30 | PDK_30s695331 | 3950 | 16030 | 402 | 12080 | 17590 | PDK_30s695331L002 | phosphatidylinositol |
| | | | | | | | | glycan anchor class o |
| | | | | | | | PDK_30s695331L003 | predicted protein |
| 29 | PDK_30s1035941 | 4891 | 17575 | 437 | 12684 | 19615 | PDK_30s1035941L001 | protein binding |
| 26 | PDK_30s950111 | 1635 | 26516 | 956 | 24881 | 42207 | PDK_30s950111L001 | flap endonuclease-1b |
| 25 | PDK_30s1095881 | 2741 | 9389 | 265 | 6648 | 16579 | PDK_30s1095881L001 | syntaxin of plants 52 |
| 24 | PDK_30s684341 | 165 | 3656 | 145 | 3491 | 3946 | no Predicted Genes | |
| 22 | PDK_30s754231 | 2990 | 13316 | 469 | 10326 | 14607 | PDK_30s754231L001 | phosphatidylinositolg |
| | | | | | | | | lycan class expressed |
| 18 | PDK_30s893961 | 11779 | 19757 | 443 | 7978 | 20362 | PDK_30s950111L001 | Flap endonuclease- |
| | | | | | | | | 1b |
| 17 | PDK_30s1023161 | 664 | 15917 | 897 | 15253 | 16271 | PDK_30s1023161L001 | partial: |
| | | | | | | | | phosphatidylinositol |
| | | | | | | | | glycan |
| 5 | PDK_30s675061 | 2706 | 7485 | 318 | 4779 | 7694 | no Predicted Genes | |
| 15 | PDK_30s925161 | 2733 | 11396 | 577 | 8663 | 31314 | PDK_30s925161L001 | NPH3 domain |
| | | | | | | | | containing, root |
| | | | | | | | | phototropism |
| 15 | PDK_30s65509204 | 180 | 11070 | 726 | 10890 | 12637 | PDK_30s65509204L001 | 40s ribosomal |
| | | | | | | | | protein s12 |
| 14 | PDK_30s667171 | 100 | 13285 | 941 | 13185 | 13542 | no Predicted Genes | |
| 12 | PDK_30s680001 | 9579 | 14002 | 368 | 4423 | 17202 | PDK_30s680001L001 | predicted protein |
| 11 | PDK_30s1004611 | 585 | 4315 | 339 | 3730 | 4471 | PDK_30s1004611L001 | predicted protein |
| | | | | | | | PDK_30s1004611L002 | predicted protein |

The number of SNPs was based on comparison of the sequenced male and female genomes. Regions of the scaffolds that show SNPs segregating with gender are documented.

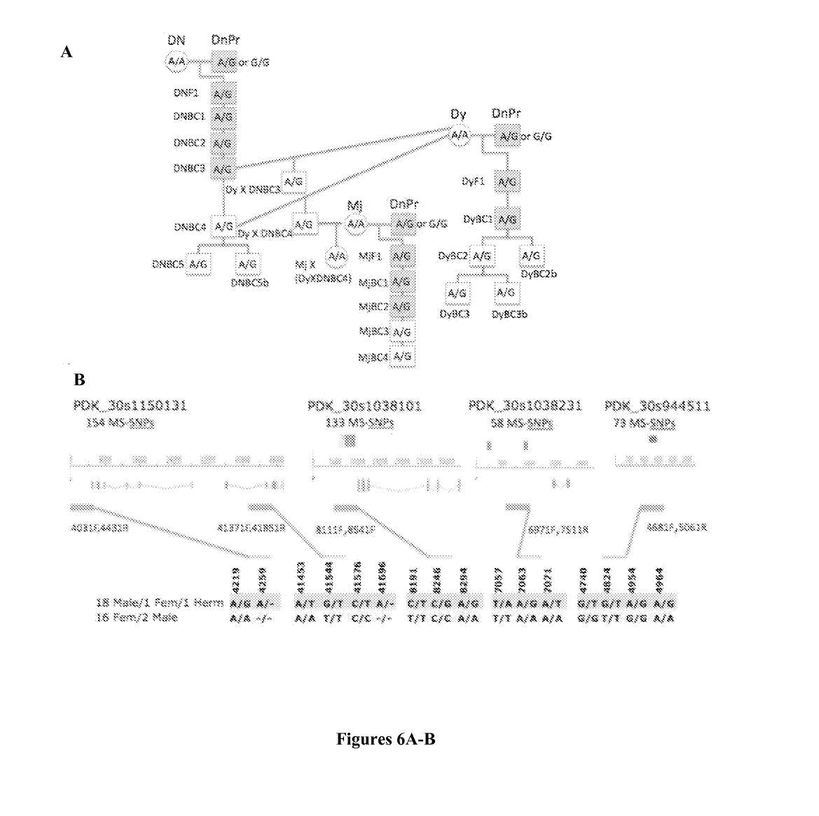

In comparing Deglet Noor and Medjool et ales to the Khalas female reference, 253 and 271 sites differed from the Khalas reference and only 24 (9%) and 19 (7%) sites were heterozygous, respectively. At the same positions, their backcrossed males showed 736 and 770 sites differing from the Khalas reference and 584 (79%) and 578 (75%) of these were heterozygous. The significantly higher heterozygosity levels (χ2=893.6 and 767.7, 1 d.f., p<0.0001) in the males represents an ˜3-fold increase in heterozygosity in these regions when compared to the rest of the genome. The females have significantly reduced heterozygosity with respect to the rest of the genome (χ2=435.9 and 410.2, 1 d.f., p<0.0001), resulting in an ˜14-fold decrease in heterozygosity in these regions versus the rest of the genome. This pattern of sequence degeneration between male and female haplotypes may be indicative of reduced recombination between the male and female haplotypes, which is a step that may be critical to the development of gender-specific regions (Charlesworth et al., “A Model for the Evolution of Dioecy and Gynodioecy,” The American Naturalist 112:975-997 (1978); Bergero et al. “The Evolution of Restricted Recombination in Sex Chromosomes,” Trends in Ecology & Evolution 24:94-102 (2009), which are hereby incorporated by reference in their entirety). In these two scaffolds, 7 exons were observed in 3 of the 4 annotated genes (FIG. 6B) that contained unusually long introns, ranging between 4 kb and 13.1 kb (compared to an average of less than 200 bp for most flowering plant introns). It has been shown that longer introns occur more frequently in regions of low recombination in Drosophila (Carvalho et al., “Intron Size and Natural Selection,” Nature 401:344 (1999), which is hereby incorporated by reference in its entirety).

To determine if the observed differences in heterozygosity were truly linked to gender, short regions from four scaffolds with the largest number of segregating SNPs were selected for genotyping in a pedigree containing 6 date palm female varieties and their 20 progeny (Table 8). Genotyping results indicate that these four scaffolds are linked to each other with no recombination between them (FIG. 6B), suggesting they likely localize to the same region of the genome. Using only empirically-determined genotypes on males and females (excluding hermaphrodites), the genotyped scaffolds significantly link to gender with a LOD score of 5.3 (recombination frequency of 0.07), with only two males showing recombination. Furthermore, as backcrossed plants were used in the pedigree, theoretically-determined recurrent parents can be included, improving the LOD score to 8.9 (recombination frequency of 0.05) (FIG. 6A). Genotyping of date palms outside the pedigree continued the trend of male heterozygosity and female homozygosity. With a total of 63 empirically and theoretically genotyped males and females, 5 did not give the expected genotype (FIG. 6B), Additionally, one male was observed to be homozygous for the male-specific allele (Table 8). Predicted genes in this region (Table 7) include an red-I homologue (“Required for Cell Differentiation-1”), a Myb family gene, and a gene that should encode a rab geranylgeranyl transferase-like protein (a prenyl-transferase). Interestingly, it has been shown that the rcd-1 gene is important in sexual development in yeast (Okazaki et al., “Novel Factor Highly Conserved among Eukaryotes Controls Sexual Development in Fission Yeast,” Mol. Cell. Biol. 18:887-895 (1998), which is hereby incorporated by reference in its entirety) and that it interacts with c-Myb (Haas et al., “c-Myb Protein Interacts with Red-1, a Component of the CCR4 Transcription Mediator Complex,” Biochemistry 43:8152-9 (2004), which is hereby incorporated by reference in its entirety). Moreover, it has been shown that cell differentiation control in date palm floral development is critical to sex organ development (Daher et al., “Cell Cycle Arrest Characterizes the Transition from a Bisexual Floral Bud to a Unisexual Flower in Phoenix dactylifera,” Annals of Botany 106:255-66 (2010), which is hereby incorporated by reference in its entirety) and that there are sets of MADS box genes, which control flower development, and require prenylation for correct function (Yalovsky et al., “Prenylation of the Floral Transcription Factor APETALA1 Modulates its Function,” The Plant Cell 12:1257-66 (2000), which is hereby incorporated by reference in its entirety). Multiple non-synonymous polymorphisms were observed between the male and female haplotypes in these genes though none were certain to be deleterious.

TABLE 8
Genotype Results at the Suspected Gender-specific Region.

| Recurrent | Accession | | | USDCS | | Obs. | Rep. | | |
|---|---|---|---|---|---|---|---|---|---|
| Parent | No. | sex | Gen. | No. | Progeny | Sex | gen. | Access. No. | Parentage |
| Khalas | PI 8753- | F | AA | 69-155-14 | KhlsBCFx | F | AA | PI555437-PL2208 | Khalasa X 61-411 |
| | RIV2193 | | | | | | | | |
| | | | | 61-411-2 | KhlsBC2 | F | AA | PI555423-PL2200 | Khalasa X 52-251-4 |
| | | | | 69-155-51 | KhlsBC2b | M | AA | PI555436-PL2197 | Khalasa X 61-411 |
| Deglet Noor | PI 4611- | F | AA | 69-150-50 | DNBC5 | M | A/G | PI555433-PL2165 | Deglet Noor X 64- |
| | RIV2154 | | | | | | | | 354 |
| | | | | 69-150-52 | DNBC5b | M | A/G | PI555432-PL2167 | Deglet Noor X 64- |
| | | | | | | | | | 354 |
| | | | | 69-150-52 | DNBC5c | M | A/G | PI55543-PL7506 | Deglet Noor X 64- |
| | | | | | | | | | 354 |
| | | | | 69-150-50 | DNBC5d | M | A/G | PI555432-PL2166 | Deglet Noor X 64- |
| | | | | | | | | | 354 |
| | | | | 64-354-22 | DNBC4 | M | A/G | PI555402-PL2144 | Deglet Noor X 60- |
| | | | | | | | | | 272 |
| | | | | | DNBC3 | M | A/G | | |
| | | | | | DNBC2 | M | A/G | | |
| | | | | | DNBC1 | M | A/G | | |
| | | | | | DNFI | M | A/G | | |
| Barhee | PI 8746- | F | AA | 70-41-30 | BarheeBC4 | Herm | AA | PI555415-RIV2132 | Barhee X 60-270-9 |
| | RIV7479 | | | | | | | | |
| | | | | 60-270-9 | BarheeBC3 | M | AA | PI555412-RIV7544 | Barhee X 55-94 |
| | | | | 74-31-50 | BarheeBC3b | M/Herm | A/G | PI555419-RIV2133 | Barhee X 66-16-1,5 |
| Medjool | RPHO1- | F | AA | 69-157-52 | MdjlBC4 | M | A/G | PI555438-RIV2219 | Medjool X M63- |
| | RIV2134 | | | | | | | | 397 |
| | | | | 69-157-52 | MdjlBC4b | M | A/G | PI555438-RIV7526 | Medjool X M63- |
| | | | | | | | | | 397 |
| | | | | 69-157-52 | MdjlBC4c | M | A/G | PI555438-2220 | Medjool X M63- |
| | | | | | | | | | 397 |
| | | | | 69-157-51 | MdjlBC3 | M | A/G | PI555439-RIV2196 | |
| | | | | 71-25-36 | Mdjlx(DyrX | F | AA | PI5427-RIV2223 | Medjool X 61-414-6 |
| | | | | | DNBC3) | | | | |
| | | | | 71-25-36 | Mdjlx(DyrX | F | AA | PI5427-RIV2222 | Medjool X 61-414-6 |
| | | | | | DNBC3)b | | | | |
| | | | | | BC2 | M | A/G | | |
| | | | | | BC1 | M | A/G | | |
| | | | | | F1 | M | A/G | | |
| Khadrawy | PI 8751- | F | AA | 63-394-25 | KhdrwBC3 | M | A/G | PI55444-RIV7426 | Khadrawy X 57-32 |
| | RIV2171 | | | | | | | | |
| | | | | 69-154-28 | KhdrwyBC4 | M | A/G | PI555435-PL7491 | Khadrawy X 62- |
| | | | | | | | | | 430 |
| | | | | | BC3 | M | A/G | | |
| | | | | | BC2 | M | A/G | | |
| | | | | | BC1 | M | A/G | | |
| | | | | | F1 | M | A/G | | |
| Dayri | PI 8567- | F | AA | 60-271-2 | DyriBC2 | M | A/G | PI555413-RIV7441 | Dayri X 50-5-10 |
| | RIV7490 | | | | | | | | |
| | | | | 60-271-7 | DyriBC2B | M | A/G | PI555414-RIV2149 | Dayri X 50-5-10 |
| | | | | 60-271-2 | DyriBC2C | M | A/G | PI555413-RIV7442 | |
| | | | | 60-271-7 | DyriBC2D | M | A/G | PI555414-RIV2150 | |
| | | | | 70-39-53 | DyriBC3 | M | A/G | PI555417-RIV2145 | Dayri X 60-271-7 |
| | | | | 70-39-51 | DyriBC3B | M | A/G | PI555416-RIV7504 | Dayri X 60-271-7 |
| | | | | 70-39-53 | DyriBC3C | M | A/G | PIR555417-RIV2146 | |
| | | | | 61-414-6 | DyxDNBC3 | M | A/G | PI555422-RIV7393 | Dayri X 55-100 |
| | | | | 71-15-50 | DyXDNBC4 | M | A/G | PI555424-RIV2233 | Dayri X 64-354-22 |
| | | | | | BC1 | M | A/G | | |
| | | | | | F1 | M | A/G | | |

Non-Pedigree Genotyped Trees

| | | | | | |
|---|---|---|---|---|---|
| 78-9 | Crane | M | AG | PI555421-RIV2478 | |
| 66-14-50 | ZahidiBC2 | F | AA | PI555407-RIV7429 | Zahidi X 60-279 |
| 66-14-52 | ZahidiBC2B | M | AG | PI555408-RIV7523 | Zahidi X 60-279 |
| 66-15-51 | ZahidiBC2C | M | AG | PI555409-RIV2236 | Zahidi X 57-29 |
| | Saqie-F95 | F | AA | | |
| | Gar-F96 | F | AA | | |
| | Lolo-F100 | F | AA | | |
| | AboMaian- | F | AA | | |
| | F104 | | | | |
| | Khoname- | F | A/G | | |
| | F05 | | | | |
| | Zamly-F106 | F | AA | | |
| | Fard-F107 | F | AA | | |
| | Khalt | M | AG | | |
| | M62 | M | G/G | | |
| | AlrijalF | F | AA | | |
| | M60 | M | AA | | |
| | AlrijalM | M | AA | | |

The number of SNPs was based on comparison of the sequenced male and female genomes. Regions of the scaffolds that show SNPs segregating with gender are documents.

### Example

### Validation of Identified SNPs

Validation of identified SNPs was carried out using PCR and sequencing. For example, PCR primers were designed against the scaffold: PDK_{—}30s1150131 at position 4031 in the forward orientation and at position 4431 in the reverse orientation. Primer sequences included:

(SEQ ID NO: 973)
PDK_30s1150131_4031F(GAGTTAATATCTCCTTGCCATCCT
(SEQ ID NO: 974)
PDK_30s1150131_4431R(GTCAAGGGATCTCCCTATTGTA.

DNA Sequencing using the forward primer results in the ability to genotype at 3 locations in the intervening sequence:

(SEQ ID NO: 975)
TATGTACACA[A/G]GTAAG[T/G]CTCCTTTTTCACTTG[C/G]
AGACATATAGAG

Genotyping of the first (bold) polymorphic position in SEQ ID NO:975 (with the possible genotype of homozygous A (AA), heterozygous (AG), or homozygous B (GG)) results in linkage of the AA to female gender and the linkage of AG (heterozygous) to male gender.

Results of the genotyping the polymorphic site in Pedigrees of Date Palm trees from the USDA collection in California below in Table 9. Trees that genotype contrary to the expected gender genotype are in bold. A significant linkage score (LOD score) of 3.2 is found between this marker and gender from this experimental data alone. Because many of the progeny are males from backrosses of multiple generations, their donor parents (fathers) genotype can be theoretically determined to be heterozygous (any progeny of a cross with an AA plant would produce AG or AA genotypes and, therefore, the male donor plants that are progeny of a cross must be AG). Using the added theoretical genotypes increases the LOD score to a minimum of 6.67, making the linkage between this marker and date palm gender very strong.

Genotyping of other locations in the mentioned scaffolds showed linkage disequilibrium to the above genotype SNP. This is most likely due to their proximity in the genome to this scaffold. Therefore, all SNPs with linkage disequilibrium to the detected SNPs would be expected by chance to be included in the present invention.

TABLE 9
Genotypes of Male and Female Trees Showing Segregation of Genotype
and Gender.

| Mother | Sex | Genotype | Progeny | Sex | Genotype |
|---|---|---|---|---|---|
| Khalas | F | AA | KhlsBCFx | F | AA |
| | | | KhlsBC2 | F | AA |
| | | | KhlsBC2B | M | AA |
| DegNoor | F | AA | DNBC52167 | M | A/G |
| | | | DNBC52165 | M | A/G |
| | | | DNBC4 | M | A/G |
| Barhee | F | AA | BarheeBC4-5415 | Herm | AA |
| | | | BarheeBC3-5412 | M | AA |
| | | | BarheeBC3B(fruiting)- | M | A/G |
| | | | 5419 | | |
| Medjool | F | AA | MdjlBC4 | M | A/G |
| | | | MdjlBC3 | M | A/G |
| | | | Mdjlx(DyrXDN) | F | AA |
| Khadrawy | F | AA | KhdrwBC3 | M | A/G |
| | | AA | KhdrwyNBC4 | M | A/G |
| Dayri | F | AA | DyriBC2 | M | A/G |
| | | | DyriBC2B | M | A/G |
| | | | DyriBC3 | M | A/G |
| | | | DyriBC3B | M | A/G |
| | | | DyxDNBC3 | M | A/G |
| | | | DyXDNBC4 | M | A/G |

Described herein is the first publicly available genome from the palm family. The date, oil, and coconut palms are important crops in several developing countries, and this sequence can serve as a vital resource for their improvement. Though short read assembly has its limitations in heterozygous and repetitive regions, gene regions with contiguity similar to other draft genome sequences (Yu et al., “A Draft Sequence of the Rice Genome (Oryza saliva L. ssp. indica),” Science 296:79-92 (2002); Ming et al., “The Draft Genome of the Transgenic Tropical Fruit Tree Papaya (Carica papaya Linnaeus),” Nature 452:991-996 (2008), which are hereby incorporated by reference in their entirety) was obtained by utilizing paired-end libraries of varying sizes. The approach focused on obtaining the gene regions of the date palm by relying on the observation that most plants have fewer repeat sequences within genes. The next step in the improvement of this sequence should be its anchoring to physical and genetic maps. However, the utility of the current assembly is revealed in the ability to begin answering pressing needs in date palm improvement.

The aim in this study was to provide a date palm genome resource with which to begin addressing the main biotechnology issues in date palm development: cultivar genetic differentiation and tree gender discrimination. Annotation of the current assembly has dramatically improved the current knowledge of the date palm gene content and allelic variation. Sequence data from multiple genomes has provided the largest resource of polymorphic markers to date. A small subset of these markers have been identified that can serve as a resource in genotyping the more than 2,000 date palm varieties.

Three of the top date palm varieties that are important in three regions of date palm production have hereby been sequenced: Khalas favored in Arabia: Deglet Noor favored in North Africa; and Medjool increasingly favored in California (Hodel et al., Dates, Imported and American Varieties of Dates in the United States (ANR Publications 2007), which is hereby incorporated by reference in its entirety). This resource will allow future comparisons of traits such as fruit quality and ripening time that vary among these favored varieties. Sequencing of the backcrossed males, a unique resource in any long-generation plant, allowed genomic-level studies of male/female date palm differences. Scaffolds strongly linked to gender were identified, and the establishment of a DNA marker-based gender test may now be feasible. These regions will be further studied to identify a specific mutation, mutations, or other gene content difference that leads to a male or female progeny outcome.

For millennia, date palm cultivation of favored female varieties has taken the form of offshoot propagation. It has been essentially impossible to grow a specific female date palm variety from seed because seedling-grown fruit quality is too different from the mother to be economically useful. By combining the findings presented here with the backcrossed genetic resources that began to be generated decades ago (Barrett, “Date Breeding and Improvement in North America,” Fruit Varieties Journal 27:50-55 (1973), which is hereby incorporated by reference in its entirety), seeds of backcrosses, identified as female at the earliest stages and genotyped to show similarity to the original mother, will now be available. The results provided herein have laid the foundation for date palm genomic-level research by providing the first genome-wide gene set, the first genome-wide multi-variety polymorphism set, and the first gender-linked regions.

### Example 12

### DNA-Based Assays to Distinguish Date Palm Gender

As described herein, regions in the date palm genome that are linked to gender have been identified (Al-Dous et al., “De novo Genome Sequencing and Comparative Genomics of Date Palm (Phoenix dactylifera),” Nature Biotechnology 29:521-527 (2011), which is hereby incorporated by reference in its entirety). Investigation of these regions revealed that the date palm employs a XX/XY sex determination system with the male being the heterogametic sex. The regions also showed significant polymorphism between the male and female alleles. This polymorphism can be used in the development of assays to distinguish the two sexes at an early stage. In this example, two approaches were employed to develop DNA-based assays for sex differentiation in date palm. The first were PCR-based restriction fragment length polymorphism (“PCR-RFLP”) approaches that require amplification followed by restriction digestion and gel electrophoresis. The second approach is a PCR-only method that takes advantage of the high heterogeneity in the sex-linked region to remove the need for the restriction digestion step. By designing primers on sex-linked polymorphisms, the process was simplified. Both approaches are presented.

Methods and Results

Samples were collected from farms in Qatar and from the U.S. Department of Agriculture Agricultural Research Service (“USDA-ARS”) national clonal germplasm repository for citrus and dates in Riverside, Calif., USA. Genomic DNA was extracted from leaves using the WIZARD DNA prep (Promega, Madison, Wis., USA) according to the manufacturer's protocol.

For PCR amplification in the PCR-RFLP assays 1 μL of genomic DNA (15 ng/μL) was amplified using AmpliTaq Gold (Life Technologies, Foster City, Calif., USA) master mix in a total volume of 25 μL containing 5 pmol of each primer. Reactions were activated at 95° C. for 5 min followed by 40 cycles of 95° C. 15 s, 56° C. for 30 s, and 72° C. for 1 min. Digestion was carried out by adding directly to the 25 μL amplified product 19 μL of water, 5 μL of the recommended New England Biolabs (Beverly, Mass., USA) 10× restriction enzyme buffer, and 5 U of the restriction enzyme (see below). The total volume of 50 μL was digested at the temperature recommended by the manufacturer. The PCR-only assay contained 1 μL of genomic DNA (15 ng/μL) with 7.5 μL of 1.5 mM MgCl₂, 0.5 μL of each female primer (5 pmol), and 1 μL of each male primer (5 pmol) in a total reaction volume of 25 μL using AmpliTaq Gold master mix (Life Technologies), Reactions were cycled for 45 cycles using previous conditions.

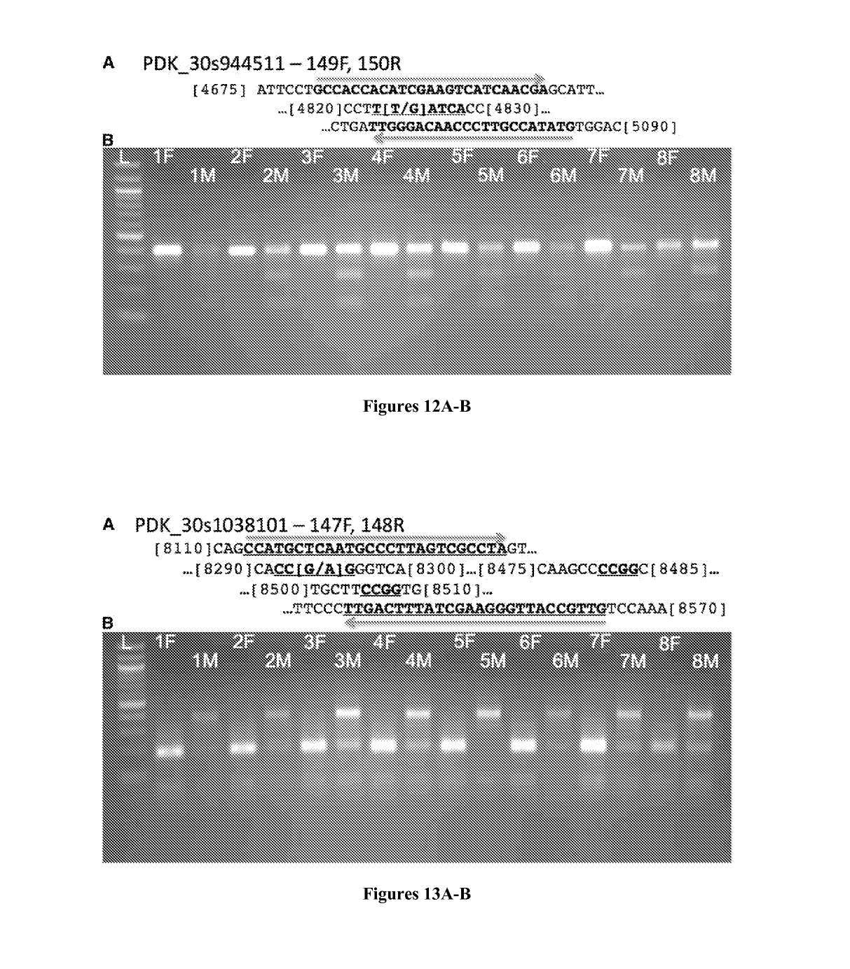

Three PCR-RFLP-based methods were designed and tested on various combinations of male and female date palms. These date palms represented 10 different varieties, as some of the males were a result of backcrossing to tested females. The goal was to test whether the assay was specific enough to distinguish sex between backcrossed males and females while at the same time sensitive enough to distinguish sex in multiple varieties (Table 10). Testing of the three assays revealed that those based on Bc/I (FIGS. 12A-B) and HpaII (FIGS. 13A-B) were capable of distinguishing sex among all samples tested. The assay based on RsaI (FIGS. 14A-B) showed that it called two males as a female (Khalt and Zahidi BC2). Sequencing of the RsaI region in a larger set of date palms revealed that the six out of 25 males were homozygous for the female allele at this polymorphism. This suggests that the RsaI polymorphism is most likely a recent polymorphism that is on a haplotype that has not spread as widely in the population as the others.

TABLE 10
Date Palm Varieties Used in PCR-RFLP Assays.

| Number | Name | Gender | Collection | ID |
|---|---|---|---|---|
| 1F | Khalas | Female | Qatar | Bio1 |
| 2F | Chichi | Female | Qatar | Bio14 |
| 3F | Deglet Noor | Female | USDA-CA | PI 4611-RIV2154 |
| 4F | Medjool | Female | USDA-CA | RPHO1-RIV2134 |
| 5F | Barhee | Female | USDA-CA | PI 8746-RIV7479 |
| 6F | Helali | Female | Qatar | F37 |
| 7F | Saqie | Female | Qatar | F95 |
| 8F | Dayri | Female | USDA-CA | PI 8567-RIV7490 |
| 1M | Khalt | Male | Qatar | M6 |
| 2M | Khadrawy | Male | USDA-CA | PI555435-PL7491 |
| 3M | Medjool BC4 | Male | USDA-CA | PI555438-RIV2219 |
| 4M | Dayri BC2 | Male | USDA-CA | PI555413-RIV7441 |
| 5M | Barhee BC3 | Male/ | USDA-CA | PI555419-RIV2133 |
| | | hermaph- | | |
| | | rodite | | |
| 6M | Deglet Noor | Male | USDA-CA | PI555433-PL2165 |
| | BC5 | | | |
| 7M | Crane | Male | USDA-CA | PI555421-RIV2478 |
| 8M | Zahidi BC2 | Male | USDA-CA | PI555409-RIV2236 |

Note:
BC = backcross;
USDA-CA = USDA-ARS national clonal germplasm repository for citrus and dates.
Date palm genomic DNA used in this project is documented with ID number used in images, variety name, gender, where the sample is maintained, and its collection ID number.

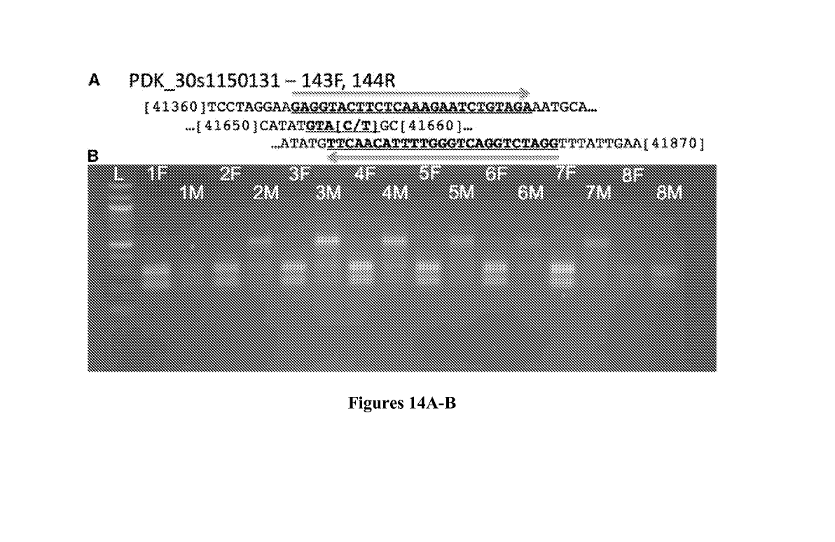

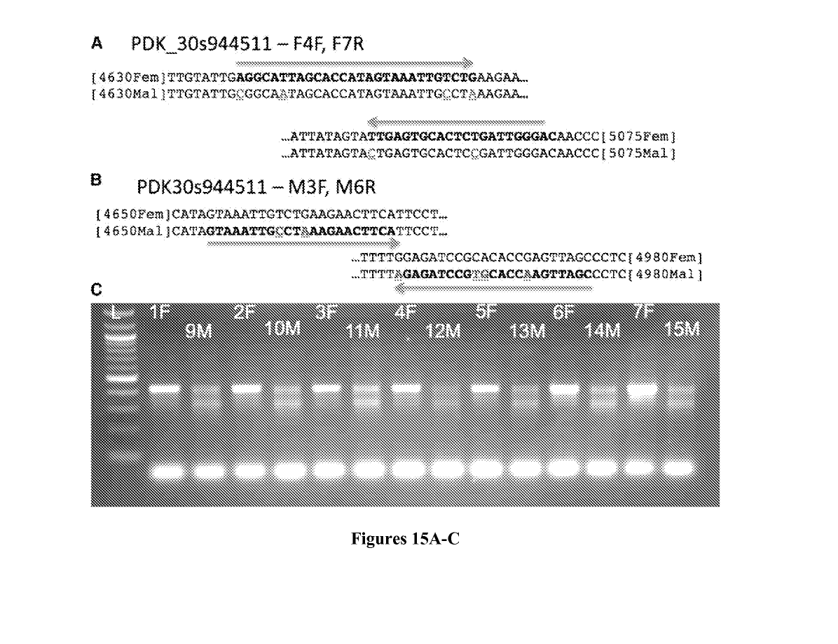

While the PCR-RFLP assay is likely to be quite specific, it was attempted to design an assay that would allow researchers to determine gender of a date palm with a single PCR reaction followed by gel electrophoresis. It was advantageous that the male and female haplotypes are quite diverged with multiple polymorphisms between them. PCR primers were designed to span multiple polymorphisms (FIGS. 15A-B) that were unique to either the male or female haplotype in the hope that would allow specific amplification of each haplotype. The male is heterozygous containing both the male and female alleles and should yield two distinct PCR products. The female is homozygous, which should yield a single band representing both copies of the female allele. Interestingly, in the design of the male allele specific primers, a single polymorphism only found in the Deglet Noor males was encountered (FIG. 15B). This did not seem to affect the ability of the primer to anneal to other males (FIG. 15C). In this case different date palm varieties were used for the validation (Table 11). The female varieties from the previous assays were used to establish the functionality of the assay across varieties. However, multiple backcrossed males were used from three varieties to demonstrate linkage of the allele to sex as would be done in a crossing program. That is to say, all females were homozygous and all male offspring shown here were heterozygous. While the assay showed some issues with primer dimers (FIG. 15C), the results clearly show that a single step, PCR-only-based method distinguishes the male and females.

TABLE 11
Date Palm Varieties Used in the PCR-Only-Based Assay.

| Number | Name | Gender | Collection | ID |
|---|---|---|---|---|
| 1F | | Female | Qatar | Bio1 |
| 2F | Chichi | Female | Qatar | Bio14 |
| 3F | Deglet Noor | Female | USDA-CA | PI 4611-RIV2154 |
| 4F | Medjool | Female | USDA-CA | RPHO1-RIV2134 |
| 5F | Barhee | Female | USDA-CA | PI 8746-RIV7479 |
| 6F | Helali | Female | Qatar | F37 |
| 7F | Saqie | Female | Qatar | F95 |
| 9M | Deglet Noor BC5 | Male | USDA-CA | PI55543-PL7506 |
| 10M | Deglet Noor BC5 | Male | USDA-CA | PI555432-PL2166 |
| 11M | Medjool BC4 | Male | USDA-CA | PI555438-RIV7526 |
| 12M | Medjool BC4 | Male | USDA-CA | PI555438-2220 |
| 13M | Dayri BC3 | Male | USDA-CA | PI555417-RIV2145 |
| 14M | Dayri BC2 | Male | USDA-CA | PI555414-RIV2150 |
| 15M | Dayri BC2 | Male | USDA-CA | PI555413-RIV7442 |

Note:
BC = backcross;
USDA-CA = USDA-ARS national clonal germplasm repository for citrus and dates.

### CONCLUSIONS

Multiple assays have been developed that will allow researchers to distinguish date palm sex at the earliest stages. The assays were shown to work across multiple date palm varieties indicating that most polymorphisms they are based on are widespread and most likely ancient. For sensitivity, the use of the PCR-RFLP approach based on both the BclI and HpaII enzymes is recommended as these offer contrasting results (restriction digestions of male product in one and female product in the other). Due to high heterozygosity in the gender-linked region (Al-Dous et al., “De novo Genome Sequencing and Comparative Genomics of Date Palm (Phoenix dactylifera),” Nature Biotechnology 29:521-527 (2011), which is hereby incorporated by reference in its entirety), a PCR-only method was successfully developed and offers investigators a faster approach, although sensitivity may be reduced. As the sex-linked region is fine mapped and a single sex-controlling mutation is identified, these assays will be modified to take into account this information. For now, it is predicted that one can achieve at least 90% discrimination levels using these approaches.

Although the invention has been described in detail for the purposes of illustration, it is understood that such detail is solely for that purpose, and variations can be made therein by those skilled in the art without departing from the spirit and scope of the invention which is defined by the following claims.

What is claimed is:

1. A method of identifying the sex of a date palm plant, said method comprising:
analyzing DNA or RNA from a date palm plant, tissue, germplasm, or seed for the presence of (i) a nucleic acid sequence that identifies the sex of the plant, tissue, germplasm, or seed or (ii) a molecular marker in linkage disequilibriumwith the nucleic acid sequence and
identifying the sex of the plant, tissue, germplasm, or seed based on whether or not the plant, tissue, germplasm, or seed contains the nucleic acid sequence or the molecular marker.

2. The method according to claim 1, wherein said analyzing is carried out to determine the presence of the nucleic acid sequence.

3. The method according to claim 2, wherein said analyzing comprises determining the presence of a male allele at the nucleotide corresponding to position 51 of SEQ ID NOs:1-972, or a corresponding RNA sequence.

4. The method according to claim 3, wherein where the plant, tissue, germplasm, or seed does not contain the male allele the plant, tissue, germplasm, or seed is identified as female.

5. The method according to claim 1, wherein said analyzing is carried out to determine the presence of the molecular marker.

6. The method according to claim 5, wherein the molecular marker is present in SEQ ID NOs:1-972, or a corresponding RNA sequence.

7. The method according to claim 6, wherein where the plant, tissue, germplasm, or seed contains the molecular marker, the plant, tissue, germplasm, or seed is identified as male.

8. The method according to claim 1, wherein said analyzing comprises detecting, in a hybridization assay, whether the nucleic acid sequence hybridizes to an oligonucleotide probe.

9. The method according to claim 1, wherein said analyzing comprises detecting, in a PCR-based assay, whether oligonucleotide primers amplify the nucleic acid sequence.

10. The method according to claim 1 further comprising:
planting or transplanting the date palm plant, tissue, seed, or germplasm a location suitable for the identified sex.

11. The method according to claim 1 further comprising:
growing a fruit-bearing plant from the plant, tissue, germplasm, or seed and
harvesting fruit from the fruit-bearing plant.

12. The method according to claim 1 further comprising:
breeding the plant whose sex is identified.

13. The method according to claim 1 further comprising:
marking the plant, tissue, seed, or germplasm based on its identified sex.

14. A method of identifying the sex of a date palm plant, said method comprising:
analyzing DNA or RNA from a date palm plant, tissue, germplasm, or seed for the presence of (i) a genotype that identifies the sex of the plant, tissue, germplasm, or seed, or (ii) a molecular marker linked to the genotype and
identifying the sex of the plant, tissue, germplasm, or seed based on whether or not the plant, tissue, germplasm, or seed contains the genotype or the molecular marker.

15. The method according to claim 14, wherein a male genotype is present in the plant, tissue, germplasm, or seed and the plant, tissue, germplasm, or seed is identified as a male plant.

16. The method according to claim 15, wherein the genotype is selected from a heterozygous or homozygous male allele at the nucleotides corresponding to position 51 of SEQ ID NOs:1-972.

17. The method according to claim 14, wherein a molecular marker associated with a male genotype is present in the plant, tissue, germplasm, or seed and the plant, tissue, germplasm, or seed is identified as a male plant.

18. The method according to claim 17, wherein the molecular marker is present in SEQ ID NOs:1-972, or a corresponding RNA sequence.

19. The method according to claim 14, wherein said analyzing is carried out with a hybridization assay or a PCR-based assay.

20. The method according to claim 14 further comprising:
planting or transplanting the date palm plant, tissue, seed, or germplasm in a location suitable for the identified sex.

21. The method according to claim 14 further comprising:
growing a fruit-bearing plant from the plant, tissue, germplasm, or seed and
harvesting fruit from the fruit-bearing plant.

22. The method according to claim 14 further comprising:
breeding the plant whose sex is identified.

23. The method according to claim 14 further comprising:
marking the plant, tissue, seed, or germplasm based on its identified sex.

24. A method of selecting a male or female date palm plant prior to flowering, said method comprising:
detecting in a date palm plant, tissue, germplasm, or seed (i) a genotype that identifies the plant, tissue, germplasm, or seed as male or female, or (ii) a molecular marker in linkage disequilibrium with the genotype and
selecting the plant, tissue, germplasm, or seed possessing the genotype or the molecular marker.

25. The method according to claim 24, wherein a male genotype is detected in the plant, tissue, germplasm, or seed.

26. The method according to claim 25, wherein the genotype is selected from a heterozygous or homozygous male allele at the nucleotides corresponding to position 51 of SEQ ID NOs:1-972.

27. The method according to claim 24, wherein a female genotype is detected in the plant, tissue, germplasm, or seed, said female genotype comprising a homozygous female allele at the nucleotides corresponding to position 51 of SEQ ID NOs:1-972.

28. The method according to claim 24, wherein the molecular marker is detected.

29. The method according to claim 28, wherein the molecular marker is present in SEQ ID NOs:1-972.

30. The method according to claim 24 further comprising:
planting or transplanting the selected date palm plant, tissue, seed, or germplasm in a location suitable for its sex.

31. The method according to claim 24 further comprising:
growing a fruit-bearing plant from the plant, tissue, germplasm, or seed and
harvesting fruit from the fruit-bearing plant.

32. The method according to claim 24 further comprising:
breeding the plant whose sex is identified.

33. The method according to claim 24 further comprising:
marking the selected plant, seed, or germplasm as male or female.

34. A kit for selecting a male or female date palm plant prior to flowering, said kit comprising:
primers or probes for detecting in a date palm plant, tissue, germplasm, or seed (i) a genotype that identifies the plant, tissue, germplasm, or seed as male or female, or (ii) a molecular marker in linkage disequilibrium with the genotype and
instructions for using the primers or probes for detecting the genotype or the molecular marker.

35. The kit according to claim 34, wherein the primers or probes detect the genotype.

36. The kit according to claim 35, wherein the genotype is a heterozygous or homozygous male allele at position 51 of SEQ ID NOs:1-972 for selecting a male date palm plant.

37. The kit according to claim 35, wherein the genotype is a homozygous female allele at the nucleotides corresponding to position 51 of SEQ ID NOs:1-972 for selecting a female date palm plant.

38. The kit according to claim 34, wherein the primers or probes detect the molecular marker.

39. A method of selecting a male or female date palm plant prior to flowering, said method comprising:
detecting in a date palm plant, tissue, germplasm, or seed (i) a nucleic acid sequence that identifies the plant, tissue, germplasm, or seed as male or female or (ii) a molecular marker in linkage disequilibrium with the nucleic acid sequence and
selecting the plant, tissue, germplasm, or seed possessing the nucleic acid sequence or the molecular marker.

40. The method according to claim 39, wherein the nucleic acid sequence is detected.

41. The method according to claim 40, wherein the nucleic acid sequence comprises a male allele at the nucleotide corresponding to position 51 of SEQ NOs:1-972, or a corresponding RNA sequence and the plant, tissue, germplasm, or seed selected is male.

42. The method according to claim 39, wherein the molecular marker is detected.

43. The method according to claim 42, wherein the molecular marker is present in SEQ ID NOs:1-972, or a corresponding RNA sequence.

44. The method according to claim 39 further comprising:
planting or transplanting the selected date palm plant, tissue, seed, or germplasm in a location suitable for its sex.

45. The method according to claim 39 further comprising:
breeding the plant whose sex is identified.

46. The method according to claim 39 further comprising:
marking the selected plant, seed, or germplasm as male or female.

47. A kit for selecting a male or female date palm plant prior to flowering, said kit comprising:
primers or probes for detecting in a date palm plant, tissue, germplasm, or seed (i) a nucleic acid sequence that identifies the plant, tissue, germplasm, or seed as male or female or (ii) a molecular marker in linkage disequilibrium with the nucleic acid sequence and
instructions for using the primers or probes for detecting the nucleic acid sequence or the molecular marker.

48. The kit according to claim 47, wherein the primers or probes detect the nucleic acid sequence.

49. The kit according to claim 48, wherein the nucleic acid sequence detected comprises a male allele at the nucleotide corresponding to position 51 of SEQ ID NOs:1-972, or a corresponding RNA molecule, and the plant, tissue, germplasm, or seed selected is male.

50. The kit according to claim 47, wherein the primers or probes detect the molecular marker.

51. A method of breeding a date palm plant, said method comprising:
providing a date palm plant having a sex determined by detecting in the plant or a seed, tissue, or germplasm from which it was derived (i) a genotype that identifies the plant as either male or female, or (ii) a molecular marker in linkage disequilibrium with the genotype and
breeding the date palm plant with a plant of the opposite sex.

52. A method of breeding a date palm plant, said method comprising:
providing a date palm plant having a sex determined by detecting in the plant or a seed, tissue, or germplasm from which it was derived (i) a nucleic acid sequence that identifies the plant as male or female or (ii) a molecular marker in linkage disequilibrium with the nucleic acid sequence and
breeding the date palm plant with a plant of the opposite sex.

53. A method of planting a date palm seed of a known sex, said method comprising:
providing a seed having a known male or female sex and
planting the seed.


SEQUENCE LISTING (994 sequences)

SEQ ID NO: 1 - DNA, 101 bp, Phoenix dactylifera
gataggaggatgagatccacccctcatgagctgtcaccgacgagcaaggarcgyagactttcaaccctccttccttgtcgggttaagtgaaaaaataggaa

SEQ ID NO: 2 - DNA, 101 bp, Phoenix dactylifera
aggaggatgagatccacccctcatgagctgtcaccgacgagcaaggarcgyagactttcaaccctccttccttgtcgggttaagtgaaaaaataggaaaga

SEQ ID NO: 3 - DNA, 101 bp, Phoenix dactylifera
tggctgccagaaatgggagaatcagtggagaaaaggaaggaaagcagagawggaaacatcctcccctgtttctggtcattacacatacgctttttagtatc

SEQ ID NO: 4 - DNA, 101 bp, Phoenix dactylifera
ttaacttaaaaagggaagcagaggaaagatatcaattaaaaaggaaagctraggaaggaaagataaatggcataacttggggctggtgattttgtatgaca

SEQ ID NO: 5 - DNA, 101 bp, Phoenix dactylifera
ataacttggggctggtgattttgtatgacattagcttctagatatttcctytgcctccyccgcttaaattcctaatggtagctcattgaatagcagaggca

SEQ ID NO: 6 - DNA, 101 bp, Phoenix dactylifera
gggctggtgattttgtatgacattagcttctagatatttcctytgcctccyccgcttaaattcctaatggtagctcattgaatagcagaggcaggagaggc

SEQ ID NO: 7 - DNA, 101 bp, Phoenix dactylifera
ttgtcccctaagcaaaagaatctaatgatacggttcacatgcactggaatwgacaagaagaccagcataaattaaatgagctgaataaacttattcatgca

SEQ ID NO: 8 - DNA, 101 bp, Phoenix dactylifera
gggccttgaggggtgaagattcttctcaagagaaatagacccctctctccrccattatttaaattattttcttcttttacttttctgcagcattgattrtg

SEQ ID NO: 9 - DNA, 101 bp, Phoenix dactylifera
ccrccattatttaaattattttcttcttttacttttctgcagcattgattrtgtggctagagaatgcaggcaaaaygatgctcaactgatggygaatgttc

SEQ ID NO: 10 - DNA, 101 bp, Phoenix dactylifera
cttttacttttctgcagcattgattrtgtggctagagaatgcaggcaaaaygatgctcaactgatggygaatgttcgagagaagaaggtgctgtctctgtt

SEQ ID NO: 11 - DNA, 101 bp, Phoenix dactylifera
cattgattrtgtggctagagaatgcaggcaaaaygatgctcaactgatggygaatgttcgagagaagaaggtgctgtctctgtttgctacagaatttatgc

SEQ ID NO: 12 - DNA, 101 bp, Phoenix dactylifera
cttgggcttgatatatgtttttcaccctttcctcgcaagctcaagtttcgygatctagaggttagttaacatatcatgagatcatcgaatcctcattcgac

SEQ ID NO: 13 - DNA, 101 bp, Phoenix dactylifera
atatcatgagatcatcgaatcctcattcgacccgatcttttgtttgttacygagtacatatcttggttaktaggagtatgrcagtcacaatccttctatta

SEQ ID NO: 14 - DNA, 101 bp, Phoenix dactylifera
tcctcattcgacccgatcttttgtttgttacygagtacatatcttggttaktaggagtatgrcagtcacaatccttctattagtaggtgaatcaatgaggt

SEQ ID NO: 15 - DNA, 101 bp, Phoenix dactylifera
taagttattagaagaagccgttctttcttatttcaagcgtttctaagttgrcaactcttgtgaacaaggactgcttctgyatgtctcccctcccactctat

SEQ ID NO: 16 - DNA, 101 bp, Phoenix dactylifera
atttcaagcgtttctaagttgrcaactcttgtgaacaaggactgcttctgyatgtctcccctcccactctatgaattagaggctccacaaggactagcacg

SEQ ID NO: 17 - DNA, 101 bp, Phoenix dactylifera
ttgttgctagagttgccggcctccaaatactggaaacctgggagagagtcstcctctgcaatgctaactatgggctttaaatccatggaggtcgagacgat

SEQ ID NO: 18 - DNA, 101 bp, Phoenix dactylifera
ggtcgagacgatcggcctggagcctgatggaacagcagtcgatatcatgcygcaatttaactcggaattcatgtcgaacagcctcgaattctgattaaacc

SEQ ID NO: 19 - DNA, 101 bp, Phoenix dactylifera
ttccgaaggggaaagtagtccattgaattggagctctcatggcttgcggcsgcgaactggtctagagagaaatctttggtggatgttacgaaattcttcga

SEQ ID NO: 20 - DNA, 101 bp, Phoenix dactylifera
aatattatcccacaaaaggaaagagaagtcggtatgattaattaaaagaaktgctctttgctatcttgatttttttttcggatgcattcttgttttaggag

SEQ ID NO: 21 - DNA, 101 bp, Phoenix dactylifera
tctcatcaggctctgagataaatgtttgctcctataatggtggggtatctrcyctcgtaaattaatggagaagcagtagcasgattwggtccccatgactt

SEQ ID NO: 22 - DNA, 101 bp, Phoenix dactylifera
tcatcaggctctgagataaatgtttgctcctataatggtggggtatctrcyctcgtaaattaatggagaagcagtagcasgattwggtccccatgacttga

SEQ ID NO: 23 - DNA, 101 bp, Phoenix dactylifera
ctataatggtggggtatctrcyctcgtaaattaatggagaagcagtagcasgattwggtccccatgacttgaaagctaaggcagcaaaaaagtaacattat

SEQ ID NO: 24 - DNA, 101 bp, Phoenix dactylifera
atggtggggtatctrcyctcgtaaattaatggagaagcagtagcasgattwggtccccatgacttgaaagctaaggcagcaaaaaagtaacattatttgaa

SEQ ID NO: 25 - DNA, 101 bp, Phoenix dactylifera
cgaggcccaaaggaaagtcctaacgcccattcgagttcctaagataatcaygtgtcagagaaaactggrcccwttttgctttttccactgatccccaaatc

SEQ ID NO: 26 - DNA, 101 bp, Phoenix dactylifera
cctaacgcccattcgagttcctaagataatcaygtgtcagagaaaactggrcccwttttgctttttccactgatccccaaatccatgatcacgttggarag

SEQ ID NO: 27 - DNA, 101 bp, Phoenix dactylifera
ctagagaaaatccatcaattgttygatccragtttagggggggattatagrgttgatactccatcacacaaaaagggaagttagcatggctttcatctaca

SEQ ID NO: 28 - DNA, 101 bp, Phoenix dactylifera
ggctttcatctacaactctatggtcggagtctgatgcatggagattatagygactgcatggctcaattataaggttcctaaagatagtagttgtctaacat

SEQ ID NO: 29 - DNA, 101 bp, Phoenix dactylifera
aactgaattgtcatcatagtctaattcagagaacttatcgatgctgaatcrtttacgttgtcattaytatattcttctcaccaccccaaactgtgtcagct

SEQ ID NO: 30 - DNA, 101 bp, Phoenix dactylifera
tagtctaattcagagaacttatcgatgctgaatcrtttacgttgtcattaytatattcttctcaccaccccaaactgtgtcagctgtctgtcaactgaaga

SEQ ID NO: 31 - DNA, 101 bp, Phoenix dactylifera
ccgccggaggcgagctttgctgacaaacatcatccagtcatgtgtttggamttaacctgctgactgacataatacagaggtatggatctactgatgttctt

SEQ ID NO: 32 - DNA, 101 bp, Phoenix dactylifera
gactgacataatacagaggtatggatctactgatgttcttaatcctctacyatgttctcatgtatagtaaagcatatgagttgacagaaggatttgaagca

SEQ ID NO: 33 - DNA, 101 bp, Phoenix dactylifera
tagcacaaataaagcagtaaataagatgaggaacacagttagaaaaatacrgtaaaaaagagagacaarccacatacaaacacaaggatttatagtagttc

SEQ ID NO: 34 - DNA, 101 bp, Phoenix dactylifera
aaataagatgaggaacacagttagaaaaatacrgtaaaaaagagagacaarccacatacaaacacaaggatttatagtagttcggtgcattcccaacacct

SEQ ID NO: 35 - DNA, 101 bp, Phoenix dactylifera
aactttacactgtttatttttccaggctcacaaacaacccaattggttttyagcaacttccaattaagacttggaagtgcctttacaagtttcagattatg

SEQ ID NO: 36 - DNA, 101 bp, Phoenix dactylifera
aataaaccaaataaatgattgctgcaactagaaaactaaagcttcataacrtataaaatgaatcaatagaagcaatgctgagggcaatgaagaggttggct

SEQ ID NO: 37 - DNA, 101 bp, Phoenix dactylifera
tcattgtggtcccagctgtataggggtcaactcatatttatccttggtcgrcacttcaaaatattgagacgactcttgctgttgtaacttcaaagggtcga

SEQ ID NO: 38 - DNA, 101 bp, Phoenix dactylifera
tgttgcttatatgggtaacatcctattccatgaaaaaattgagatgccttratcccataggatttaaaattttggtacttggttcattacttgcatgaatg

SEQ ID NO: 39 - DNA, 101 bp, Phoenix dactylifera
ctcggggcattagagagataaaatggcatatagagccactcataaagcttrtcytgtgccttaaacacagtttttcactcatcacctaaaaggattggaat

SEQ ID NO: 40 - DNA, 101 bp, Phoenix dactylifera
ggggcattagagagataaaatggcatatagagccactcataaagcttrtcytgtgccttaaacacagtttttcactcatcacctaaaaggattggaatttg

SEQ ID NO: 41 - DNA, 101 bp, Phoenix dactylifera
ggtacttcatcatgatcttgttgatggtgtgaatatccacacacatctttwggttctatctttctttggagtgatcaaggtaggcacgacacagagactta

SEQ ID NO: 42 - DNA, 101 bp, Phoenix dactylifera
aggtaggcacgacacagagacttatactctcacaaatcaagcctttccaawaagagctcattgacttgcttttgtaactccacatattcctttgatgggat

SEQ ID NO: 43 - DNA, 101 bp, Phoenix dactylifera
aataatattagttcaaagttcatcctcttttttaactttagatgcatgcawcatttaatgcaagagaaggttttggccttgatctgcttcaagcttgatgg

SEQ ID NO: 44 - DNA, 101 bp, Phoenix dactylifera
gaaggttttggccttgatctgcttcaagcttgatggtgacttcatcacttkgtttgtcgttatagatgggttcaccaaccaactctcatctatcaatgcca

SEQ ID NO: 45 - DNA, 101 bp, Phoenix dactylifera
gggttgtcaccttgggcaacagccatgctctttatactcaccatcccattyataacaagrtggactttagtagggagcaccactaaggactcaaatatata

SEQ ID NO: 46 - DNA, 101 bp, Phoenix dactylifera
ccttgggcaacagccatgctctttatactcaccatcccattyataacaagrtggactttagtagggagcaccactaaggactcaaatatataattccttgg

SEQ ID NO: 47 - DNA, 101 bp, Phoenix dactylifera
ggctagcttgtggtctccttccaatgctagaataggcaagttgtagtgtcytaaaggaggaagggtagcacatgttatggcacatacaaaggtggatccct

SEQ ID NO: 48 - DNA, 101 bp, Phoenix dactylifera
gcttactcgagcactagatattctcccaccatatatatatacatgccctcrtaatttgaaggccacaatctccactttcttattatcaaagatgtctatag

SEQ ID NO: 49 - DNA, 101 bp, Phoenix dactylifera
tccactttcttattatcaaagatgtctatagcttcaaaaaatcttttgatrgtggtgatatagatgagaaagtttgcatatttcagcctcctataaaactc

SEQ ID NO: 50 - DNA, 101 bp, Phoenix dactylifera
agatgagaaagtttgcatatttcagcctcctataaaactcagataggttgytttgatatggaagagcctttcatgatcttatgagactctttcatcaagca

SEQ ID NO: 51 - DNA, 101 bp, Phoenix dactylifera
gtactgagatgttgtgagtattttagtgaaccatagcttcaatattaatgyagaatacaacaagcaaatcatgctcactctaatatcaaaatagtgcacat

SEQ ID NO: 52 - DNA, 101 bp, Phoenix dactylifera
gtgaccctcaccatatattaaagttatctttcaagcctgaaggttgggttkgttggttaagtttgtaatttggaattattcttatggatgtcagccacatc

SEQ ID NO: 53 - DNA, 101 bp, Phoenix dactylifera
tgaatacccgtcttattaatttgtaatgagcttttcctttctgactttacyttctgatagtttgtttcttcatttgcttccagtttttaggtgaggcctta

SEQ ID NO: 54 - DNA, 101 bp, Phoenix dactylifera
atatgtaccagacgtctttcagaggtaaaattgaaagccaaaaaccagccrgtatatgtttaagcttgataagaatacttatactctccttgtaactgttc

SEQ ID NO: 55 - DNA, 101 bp, Phoenix dactylifera
gcattagtcatgaatcctgaaatgttgagaatactggaaaagggtcctatrgtatgcaaacattattacaggaatgacaatattggaactataaaaaargt

SEQ ID NO: 56 - DNA, 101 bp, Phoenix dactylifera
atrgtatgcaaacattattacaggaatgacaatattggaactataaaaaargttcatgcgtggcagttgaatttacaaagatgtctgatatacctaattgg

SEQ ID NO: 57 - DNA, 101 bp, Phoenix dactylifera
ttcagtctgatctaaaattggatttgtaattgtaaggttttaatctttgtrtctattttaccttaattggtactggaagtgaagatcatcgagttaagaat

SEQ ID NO: 58 - DNA, 101 bp, Phoenix dactylifera
tccaaatttatgagggttgaagttctgacatgcctgagcattctaaatttkaagttatatcagttaaacttctgctgcaacaacttgttgcttggggttta

SEQ ID NO: 59 - DNA, 101 bp, Phoenix dactylifera
tccacatgcataacctggcttgctttcagatgtgggaaaataaagggtaawctagaataggaattgctgttgatgtatggcctaattctttttcttggctt

SEQ ID NO: 60 - DNA, 101 bp, Phoenix dactylifera
ctgttgatgtatggcctaattctttttcttggcttcagtagttttagaaayatataattcactgatggactttcattagtctaaacaaaatttatttgtta

SEQ ID NO: 61 - DNA, 101 bp, Phoenix dactylifera
atataattcactgatggactttcattagtctaaacaaaatttatttgttawttatttttcagtagaaaaagtttwtaggaaatgggctacayggtgtgggt

SEQ ID NO: 62 - DNA, 101 bp, Phoenix dactylifera
ttagtctaaacaaaatttatttgttawttatttttcagtagaaaaagtttwtaggaaatgggctacayggtgtgggtagcccttgtccaaatacttgtaac

SEQ ID NO: 63 - DNA, 101 bp, Phoenix dactylifera
tatttgttawttatttttcagtagaaaaagtttwtaggaaatgggctacayggtgtgggtagcccttgtccaaatacttgtaacctctcaaacacttggtg

SEQ ID NO: 64 - DNA, 101 bp, Phoenix dactylifera
aatacaaggcattgtcttgcccaaggttcgccgtttcggtaccagactccrtacaggtaggcatcttattatagtgttggtatgcggtatggtatgagacg

SEQ ID NO: 65 - DNA, 101 bp, Phoenix dactylifera
caggtaggcatcttattatagtgttggtatgcggtatggtatgagacggtraggcgtaccgagtgttggcacggtacgagactgcgtaccggttcggtact

SEQ ID NO: 66 - DNA, 101 bp, Phoenix dactylifera
ttaaaaggggccttctgtagattgctgtttcatgtattgaaaccagtggayatcttggtgcagtacttaccatcgtgtgatggggaatgcaatattacaaa

SEQ ID NO: 67 - DNA, 101 bp, Phoenix dactylifera
caaaatgttaatctcttcattgtttgtagtgttagaatctccactaggaarttatgagaaatacctatcgacattggtgacagccttgcattggggcaaag

SEQ ID NO: 68 - DNA, 101 bp, Phoenix dactylifera
attggtgacagccttgcattggggcaaagcaaacagcagaagttgtatggmtgccccttatcctgcattttttctttatttgtatttttgtaagttttact

SEQ ID NO: 69 - DNA, 101 bp, Phoenix dactylifera
gtaactttattctaggtttttcagtataaggggtttgacacctctcacgtygttgctggatcattgtcaagcattgctaaagtttaagagaaattcatacg

SEQ ID NO: 70 - DNA, 101 bp, Phoenix dactylifera
caatgacaacactttgatgttaaaatgcagtttcgtgggaagattatttcrtcatgcactggaagattcgagacctaaatctgtgctggtgcactcattgt

SEQ ID NO: 71 - DNA, 101 bp, Phoenix dactylifera
atcaagaaattatttagtgtgtcctcaatttagaaattgtgttggtgatgrgttcacattattggactgcaataaattctcatccatcaagccaacccaag

SEQ ID NO: 72 - DNA, 101 bp, Phoenix dactylifera
agtagatggttttaatgcttgctggttaaggtttatgccatctaaaatgtrggttaggaagaccaatgtcgagtgaaattaacaaacttgtggatcagatc

SEQ ID NO: 73 - DNA, 101 bp, Phoenix dactylifera
cgtgactaacggagcctcaacctaaaaataataatagaaagatattatttraatttgattttatataaataactaaaatttatttattgtatagttgatat

SEQ ID NO: 74 - DNA, 101 bp, Phoenix dactylifera
tgaaccgcttctgcattggctatagctagatcaaactatataatttgtcayaactcaagattttatttaaaaacgacgaggatcgaaaggtattatttaat

SEQ ID NO: 75 - DNA, 101 bp, Phoenix dactylifera
ataccaaatggtgcatattgtaatggttagtgcacggtaacacatctagcytttttatgtttaagatctaattccatacatatcatacaaaagtatataaa

SEQ ID NO: 76 - DNA, 101 bp, Phoenix dactylifera
aaaaaaaccctaaagatgactttcatgcaagcgtaagattttcatccttcstaatcttatccctatgcatagaacctagagatgagtttgcatatttccta

SEQ ID NO: 77 - DNA, 101 bp, Phoenix dactylifera
ttagagagggtctggaaacatacttttgagcgaaagctagtaccttttctrtcaattgcatggttcccctcccatgcgargtgccgccggagagtcgggga

SEQ ID NO: 78 - DNA, 101 bp, Phoenix dactylifera
gcgaaagctagtaccttttctrtcaattgcatggttcccctcccatgcgargtgccgccggagagtcggggaaggagctgggctcgtacaatctaccgcag

SEQ ID NO: 79 - DNA, 101 bp, Phoenix dactylifera
attagaaatcgattcaatgatttgtttacgagtggcaaacaaacacttaayrgctcaagttagccaagaagaattcataaagccaaccctagaaagatggc

SEQ ID NO: 80 - DNA, 101 bp, Phoenix dactylifera
ttagaaatcgattcaatgatttgtttacgagtggcaaacaaacacttaayrgctcaagttagccaagaagaattcataaagccaaccctagaaagatggca

SEQ ID NO: 81 - DNA, 101 bp, Phoenix dactylifera
tgtggaatttctgaggtatgtctatgtcggggataccatacaacagagggmtcacagtaaagctttaaaagagagcagccaggttgaagatgttttcaggg

SEQ ID NO: 82 - DNA, 101 bp, Phoenix dactylifera
ctgtcattggaaagtacaagcttagcatcctttgcagatgcttcaattttrttgttcttaaggtggtgactaagtgcaaagccaactattttatccacatc

SEQ ID NO: 83 - DNA, 101 bp, Phoenix dactylifera
ccactttcgtccagctttcctagatcatgccatcatctcaattaaaatctsaaataaatcagatttcaaatgattttcatagtatctgcaagataaattta

SEQ ID NO: 84 - DNA, 101 bp, Phoenix dactylifera
gttaaattcaaattcaaggagtcccatgcaatgtgcgatgtctaacattakaatactaaactggttgcatttgatcaatcaccagaggtgggaccagagac

SEQ ID NO: 85 - DNA, 101 bp, Phoenix dactylifera
gctgctacatgaggagatcaagccatttatagacctcaatattctgcgaayagaaataattttctgtcgcacaactctcagttaaaacaatggtgtcaacc

SEQ ID NO: 86 - DNA, 101 bp, Phoenix dactylifera
agtttatattttatatatagaaggagaaccaaattagaaaaaatagatttrgcggacaaagagattttggccacttgaataaagaagtggtttcaatattt

SEQ ID NO: 87 - DNA, 101 bp, Phoenix dactylifera
tacaaagcgttaagctagtccaatttatgccaagctctcccaaaaataatwagrcaatagtcaaataatcaagaattgcaaatcctccatactcttctgca

SEQ ID NO: 88 - DNA, 101 bp, Phoenix dactylifera
aaagcgttaagctagtccaatttatgccaagctctcccaaaaataatwagrcaatagtcaaataatcaagaattgcaaatcctccatactcttctgcacag

SEQ ID NO: 89 - DNA, 101 bp, Phoenix dactylifera
agatcactttcatcttacttccaagtgtgcctatgcaactaacatgtttcygtaatttccagataaaatwgggaggaaagaaagtgggcatgtgaccagcg

SEQ ID NO: 90 - DNA, 101 bp, Phoenix dactylifera
tccaagtgtgcctatgcaactaacatgtttcygtaatttccagataaaatwgggaggaaagaaagtgggcatgtgaccagcggtattcataagtgagatta

SEQ ID NO: 91 - DNA, 101 bp, Phoenix dactylifera
aacaaactgtttcagattcaaaatggaagaactaaaacattttagaaaatmtgattgatagactgcttctcctcctttctgacataccaatataccttgca

SEQ ID NO: 92 - DNA, 101 bp, Phoenix dactylifera
tacaaagatactcaagaaattttttttcagaaagcttcatgattaattggratgttaatcgtgtccaacaataccwttagcggtgttcttgttttccttta

SEQ ID NO: 93 - DNA, 101 bp, Phoenix dactylifera
ttcagaaagcttcatgattaattggratgttaatcgtgtccaacaataccwttagcggtgttcttgttttcctttaatttcccattttttgaggagaytta

SEQ ID NO: 94 - DNA, 101 bp, Phoenix dactylifera
accwttagcggtgttcttgttttcctttaatttcccattttttgaggagayttaggcaaaaaaacgctaaattaaatatggaatactgtattttataaata

SEQ ID NO: 95 - DNA, 101 bp, Phoenix dactylifera
caaattttacaaactaagtaatttagcaatttaaagagaggaaatattacrgttttaagatacaagagatgggaggcatagggtagttataagtgtcaata

SEQ ID NO: 96 - DNA, 101 bp, Phoenix dactylifera
ctcacgaacatctagtatggcaatctttgatcaccttaaaatactattctwttctcagagctataattctgaacaagaaacgaaataaaaggcagaagcaa

SEQ ID NO: 97 - DNA, 101 bp, Phoenix dactylifera
ggcccaaatttggccaggaaaggcccataggctatgttttggtcgcatagyatgaacatagtgactggtttttttttcagaatttatgaaatataacgtat

SEQ ID NO: 98 - DNA, 101 bp, Phoenix dactylifera
ctacaagcaatcaattccatatgtacctttcttggacttctcttttatttrtttaytaattactatcattatataataattctaagaaaatagagaagtta

SEQ ID NO: 99 - DNA, 101 bp, Phoenix dactylifera
agcaatcaattccatatgtacctttcttggacttctcttttatttrtttaytaattactatcattatataataattctaagaaaatagagaagttagagtt

SEQ ID NO: 100 - DNA, 101 bp, Phoenix dactylifera
tttgttttttttttttatttccattctttaaaagaaaaatggagactgcarccatttgctttgtcaagcacaaaaacgagattctaagatcgccatggatc

SEQ ID NO: 101 - DNA, 101 bp, Phoenix dactylifera
caatggtaaggttgcttcattataacttaggtgggttcaaaacatggaaasaacttctctatatacgggggtaaggttgtatacatttggccatcctcaga

SEQ ID NO: 102 - DNA, 101 bp, Phoenix dactylifera
aaaatgaatgtggagagtgataaacatatttccaatacaaaggcaaaattrtagttttagcccatgagcattctttggttgatgatgatcattgttgttat

SEQ ID NO: 103 - DNA, 101 bp, Phoenix dactylifera
tgttgttatatctcctggattcatggtacattccgttgctttacctttctrttggccaaaatctcctccatttgttctataattttggactaaaaatgagt

SEQ ID NO: 104 - DNA, 101 bp, Phoenix dactylifera
tccatttgttctataattttggactaaaaatgagttagatttaatctgacygaatctatttccatatctgaagtgtatcagattcgaacacaaattttgat

SEQ ID NO: 105 - DNA, 101 bp, Phoenix dactylifera
ccttggaccctgcctaatagagaaacatcactagatatggtgcacatactycttggctctcgctacttctcagtgcaattttggtctctcaaagatataac

SEQ ID NO: 106 - DNA, 101 bp, Phoenix dactylifera
gttctcttatctacctcttactccttcaataacctagcatccaccaatgcyacctgcacaatcaatgccaccctacccccagcgctagcgccatcgcccgc

SEQ ID NO: 107 - DNA, 101 bp, Phoenix dactylifera
ccccagcgctagcgccatcgcccgccatcccttaatcccctccttcgatcmggacttcatcttttgtttcatggtcaccttgctatctataccatcaccat

SEQ ID NO: 108 - DNA, 101 bp, Phoenix dactylifera
gcttttccaattttgagattgagctttatagtaatgtcaatcttacactayatctttctctccaaagaaacctcttaaaacccctccaagatcccattttt

SEQ ID NO: 109 - DNA, 101 bp, Phoenix dactylifera
taagcttgaaaagaaaatttcccagatttgctacaagaaaattatatgtarataagcatcactagaacatattctgacaactgtagatagcttaaacaaca

SEQ ID NO: 110 - DNA, 101 bp, Phoenix dactylifera
aaaacataaataccaccatttgcccttatacttgaatagatgctaccaaartcctagtttcccatcatgtgatctagtctagcatgaaggttgggtacccg

SEQ ID NO: 111 - DNA, 101 bp, Phoenix dactylifera
atattagttataatacatataaaagtttcaatacgaggttgcaagatcaarctacggcaacaaagattgtgatataagggggtgacgaggtatcataacaa

SEQ ID NO: 112 - DNA, 101 bp, Phoenix dactylifera
aaaaaaaaagagaaattttccatgctgaattccaaggttatcactggccaragatggcatcacattttcagcataaaaatacattttcaagttatcactga

SEQ ID NO: 113 - DNA, 101 bp, Phoenix dactylifera
atagttaattattaaaatgtaaaaatacaccagcatccgatcaaatttgawctgtttcaataataaaaacatcagcataatcaaagcattcaagggcatta

SEQ ID NO: 114 - DNA, 101 bp, Phoenix dactylifera
ttgcagcatatgtcatagcccatgagcatgataaagacaacagggttttargtcgcactgacatgtcatacacaaaaagcatggacaaagcaacatattag

SEQ ID NO: 115 - DNA, 101 bp, Phoenix dactylifera
gtcgcattactactaacagcaaccgctaggttccaggtaaccagtgctaaraaaaaaaagtgaaataaaaaatgttacttcattatttgccaataatataa

SEQ ID NO: 116 - DNA, 101 bp, Phoenix dactylifera
aattgattttctcaatcattagtccttacaatgctgactccagatacttgrtggaagcatatttacaggtcagcacccatatatcatctttatcctaaaca

SEQ ID NO: 117 - DNA, 101 bp, Phoenix dactylifera
ctttcttcacaataatggtgagataactgctgaatcacggtcctaagaacmaaattcgttggaataagttcctacaataacatgttttaaaatgaataaac

SEQ ID NO: 118 - DNA, 101 bp, Phoenix dactylifera
acatccaaagtctagaatacacaatatcaataagtctatttgtgtgcaaarggagaaaataccaaaggatcaaaaaatttgtgtccgttcagagttytttt

SEQ ID NO: 119 - DNA, 101 bp, Phoenix dactylifera
caaarggagaaaataccaaaggatcaaaaaatttgtgtccgttcagagttyttttccctgaattggataacttttcaagatgtacgatgtagcatttagcc

SEQ ID NO: 120 - DNA, 101 bp, Phoenix dactylifera
tttagaaacttttgaccaagctttatcttccaatccaaggggagttaggcracatgatagaaggaaaagttgatatagaagttgagatccatctctaagaa

SEQ ID NO: 121 - DNA, 101 bp, Phoenix dactylifera
aattcatttgcactccatacggaaatctagaagccatcaatgtctcttgcrgtgtaagcttattaagccacttgcaaggttatcacgcctctttcagcgtc

SEQ ID NO: 122 - DNA, 101 bp, Phoenix dactylifera
ttatttgagtggctcctcaagtggctgtgagcgcaactggtttaccttcgycctcatccacaacaaacagaggaacyatttgacacagaagcgtcgcaacg

SEQ ID NO: 123 - DNA, 101 bp, Phoenix dactylifera
gtgagcgcaactggtttaccttcgycctcatccacaacaaacagaggaacyatttgacacagaagcgtcgcaacgaccttgtatatgttcactacaatctg

SEQ ID NO: 124 - DNA, 101 bp, Phoenix dactylifera
cgccttcacgaccagccaactttgtagccagcgaggctagggtcgatgcarggcaataggctgaacgcaatatgtcattcattcctacagctgatcagcca

SEQ ID NO: 125 - DNA, 101 bp, Phoenix dactylifera
tcattcattcctacagctgatcagccacaaccacaggagagccagtaaccrcatgactccttatctaaaacatcctcttagagatataatcaagagatgat

SEQ ID NO: 126 - DNA, 101 bp, Phoenix dactylifera
gggtaagaaggcagctacccagcacaccaaaaaagaaaaacaaaaagcacstgcaacctcaatgaagagtgtcaagaagttgtcctrgagcagcagcgata

SEQ ID NO: 127 - DNA, 101 bp, Phoenix dactylifera
aaaacaaaaagcacstgcaacctcaatgaagagtgtcaagaagttgtcctrgagcagcagcgataacagttttggtggtcataaatctgctggccacagag

SEQ ID NO: 128 - DNA, 101 bp, Phoenix dactylifera
ttctcgaggtcattcaaaaggatacgatatagttgcagatgacctcgcacrtggagtaggatccaaagacgtatcaggttccgartcgtatactggattct

SEQ ID NO: 129 - DNA, 101 bp, Phoenix dactylifera
gcagatgacctcgcacrtggagtaggatccaaagacgtatcaggttccgartcgtatactggattctattatccgtagccacagacaatctatgacccata

SEQ ID NO: 130 - DNA, 101 bp, Phoenix dactylifera
tatccgtagccacagacaatctatgacccatacggatacgaatatagatarggtgcatctgagacatcttccagtrgctactatcctacgcagcccagagc

SEQ ID NO: 131 - DNA, 101 bp, Phoenix dactylifera
acccatacggatacgaatatagatarggtgcatctgagacatcttccagtrgctactatcctacgcagcccagagcatctcacagatcaaattttgctgcc

SEQ ID NO: 132 - DNA, 101 bp, Phoenix dactylifera
cacagtcgtattatcaaccagaggacacctctcaaagccagagcttcagcraaaaatccgaaagtacttataacacaacgcgtgtttcttataggatgaac

SEQ ID NO: 133 - DNA, 101 bp, Phoenix dactylifera
cttggttggaggggtaggttgatgtgccttctgattatactgataatccaratatctatgagaggcatcgccgctcgaccccattttagatatcagtatgt

SEQ ID NO: 134 - DNA, 101 bp, Phoenix dactylifera
accccattttagatatcagtatgtatattgaactttaaaagtatgtaattrattgtatcttgatttaagaatattttatgttgtttattttataatttttt

SEQ ID NO: 135 - DNA, 101 bp, Phoenix dactylifera
cttcaagctcacatcctttccttttttcatgttgtatagagaagcaagatwcttagatctaatgcggtcttttggttgcttgttagatggactgattatta

SEQ ID NO: 136 - DNA, 101 bp, Phoenix dactylifera
ctcacacaactagcttatatggctgggttagggcgtgaacaattagcatastgagcctttaggaagttactttcatcatatcacaaactaacctcaccaaa

SEQ ID NO: 137 - DNA, 101 bp, Phoenix dactylifera
gcaatgtgcacaagagtgcaagacacttgcatttcacaagaggcaaacaaygaatctacaaattatccaagaacatccatccttgcagggtaattatataa

SEQ ID NO: 138 - DNA, 101 bp, Phoenix dactylifera
aagtaaaaatcaaatgaaaaagaatcagatgatgctcataatatttaattstataatggttattttcctttaagtgtcacaagtcacaaccaattttcaca

SEQ ID NO: 139 - DNA, 101 bp, Phoenix dactylifera
caaagcttgcaaaatttaatttaacgaagagaaaatgattccgacatttaragaaatgaatgtcacataaaagataaataaacatcccatatgcacattga

SEQ ID NO: 140 - DNA, 101 bp, Phoenix dactylifera
gccccgctgtcttagtattggaagacccatttagcaaaccaactggtcctractagtaccagttgagatggcggcaaacagctcaaacgtcctggatcatg

SEQ ID NO: 141 - DNA, 101 bp, Phoenix dactylifera
gagcaacctggtatctcaggagtgtcccaggtatcattctctcaatggatstgtcattaccatcggaaatggaatccytggagaaaactatttaacagata

SEQ ID NO: 142 - DNA, 101 bp, Phoenix dactylifera
caggtatcattctctcaatggatstgtcattaccatcggaaatggaatccytggagaaaactatttaacagatactacaaaaggagctagaccctcctgat

SEQ ID NO: 143 - DNA, 101 bp, Phoenix dactylifera
gtggaacaccggaatagctctattattacatatgcttccatgtatcactargaccaaaaattcaaggttttggcatctagtagcaatctccaatcacatgc

SEQ ID NO: 144 - DNA, 101 bp, Phoenix dactylifera
cacatgcaatcaaatctcattttcaatctaatcccctgttacaaattcacyaagtcagagttaaaggaatgatggaccattgatcatgtcattcagatcaa

SEQ ID NO: 145 - DNA, 101 bp, Phoenix dactylifera
aaccttcattgctacagcattgcttgagcaagccaaaaacatttttatcawacatgtatggtggctaaaataagccaagtttggttgtttaagctaaggtt

SEQ ID NO: 146 - DNA, 101 bp, Phoenix dactylifera
tgactcgaatatcaggcaataaagagggatcagaaaaacaagttaagcatwtcaaaaataagaacattaatatggatttctggatgaactagaaagaataa

SEQ ID NO: 147 - DNA, 101 bp, Phoenix dactylifera
gaattgattgatatagtatggcatgtacaacagacacctgagggggctccrgttaaaaaaaaatggacattttttgttgaaagatgtaggagagaacgaca

SEQ ID NO: 148 - DNA, 101 bp, Phoenix dactylifera
gcttcaaattagatcaaagctggacctaaacaggaattcttaggaaatgargatccatcaagctgggccaaaatagttagaataaggyttaatgtttatgg

SEQ ID NO: 149 - DNA, 101 bp, Phoenix dactylifera
tcttaggaaatgargatccatcaagctgggccaaaatagttagaataaggyttaatgtttatggttattgatatggttatgggctgatctcagctacaggt

SEQ ID NO: 150 - DNA, 101 bp, Phoenix dactylifera
acatattacccgagcctaaagtgcaacttatgcttggcccattctataagygaacaaattaraacaggcccattatcaagcaagcctaccctactcataca

SEQ ID NO: 151 - DNA, 101 bp, Phoenix dactylifera
gagcctaaagtgcaacttatgcttggcccattctataagygaacaaattaraacaggcccattatcaagcaagcctaccctactcatacatcagtcaattt

SEQ ID NO: 152 - DNA, 101 bp, Phoenix dactylifera
ctcatacagcctaaaatctagttaataatgctccaaacgaacaagcgatayggtgaaaaattagacgagaatgaaaacccaytcaaggtgcygcttaagca

SEQ ID NO: 153 - DNA, 101 bp, Phoenix dactylifera
tccaaacgaacaagcgatayggtgaaaaattagacgagaatgaaaacccaytcaaggtgcygcttaagcatatgcagttatgcactaaagctaattaggca

SEQ ID NO: 154 - DNA, 101 bp, Phoenix dactylifera
caagcgatayggtgaaaaattagacgagaatgaaaacccaytcaaggtgcygcttaagcatatgcagttatgcactaaagctaattaggcaattcagtcac

SEQ ID NO: 155 - DNA, 101 bp, Phoenix dactylifera
attgtacccaaaagggaaatgtcatccaagtatcaatatcacttgttcctyacattacttatattcatttacttttgtaccaccaaagtttccaagagcct

SEQ ID NO: 156 - DNA, 101 bp, Phoenix dactylifera
accagcccagtgtgcttatgagtgcaaatctggacttattacaactggaasgaaaatttcaacattttataatataaaaaaacttagatacgtagaatctg

SEQ ID NO: 157 - DNA, 101 bp, Phoenix dactylifera
aagttgtcctacatagtagattatgagatttggaaacagtatcatgccgayactcctgagcacatgatggcaatcatgaatcttaaaatctgtcatgtatg

SEQ ID NO: 158 - DNA, 101 bp, Phoenix dactylifera
gaatgagtttcaacttgataacagaccaatatcacatcctgtgatacaackttttcttcgcagatattaaaaaagaaacatatttaaatgctcaaaataag

SEQ ID NO: 159 - DNA, 101 bp, Phoenix dactylifera
gttaggatggatggtgggatcatcaaggagttcaaggatagatgtggaagyctaaaacattttgaaaagaaaaccaaatgggtaatttcccttgggcatga

SEQ ID NO: 160 - DNA, 101 bp, Phoenix dactylifera
tcgatagtagaagaatgaggtagaagagaggaaaggatatgaaaagcaagrgaagagttttatttttgttctttctttcggagatgggggcatgacataga

SEQ ID NO: 161 - DNA, 101 bp, Phoenix dactylifera
gtagcatatattttattaggtgctttatatattattacaaggtaataatawatttaaacttcaagcaatacataagatggactagaggtggccaatatagg

SEQ ID NO: 162 - DNA, 101 bp, Phoenix dactylifera
agacacaagaaagattgaagctgatgcaaagctctgaagacactacaaccrtcaaaattttgtttcggagcatgtaaactgcatgttaggtgtgggcctaa

SEQ ID NO: 163 - DNA, 101 bp, Phoenix dactylifera
accaataaaatttgtcttttacagttgtcaaagaataaacagtaaagaacygatgcaacatattaaactatgtcagaaaaaccaatgataacaatgacaaa

SEQ ID NO: 164 - DNA, 101 bp, Phoenix dactylifera
ttcaagagtccctcgagcactgacaattcaagaaataaacaatatatcatratgccttcatttaacatagggatcctagtttaggctattgtcagcctata

SEQ ID NO: 165 - DNA, 101 bp, Phoenix dactylifera
cattatccaagatttgtggaggttgtcttaacattattagataattctgcsccatgaaaattcattgttgctaaaccataccttaggctttcatagttcct

SEQ ID NO: 166 - DNA, 101 bp, Phoenix dactylifera
agttcctttcatcagtaccaattaaatgtagcatgcaaagttcaatatgcyttaaccaaatcaacacatgcatctcaaaggctacaaaaccgcmaacaaat

SEQ ID NO: 167 - DNA, 101 bp, Phoenix dactylifera
aatatgcyttaaccaaatcaacacatgcatctcaaaggctacaaaaccgcmaacaaattctcaagcctagacttaggatgaagctataaccataggattga

SEQ ID NO: 168 - DNA, 101 bp, Phoenix dactylifera
atggctctagatgcccatatacctgtgacgttaccttctcagaccaataartcatctgcattaccttaaccagcagctgttatcccatttccatccttcag

SEQ ID NO: 169 - DNA, 101 bp, Phoenix dactylifera
ttcctactttattagctattcaaaagcttgtgcaaacatcaagcaaccttragtggttcctgacatgcttagaaaaattttcaagcccctaaatttacatt

SEQ ID NO: 170 - DNA, 101 bp, Phoenix dactylifera
aatttacattggcaactagttacttctcacaacctccatataaaggaaggkttggagaagaaaagaaatggattctttccttcttcaaaaatcaatcaaaa

SEQ ID NO: 171 - DNA, 101 bp, Phoenix dactylifera
gcatcaatgtgcagttgtggcattacctaaattctagggtgattgtagtckaasgatggaccagaccaagtgtgaaattgggtaacttgagtttcagttgg

SEQ ID NO: 172 - DNA, 101 bp, Phoenix dactylifera
tcaatgtgcagttgtggcattacctaaattctagggtgattgtagtckaasgatggaccagaccaagtgtgaaattgggtaacttgagtttcagttggcat

SEQ ID NO: 173 - DNA, 101 bp, Phoenix dactylifera
gccaagaagttccccttgcactgccaaaacaaaattataaaaactaggttrgatccataacaagcagtatcgagctacatcaacccatgccaaaccagcat

SEQ ID NO: 174 - DNA, 101 bp, Phoenix dactylifera
aatacctcagtctctctctcttgttagggctctctctcccatcaacggtcrgccatcatgactgtgactacaccccgtccctagatgtgactattgctcaa

SEQ ID NO: 175 - DNA, 101 bp, Phoenix dactylifera
actacaccccgtccctagatgtgactattgctcaatcctcccaaaccatcrgccattgaccgtccgtgggcatctcccatcgcgatcgatcaccataattc

SEQ ID NO: 176 - DNA, 101 bp, Phoenix dactylifera
ttctgaaatgactggatcgataagttaaggcttcagagtaggattttccgracttctcttgttgtaatatttggcgtaaaacttttttaagttgctctctg

SEQ ID NO: 177 - DNA, 101 bp, Phoenix dactylifera
tcactttactagtcgaccgcttagtttaccggaaattggtgattggtttasttagaaatcctagaatgccacttaaaaatccgatctagacccgacccgga

SEQ ID NO: 178 - DNA, 101 bp, Phoenix dactylifera
ttaagaaagtgaagcagaactacaatcctgaagcttcatttgggaaaaatrtaggtattatcacaaacataatgaaccaaattttaattcatccaataaca

SEQ ID NO: 179 - DNA, 101 bp, Phoenix dactylifera
acataatgaaccaaattttaattcatccaataacaaaatttttgcactagsaccaaagttgttaaccaatgtagcaccataatcgacatctcgatcaaaat

SEQ ID NO: 180 - DNA, 101 bp, Phoenix dactylifera
ttttttttcccttttaaagaaggaaaataaaaaatgacaaagaacttcaayagagcaagatatacagagamcaaggtaaatcaggatagacattcactata

SEQ ID NO: 181 - DNA, 101 bp, Phoenix dactylifera
aggaaaataaaaaatgacaaagaacttcaayagagcaagatatacagagamcaaggtaaatcaggatagacattcactatagcaataaaaaaataaatatg

SEQ ID NO: 182 - DNA, 101 bp, Phoenix dactylifera
taggactaaacacacgcccattcaaatctaatcacaagcactacgatcagmtcgtggtcaggacctcacccaaaaagactagccagaaagggtattattta

SEQ ID NO: 183 - DNA, 101 bp, Phoenix dactylifera
agggtattatttaggttccttagttctgtataagtacccaagatctcctcrgcaaataaccgatatgggactaaacacacgccgggtcctcacaaaataga

SEQ ID NO: 184 - DNA, 101 bp, Phoenix dactylifera
cgtggcagctcctcgcttgctgtggtatttgaggaacctggatcgaccgtycctcatggttggaccgggatgggatggacttctctcttgggaaccggttc

SEQ ID NO: 185 - DNA, 101 bp, Phoenix dactylifera
tctgtaaaactcggacctgttattccatctaacctgtatttttttaagagyctatccaaatccagcacagaccatagcttttgggatgcatgaacaaaaga

SEQ ID NO: 186 - DNA, 101 bp, Phoenix dactylifera
cagcacagaccatagcttttgggatgcatgaacaaaagatatagggtggtmtttttttcacggaatttgggccggatagtatttagttgatacaggatcat

SEQ ID NO: 187 - DNA, 101 bp, Phoenix dactylifera
tccttttaaatggaattcactgttttatgacgctttggttcaaaaaagccrcttaagtttaattaggatgccattcctaccggtcatttatgtgagagctg

SEQ ID NO: 188 - DNA, 101 bp, Phoenix dactylifera
ccctaattaatcttgcttcctatcttctcaagtcctaagattgtgaattcyggctrgatggttgtcggagttggttggaaaatttgatgccctatttcagt

SEQ ID NO: 189 - DNA, 101 bp, Phoenix dactylifera
attaatcttgcttcctatcttctcaagtcctaagattgtgaattcyggctrgatggttgtcggagttggttggaaaatttgatgccctatttcagttttga

SEQ ID NO: 190 - DNA, 101 bp, Phoenix dactylifera
cacaaataggtttatataagtcctctaccaagttttcgaacccaagggaaraaagaaaaagtttgcaagtaaaaaaaggcggtgtatggtctcaagaagtc

SEQ ID NO: 191 - DNA, 101 bp, Phoenix dactylifera
tctattgcttactttcaataggggcctttttcttctttatccttacgaacygaattatagaggttaccttaccatataaaatagaaagcaccttatcacaa

SEQ ID NO: 192 - DNA, 101 bp, Phoenix dactylifera
gtcactctcgatttctattgagcaactatacccaagttcaatgatgatcaratgaaaatgatcttgattagactgactgggatgtcgagcacttccacttg

SEQ ID NO: 193 - DNA, 101 bp, Phoenix dactylifera
ataattcttcaatattaggttgtggaggagatctttggagttaattggatytgttagtcgattccacttgcaaggtaagtaatgctccactttttctagat

SEQ ID NO: 194 - DNA, 101 bp, Phoenix dactylifera
tgatttgatttcgataccacatgattacatgtattgttgttaaaccaaaawrtgaaaccacatgattgtttcggacactcagcctcactttgttaaggatc

SEQ ID NO: 195 - DNA, 101 bp, Phoenix dactylifera
gatttgatttcgataccacatgattacatgtattgttgttaaaccaaaawrtgaaaccacatgattgtttcggacactcagcctcactttgttaaggatcc

SEQ ID NO: 196 - DNA, 101 bp, Phoenix dactylifera
cttgtaattttaggttacgagtatgacactaagtgttgcctgagtaacacytgtccaaaaaaatatagattcatatagattcctgggtaagtttcatgccc

SEQ ID NO: 197 - DNA, 101 bp, Phoenix dactylifera
tcaacgttgcccagatcatttagtccctgggtatgcacacccaacaagtasggatgggcatcagtacaaaatttgggtactgcctgcaagcagaaawctta

SEQ ID NO: 198 - DNA, 101 bp, Phoenix dactylifera
agtasggatgggcatcagtacaaaatttgggtactgcctgcaagcagaaawcttaactatcataaggttcctagactaaacaagcatccgcatccaataca

SEQ ID NO: 199 - DNA, 101 bp, Phoenix dactylifera
aactggcttagactgtacatcaacttcaagatgcaaatatcaatataggtrrttcaatatcatacacacctgacctctacgaaacagaaggaaaccatgtg

SEQ ID NO: 200 - DNA, 101 bp, Phoenix dactylifera
actggcttagactgtacatcaacttcaagatgcaaatatcaatataggtrrttcaatatcatacacacctgacctctacgaaacagaaggaaaccatgtga

SEQ ID NO: 201 - DNA, 101 bp, Phoenix dactylifera
tcaatatcatacacacctgacctctacgaaacagaaggaaaccatgtgatrgttaaaatattatgtctgaagaagaatgaacacagcaataatcatctgaa

SEQ ID NO: 202 - DNA, 101 bp, Phoenix dactylifera
ctgaagaagaatgaacacagcaatacatattgatcatcactggaattattscawaatgcaataacagactaacgattcacactttrggtgcaatatcgagt

SEQ ID NO: 203 - DNA, 101 bp, Phoenix dactylifera
aagaagaatgaacacagcaatacatattgatcatcactggaattattscawaatgcaataacagactaacgattcacactttrggtgcaatatcgagtatc

SEQ ID NO: 204 - DNA, 101 bp, Phoenix dactylifera
atcactggaattattscawaatgcaataacagactaacgattcacactttrggtgcaatatcgagtatctaagaaatttaatgacaggcacaagaataatt

SEQ ID NO: 205 - DNA, 101 bp, Phoenix dactylifera
gaactccactatcaagattgtaagcaatgcaccttaactaggtaatggttrcaatttaattgttaaacttgcatggaggccgatttgaccaaggttttaat

SEQ ID NO: 206 - DNA, 101 bp, Phoenix dactylifera
tcaaaaaaagagacaaaattaagcactatagtctaacgaaaatttgaagawttttctaatagccagtctggtgaattctcaccatagagatgyagatggtg

SEQ ID NO: 207 - DNA, 101 bp, Phoenix dactylifera
tttgaagawttttctaatagccagtctggtgaattctcaccatagagatgyagatggtgattcattttaaaagtggagatcaccaaggttagttgtgaagt

SEQ ID NO: 208 - DNA, 101 bp, Phoenix dactylifera
gatcaccaaggttagttgtgaagttactgaagtctaggatggcaattcgtkmtctggtatgataaaatttgaggggcaggtgttagacacagtaatgcgat

SEQ ID NO: 209 - DNA, 101 bp, Phoenix dactylifera
atcaccaaggttagttgtgaagttactgaagtctaggatggcaattcgtkmtctggtatgataaaatttgaggggcaggtgttagacacagtaatgcgatg

SEQ ID NO: 210 - DNA, 101 bp, Phoenix dactylifera
tgataaaatttgaggggcaggtgttagacacagtaatgcgatgatatagcyarcatggtagtaattgcacaacatatatacttgtatcggtcttgagcccc

SEQ ID NO: 211 - DNA, 101 bp, Phoenix dactylifera
ataaaatttgaggggcaggtgttagacacagtaatgcgatgatatagcyarcatggtagtaattgcacaacatatatacttgtatcggtcttgagcccctc

SEQ ID NO: 212 - DNA, 101 bp, Phoenix dactylifera
aattttatatttttttaatattctccgaagcaaggtccaccgtcttggtaycggactctataccagtatcattctattatagtatcagtatgcagtacgat

SEQ ID NO: 213 - DNA, 101 bp, Phoenix dactylifera
gtacgatacgagactacataccaattcggtactgatatggtacggtacatycggtacgatatggtatcgaccaatatagtgaaccttgctccgaagcactc

SEQ ID NO: 214 - DNA, 101 bp, Phoenix dactylifera
attttgttctcaatcccatgaatgaattgatgtatgcctctctctagaatycatttcctataacatccaagccatgcaaaaccaatgtgatgaactcttct

SEQ ID NO: 215 - DNA, 101 bp, Phoenix dactylifera
catggatccacaccttcttgataataaaaattgattatccaaaatcttgtrattttttgggggtaaccatgacaacaccctattatgatcgtgccctttgc

SEQ ID NO: 216 - DNA, 101 bp, Phoenix dactylifera
cttgtaggtagatgtgatccatcattttcagttgccatccaataaaacatwagtcttatacagtgttgctaaagggactttggttcatcaagaggcacccc

SEQ ID NO: 217 - DNA, 101 bp, Phoenix dactylifera
gaggcacccctttctcagtgcatgtgccaaggagagctttggatccagaakttggcattctaatcttccaccatccacccctycaaaacaaggagaaaaag

SEQ ID NO: 218 - DNA, 101 bp, Phoenix dactylifera
agagctttggatccagaakttggcattctaatcttccaccatccacccctycaaaacaaggagaaaaagaagaagatgctaaccaaggcacaagaaagcat

SEQ ID NO: 219 - DNA, 101 bp, Phoenix dactylifera
caaaacaaggagaaaaagaagaagatgctaaccaaggcacaagaaagcatkaagtcctgtttccaaagaccaacatatgtacgcaaacatgggcaaccaag

SEQ ID NO: 220 - DNA, 101 bp, Phoenix dactylifera
taataagccatatatggataaggctgcaaatgggtcaattgacccatggcscaacctgatccatttagacccgatctgacccatttttagagactcgtagg

SEQ ID NO: 221 - DNA, 101 bp, Phoenix dactylifera
ttccaccgttcatctcttcccttcctttaaaaccctagatccattctcacsttgttcccgtttgtccatcgaccttccccctcctctgtgttgawctcctg

SEQ ID NO: 222 - DNA, 101 bp, Phoenix dactylifera
tctcacsttgttcccgtttgtccatcgaccttccccctcctctgtgttgawctcctgagggactttcctcaatccttaayggtccagcaaagtcctccaac

SEQ ID NO: 223 - DNA, 101 bp, Phoenix dactylifera
cttccccctcctctgtgttgawctcctgagggactttcctcaatccttaayggtccagcaaagtcctccaaccttcaagcagccagcgaagtcctctgagt

SEQ ID NO: 224 - DNA, 101 bp, Phoenix dactylifera
tgccacatgatttatgatgtcacaagggaatggggcagcctataaaaaaawttgacataatttgtcacatttcttatatggcagtgattttggtgttgtta

SEQ ID NO: 225 - DNA, 101 bp, Phoenix dactylifera
gtgttgttaattactggactctggtgatttctactagtgtcatgctcatcrtggtcatccagtgagagtgacattttttaagaaaaattccttcttgcttt

SEQ ID NO: 226 - DNA, 101 bp, Phoenix dactylifera
gattttgcgcaagatttgctgtctcggtaccagactccgtaccagtaccayaataatatagtgtyggtacggtacgatayggagtttttttgtataccaag

SEQ ID NO: 227 - DNA, 101 bp, Phoenix dactylifera
tttgctgtctcggtaccagactccgtaccagtaccayaataatatagtgtyggtacggtacgatayggagtttttttgtataccaagtgtcggtacgccat

SEQ ID NO: 228 - DNA, 101 bp, Phoenix dactylifera
ccagactccgtaccagtaccayaataatatagtgtyggtacggtacgatayggagtttttttgtataccaagtgtcggtacgccatccatactaggtaccg

SEQ ID NO: 229 - DNA, 101 bp, Phoenix dactylifera
acttggctccgcatccaaaaaatgggttagatgcattgactcgaaccagayccatactcgaattgggttgggatcaggtcatacattgcccaacctraccc

SEQ ID NO: 230 - DNA, 101 bp, Phoenix dactylifera
cagayccatactcgaattgggttgggatcaggtcatacattgcccaacctracccgatcttctattgggctgggtctaggtccaacatcaagagctgaaca

SEQ ID NO: 231 - DNA, 101 bp, Phoenix dactylifera
aggtcgggtcagggtcaatcacgcctggatctgacccaacctatttgcagycctacaaacgcatgagatggaaattacacatatatgcatgcaactactag

SEQ ID NO: 232 - DNA, 101 bp, Phoenix dactylifera
gaaaagagacaaatgccaaacttttaatctccaccaagtgcaaaagttgtrgcaaaaccatttactaccttcactcacggccagcaaccgatgcattaagt

SEQ ID NO: 233 - DNA, 101 bp, Phoenix dactylifera
cattaagtatgaagattaggagagactaagagagatgtggtttagggctgyaaacaagctgaaatgtacatgaacaactcaagattggctcaataacaggc

SEQ ID NO: 234 - DNA, 101 bp, Phoenix dactylifera
aacaagctgaaatgtacatgaacaactcaagattggctcaataacaggctyattcaaacttgttcgactaaaatcaagcaaagatagaactttggttagaa

SEQ ID NO: 235 - DNA, 101 bp, Phoenix dactylifera
aggtttaaactgtaggcttacttcaggcctaggctcgatccagcctgaccragcctattggcacccctagcatagctagtgaaaatttcaatatcaaacag

SEQ ID NO: 236 - DNA, 101 bp, Phoenix dactylifera
agcaaaaatggttgaaaagttttcatggatcaaatgttaagaaacaaggtyagagatgtccaacctgcaattgatcgaaggttgttgtggcttagatctag

SEQ ID NO: 237 - DNA, 101 bp, Phoenix dactylifera
gtttcatcagaaactcttcatccaaagtcacctgcaaaatcattcagatawaaatattctagaacaatttgcatagatccgaaagtagctttagcacataa

SEQ ID NO: 238 - DNA, 101 bp, Phoenix dactylifera
accagcaaaaaattcaataaatgcacagttattttgcaacatcataggttrtctacttccaatgcaccagtaaccaatacttcatgatgtaagaataacaa

SEQ ID NO: 239 - DNA, 101 bp, Phoenix dactylifera
aataacaacacagataaactaaagtagtatctgaatgaaaagttattgaawcattattattgcgaacaaggttcgtcgtcttggtaccggaccccataccg

SEQ ID NO: 240 - DNA, 101 bp, Phoenix dactylifera
gcaccatcctattattatatcggtacgcccggtacaagggccaaatcggcmtaccgacattcggtacacccaycataccgcataccaattcagtatcggta

SEQ ID NO: 241 - DNA, 101 bp, Phoenix dactylifera
gtacgcccggtacaagggccaaatcggcmtaccgacattcggtacacccaycataccgcataccaattcagtatcggtacagtatagttagtacacacagg

SEQ ID NO: 242 - DNA, 101 bp, Phoenix dactylifera
ggtgacacttgccatcaagataaatttaatgatactgcataaatgcttcaygtcattttttgttgcatttcatatttcaggctcagattcacaattaaaat

SEQ ID NO: 243 - DNA, 101 bp, Phoenix dactylifera
gtcattttttgttgcatttcatatttcaggctcagattcacaattaaaatwaaagatacgttatcatgcttggcctacaaggccatccacaacaactagtt

SEQ ID NO: 244 - DNA, 101 bp, Phoenix dactylifera
tgtgaacaatattctattacatactgagttacatcccatagtctatgcctktgtgtgtgtctgtgtctatatatatccatgacatcgctgttaagaaacaa

SEQ ID NO: 245 - DNA, 101 bp, Phoenix dactylifera
atatccatgacatcgctgttaagaaacaataaaatgcaagcataggatgcrtattgtgatttcccaactacaggtgatattccactactcattttattgta

SEQ ID NO: 246 - DNA, 101 bp, Phoenix dactylifera
cttcatagttccagacaaagagctcctgaatggattctccatcaaattgtwctagattgttattattcaattttatagtatacttgaactgcaaaggatat

SEQ ID NO: 247 - DNA, 101 bp, Phoenix dactylifera
aatctcaaccgagtaagcttgcaaactactggaatttacatcaggaatatkaagataagtcacccaacaatgagcttctcttgaagttgttgttgataaag

SEQ ID NO: 248 - DNA, 101 bp, Phoenix dactylifera
gttgataaaggcttccaaataagattctcatttgcaccaaacatggaattwatggtcacagttgacgaactcacacctttgacagcttgattaaagtaaag

SEQ ID NO: 249 - DNA, 101 bp, Phoenix dactylifera
aaaatttggtagatcatatgttatatatcaagaagttactgctgataatamytttgcaaccaaaacttgatcggaaacaaccaaaataagcactgtagtag

SEQ ID NO: 250 - DNA, 101 bp, Phoenix dactylifera
aaatttggtagatcatatgttatatatcaagaagttactgctgataatamytttgcaaccaaaacttgatcggaaacaaccaaaataagcactgtagtagt

SEQ ID NO: 251 - DNA, 101 bp, Phoenix dactylifera
gtttattcaactgtgttcaactctgtaacttcatagttaagggaagcatcwttaagcaagtaattgcatggaaccaaactctgcagttaacttctccagaa

SEQ ID NO: 252 - DNA, 101 bp, Phoenix dactylifera
agaagactcaagggtttttatcatgcagaagtagaatgaatgtgaccaaawttttttctcactaggtaagtcataactgccttaaytttaaacttttttga

SEQ ID NO: 253 - DNA, 101 bp, Phoenix dactylifera
atgaatgtgaccaaawttttttctcactaggtaagtcataactgccttaaytttaaacttttttgaactacagaattagaaatgatatatacaattacact

SEQ ID NO: 254 - DNA, 101 bp, Phoenix dactylifera
taattttgagttgttcagaagtttaagctgacttacacatttaatttatcmcccaatcttcaactgttttatcttgtagagacaagaacgccactattgga

SEQ ID NO: 255 - DNA, 101 bp, Phoenix dactylifera
gtggtctgttggtagattgtccttcggcaatagaagatcccawagaggctrccatgacttttaactcttgattgttatatcaataagtagttgtgagtatc

SEQ ID NO: 256 - DNA, 101 bp, Phoenix dactylifera
ccaaaagctttgccaaatggggcctaactcatcaattgtagtttatcttarccaagagatcggtgttggccatcttagcaaggagggcgagaaaccaagac

SEQ ID NO: 257 - DNA, 101 bp, Phoenix dactylifera
tggccatcttagcaaggagggcgagaaaccaagacacatagtcttaaaatytgatacatgcttcagccaaaacttgcagcttcacttcgtatacccccttt

SEQ ID NO: 258 - DNA, 101 bp, Phoenix dactylifera
acaatgtacttgaattttccaaattgtaaggagatgatagaatatcaacastggagatgactaaatatcaatagtttgtgtatacaccatttgaaattttc

SEQ ID NO: 259 - DNA, 101 bp, Phoenix dactylifera
taataacaaatcttcagaaggggcattttttacagaccgaataagatgttytagatggaagtcaacctttgatcagataccaaggtacagactgagaacat

SEQ ID NO: 260 - DNA, 101 bp, Phoenix dactylifera
aatatcaagaatgtaggctgatacactaagggtgcaggccgatatgctaargatgtagactgatatgctaagagtgcaagctgatatgcctcggtatgtaa

SEQ ID NO: 261 - DNA, 101 bp, Phoenix dactylifera
agttattatcctttagtttttatagtagtttctacagtagagttaggcagyattctgttatcctcaaaattacttgaagtttcaaggcacccgaaccctat

SEQ ID NO: 262 - DNA, 101 bp, Phoenix dactylifera
acaatatagtcatcaagcccaaccaccctcatgtcgatcgctctagctccrtcttcatatgctatctcaaaacctaggtcttctcctcctccaaccttaat

SEQ ID NO: 263 - DNA, 101 bp, Phoenix dactylifera
cagaagactcaaatggaggagacacactagtctcctgccttatatagtctyagatggcttggattggcatgacgattcacctacctaggtcgatcttcacc

SEQ ID NO: 264 - DNA, 101 bp, Phoenix dactylifera
ttcacctacctaggtcgatcttcaccatgtgtcgcccagacattcaccctyggacgatcactcgaatcgctattaacaaaagcatctaaccattgctcaaa

SEQ ID NO: 265 - DNA, 101 bp, Phoenix dactylifera
ctcgaatcgctattaacaaaagcatctaaccattgctcaaaaactcccgcyatgaatgatactacttcagggagagcagacacatttgtcataaatgaagt

SEQ ID NO: 266 - DNA, 101 bp, Phoenix dactylifera
gtgatcacctttgctgggatggagaaatcttcacaaccaaggttctatacwctctggatgccgcactggttcctcgagttgatacttgacaaaacccgtac

SEQ ID NO: 267 - DNA, 101 bp, Phoenix dactylifera
gaaaagaaagggaaagccctcgctcccttcctggagtttggactctatttygaaggcccctcccttttttccccgagctcctcccccagctcccccgccgt

SEQ ID NO: 268 - DNA, 101 bp, Phoenix dactylifera
ctgaagaaacaagagcttgcaaagaggtatagtttaagaagtttgctttakatagctttgatattttaagctacatktggaaggtgcgtggcataaagttt

SEQ ID NO: 269 - DNA, 101 bp, Phoenix dactylifera
gtatagtttaagaagtttgctttakatagctttgatattttaagctacatktggaaggtgcgtggcataaagtttggtaagctattgcattcttcaggttc

SEQ ID NO: 270 - DNA, 101 bp, Phoenix dactylifera
taccacagtagcatgatgttgtcttgttgaatgatagaaaatactgtaaakacaaagcagcagggaatctttattttcttgactgtattcaatgtaactta

SEQ ID NO: 271 - DNA, 101 bp, Phoenix dactylifera
gacccttggtgcaacgataaggttgctccattgtgacttggaggtcacggrttcaaaacataaaaatagcctcttcgtatgcaagaggtaaggctgtgtac

SEQ ID NO: 272 - DNA, 101 bp, Phoenix dactylifera
catttaatctttgcattatatgtgaatgaagacaagggttgtattgggcayttcacacacctttgcaaatgcwcaaaatgccttacgtttatcaacagccc

SEQ ID NO: 273 - DNA, 101 bp, Phoenix dactylifera
tgaatgaagacaagggttgtattgggcayttcacacacctttgcaaatgcwcaaaatgccttacgtttatcaacagcccctgtatagatactaaaacatat

SEQ ID NO: 274 - DNA, 101 bp, Phoenix dactylifera
gctcacttgttttgaatagaatgaacttcgatatgctgctcttctgtcatwtgcagaaaatgtacgagtgcacatgtgaacctgtcgatagcttgtgcaag

SEQ ID NO: 275 - DNA, 101 bp, Phoenix dactylifera
cagggctgcaattctgatgtcattaggtgatatctatgggcatctgttttrgcaaatgatgccgacacaattcaattgatccttggaagaaactgatgaat

SEQ ID NO: 276 - DNA, 101 bp, Phoenix dactylifera
gccgacacaattcaattgatccttggaagaaactgatgaatggtttatttwatatgttaagattaaaatttgtaaactacatccttgcatgggaagttcct

SEQ ID NO: 277 - DNA, 101 bp, Phoenix dactylifera
ttctaatgaactctactcgttatgatattgtatatgttatgagtaggctcrctagatatgcacataatcctagtagtggtcattggattccattatgtaga

SEQ ID NO: 278 - DNA, 101 bp, Phoenix dactylifera
aggcgaggtcttgatgcatgtacaggcatgccactaatatcgggttagctrgaaagatgtagctcaaatggaggataaagataagcatggatccaaatacc

SEQ ID NO: 279 - DNA, 101 bp, Phoenix dactylifera
gcatgagatcggctgctttcaactagctagtaaccctgattctactttgtygtatgtctaacataagacgatggagcaataatatattggaatgaaagtct

SEQ ID NO: 280 - DNA, 101 bp, Phoenix dactylifera
tgcaagcagtcttcacaaacaacagtggcgaacagtttaatgttcccgaaraatgttgaatagcattggctgatttaatttttattaaagaattaatacaa

SEQ ID NO: 281 - DNA, 101 bp, Phoenix dactylifera
gatttcttcatgcaaagattttagttgagaaaggttggatagacaagtacrtgattaaggaatattatccgggaatgaagtggagacaaactactgaaatt

SEQ ID NO: 282 - DNA, 101 bp, Phoenix dactylifera
cggtacaaaactgcataccaattcagtaccggtatggtacagcatgcctayggtatggtactgagcagtacggcaaaccttttcaggaatattcataattc

SEQ ID NO: 283 - DNA, 101 bp, Phoenix dactylifera
gagtctcatggcaggacgaggcgggtagagtctcagggaaagatggggcgragaaaatctcggagcatgataggagaggtaaagttagaatgggttggaca

SEQ ID NO: 284 - DNA, 101 bp, Phoenix dactylifera
tacaattgagttgcaagttattctactagcatttttgaacttgtgcatgtycaacaatatctgttgaatcaaaatgcatctcatagcgaacatttatgagt

SEQ ID NO: 285 - DNA, 101 bp, Phoenix dactylifera
aactaaggttaatttcaggggaagtcaatccaactcattctaccctataaraaaggcaccaaaatgggattaatacttgaaaaaatctaactagagaatac

SEQ ID NO: 286 - DNA, 101 bp, Phoenix dactylifera
agcagaactagctctcctcgaaaaagggatgaagctaaagatgtgaaaacygaggtgcgaaaattatctgcaattattggaaggtataaaggagaaaattc

SEQ ID NO: 287 - DNA, 101 bp, Phoenix dactylifera
cctaaaacattgaatcagagggtagtttcagagctttgaaactttgagacygagttcagtttcaaagcttgaaactttgagactaagcttagttgtagaac

SEQ ID NO: 288 - DNA, 101 bp, Phoenix dactylifera
ctttctttctttaacctgttctggtttcaccggagccttgctcatgctacygccactcattgccttgaatccttctccttatggacagctttacttgcctt

SEQ ID NO: 289 - DNA, 101 bp, Phoenix dactylifera
attgtattgaaagtcataccttcacaaaacttgacatccctcatgtcaatmtaggggtatcaaactctaaagaatcaagatacacactgtagtatccggta

SEQ ID NO: 290 - DNA, 101 bp, Phoenix dactylifera
ggcatcagaaatgtcacgccccaaatccggtccgtcacacggccgtgctaytgcaagctattgcccacaataacacgaagccacaataacaattcataaca

SEQ ID NO: 291 - DNA, 101 bp, Phoenix dactylifera
ttgcaaaccctaaagtcttcaggctccctgctcctagcaaccttatttatstgaaaaaaacaaccggagtcatatgagccatgcggctcagwaagtaacca

SEQ ID NO: 292 - DNA, 101 bp, Phoenix dactylifera
cttatttatstgaaaaaaacaaccggagtcatatgagccatgcggctcagwaagtaaccatgcatcatcttaccaaatcaagcataacatttcacatgccc

SEQ ID NO: 293 - DNA, 101 bp, Phoenix dactylifera
catcatcttaccaaatcaagcataacatttcacatgcccatgtattatttwaaaaaattaacaattatagcaaagtaaatcatttcatagacaatatatcc

SEQ ID NO: 294 - DNA, 101 bp, Phoenix dactylifera
agatggagatgcaatgtaaaattctgccattatttcatgctttaaaaatcrtcctctcagacggtagtgcagctatggtcactataatccgtgacatggcc

SEQ ID NO: 295 - DNA, 101 bp, Phoenix dactylifera
agcaacaagcagtgcaagtggttcggatcatatgggtcatggatgatcccsactcaatctgattctttggaatctaattttagacccaaatataatttaat

SEQ ID NO: 296 - DNA, 101 bp, Phoenix dactylifera
ttaataaaaatcgaatcgaatcgatccatcattcggagttgggtcataacyatgtataacctatatccaattcaaaaaatttggattcgattcggatccgg

SEQ ID NO: 297 - DNA, 101 bp, Phoenix dactylifera
aattgactatcattctctgagtcctcattttcatgcatttgtcatgattgwattatatgctcaactaaggattgccataatgttctctgggagaaataaaa

SEQ ID NO: 298 - DNA, 101 bp, Phoenix dactylifera
gttctctgggagaaataaaatgtctagaagagagaattagaagatgcttayattaaagtagccatatgttgctagttttgcaggaactgaaatggttaccg

SEQ ID NO: 299 - DNA, 101 bp, Phoenix dactylifera
ttagaattgctgcccagtaccactaggctaccaccttggtggctctacaarggttttttgctaaaatttggttcacatacggattggtcattattttgcct

SEQ ID NO: 300 - DNA, 101 bp, Phoenix dactylifera
acccttagatcccggcaatcttaaactcaattgacgagccctgcataagtyagaataatcccacccaataatacrtgcaccgagaactagagcacttcaca

SEQ ID NO: 301 - DNA, 101 bp, Phoenix dactylifera
actcaattgacgagccctgcataagtyagaataatcccacccaataatacrtgcaccgagaactagagcacttcacaaacaacaggtcatagagactctct

SEQ ID NO: 302 - DNA, 101 bp, Phoenix dactylifera
gaaaagagggggataagggggcttaggagaagggttcggctcatatattawtacgaaaaaaaaaaataaagcgagaaaggaaaattttaatatacttcatg

SEQ ID NO: 303 - DNA, 101 bp, Phoenix dactylifera
gtgaattaaaaaaacactcagacgatagttatcatagcattgaaaccatarcccagggcratatagatttaggagtgttcctctactcctcccctacatat

SEQ ID NO: 304 - DNA, 101 bp, Phoenix dactylifera
aaaaacactcagacgatagttatcatagcattgaaaccatarcccagggcratatagatttaggagtgttcctctactcctcccctacatatattggagtt

SEQ ID NO: 305 - DNA, 101 bp, Phoenix dactylifera
ctatttgaggaattccatgaggcgatcgagctttatcgaggaggggatcargatgatatacttggcctatcacatttggttggacatgaatactcgtgtct

SEQ ID NO: 306 - DNA, 101 bp, Phoenix dactylifera
gacatgaatactcgtgtctttgagggcagtaggtcggcgccgaggacgatrgtgggcaggatgtttcatcatgcaatggagatcatcacggtgaccgtgct

SEQ ID NO: 307 - DNA, 101 bp, Phoenix dactylifera
aggtggagcttcgggccgcctaaaagggtatttcctttgcgaggagagttyttggagcggacatgttccacctcgaggaggacttaggagtggaccgctat

SEQ ID NO: 308 - DNA, 101 bp, Phoenix dactylifera
tactcaagagagtaccccaagcatattgctagacatttgttttgtttgctyggcttatttggtggaaattctggaaattggtttatcttattggataatta

SEQ ID NO: 309 - DNA, 101 bp, Phoenix dactylifera
ggtctctacctcaatgacgaagctatcatcctatttatatagcggcacaaygagtgtaggcctaaaatatataactcggcctagaacaacatgagttaaya

SEQ ID NO: 310 - DNA, 101 bp, Phoenix dactylifera
aygagtgtaggcctaaaatatataactcggcctagaacaacatgagttaayaaggaactgaaagaccaccactccatgcctataaatcattaatcatcggt

SEQ ID NO: 311 - DNA, 101 bp, Phoenix dactylifera
cagagtaagtatatggatcattatccaaatcctctgattccaattgtaaamtctcatttaacattgagtatctcatcggaggtctaattactctctcactc

SEQ ID NO: 312 - DNA, 101 bp, Phoenix dactylifera
gtacctgctctcacccgaagtaaagcactataataaagtaagtttctttcyagttcgacttttctttgctctggttgggacctaccatctggtatcagagc

SEQ ID NO: 313 - DNA, 101 bp, Phoenix dactylifera
ctataagcagagatgggcgccatgcagaaggggttacgtggcttggaaacrcgagtggaggggtgcttcactgaagtcttggaggttatttgcaacctttt

SEQ ID NO: 314 - DNA, 101 bp, Phoenix dactylifera
ttggattttctatggttcaccgagtcaaggcaccccagctaactaatgggyatccacaacctcattttacttggatgaggaagcgaaccactggtggaagt

SEQ ID NO: 315 - DNA, 101 bp, Phoenix dactylifera
gattgctcgcattagggaggaaaggttggagcagaagtggaatgcctcacrtagcatcgggcctcrtattactagtgccccaatagccccaaccatgccaa

SEQ ID NO: 316 - DNA, 101 bp, Phoenix dactylifera
ggaggaaaggttggagcagaagtggaatgcctcacrtagcatcgggcctcrtattactagtgccccaatagccccaaccatgccaacaaatgtactgcgac

SEQ ID NO: 317 - DNA, 101 bp, Phoenix dactylifera
ctcgctacatgcactatctagatggaacataccaaaaataatgtgggtgayggctcacatccaaggacaactgctracagtccttattgacagtgggtcaa

SEQ ID NO: 318 - DNA, 101 bp, Phoenix dactylifera
aacataccaaaaataatgtgggtgayggctcacatccaaggacaactgctracagtccttattgacagtgggtcaacacataactttataagcgatcgcat

SEQ ID NO: 319 - DNA, 101 bp, Phoenix dactylifera
tgggtcaacacataactttataagcgatcgcattgcaagctgctttagctrcttgtagatgccacgrtgaaatttggggtccgagtcgcaaatggagaaac

SEQ ID NO: 320 - DNA, 101 bp, Phoenix dactylifera
tttataagcgatcgcattgcaagctgctttagctrcttgtagatgccacgrtgaaatttggggtccgagtcgcaaatggagaaaccttccaatgccaagaa

SEQ ID NO: 321 - DNA, 101 bp, Phoenix dactylifera
ttggggtccgagtcgcaaatggagaaaccttccaatgccaagaaatgtttmgatctgtggaggttgagctacaagaaaagaagtttctggtgaccttttat

SEQ ID NO: 322 - DNA, 101 bp, Phoenix dactylifera
cggacctacgaaagttgatgggaaactactccaatctttttgaggaacctyaagggctaccaccggagctagcatttgtccaacggatttctttacaagaa

SEQ ID NO: 323 - DNA, 101 bp, Phoenix dactylifera
gcatttgtccaacggatttctttacaagaaggaactttggctatcaatgtwtgactgtatcggtatgcttattactaraagaattagattgagcrccaggt

SEQ ID NO: 324 - DNA, 101 bp, Phoenix dactylifera
gaaggaactttggctatcaatgtwtgactgtatcggtatgcttattactaraagaattagattgagcrccaggtagctaacatgctacaaagtgggattat

SEQ ID NO: 325 - DNA, 101 bp, Phoenix dactylifera
caatgtwtgactgtatcggtatgcttattactaraagaattagattgagcrccaggtagctaacatgctacaaagtgggattatcaaggagagctagagtc

SEQ ID NO: 326 - DNA, 101 bp, Phoenix dactylifera
aagaaagatgatacctagaggttttgcacggattatcgagcactcaatgcygtcgccctcaaagayaggttccctattccaattgtcaatgatatgctcaa

SEQ ID NO: 327 - DNA, 101 bp, Phoenix dactylifera
tagaggttttgcacggattatcgagcactcaatgcygtcgccctcaaagayaggttccctattccaattgtcaatgatatgctcaatgaactacatggaga

SEQ ID NO: 328 - DNA, 101 bp, Phoenix dactylifera
ttccctattccaattgtcaatgatatgctcaatgaactacatggagaacgmtattttatcaagcttgaccttaggacggrttatcaccaaatccaagtcca

SEQ ID NO: 329 - DNA, 101 bp, Phoenix dactylifera
caatgaactacatggagaacgmtattttatcaagcttgaccttaggacggrttatcaccaaatccaagtccaccctgaagatatccataagacaaccttty

SEQ ID NO: 330 - DNA, 101 bp, Phoenix dactylifera
rttatcaccaaatccaagtccaccctgaagatatccataagacaacctttygcacacataacggccactttgagtaccttgtcatgcccttcgggttrtgc

SEQ ID NO: 331 - DNA, 101 bp, Phoenix dactylifera
tttygcacacataacggccactttgagtaccttgtcatgcccttcgggttrtgcaatgccccatcgacattccaggccgccatgaattccatcttcagaag

SEQ ID NO: 332 - DNA, 101 bp, Phoenix dactylifera
ggtatttgcgtaaatttattttggtatttttcaatgatattttggtgtatrgtrccacatggcagcttcatttgtctcatcttgacattacactcaagatt

SEQ ID NO: 333 - DNA, 101 bp, Phoenix dactylifera
atttgcgtaaatttattttggtatttttcaatgatattttggtgtatrgtrccacatggcagcttcatttgtctcatcttgacattacactcaagattctt

SEQ ID NO: 334 - DNA, 101 bp, Phoenix dactylifera
tctcatcttgacattacactcaagattcttgctgatcaccaattttacttraaaccatccaaatgcttgyttgggcaacaagtcgagtatcttgcccacat

SEQ ID NO: 335 - DNA, 101 bp, Phoenix dactylifera
tcaagattcttgctgatcaccaattttacttraaaccatccaaatgcttgyttgggcaacaagtcgagtatcttgcccacattgtttcagctaatggagtg

SEQ ID NO: 336 - DNA, 101 bp, Phoenix dactylifera
gccttccaccttctaaagcaagcaatgatgcattagacagcctccgtgctygccattccaaactttttgaaaatatttgtgatcgaaactgatgtatcagg

SEQ ID NO: 337 - DNA, 101 bp, Phoenix dactylifera
gcctatagctcacatgagcaaggcgctaggctcgactaagaaggggtggtycacttacgccaaagagatgttggaaatcatggaggcratacggatctgtt

SEQ ID NO: 338 - DNA, 101 bp, Phoenix dactylifera
aagaaggggtggtycacttacgccaaagagatgttggaaatcatggaggcratacggatctgttgggaaggaarttccaaatatgcactratcaaaagagt

SEQ ID NO: 339 - DNA, 101 bp, Phoenix dactylifera
caaagagatgttggaaatcatggaggcratacggatctgttgggaaggaarttccaaatatgcactratcaaaagagtttgtggcacattttggagcaacg

SEQ ID NO: 340 - DNA, 101 bp, Phoenix dactylifera
atcatggaggcratacggatctgttgggaaggaarttccaaatatgcactratcaaaagagtttgtggcacattttggagcaacgagtgagtacatcggag

SEQ ID NO: 341 - DNA, 101 bp, Phoenix dactylifera
atttgacatttggggtgaaatttggattgccaacagtgaagacccttattygtgacacaagatccaacaattagaagccacacccaatggcatgaagaact

SEQ ID NO: 342 - DNA, 101 bp, Phoenix dactylifera
cacctcaagaaaggaatcttgtactacaaagacaaggtcctaattccaccrgggtcaccactttgtacaaagttgatgggagagttccataacacaaaaat

SEQ ID NO: 343 - DNA, 101 bp, Phoenix dactylifera
caaacgagtttcccaagcctttcgttgggaatcgatgaagaaggatatacrgagatttgtggcagaatgtatggtttgtcagcaacataagtacaaagctc

SEQ ID NO: 344 - DNA, 101 bp, Phoenix dactylifera
actcacattttttggctctctctcacccatatactgcaaaagaagtggcgraactttttgtwagtccagtggcccacttgcatgggatgccraggtcagtc

SEQ ID NO: 345 - DNA, 101 bp, Phoenix dactylifera
ttggctctctctcacccatatactgcaaaagaagtggcgraactttttgtwagtccagtggcccacttgcatgggatgccraggtcagtcgttagtgactg

SEQ ID NO: 346 - DNA, 101 bp, Phoenix dactylifera
gaagtggcgraactttttgtwagtccagtggcccacttgcatgggatgccraggtcagtcgttagtgactgggactctgtgtttgtgagtaccttctagca

SEQ ID NO: 347 - DNA, 101 bp, Phoenix dactylifera
gtctgtgtagtcaatttccaaagacatgggagcaaaacctggctttggcaragtattggtatagcaccgcctttcactccgccgctgagatgacgcccttt

SEQ ID NO: 348 - DNA, 101 bp, Phoenix dactylifera
agagagttgaaaagtaacttggagaagacgcaatgccacatgaaacaacakgcagacaagaagaggagggtggttacacttgcaraaggagaatgggtgtt

SEQ ID NO: 349 - DNA, 101 bp, Phoenix dactylifera
gccacatgaaacaacakgcagacaagaagaggagggtggttacacttgcaraaggagaatgggtgtttctaaaactacctccttatcgacatcatagccga

SEQ ID NO: 350 - DNA, 101 bp, Phoenix dactylifera
tttcactaatcacttctcaaaaagaggcttggagatgggcaagttccttgyaygggaccaccaccatctggtgcaggtgatttcagctaatgcactgcatt

SEQ ID NO: 351 - DNA, 101 bp, Phoenix dactylifera
tcactaatcacttctcaaaaagaggcttggagatgggcaagttccttgyaygggaccaccaccatctggtgcaggtgatttcagctaatgcactgcattct

SEQ ID NO: 352 - DNA, 101 bp, Phoenix dactylifera
tctgttccagtttacttgctgttgcgatgctcaagatgtttctcttacaayattgcaggttgggttctatgttgaatgtgaatatgtttgggatgtcagct

SEQ ID NO: 353 - DNA, 101 bp, Phoenix dactylifera
gtatgaaggcagaccaaacgacaaaaagaaaaaatcgagaaccaaatgacmtttctattcacttctcttccatttacattgtaygctacgagccaagaatt

SEQ ID NO: 354 - DNA, 101 bp, Phoenix dactylifera
atcgagaaccaaatgacmtttctattcacttctcttccatttacattgtaygctacgagccaagaattgacagaaggaaaaagatggaatttgcatgcact

SEQ ID NO: 355 - DNA, 101 bp, Phoenix dactylifera
aggtcaaacaactgcccaaagttttaatgccaatggaggtgctactgacascagagcaattttcccttttttcagctttcatgttaatctcctatctgtaa

SEQ ID NO: 356 - DNA, 101 bp, Phoenix dactylifera
tgcaattgagagttacgtgctcgaatggaaaagagagaaggaaaataactkaaggttcacattgtcagcgattgcatgatagtatcatgacaacatctgta

SEQ ID NO: 357 - DNA, 101 bp, Phoenix dactylifera
acaagagattctacgcaaccaatgatgtgaacaattcagctacttctgctktagacaataatcttggcatcagacagatggtgttcaaaagcatgtcaaca

SEQ ID NO: 358 - DNA, 101 bp, Phoenix dactylifera
gctagaaatagataatgtggctgaatggttattgtgttacttgagtagccwgaccatggaaaaagctgataaattcattgaaatacacaaaaagaggaata

SEQ ID NO: 359 - DNA, 101 bp, Phoenix dactylifera
cctagtctttattgcttaaagttaataccatcttcgccaaaatttatagtratcatgtcaaattccatggttttcagttgtaataattatacggtccattt

SEQ ID NO: 360 - DNA, 101 bp, Phoenix dactylifera
aggttcatttatgctgatatatctgacccatggtgaaaaacaatatcagarattgaaaatattatggaaaaaacaaatttctcaaagccagacaccattaa

SEQ ID NO: 361 - DNA, 101 bp, Phoenix dactylifera
acaaatttctcaaagccagacaccattaagaggcgccatttctaccactawatgattatagattggaggtcyacacaccttaggttgaataggatgagcac

SEQ ID NO: 362 - DNA, 101 bp, Phoenix dactylifera
accattaagaggcgccatttctaccactawatgattatagattggaggtcyacacaccttaggttgaataggatgagcaccagcgtaaaccttggagatgc

SEQ ID NO: 363 - DNA, 101 bp, Phoenix dactylifera
aagaacacctccaaccataacaagtgggaatggatcctttccatctgcaastaaaaagcaccaattaaaatctcaacacctgtaagcaasccaaatgagta

SEQ ID NO: 364 - DNA, 101 bp, Phoenix dactylifera
tccatctgcaastaaaaagcaccaattaaaatctcaacacctgtaagcaasccaaatgagtaaaacctwtgtggatcatcatggtttcctgcacctttgcc

SEQ ID NO: 365 - DNA, 101 bp, Phoenix dactylifera
gcaccaattaaaatctcaacacctgtaagcaasccaaatgagtaaaacctwtgtggatcatcatggtttcctgcacctttgccacacaatctaagtcttcg

SEQ ID NO: 366 - DNA, 101 bp, Phoenix dactylifera
taaaaaaacatgaaagagggtatttataagaaaaaaacaaataaatctaaragggaaacaygattcacacttgacattctacatccgtaatactaatcatc

SEQ ID NO: 367 - DNA, 101 bp, Phoenix dactylifera
tgaaagagggtatttataagaaaaaaacaaataaatctaaragggaaacaygattcacacttgacattctacatccgtaatactaatcatccaaagaaaag

SEQ ID NO: 368 - DNA, 101 bp, Phoenix dactylifera
caaaggcaatctggcacttgcatgtataagctagttaatgcatgcatgtayacctgaaaacttgacaactagaatatgacaagtagatatgtttgtgcatt

SEQ ID NO: 369 - DNA, 101 bp, Phoenix dactylifera
cttccttgtcaatcatccttccctttcacaaaaattcttttttccccaatrctttcccctctctcctcatgattttccctgaattttttagaattacagct

SEQ ID NO: 370 - DNA, 101 bp, Phoenix dactylifera
cagacttagatacccacatggagaggactattgttttggttttcaataacraaattggcatattttcatttcaatagcatgcatatccagcatgaatcata

SEQ ID NO: 371 - DNA, 101 bp, Phoenix dactylifera
cgatgaagagataaagaagccaattttgaaggagttcattgagaaagttgraaggaaacaagcaatacattttcaaacatgaatatgttcccctgaaaagt

SEQ ID NO: 372 - DNA, 101 bp, Phoenix dactylifera
caaagtccaaactgtcttctccagtccaatcataatgaaaacatcaaacgwtctggagccttacacaacaaagaacaggctcacattgatgagtacctgtt

SEQ ID NO: 373 - DNA, 101 bp, Phoenix dactylifera
atcaatagttaggtgatggtgggtattgcacctaaaagatatgagactgtrctgtcctccaatctgcagtttaacttcactagtgtcttatattatgtcaa

SEQ ID NO: 374 - DNA, 101 bp, Phoenix dactylifera
ataatcaataggcataagagaacaacaattgtgagtagtcgttggcgacawwtgagttagttaccaagcatcatggatctacgccaagtttcttagttctt

SEQ ID NO: 375 - DNA, 101 bp, Phoenix dactylifera
taatcaataggcataagagaacaacaattgtgagtagtcgttggcgacawwtgagttagttaccaagcatcatggatctacgccaagtttcttagttctta

SEQ ID NO: 376 - DNA, 101 bp, Phoenix dactylifera
accagaatataacaaataaaaggacacagtgatcacaatggcctagagatrctctaaagtgattacttgctacttctctcatmtgaaaggacaaaaaaaag

SEQ ID NO: 377 - DNA, 101 bp, Phoenix dactylifera
tcacaatggcctagagatrctctaaagtgattacttgctacttctctcatmtgaaaggacaaaaaaaagggaatatatttccactatagccagattaacat

SEQ ID NO: 378 - DNA, 101 bp, Phoenix dactylifera
atatatttccactatagccagattaacatggattaagcgaaaattgtgaayctaaagggttctgtgatcactgaatgccagctaaacttagggaaaatcta

SEQ ID NO: 379 - DNA, 101 bp, Phoenix dactylifera
ctagcattactgacttgtcattgctctttgttaacagaatcatatctgaayatacttatggaagttatagatctgaatctagtaatctaaacagtgaaata

SEQ ID NO: 380 - DNA, 101 bp, Phoenix dactylifera
aatctgatagacaccagatataactgttgaaataaagaccaggtaataatktttttagtattttgttacagcattgacaagtatggtatgtcgtaccgctc

SEQ ID NO: 381 - DNA, 100 bp, Phoenix dactylifera
ttttgttacagcattgacaagtatggtatgtcgtaccgctcgaatcagacrtacggagcgtaccgtaccgtaccagtttggyactggtatccgatatgga

SEQ ID NO: 382 - DNA, 101 bp, Phoenix dactylifera
gtaccgctcgaatcagacratacggagcgtaccgtaccgtaccagtttggyactggtatccgatatggatggcataccgatatttggtacgccaaaaaatt

SEQ ID NO: 383 - DNA, 101 bp, Phoenix dactylifera
ttttataccgtattgtaccgtaccgacattgtggtagtgtggcactggtayggagtccaataccgagacgacgaacatccgacgccactccactacgatcc

SEQ ID NO: 384 - DNA, 101 bp, Phoenix dactylifera
actccactacgatcctctctccctctcatcccgcattcgatgctcccgctrctctcctctccctctcacatcttctcctccatcacaactgtcaagtaagc

SEQ ID NO: 385 - DNA, 101 bp, Phoenix dactylifera
agccaccggcgcatctctttctccctcattctattacccaaagggtgagcycatggggttcttggacaggaaaaaaaaaaagaaagaaaaaaggggagggc

SEQ ID NO: 386 - DNA, 101 bp, Phoenix dactylifera
tggaactttggaattttgagggaaagtttaaaatttttttgaaattttgawgtagccctaattagatatatgtgtgttttggggatgattaggttccgagg

SEQ ID NO: 387 - DNA, 101 bp, Phoenix dactylifera
ttaagtgcggccatagttattggctgtagagaaaaataaactatatacccragtataaatatgtgaattagtacatgaatgcaagcatagtataagtatga

SEQ ID NO: 388 - DNA, 101 bp, Phoenix dactylifera
ggatcaagtttgggcaagtatcagtttttgaggcatgtgaataatagtaawgccatcttgcaagtcagtatgttgggtttyaacgattttatgtataaaaa

SEQ ID NO: 389 - DNA, 101 bp, Phoenix dactylifera
aggcatgtgaataatagtaawgccatcttgcaagtcagtatgttgggtttyaacgattttatgtataaaaacataaatatggaaagttgagttgtaatttt

SEQ ID NO: 390 - DNA, 101 bp, Phoenix dactylifera
tatgacttaaggtggtatttatgtatactttatgtgatgcatcagcttaayagtatagtggttctgataatcaccactgcctgcatatgaactatgtgatt

SEQ ID NO: 391 - DNA, 101 bp, Phoenix dactylifera
tagtggttctgataatcaccactgcctgcatatgaactatgtgattgccargttatattgatacattgacaagtgaaatgccttgcatgcttatagggtca

SEQ ID NO: 392 - DNA, 101 bp, Phoenix dactylifera
gtgaaatgccttgcatgcttatagggtcaacgccttatgagcgtgcagcarctgccacgcccttgtatcgtgaatacaaggtcgagggtgtgacaattaga

SEQ ID NO: 393 - DNA, 101 bp, Phoenix dactylifera
gggtagttatttggatcaaagcataattcttatttgttatgttctaagtgrttaataacctcaaatcttagtttatttgaaaccttataaggaacttttgt

SEQ ID NO: 394 - DNA, 101 bp, Phoenix dactylifera
gtgctacaacctctgccggaaatatccagattgaacaacaatagcaaccarcaatgcaaagaatgtcgaactgggccaaaccgttcggcccagttcggccg

SEQ ID NO: 395 - DNA, 101 bp, Phoenix dactylifera
ttggaagtgcaactaatgcaacacagagttcaaaagaaaagggataaccarattgaagcagcacttagctggtggttacctgatgtatctatgtgctagaa

SEQ ID NO: 396 - DNA, 101 bp, Phoenix dactylifera
agatccaaagttcaagaaaacctcaatcaggaagaccgtcagcaaaggcgrtggatgtattgcatctatactagaggcttttggtagcaaaaaratatttg

SEQ ID NO: 397 - DNA, 101 bp, Phoenix dactylifera
aaaggcgrtggatgtattgcatctatactagaggcttttggtagcaaaaaratatttgtatgtatattgtactttaaatatgtgtaattgatkatattgtt

SEQ ID NO: 398 - DNA, 101 bp, Phoenix dactylifera
agcaaaaaratatttgtatgtatattgtactttaaatatgtgtaattgatkatattgttttaaaatttttaccttactacttgatttaaggatattttatg

SEQ ID NO: 399 - DNA, 101 bp, Phoenix dactylifera
atggagagcgtagttggaatactacaacaacagttgcaatttcaacggtgratgcaataacaatttctacaacagcaacaacaattgttgcaacaaaagga

SEQ ID NO: 400 - DNA, 101 bp, Phoenix dactylifera
tttcagaatttaagaaggtagcactaccatctttcaaaggtactactgaaytattggaggcwaagatttggctagaagagatggagaaagcctttacgatt

SEQ ID NO: 401 - DNA, 101 bp, Phoenix dactylifera
aagaaggtagcactaccatctttcaaaggtactactgaaytattggaggcwaagatttggctagaagagatggagaaagcctttacgattataaaatattt

SEQ ID NO: 402 - DNA, 101 bp, Phoenix dactylifera
ccttgcctctagtttatggagagcgtagttggaatactgcaacaacaattrcaatttcaacaacggakgcaacaacaatttctgcaacagcaacagcaatt

SEQ ID NO: 403 - DNA, 101 bp, Phoenix dactylifera
ggagagcgtagttggaatactgcaacaacaattrcaatttcaacaacggakgcaacaacaatttctgcaacagcaacagcaattattgcaacaaaaggaat

SEQ ID NO: 404 - DNA, 101 bp, Phoenix dactylifera
ggctagaggaaatagaaaaagcctttacgattataaaatatttggaagaakataacatagcctatgcctccyacatgctacaggtagaagcctttgattga

SEQ ID NO: 405 - DNA, 101 bp, Phoenix dactylifera
cctttacgattataaaatatttggaagaakataacatagcctatgcctccyacatgctacaggtagaagcctttgattgatggagatatggaacaaaccaa

SEQ ID NO: 406 - DNA, 101 bp, Phoenix dactylifera
atggagatatggaacaaaccaagcatacctctgagacaaagcgcttcactkgrgagaaattctgtaaggcattttaggaaaaatattttctttccagtgtt

SEQ ID NO: 407 - DNA, 101 bp, Phoenix dactylifera
ggagatatggaacaaaccaagcatacctctgagacaaagcgcttcactkgrgagaaattctgtaaggcattttaggaaaaatattttctttccagtgttgg

SEQ ID NO: 408 - DNA, 101 bp, Phoenix dactylifera
ggtagaggacgaagagactcgagcctgcaagttccaaatgtgtttaagagyagaaattaggaaaggggtttcacctttgcagttgactacttatggggaaa

SEQ ID NO: 409 - DNA, 101 bp, Phoenix dactylifera
caagcagagcccctactgtacaaagtaaaagagaaaatcctaaaagaagtktctaacaccagttatacaatatcctagcggcactaaaatttataggaata

SEQ ID NO: 410 - DNA, 101 bp, Phoenix dactylifera
ataagttaggcagtataaaactaagatcatagtcaatcgacggaatggagrcctatgcatgtaagactaagatttgaagttcaaagcaaaaggaattgttc

SEQ ID NO: 411 - DNA, 101 bp, Phoenix dactylifera
aaaccaacttatggacatgaacttcacaatgggcagtatgtaatgtttgcrgatattgtcgtgagctgggaggaagggccacgtgagattgttagactatg

SEQ ID NO: 412 - DNA, 101 bp, Phoenix dactylifera
caacaagagtcagaagagaaatcagtcacttttcgtattaaccaacccaaraatcaatttaatttagaaattaaygatataaattttgaggataaagagca

SEQ ID NO: 413 - DNA, 101 bp, Phoenix dactylifera
gtcacttttcgtattaaccaacccaaraatcaatttaatttagaaattaaygatataaattttgaggataaagagcattacaggtatgcgtaccaaagaat

SEQ ID NO: 414 - DNA, 101 bp, Phoenix dactylifera
atcgatgccagctgtgaggacttatgatcggaacatcgatagttaatatgrgagtatgatgtatagatgaacagataccaaaacctaatggatgaatgcaa

SEQ ID NO: 415 - DNA, 101 bp, Phoenix dactylifera
attgtgatgtgatcaatcttaggaattacaagattaaagcagatatattaygtcctaattattttcagtcttcagtacataaatttcraggacgaacttta

SEQ ID NO: 416 - DNA, 101 bp, Phoenix dactylifera
agcagatatattaygtcctaattattttcagtcttcagtacataaatttcraggacgaactttaattaggagaggattatgtgaggcccaggcccaggatt

SEQ ID NO: 417 - DNA, 101 bp, Phoenix dactylifera
aacaagctcattaaaatctaatctaacataccatttaaattcaaatctatytacaatgtagaataaacttattgctagcatgtgttgggcaatattgttac

SEQ ID NO: 418 - DNA, 101 bp, Phoenix dactylifera
agaataaacttattgctagcatgtgttgggcaatattgttactgctcacayttcaaagcaagtacygttggagcaatcatagaagatggatcaccctaagc

SEQ ID NO: 419 - DNA, 101 bp, Phoenix dactylifera
ctagcatgtgttgggcaatattgttactgctcacayttcaaagcaagtacygttggagcaatcatagaagatggatcaccctaagcacctttgatacatac

SEQ ID NO: 420 - DNA, 101 bp, Phoenix dactylifera
tggagctttcccatcgtgccacttgccagtgctgccacagcatcattttcyacgtagaaccggacatggcttggaaatatgtctctggtacaaggaacaac

SEQ ID NO: 421 - DNA, 101 bp, Phoenix dactylifera
tgtctctggtacaaggaacaacaaatttaggtgtcacaaagtttcaatttycaaatcatttgaaaggtgcagcaaggcatggctaaataatgtttccataa

SEQ ID NO: 422 - DNA, 101 bp, Phoenix dactylifera
aataatgtttccataaaaatcaagatgaaagaattggaaaagatgaaaaaktcgggatatgccgaaagatgcaagtagtgatagatttaacaacagcagag

SEQ ID NO: 423 - DNA, 101 bp, Phoenix dactylifera
tgatagatttaacaacagcagagaattttcgatcatcttttagttacctakattgatgcaaacaataaaaaacagcccactcccaacgatgaacaaaaaac

SEQ ID NO: 424 - DNA, 101 bp, Phoenix dactylifera
aatgctgaaagaggtgtgaattcagccagtgtagtgaaagacctgcaaatrtggtagaccaatcaatcagcaaagaagaagaagaagaagaagaaaggggg

SEQ ID NO: 425 - DNA, 101 bp, Phoenix dactylifera
taaaaaagttgaccgtgaaatgtttcaaaaattatgaaaccccaactgtcrtgagttgcctttgtcgtccgaaagttaaatttctattaggaagaaaatat

SEQ ID NO: 426 - DNA, 101 bp, Phoenix dactylifera
tttaactaaaccaaaactgtagcacttaagtctaagaactgaccaactctrgcagctgggaggaaatatcacaaaagagagggaagaaatatatacctcct

SEQ ID NO: 427 - DNA, 101 bp, Phoenix dactylifera
actgcctggacagctgcacgattagagcaagcctttgaaagggcttgagcyataacctcctctagggtctctctggctgcagtttctgtggaactcatgat

SEQ ID NO: 428 - DNA, 101 bp, Phoenix dactylifera
ttgtgaatgacaagctgtcatccatttctttcaaaaggtcatatcctttgstaatgcaaacttcctttaattctcatgcaccttaattttgggtattctta

SEQ ID NO: 429 - DNA, 101 bp, Phoenix dactylifera
gaatgatatgatatccgattcctaatgatgtataatctatatggtgatcarcaacacgagcaattcataagcaactaatttcataatgtastaaaaattct

SEQ ID NO: 430 - DNA, 101 bp, Phoenix dactylifera
atggtgatcarcaacacgagcaattcataagcaactaatttcataatgtastaaaaattctttaaagtcccagccaccacattagatgcatctaacgccta

SEQ ID NO: 431 - DNA, 101 bp, Phoenix dactylifera
aaaccataaataccccatgtgctttacataatctatcatcatgattgatgragttttaaatttcttcttaaatttacttgtcatgtaacactgtgtgagaa

SEQ ID NO: 432 - DNA, 101 bp, Phoenix dactylifera
tggttcagaagaagatgcacgagaggccatgatatttcaactgaggccctrtgaaggtaaaggttaagcaagaaaaggtgtaaggccttttattcatccta

SEQ ID NO: 433 - DNA, 101 bp, Phoenix dactylifera
acaatcatgcaatgtaacagtatgacgaatgaaccaaggtatgccatattrtattgaatcggacagttctgaccgtaccatatcgtaccggtccggtacta

SEQ ID NO: 434 - DNA, 101 bp, Phoenix dactylifera
cgacagtatgctgaattggctccataccaagcgtccaatacagtaacaaartggcactagtaaagagtacggtaccgagatggcatactttaaaaacacaa

SEQ ID NO: 435 - DNA, 101 bp, Phoenix dactylifera
atatctccccattgcgataccttctcatctccactactctctcgatgcaawaggcaacgatcccaaatrttcttgkgatctctctctccctcccctgttcc

SEQ ID NO: 436 - DNA, 101 bp, Phoenix dactylifera
accttctcatctccactactctctcgatgcaawaggcaacgatcccaaatrttcttgkgatctctctctccctcccctgttccaaacaagcggaattggag

SEQ ID NO: 437 - DNA, 101 bp, Phoenix dactylifera
catctccactactctctcgatgcaawaggcaacgatcccaaatrttcttgkgatctctctctccctcccctgttccaaacaagcggaattggaggggaagg

SEQ ID NO: 438 - DNA, 101 bp, Phoenix dactylifera
tattgagggattgatggattgatggattcgaatttggatcgaaccagaaasagaggggtttcgagattggggcgcaaatgggccagcggaaggagatcgag

SEQ ID NO: 439 - DNA, 101 bp, Phoenix dactylifera
tatcatacatcacatattatatatcatccaatcaacaatttcattcaacartatttcagtgagattacttatcgctatatagttagcaaaggttagtttcg

SEQ ID NO: 440 - DNA, 101 bp, Phoenix dactylifera
tagattttgtggcgaagatagcatcttggccgcaaggtgttgactagttaytcatcttcttttttrgaatttattggtggtttctgatatccgggagctgt

SEQ ID NO: 441 - DNA, 101 bp, Phoenix dactylifera
agatagcatcttggccgcaaggtgttgactagttaytcatcttcttttttrgaatttattggtggtttctgatatccgggagctgtcgtagatttagggtt

SEQ ID NO: 442 - DNA, 101 bp, Phoenix dactylifera
tggctttttcaggttatttggctgatttttcagctggcttttgacatttarcggcgttggggggatgaagtttcaggatttatcatgaatttttggtttga

SEQ ID NO: 443 - DNA, 101 bp, Phoenix dactylifera
ttgatgaagtctcataaatcttagaagaaaagctatccttcaaaagaacargcattatttgatagaaaaatgcttgaatttagggatgatggccataaatt

SEQ ID NO: 444 - DNA, 101 bp, Phoenix dactylifera
gatttttttcttatcaagttgccagctttgttctagaggaagattgaatgwctctggatgtgagatgtaatgttaagtcatccttaaagttctttttacct

SEQ ID NO: 445 - DNA, 101 bp, Phoenix dactylifera
gtcgtggtgtagtaggggcggcagtgatggtggtgtcgtagcggaagcagyggcgatgatgggggtgatggaggaccaayaatgtgacagaggcaacaatg

SEQ ID NO: 446 - DNA, 101 bp, Phoenix dactylifera
gtggtgtcgtagcggaagcagyggcgatgatgggggtgatggaggaccaayaatgtgacagaggcaacaatgatggtagtggtgatatggtagaggtggtg

SEQ ID NO: 447 - DNA, 101 bp, Phoenix dactylifera
tctttctggtgctatcagtgcttattgttgaggttgttaatcatcaattawgccccaaatagcactgcagtgaaacatgcgtactcttgttaaacctctta

SEQ ID NO: 448 - DNA, 101 bp, Phoenix dactylifera
cttacatacattcttatgtgcttatgtacatacatacatacattaattaaytattgatcccacactaaatgttcgattgcatcccccacaatacatacata

SEQ ID NO: 449 - DNA, 101 bp, Phoenix dactylifera
tcagtggtgtctaccccactgagaacctgcacctctataacccctaccacygttgtggtctttgcatcatcttctacctccactagtcctgccatcttcgc

SEQ ID NO: 450 - DNA, 101 bp, Phoenix dactylifera
ttgtgatgattgaaaagaaaaattcttgctgctgacttgtagggtgatggrtgtgaaatccttgcttcactaatgctacgctattgaaggagaggaataga

SEQ ID NO: 451 - DNA, 101 bp, Phoenix dactylifera
tgctatggtgtgaagttggaactgactgcatcaaatgttgtctatgtacgwtgtgctgcagagcaccttcagatgactgaagaaattgctgagggcaactt

SEQ ID NO: 452 - DNA, 101 bp, Phoenix dactylifera
gaaatccattattagtttgctttcaggtatacaaaccaagtacctgctttyctagattatctaattaatcaatgacttggttaccctctttaaacttgtgt

SEQ ID NO: 453 - DNA, 101 bp, Phoenix dactylifera
ttgtgttcaaagcagcagtgaacaaaactcccaatcagccctctggaattrcttatgacagattgcttctaccggggtgtttgacaagagatatttcctga

SEQ ID NO: 454 - DNA, 101 bp, Phoenix dactylifera
acttaggattgggcctgaatattacgggctatctctcggacttagagtagyagcataagatcatattttaggttcgatgtattatactagagacctaggat

SEQ ID NO: 455 - DNA, 101 bp, Phoenix dactylifera
tgacattagtaatggcggatgaccggcatgtaggaagatgactcttatttwtatcttattaattggaaaacatgtgcttaccatattctttatttccattc

SEQ ID NO: 456 - DNA, 101 bp, Phoenix dactylifera
attgtagttgtcaagtcgagagaggtcgacttgcagatttctgggttcgamtcgaaattttcaggagttaactcataatcttcaggagttggctcgagctt

SEQ ID NO: 457 - DNA, 101 bp, Phoenix dactylifera
aggaatatcaacttgaatatttaaggcaggatcaacattttcagtagaaasaatcaattcccatctgaccgtcttatatgtattagaacaattttgctaaa

SEQ ID NO: 458 - DNA, 101 bp, Phoenix dactylifera
cattatggtaattcctactccatagaacacggttccttcataaccaatctmttctccattcactcgttcgatgctcacctggagtcgagcgcggggaacat

SEQ ID NO: 459 - DNA, 101 bp, Phoenix dactylifera
gtatctaagaagtacatctagcatagaattatggtactctggagattcctrttttgaacttattgcatactcagatgctgactttgctggatgcaaactag

SEQ ID NO: 460 - DNA, 101 bp, Phoenix dactylifera
agttaaactactcatacatctatcatctaaattaatgtccaaaaaatttgraaaacccaattcacccctcctcttgggttgttatcttgggcaacaataac

SEQ ID NO: 461 - DNA, 101 bp, Phoenix dactylifera
gttagcttcaggattcctcaatgagtcgactcttgtatttgttgagtcgayccttgggtttgaagagtcgaccctttgaagctattgctggatgagccgtc

SEQ ID NO: 462 - DNA, 101 bp, Phoenix dactylifera
agaaatccaatttttggatgaacagtggtcgagtcgactctaggagacagygagtcgactccctgagaaaacaacagaagagcgattttcggaggaacagt

SEQ ID NO: 463 - DNA, 101 bp, Phoenix dactylifera
cggaggaacagtggccgagtcaactctagaaatctttgagccgactcactyaaagagaagagtaaaaagcaagctcagatgaacagttagctgagtcgact

SEQ ID NO: 464 - DNA, 101 bp, Phoenix dactylifera
tactatatgcatttgcagcatccatctctattttttgtaactccacgcaayaaaaggacaggattgtgatactctgtaatcatcatgaacatcattgtaat

SEQ ID NO: 465 - DNA, 101 bp, Phoenix dactylifera
ctatgtttggaagaagctcttgattgctttgtgcattgcataattactatmtgtgatgatgactttggatattttgaaatattgctctcaaaaatttaact

SEQ ID NO: 466 - DNA, 101 bp, Phoenix dactylifera
agttggaaagatcatttaccatagccaccttctttgtgctctatataatcyaaaatgtttcttacgaatgtctacagctactaagctaaaaacttttagaa

SEQ ID NO: 467 - DNA, 101 bp, Phoenix dactylifera
agtctctcccaaactctagcctggccctgctgattgttgcaccatgtaaayctagagcccgagaaccccaggtttaycaagccattcgatgtcagaaagtc

SEQ ID NO: 468 - DNA, 101 bp, Phoenix dactylifera
ctgctgattgttgcaccatgtaaayctagagcccgagaaccccaggtttaycaagccattcgatgtcagaaagtcttggaactccttaaccttcctctcat

SEQ ID NO: 469 - DNA, 101 bp, Phoenix dactylifera
ttacatgagctttggttgcacctagagttactcaactggcaggccgggttkggttcaggccaagctcaactcaagatattagataaatcagttaatacttg

SEQ ID NO: 470 - DNA, 101 bp, Phoenix dactylifera
ggttgattaagcatataaaatctaaattggaaaaaaacagtgctagcattrtggaaatataggtcagataattyatagtggtgtgcagcattatgatgtgg

SEQ ID NO: 471 - DNA, 101 bp, Phoenix dactylifera
aaattggaaaaaaacagtgctagcattrtggaaatataggtcagataattyatagtggtgtgcagcattatgatgtggarctaacaatgcaagtatgtaag

SEQ ID NO: 472 - DNA, 101 bp, Phoenix dactylifera
ggaaatataggtcagataattyatagtggtgtgcagcattatgatgtggarctaacaatgcaagtatgtaagtttgtcgtagtattatgatgtgcaaccat

SEQ ID NO: 473 - DNA, 101 bp, Phoenix dactylifera
attatgatgtgcaaccatcaccttataactcttttcaagacaatcacataytatagtaattttaagcttttagctcggccattgtttcaacggatgttgga

SEQ ID NO: 474 - DNA, 101 bp, Phoenix dactylifera
ttagttttctcttcaaaattaaatctaaaaggactgaccttaataactagyagtaatcctagataatacttggtatagatcgaaaaatagattagaaatag

SEQ ID NO: 475 - DNA, 101 bp, Phoenix dactylifera
ttaccaaagtttgggcactatcaagggcattagtaaataaagccacccctkgaacattcctaggatcttcctaagcytgtactcagcacctaaattagggt

SEQ ID NO: 476 - DNA, 101 bp, Phoenix dactylifera
gcattagtaaataaagccacccctkgaacattcctaggatcttcctaagcytgtactcagcacctaaattagggtcaaaagggtgagctcctacacaatag

SEQ ID NO: 477 - DNA, 101 bp, Phoenix dactylifera
aagctttcagtgacatccctagcccagattgctatcttgggcttcacgatrtgctctagggttccataggaagtggattaagtgttgtgactaaaagaact

SEQ ID NO: 478 - DNA, 101 bp, Phoenix dactylifera
gcttatagcttgaatggatttacccatcttggtccacccggcccctaagaytcattcactctagcctctacgactacctctgcattaaccttccttacgag

SEQ ID NO: 479 - DNA, 101 bp, Phoenix dactylifera
gactacctctgcattaaccttccttacgagtccatgggcttccttaactastctttgtttcactctgcactgctacgacaacagtagcaaaagggacctcc

SEQ ID NO: 480 - DNA, 101 bp, Phoenix dactylifera
tacctagagaattttgccaaggatttcaacctctaaataaaagctagtctrgttttggatgaggtggtccaggtggagagggaggacttgaacattggatg

SEQ ID NO: 481 - DNA, 101 bp, Phoenix dactylifera
ttaaagttctattggacttggataattattagtgcagcagatagtgaactrttggagtttttgtgtttgcttctccacaaattagtaaaataaggtgtaag

SEQ ID NO: 482 - DNA, 101 bp, Phoenix dactylifera
atgtccccatcacaaacactgtttaacaaaaaaatccttggtacctaaaaytatttacgtacaattctgaaaataattttttttacaacgcatggctttca

SEQ ID NO: 483 - DNA, 101 bp, Phoenix dactylifera
tcatagtctccaacaagttactaaaggggttaattccatgtagtttgccaygaagctagcacccttttctttctccatagcatcstaagaagcaatggtgg

SEQ ID NO: 484 - DNA, 101 bp, Phoenix dactylifera
tccatgtagtttgccaygaagctagcacccttttctttctccatagcatcstaagaagcaatggtggcagctcaactgttagccttcaaggaaaactttga

SEQ ID NO: 485 - DNA, 101 bp, Phoenix dactylifera
ccttgttttatcaatgttatcaatcatgcaaatggaagtttggaaatagayattcacatcagaaggttagaaaggagaaagttaactatcttgatgttgcc

SEQ ID NO: 486 - DNA, 101 bp, Phoenix dactylifera
tttttttttatccccagtgatattggtatgtattatcaaacttttgtgcgktatgcatgttccatatgacaccaattccttaccagcatgggactatttgt

SEQ ID NO: 487 - DNA, 101 bp, Phoenix dactylifera
accagcatgggactatttgtacatacaataccttacacagtataatctcakacctcaatcattaaattattccctcaaggttcccttaaggttgtccttca

SEQ ID NO: 488 - DNA, 101 bp, Phoenix dactylifera
aatatgtcagattcttaattgtttaacatatcatagaaaatgcccccatastcatggtttctccagcctggctcattttcttgcgttcagtaagttccttg

SEQ ID NO: 489 - DNA, 101 bp, Phoenix dactylifera
tggtcacacaacgccctatgttacatggacttggatttgtgtgcttggtgygggtgtacgtttaagcatcagatccttttaatattttagaatatcttcgc

SEQ ID NO: 490 - DNA, 101 bp, Phoenix dactylifera
gtaatgatcgttcttgtggttcatgagattactagaatagctttatatgcmgtctgttgggctatgatactttttggtatccgggcaaagcttggcatcaa

SEQ ID NO: 491 - DNA, 100 bp, Phoenix dactylifera
ggaactgcgaagatagaaatagctacatctttcgcagcagcactggatcastgtagttctaatttacctctcaagttatgttcaaatcgtaatttatgtt

SEQ ID NO: 492 - DNA, 101 bp, Phoenix dactylifera
gcctgctccacaaactttgtcaaccacccatactggttgacctccagggawcctggaactctttttaacgttttgtttggattcaggttgaaaccataccg

SEQ ID NO: 493 - DNA, 101 bp, Phoenix dactylifera
cgtctgccaacgtgattggcaccaatcaatttcaaaaattggaaagaaatkaaaatatagaagaagcttcaggagatcttgctaatactttccgaggttag

SEQ ID NO: 494 - DNA, 101 bp, Phoenix dactylifera
gggaataacagccttgaccatacctcatcatctcgttcaagtgagccttgmgcctgcatccattaaccatgacactaattatttttttgggtacgaacaac

SEQ ID NO: 495 - DNA, 101 bp, Phoenix dactylifera
atataacaaatgtatttgcacaattcaggaatagctaaggagtagttgtgrgtacaaagtatgcaagaaaccttgattttcaacctcatttactatttgga

SEQ ID NO: 496 - DNA, 101 bp, Phoenix dactylifera
gaaaaagagcatctactaggtcactaggaagacctgcaataccatgttgasattacatactgcatactagaacaagttctattttaggctcatactgattg

SEQ ID NO: 497 - DNA, 101 bp, Phoenix dactylifera
aataccctaaaataatatctttcaaggaaacagaattattttagaagtttyataggagaatctgtcactgatgcttttgcagcagatcagaaaaaagaagc

SEQ ID NO: 498 - DNA, 101 bp, Phoenix dactylifera
actgttgacaaaaaaatctttactgactcttgaagcataacactagttcawgcaaaatttagggacacaaaatctaagcaaaagcactgcctctgatgatt

SEQ ID NO: 499 - DNA, 101 bp, Phoenix dactylifera
caagcagggctcaggctgcaaggaagcccaatccagcccagcctggtgccraccctagtttcgaacaaaactaccaatcaatttcggctgaaaccaccaaa

SEQ ID NO: 500 - DNA, 101 bp, Phoenix dactylifera
accaatcagaatatttacctgatcaattatatggtttaaacaaatgcaacmaccatccaggatgcttgtagacaatgcagactaaagagatgagaagatta

SEQ ID NO: 501 - DNA, 101 bp, Phoenix dactylifera
taccaattgtcaataaaacagagatgaactccacaatctgaaaaataaaamaaccattttgagttataatatcatagaaattctatgcaatagtctatcat

SEQ ID NO: 502 - DNA, 101 bp, Phoenix dactylifera
ctaagatagtaatccatacttctgactaaaacataagtaatccaccatatrcagacttgcagaggctgatatcacaaggtatctaagagtatgctgctgtt

SEQ ID NO: 503 - DNA, 101 bp, Phoenix dactylifera
ggtctatttttccatcgttaggaatgaaaatgtatgtgtaaaaagagattwattaaatatcacggttgagtggctaatttcacacagacaactataattta

SEQ ID NO: 504 - DNA, 101 bp, Phoenix dactylifera
tattagaaaggattgcaaagttaaaacacattatccaacaccatatataaraaagacaagaaaatcagtccaagactttttgactagttgagaccttcaaa

SEQ ID NO: 505 - DNA, 101 bp, Phoenix dactylifera
atgatgaacaatcgtgtttaatacactataaactttagaaaagaagatackgaaggctgataaatatcatcagtawttatggctgactgtgggaaattgcc

SEQ ID NO: 506 - DNA, 101 bp, Phoenix dactylifera
ctataaactttagaaaagaagatackgaaggctgataaatatcatcagtawttatggctgactgtgggaaattgccatattaggttataatggcacctaaa

SEQ ID NO: 507 - DNA, 101 bp, Phoenix dactylifera
gttaggtgaactaaactacttccttgggcttcaaatcaaataattaaaaawagaaatcttcatcaatcaagcgtggtacatcaagaagatgctaaacaagt

SEQ ID NO: 508 - DNA, 101 bp, Phoenix dactylifera
agcaattggcacacctatgagttcttcatgcaaataagataaagatgaatyaggtaagagcatagatcaaaagttgtatagaggcatgattggatcacttt

SEQ ID NO: 509 - DNA, 101 bp, Phoenix dactylifera
tgggcaaacgatttttttggacaagagctgatattggtaatggaaaagagytgtctctattaaagcttgagctaactcagtgatgggacattcactttaaa

SEQ ID NO: 510 - DNA, 101 bp, Phoenix dactylifera
ctgacctagttagagaattttatagttccttgaagaaggataccaagacargaacrttaattagctatgttagagggattgaaatgattgtgaccgaacaa

SEQ ID NO: 511 - DNA, 101 bp, Phoenix dactylifera
ctagttagagaattttatagttccttgaagaaggataccaagacargaacrttaattagctatgttagagggattgaaatgattgtgaccgaacaattgtt

SEQ ID NO: 512 - DNA, 101 bp, Phoenix dactylifera
ctttcatcgtctgacattccctatacctcaccttcagaatcaacaggacarccgactgcatcttctatgagattggacaatgatcagatgaggaggatgat

SEQ ID NO: 513 - DNA, 101 bp, Phoenix dactylifera
gactgcatcttctatgagattggacaatgatcagatgaggaggatgatcaraatgattacagatgaagtagataccagactgatagaaagaatagaacagg

SEQ ID NO: 514 - DNA, 101 bp, Phoenix dactylifera
aggactacttgtatgcctactattttgattgctatcacatgtatagatccrttagaatttttactatcctttgatgaacatttkgtactcacttatgcatt

SEQ ID NO: 515 - DNA, 101 bp, Phoenix dactylifera
atcacatgtatagatccrttagaatttttactatcctttgatgaacatttkgtactcacttatgcattacagatgtaatcacccttttgcaatgcartgaa

SEQ ID NO: 516 - DNA, 101 bp, Phoenix dactylifera
atttkgtactcacttatgcattacagatgtaatcacccttttgcaatgcartgaaactccctcttcagcaattcgatgtgtgtatctctcttattcatatt

SEQ ID NO: 517 - DNA, 101 bp, Phoenix dactylifera
gaatgtgctagaatgacttaaaaaattggttcaagggttttaaactcaacytttactatgtttggaaccatttcatcttcgccctgcactcaagtcgactc

SEQ ID NO: 518 - DNA, 101 bp, Phoenix dactylifera
agttaactccaactgtagatgagccgactccaacaagaaagatagaaagtyagtttctaatacactaagatcgagtcgactccaactgaaatcgagtcgac

SEQ ID NO: 519 - DNA, 101 bp, Phoenix dactylifera
taaaggaaaagaggaataactacgcttacattgatttcagaagtactcctyacattcaaggcttaattatctgatcctcaaccaccagcgcattcaagaga

SEQ ID NO: 520 - DNA, 101 bp, Phoenix dactylifera
gtaatcatagaaattataatgtactcccaagtgagccttggggagtagacrtaggagcagggttggctccgaaccactataaaattccgtgtrtctatgtt

SEQ ID NO: 521 - DNA, 101 bp, Phoenix dactylifera
gagtagacrtaggagcagggttggctccgaaccactataaaattccgtgtrtctatgttgtrgttctttgagcatcttttctatattgttttattaatagg

SEQ ID NO: 522 - DNA, 101 bp, Phoenix dactylifera
ggagcagggttggctccgaaccactataaaattccgtgtrtctatgttgtrgttctttgagcatcttttctatattgttttattaataggttgatttatct

SEQ ID NO: 523 - DNA, 101 bp, Phoenix dactylifera
taatgtcctcagggcaggttaggatcaatccagctttgtgtctttctgcaytagaagatccataaayatggagtatccagaaaggttgtgcctctgacttg

SEQ ID NO: 524 - DNA, 101 bp, Phoenix dactylifera
ggttaggatcaatccagctttgtgtctttctgcaytagaagatccataaayatggagtatccagaaaggttgtgcctctgacttgtattgtttaggctttg

SEQ ID NO: 525 - DNA, 101 bp, Phoenix dactylifera
cttcctccggcattgtgcactcgaccatgatgtcaatggtgcttgaccaartggctagaagtctctacttctatagtgcctactttaatggttgactggtc

SEQ ID NO: 526 - DNA, 101 bp, Phoenix dactylifera
ctggtcatcactatgatagtatgagcttggaaatacgatcgcaatctgagytgagataattagggcataggccattttctccattcttagatatcttgttt

SEQ ID NO: 527 - DNA, 101 bp, Phoenix dactylifera
tcccaaggtgactccatatgcacactttctcggatcgagcttcatattgtrttttcttwgtgtctagaaggctttgtgtaggtaggcattgtgatgttcag

SEQ ID NO: 528 - DNA, 101 bp, Phoenix dactylifera
tgactccatatgcacactttctcggatcgagcttcatattgtrttttcttwgtgtctagaaggctttgtgtaggtaggcattgtgatgttcagccttctga

SEQ ID NO: 529 - DNA, 101 bp, Phoenix dactylifera
atacttccatgttccaatcgatctgatccttgaagacacaattgactagcyrttggcacgtggcttcagcattcttcaactcaaatggcatcatctggtag

SEQ ID NO: 530 - DNA, 101 bp, Phoenix dactylifera
tacttccatgttccaatcgatctgatccttgaagacacaattgactagcyrttggcacgtggcttcagcattcttcaactcaaatggcatcatctggtagc

SEQ ID NO: 531 - DNA, 101 bp, Phoenix dactylifera
ttcggacagatcttattaaagttgatgaagtcaatacagactctctaatgyccattagcctttttgacaagtaccacattggctatccattctatgcaatc

SEQ ID NO: 532 - DNA, 101 bp, Phoenix dactylifera
caatcgacttcacggatgatcccatcctcccaaagcttaccaacctcttcrttgaaggcatattgtcgcttttggccgaagctcctctttttctatcatac

SEQ ID NO: 533 - DNA, 101 bp, Phoenix dactylifera
tactcagctatctccttatcaaaaagtgggatggagatgagcttcttaatrggttttyctcactattccttctatccatgtcgaacatccaaaccttcaat

SEQ ID NO: 534 - DNA, 101 bp, Phoenix dactylifera
ctatctccttatcaaaaagtgggatggagatgagcttcttaatrggttttyctcactattccttctatccatgtcgaacatccaaaccttcaatgggcaat

SEQ ID NO: 535 - DNA, 101 bp, Phoenix dactylifera
cttaacgaccagaaagtctagcatgatggttgtctatcaagattcttgacygattgtgacgraaagggtgattactccttccacatatacaaggtcttcag

SEQ ID NO: 536 - DNA, 101 bp, Phoenix dactylifera
gaaagtctagcatgatggttgtctatcaagattcttgacygattgtgacgraaagggtgattactccttccacatatacaaggtcttcagaaaaactgatc

SEQ ID NO: 537 - DNA, 101 bp, Phoenix dactylifera
cttcttagacaatatgttggtaagttcattttctagaattctccatagaawaaaatatctgtacagcttccattatccactaaaactcttcttaaatcaaa

SEQ ID NO: 538 - DNA, 101 bp, Phoenix dactylifera
atcatgggggtcagcaaccttcatatcaatgtccgagaaagatataacttyctaggatcttggtctcttcagggctgaatctcctgctcgggctgctctag

SEQ ID NO: 539 - DNA, 101 bp, Phoenix dactylifera
gctcgggctgctctagtgtatcttctgagtcgtcgaacttaaccctcctgytatagatccacttgaaatggcattaatgactctcaccatggactggaatt

SEQ ID NO: 540 - DNA, 101 bp, Phoenix dactylifera
ttaatgactctcaccatggactggaatttcaacctctcaggcgagtgcctrttcccctatgcttcctcttctcggcattagttttttacatactaaccaag

SEQ ID NO: 541 - DNA, 101 bp, Phoenix dactylifera
ggatgagagcttcaatcttgctcttcaactaaacacttctttattgttgtrgctgtgatccttgtgaaaccagtaatactttctggggtttcagctagccg

SEQ ID NO: 542 - DNA, 101 bp, Phoenix dactylifera
tagaggagtgtacctctcaaacctcaatggtgggctcctcggaggtcggcrgttgtcctcttcatgtctaggcctctttctccatccatctctttcttctc

SEQ ID NO: 543 - DNA, 101 bp, Phoenix dactylifera
gttgtcctcttcatgtctaggcctctttctccatccatctctttcttctcygccccttcttctgccattttcatctttattttcctccccctgagctacca

SEQ ID NO: 544 - DNA, 101 bp, Phoenix dactylifera
tggctcgagacaacatcttcactaggtccttgggagtcctcttctccaagragaatgttggcggaggcctgaagggccgagtcagtcctgccgcctgccag

SEQ ID NO: 545 - DNA, 101 bp, Phoenix dactylifera
tcttccctctgcttcactatgaacaaaccgacggtgtccttcttctgcctytggctactcacaaagtgagccacaaactagcrgctaagctgatcgaaaga

SEQ ID NO: 546 - DNA, 101 bp, Phoenix dactylifera
ggtgtccttcttctgcctytggctactcacaaagtgagccacaaactagcrgctaagctgatcgaaagaataaatagtttttggtcgraggctagaatacc

SEQ ID NO: 547 - DNA, 101 bp, Phoenix dactylifera
gccacaaactagcrgctaagctgatcgaaagaataaatagtttttggtcgraggctagaataccaaagatgagctacttttcttagaatggccgaaaaagc

SEQ ID NO: 548 - DNA, 101 bp, Phoenix dactylifera
ccatccgaaatcaaaaccatctcatcgactcccttctccaccgtaggcatmccttctccctctcttccatttgctagagttctcgccactggaatctaagt

SEQ ID NO: 549 - DNA, 101 bp, Phoenix dactylifera
tctcgccactggaatctaagtctatctcattcctatcaaagttccctcttyattttatcctttttaacataaaaatggttaggtcctgcctttagcgtttt

SEQ ID NO: 550 - DNA, 101 bp, Phoenix dactylifera
tttagagtatgggatttgcaatgtttattttttagagctgaaaattttttkgtgtgcaaactagtagctgatttaagattgttgtgacaagatagatctca

SEQ ID NO: 551 - DNA, 101 bp, Phoenix dactylifera
aaagcttgactattgcaatctattatgtgctttttttttggtacatcggcragttgcccaaaaagtcagaaagtaaaaaagaccgcaagagagacgggata

SEQ ID NO: 552 - DNA, 101 bp, Phoenix dactylifera
gtaattttaatttaatttgcaaaaaagttgattagaaagccagatattacytgaccaccaaattgtaattactcaaattttgagaggcaatttgttaacat

SEQ ID NO: 553 - DNA, 101 bp, Phoenix dactylifera
gccaccatgcatgtaacgtagcattctagatagggaagcgataacatggcygtcctccgtgtttgtccctgatccaattattggtaatattttctttttag

SEQ ID NO: 554 - DNA, 101 bp, Phoenix dactylifera
attggtaatattttctttttagaacaataatttcggaaaagctctcaacaygatggcagtagctctttattttattgtcactatggcctcgtaagagcaga

SEQ ID NO: 555 - DNA, 101 bp, Phoenix dactylifera
tcgcagcatgcaaaatagattgcaaggtcaaattgcattagtgcagttgayctacaagggttcatcattgccaagctttttccaacttgaagctgttggag

SEQ ID NO: 556 - DNA, 101 bp, Phoenix dactylifera
acacctgtaaaaggacttagtgcagtccatcgagtttcaacgctgttcagmmtaattcacctgatcatacatgtgatttgcatcctatcttatgattgtct

SEQ ID NO: 557 - DNA, 101 bp, Phoenix dactylifera
cacctgtaaaaggacttagtgcagtccatcgagtttcaacgctgttcagmmtaattcacctgatcatacatgtgatttgcatcctatcttatgattgtctt

SEQ ID NO: 558 - DNA, 101 bp, Phoenix dactylifera
tctttgttcaattgaatcaaattaaccaaatagagctaccataatacagcyagaatttacctgctcaaaggatgacatgaatcggtgccttggcttagcac

SEQ ID NO: 559 - DNA, 101 bp, Phoenix dactylifera
aacggcattcaactgatttaagtatgtccactgatgtatagtaatgaaagytaaaacaataaagacaattcttttttctaagtaacttgcctactgacacg

SEQ ID NO: 560 - DNA, 101 bp, Phoenix dactylifera
ttccagagaaagaaacctcaatcaaggtggactcaggaatggagacatacyatgcggtgaagaaccagtctgaggtccaacaagagtgcaatatctaatta

SEQ ID NO: 561 - DNA, 101 bp, Phoenix dactylifera
cgtgcagagaggctacttcagtgttttgaacttatgatctctaggtcatartgaagcaaccttaccattgcaccaaagctcacccatatgacaacaggaat

SEQ ID NO: 562 - DNA, 101 bp, Phoenix dactylifera
gaaaccactgatactgagtgatacacactgacatagccagtaaataccagyggcacagctgtttcgacatcccaagacttgtatagatgcatgttttgtgc

SEQ ID NO: 563 - DNA, 101 bp, Phoenix dactylifera
ctcgtggagtactgacaaagaggagccaaagagggaagaggagagggagamggaaagagaggaagaaagaacgggagggggtgggtaggaaagggaagaaa

SEQ ID NO: 564 - DNA, 101 bp, Phoenix dactylifera
ttaatcaccctaacgttcagtatttcttaaaaaaattggcatgattaaaayatattaatcagcatctcttatctagacataatgctttgatagtgttttca

SEQ ID NO: 565 - DNA, 101 bp, Phoenix dactylifera
gctcttgaagaactccatactttattaatttatgaaaaaattagtctcgcrgaagaggcctctatgcaatctactactgcatatattgcctcaagacctac

SEQ ID NO: 566 - DNA, 101 bp, Phoenix dactylifera
tgtcaaatctataataaggttgaacatattgccattgtttgctattattgyatgaactatgcatatcaagattgccatcctctagagaaatctgcagctat

SEQ ID NO: 567 - DNA, 101 bp, Phoenix dactylifera
taataattgctattcttacttgatgaatctaactttgttgtcaagaacaargatacaaaggcaatgtcgtaccgctagatgactaagaattgacttcatct

SEQ ID NO: 568 - DNA, 101 bp, Phoenix dactylifera
actttattcatggcttcttcccatgtgttacaaatttagtttctttgacakttttctcttttaaagcacaagttgaaaaaaaatttaataaagtcaaaacc

SEQ ID NO: 569 - DNA, 101 bp, Phoenix dactylifera
cccatactttggaacaaaatagcgttgtcgaaaaaaaacatcgtcacactrttgaaaccagactttacacttcttgctcatacatctctttctcgaaaatt

SEQ ID NO: 570 - DNA, 101 bp, Phoenix dactylifera
cgtgacgtcatctttaatgaacatgtcttccctttttcaaccatatcactrccttaatcaacttttcacaaccccacttctgctcttgattctctttctat

SEQ ID NO: 571 - DNA, 101 bp, Phoenix dactylifera
aactcaactactacaccatatgacaacacaagcattccaatcgagctcatsccaccacctcaatacaagaccacctcttaccttccctccctcgaatttcc

SEQ ID NO: 572 - DNA, 101 bp, Phoenix dactylifera
ttcaagaaacgatttttatgtctcaacatcctagttatattgatccatcaraacccactcatgtctgtcagatgcgcaaatcaatttacggacttcagcaa

SEQ ID NO: 573 - DNA, 101 bp, Phoenix dactylifera
ccctttggtcgttgtcgattttgagagattttccctagtttttcatcaagrattttactggaccaccattgatacctcgtcgaaatcaaggtatgaccttt

SEQ ID NO: 574 - DNA, 101 bp, Phoenix dactylifera
cctttaatccttcccctcctccctttttccgtctcaatgtcattatcactrgatgattggaagtggcgagtcattggattttcattaaatagagtcttcct

SEQ ID NO: 575 - DNA, 101 bp, Phoenix dactylifera
tccttgattctgccacactcatgtttgccctttatttcttccatcgttggycacaaccgacaatgttctgccatagaccttactgaagtctagctttctag

SEQ ID NO: 576 - DNA, 101 bp, Phoenix dactylifera
caaccgacaatgttctgccatagaccttactgaagtctagctttctagccratgatgtcgagatcactgatggctctccattgtggattgtgcatcgagcc

SEQ ID NO: 577 - DNA, 101 bp, Phoenix dactylifera
ttccttcactagtcaagtactatcactgggttgccactacagcctgttgcyaccatggatgccaaccccaagccaccacctcttgccrctacaattgcttg

SEQ ID NO: 578 - DNA, 101 bp, Phoenix dactylifera
tacagcctgttgcyaccatggatgccaaccccaagccaccacctcttgccrctacaattgcttgacaatccctcttttctccttcctctattttgaaagag

SEQ ID NO: 579 - DNA, 101 bp, Phoenix dactylifera
cccttcgcaccattaagccttataaagaaagaaattgctttctctagcccratgaaaaaaatatttttctcgctctttcagctttgattgtgtaatagctt

SEQ ID NO: 580 - DNA, 101 bp, Phoenix dactylifera
aataattgagtaaaaagttcgctaatcagttattgggagtgtactaaaatwtttgcatgaagacaagaagaaatttttcagtaactatcaatatggacaaa

SEQ ID NO: 581 - DNA, 101 bp, Phoenix dactylifera
aaagatttgacttcaagaacaccttgaagagaccctttaacactcacatgyaatcataacaaatttcaacttaggctatctcaagagtcgaccctagcata

SEQ ID NO: 582 - DNA, 101 bp, Phoenix dactylifera
aatttcaacttaggctatctcaagagtcgaccctagcatactcttacagaytgatacgccttcatgagtcgacacatgaagataacgaatcgatttttgtc

SEQ ID NO: 583 - DNA, 101 bp, Phoenix dactylifera
ttctcattgaaaggccaagggtcggctacaaaatggccgaccccaaggtcrggctaagtacaaaagggccaaaatggcctatttaacaaaagcaaaaagag

SEQ ID NO: 584 - DNA, 101 bp, Phoenix dactylifera
gcccaacgcagcccctgctatgcaagataggcctggacctaagctatacakttcggattttgataactggacacaaagcaggacgagctcgaatgcctagc

SEQ ID NO: 585 - DNA, 101 bp, Phoenix dactylifera
agccctgccaacggacgtacccatatgcgtgcctacgccctcctacgtggycgtacatagcgacgccaccatgccatgctttgcttgctggcccacgacag

SEQ ID NO: 586 - DNA, 101 bp, Phoenix dactylifera
cggacaggatttgggtcaaaaagtgggccaaaccctctaacaggtcggatycaagwactcagacccgacccgcacctcaatccattaacaggtgatcggcc

SEQ ID NO: 587 - DNA, 101 bp, Phoenix dactylifera
aggatttgggtcaaaaagtgggccaaaccctctaacaggtcggatycaagwactcagacccgacccgcacctcaatccattaacaggtgatcggcctcaat

SEQ ID NO: 588 - DNA, 101 bp, Phoenix dactylifera
attaacaggtgatcggcctcaatggaaaccctagtcgatggctataaaagscgaagttagggccatctagggtttcagagaggttaaacagacggccaccg

SEQ ID NO: 589 - DNA, 101 bp, Phoenix dactylifera
ttcttcttcccctttgctaggttagcgtttggtttttttcccttccatccyttttagcgtttctctgcagccatgacggtcaagctctccaagtcygagaa

SEQ ID NO: 590 - DNA, 101 bp, Phoenix dactylifera
catccyttttagcgtttctctgcagccatgacggtcaagctctccaagtcygagaagaaggtcgagtatgacaagaagctgtgcagtctcctggacgagta

SEQ ID NO: 591 - DNA, 101 bp, Phoenix dactylifera
ctccccttgcttgtggtaaagattcttgtctcgggcctaagatttcggtakatatgttttgatttttaatcgcgttttgcctgtgctgttttttttccccg

SEQ ID NO: 592 - DNA, 101 bp, Phoenix dactylifera
ataataaactaaaaatcagttttttatgatactatgagctgtgagttgctrggatctagtartatttgtgttgcatttgttrttgtattactaggttagag

SEQ ID NO: 593 - DNA, 101 bp, Phoenix dactylifera
aaaatcagttttttatgatactatgagctgtgagttgctrggatctagtartatttgtgttgcatttgttrttgtattactaggttagagggcttttgata

SEQ ID NO: 594 - DNA, 101 bp, Phoenix dactylifera
ctatgagctgtgagttgctrggatctagtartatttgtgttgcatttgttrttgtattactaggttagagggcttttgatagcagatggcatgccttatat

SEQ ID NO: 595 - DNA, 101 bp, Phoenix dactylifera
tctgttcacatagttagttcgtgtataagttattattcagcggtttgtgtracattcttcttcatgaaggttcacattcttgattttgagcattttgtatc

SEQ ID NO: 596 - DNA, 101 bp, Phoenix dactylifera
tctttttttttcttctcctcctttatatgatgtggtctggttatgttgctkcttatgttgcttttttaatgcagggaaatgtgggtttgattttcacgaag

SEQ ID NO: 597 - DNA, 101 bp, Phoenix dactylifera
aaggtttgctatatcattcttactaacattgtgtgcgtgtgttttcaccgytgtgcttcgtaaatgatctgataggcctttagggttcccaagtagtgcat

SEQ ID NO: 598 - DNA, 101 bp, Phoenix dactylifera
agatgtcatgaagatattctaattgttatcagaaatttaaatatcaggaawcaaattttaacattttgtttgacaaatatgttttgagcgagagaaatgag

SEQ ID NO: 599 - DNA, 101 bp, Phoenix dactylifera
atttgttgagatcaggaattatggtgaaggcatgacttgaactcaggtcaytggcacccaactgtcaatgtttataccaatgaattgggtggcagcctcat

SEQ ID NO: 600 - DNA, 101 bp, Phoenix dactylifera
aggcctggcaacatggatgcattataataagattactgcactttgatgatrgcaaggttaaatcctatgatatttcctatcacatttgtgttatgttggcg

SEQ ID NO: 601 - DNA, 101 bp, Phoenix dactylifera
ttactgatttctcagcctattgcagctttcggtgagctgctattccttgaytttctgttttattgctttctgcctttcttgttaagttcttgattaactga

SEQ ID NO: 602 - DNA, 101 bp, Phoenix dactylifera
tttatatatgatgcttgattggtctagcatagttactttgtaatgtgcttstcttggactggtttcagattctaatttgaacaagatatgctatcactccc

SEQ ID NO: 603 - DNA, 101 bp, Phoenix dactylifera
ctaatttgaacaagatatgctatcactccccctcccctacctcaactcccytccccaggccccagtaaaaagcataaatttgaacttgcaactgaagacca

SEQ ID NO: 604 - DNA, 101 bp, Phoenix dactylifera
aacattgcttttatgagattttaatgatagaattgcaggtagtgcttcatrattatgaacttttatgcttatatttgaatcaattgcaaaaaaattatgga

SEQ ID NO: 605 - DNA, 101 bp, Phoenix dactylifera
ggggaaaaggttggctcttctgaggctgctcttcttgctaagcttggcatmagaccattctcatatggtctcatcatccagtctgtgtacgacaatgggtc

SEQ ID NO: 606 - DNA, 101 bp, Phoenix dactylifera
ccctgaggttctggacctcactgaggatgacttggttgagaggtttgcagytggagtctctatmgtcacttcactgtccttggccctttcataccctacta

SEQ ID NO: 607 - DNA, 101 bp, Phoenix dactylifera
gacctcactgaggatgacttggttgagaggtttgcagytggagtctctatmgtcacttcactgtccttggccctttcataccctactatagcagctgcacc

SEQ ID NO: 608 - DNA, 101 bp, Phoenix dactylifera
ctggcagttgctatcgcgactgagtatactttcccacaagctgagaaggtsaaggagtttttgaaggtaggttatgttctcttctgcagggccatagtaat

SEQ ID NO: 609 - DNA, 101 bp, Phoenix dactylifera
ttttagttctttttgatagctggaatattgaagctttacagtttgctatgktaggccggttttgaccagagattctggattgaatttgctttcttctagat

SEQ ID NO: 610 - DNA, 101 bp, Phoenix dactylifera
tgttgaatttgctgagaatatgatctgttctttaatgatatatgagtatgrtcttcgtttagtaaaattcttttcctattttggtaatgtggttttcctat

SEQ ID NO: 611 - DNA, 101 bp, Phoenix dactylifera
tatctagttttcacttctcatgggatgtgctgggatgatgggggtgattamatataccctatggctgcccttagttccttggtcactgtaatagacaccaa

SEQ ID NO: 612 - DNA, 101 bp, Phoenix dactylifera
accctatggctgcccttagttccttggtcactgtaatagacaccaaatatktagtgttaacgatatttatgtcatattgggcagggtaaacattgtttctt

SEQ ID NO: 613 - DNA, 101 bp, Phoenix dactylifera
gtgagctttataaatcaagtaacagtcatgtagaattgatttagcattatktagtttaaccagcatttgcatgaatcctcttagcttcttccaaccaaacg

SEQ ID NO: 614 - DNA, 101 bp, Phoenix dactylifera
tagtttaaccagcatttgcatgaatcctcttagcttcttccaaccaaacgkatctctgctcagactatgttagcagcgggtagccctataatctgaaatgg

SEQ ID NO: 615 - DNA, 101 bp, Phoenix dactylifera
ctggttgggtctttttgtataactgctcttatttaaagtttgatgcctacytaattkcattaaattgggtacatcagaggtaaattcaaatctatattttt

SEQ ID NO: 616 - DNA, 101 bp, Phoenix dactylifera
gggtctttttgtataactgctcttatttaaagtttgatgcctacytaattkcattaaattgggtacatcagaggtaaattcaaatctatatttttttaaaa

SEQ ID NO: 617 - DNA, 101 bp, Phoenix dactylifera
caccataggttacttatatattgattagattttgtcctgttttgaaaagtrgaggtctgaaaaccaagagggatgtaacaactaaaatgatatgtaaaagc

SEQ ID NO: 618 - DNA, 101 bp, Phoenix dactylifera
atattatgacgagcaaagacaaatttttttactaatccgtcaggaagagcmgattccttgagtttgcctagattttgttcagacaatacttgaagaggaag

SEQ ID NO: 619 - DNA, 101 bp, Phoenix dactylifera
tgttgccatcaggttattttatatgaaaagtttggtccttctgatgattgytttaakactagtaggtatttgataaccatcattctattacattgtaatgc

SEQ ID NO: 620 - DNA, 101 bp, Phoenix dactylifera
catcaggttattttatatgaaaagtttggtccttctgatgattgytttaakactagtaggtatttgataaccatcattctattacattgtaatgcaggtca

SEQ ID NO: 621 - DNA, 101 bp, Phoenix dactylifera
gccactttgtctatccttgacttgtgagtttgtctctccagtccctaggtrcaaactatcaaagtctgggtgcggtaggggaaaaatatagataaaaatca

SEQ ID NO: 622 - DNA, 101 bp, Phoenix dactylifera
cttccatcaaagaaaacaatgtctggtgaaaataaaattcaagtctttttrcacctgaggttgatgtaacmtagtctatccaatatcaatacgcctctcaa

SEQ ID NO: 623 - DNA, 101 bp, Phoenix dactylifera
gtctggtgaaaataaaattcaagtctttttrcacctgaggttgatgtaacmtagtctatccaatatcaatacgcctctcaatagtcgaggtattatccagc

SEQ ID NO: 624 - DNA, 101 bp, Phoenix dactylifera
tctatccaatatcaatacgcctctcaatagtcgaggtattatccagctccwtcatcactttggtagtccatgcggtcgtcgtgggatacttggatttaagg

SEQ ID NO: 625 - DNA, 101 bp, Phoenix dactylifera
gagtcagaaattatcatccctatttgaatacttgaatctttcatcaaatcsctaaactttgcgagtttatattatatagatgaatatggaccatacttttt

SEQ ID NO: 626 - DNA, 101 bp, Phoenix dactylifera
ggatggaaggatatcggtcaaaattgagtgtgggcaggatttgaaccaagrtcattggtatcaaggggtgtaaaccagctgaacttgggtgagctagacca

SEQ ID NO: 627 - DNA, 101 bp, Phoenix dactylifera
tgagtttaaaatcttactcaaactcagcaccttagaaacaaattcatatcragccaaatttcaggcggaacacaagttgctcatgaacaacatatttgttt

SEQ ID NO: 628 - DNA, 101 bp, Phoenix dactylifera
cagattcagccaagtggcacccttgagttcatatttcctttctttcgtagrgaagggattgcatcttaattaactacygaaaggaagggaatcatcggaat

SEQ ID NO: 629 - DNA, 101 bp, Phoenix dactylifera
ttcatatttcctttctttcgtagrgaagggattgcatcttaattaactacygaaaggaagggaatcatcggaatgaaatataattaagagctacatatgaa

SEQ ID NO: 630 - DNA, 101 bp, Phoenix dactylifera
ttgttgtaaatgccattctgaacttttcttgaccaaaatactcctcgctgytccatcttggttaatagatrgaagaaagagggagtgggatgaaactrtac

SEQ ID NO: 631 - DNA, 101 bp, Phoenix dactylifera
aacttttcttgaccaaaatactcctcgctgytccatcttggttaatagatrgaagaaagagggagtgggatgaaactrtacaatatatatgaaataaaata

SEQ ID NO: 632 - DNA, 101 bp, Phoenix dactylifera
ctgytccatcttggttaatagatrgaagaaagagggagtgggatgaaactrtacaatatatatgaaataaaatacaaaacaaagatagcttaaaatagtca

SEQ ID NO: 633 - DNA, 101 bp, Phoenix dactylifera
aatgatatccatcaccacctaccatagcctacaatctctaccacctaataygtgttatgtaatagggtgaatagatatactcgtcaatctaacagctatct

SEQ ID NO: 634 - DNA, 101 bp, Phoenix dactylifera
tatagaggtagaaataatagctataggttaggctcctcccttggcatcctrgcatcaggtctcactaatcttggtgtatcaaaacaaagatagcttaaaat

SEQ ID NO: 635 - DNA, 101 bp, Phoenix dactylifera
caatctctaccacctgatatgtgttatgtaatagggtgaatagatatactygtcaatctaacagctatcttctggaggttatttaagtagatcttcatatc

SEQ ID NO: 636 - DNA, 101 bp, Phoenix dactylifera
gggttcagttaagagctatgcagagaaaaagaatgttgaaattcgtctttrtagggtcatctatgattttattgatgacatgcgaaatgcaatggaaggac

SEQ ID NO: 637 - DNA, 101 bp, Phoenix dactylifera
tggtcaccaaagggaaagtggtgaaagattgtggtgttcggattgttcgamatgggaagacagttcactctggaaatattgattctctgagacgagtgaaa

SEQ ID NO: 638 - DNA, 101 bp, Phoenix dactylifera
gctcaatttgttgttgaataaaagaaattggaaaactatagctatcctagraatccaaagagaattttgttgttctaggaatatcacgggatacaatgaag

SEQ ID NO: 639 - DNA, 101 bp, Phoenix dactylifera
caatgaagtatgaatttggttgcagtgctggcataccatggtcttaccctytaatggtgatagtgttattttgttttggtgaaaagatatttgcccttgat

SEQ ID NO: 640 - DNA, 101 bp, Phoenix dactylifera
cactaataagtaggtggaaaaagcttggtacaacctgttagtacctttttwttccatgatatctaggaaatagcctttatcctggctaaggctatgtcgat

SEQ ID NO: 641 - DNA, 101 bp, Phoenix dactylifera
ttcatttgcagaccaaatcttgacccgagcatgaatcatttgatatatagwacttggatgagtaaagttgcaaagcattaatatagaaaacaagatagtct

SEQ ID NO: 642 - DNA, 101 bp, Phoenix dactylifera
tttcctggacaagtaggaaatctcttgccatcagtcaatttataagcttcsaatattgcagggctaggtgatgatatagagctgtcattgggaatttacct

SEQ ID NO: 643 - DNA, 101 bp, Phoenix dactylifera
ggtgatagtaagctacattatccatgatttctacagtatactattctcatrgtggccataaagggaaaascaatgtgagaaacaatatgaaaatgagaaac

SEQ ID NO: 644 - DNA, 101 bp, Phoenix dactylifera
atccatgatttctacagtatactattctcatrgtggccataaagggaaaascaatgtgagaaacaatatgaaaatgagaaacgggaaatgatgatcaargg

SEQ ID NO: 645 - DNA, 101 bp, Phoenix dactylifera
aascaatgtgagaaacaatatgaaaatgagaaacgggaaatgatgatcaarggaagtccttagttgcggaaacaagaaacttgctcatgtagctattatgg

SEQ ID NO: 646 - DNA, 101 bp, Phoenix dactylifera
gattgccacccttgccaataagctcgtggaactcaaaataaccctgattcrgaactgtttagggctctaagatgctaaatcaaccttgacctactatttat

SEQ ID NO: 647 - DNA, 101 bp, Phoenix dactylifera
tgtttagggctctaagatgctaaatcaaccttgacctactatttatagtaracattgaccggagtgtaactcataatctatgaacaattgggcaatgcaat

SEQ ID NO: 648 - DNA, 101 bp, Phoenix dactylifera
aagcgtggggacttgtgcatgtgtgcgtttaatcccacattgattgtaccytggaaagatcttagatacttatatagggccaaaaccctgtataataactt

SEQ ID NO: 649 - DNA, 101 bp, Phoenix dactylifera
tttttgggctgattgtgggccgattgtggtgtttgtaattagatttagttrgatttagactcttagcctagagygaactccagtgcttaaatgagaggagg

SEQ ID NO: 650 - DNA, 101 bp, Phoenix dactylifera
ttgtggtgtttgtaattagatttagttrgatttagactcttagcctagagygaactccagtgcttaaatgagaggaggtgaggactcgtgtgggtgtgtgc

SEQ ID NO: 651 - DNA, 101 bp, Phoenix dactylifera
agggccaatgaatctaaataataacttttggctagttttttgggtgaggtystaatttgttacaccagggacaattcaaaatatttatcgagtattaagag

SEQ ID NO: 652 - DNA, 101 bp, Phoenix dactylifera
gggccaatgaatctaaataataacttttggctagttttttgggtgaggtystaatttgttacaccagggacaattcaaaatatttatcgagtattaagagt

SEQ ID NO: 653 - DNA, 101 bp, Phoenix dactylifera
aaaggatggtaagattctttgaacagctgccattgctgcctatgttctatyatgcattttgttatttaatatcagtgcaaaatggatagagtggaaatatg

SEQ ID NO: 654 - DNA, 101 bp, Phoenix dactylifera
actatgtgatggtgtacagcttgcaagggatagtcggcttgggcaaggcaygtactaaaaatacatactccargagaacttgaagaactatagttatcgtt

SEQ ID NO: 655 - DNA, 101 bp, Phoenix dactylifera
gcaagggatagtcggcttgggcaaggcaygtactaaaaatacatactccargagaacttgaagaactatagttatcgttttgagtaaggcctrggatttga

SEQ ID NO: 656 - DNA, 101 bp, Phoenix dactylifera
atactccargagaacttgaagaactatagttatcgttttgagtaaggcctrggatttgaaccttggacctccttaagggcaagcctttagggctcaagtgc

SEQ ID NO: 657 - DNA, 101 bp, Phoenix dactylifera
accttggacctccttaagggcaagcctttagggctcaagtgccaaccgtcwtgtttaccaatcacgcatctaatatataatatataaactatttaaatgta

SEQ ID NO: 658 - DNA, 101 bp, Phoenix dactylifera
cgagcaatctttacccagctatctatttttttccattacactaggtgggtkctggactagaatgtggtattggtgtgagtggcttcaatgaatgggaggtt

SEQ ID NO: 659 - DNA, 101 bp, Phoenix dactylifera
agaagcaacgaacacttgaagaggcatcagcttcgatgacagctgcattarttggtgcaggtgttgggttatgaaacaggctagctggtgcagaacatctg

SEQ ID NO: 660 - DNA, 101 bp, Phoenix dactylifera
ggttatcagctggatcatcgaattgtattgaatgaacaaattataccattmtacttagcrtggcatgatttgatgctaatatctgttgaacaagatggatg

SEQ ID NO: 661 - DNA, 101 bp, Phoenix dactylifera
ctggatcatcgaattgtattgaatgaacaaattataccattmtacttagcrtggcatgatttgatgctaatatctgttgaacaagatggatggagtttctt

SEQ ID NO: 662 - DNA, 101 bp, Phoenix dactylifera
acatttataaattcttttatttttatacttcaataatatttttatttgtartgcttgttatgatctatacatgtcctttttttccttcttgcctctttgcc

SEQ ID NO: 663 - DNA, 101 bp, Phoenix dactylifera
tgtcctttttttccttcttgcctctttgccttcaagattacctatcatagytgtgcagctgagcgaacatgtttcttctgtgtcccagctgcctaaaatag

SEQ ID NO: 664 - DNA, 101 bp, Phoenix dactylifera
cttcaccagaggattgctctcttcctctagaaggtggcttgggagcgtctrccgacatgctcagtgttgagtagacgaggtatgagccttgcccccctatg

SEQ ID NO: 665 - DNA, 101 bp, Phoenix dactylifera
tgagtagacgaggtatgagccttgcccccctatgtctggattgtggagtgracgagtcagtggatcatatgctattctagtgcgtgtgggcgagaggtgtt

SEQ ID NO: 666 - DNA, 101 bp, Phoenix dactylifera
ctcctggtgtcagcaccatcatggccggcaagcatctgatatttgtccttycgcgtgaaattggtgcactcgaacccaattacttgtccaatcaaccgttg

SEQ ID NO: 667 - DNA, 101 bp, Phoenix dactylifera
acatggtaccctcactgtaacagccaccggaacgatgttgtccgcgatctyggtgaacaaggggctgaattagaggaggtatggctgccagaggataacag

SEQ ID NO: 668 - DNA, 101 bp, Phoenix dactylifera
ttataaatattctaatttcatgtttctaggtcaaagtttttaaactatatrtcaaaaaccaatataaatatccagtgaacttgctgtttatatcatacaag

SEQ ID NO: 669 - DNA, 101 bp, Phoenix dactylifera
attaaatcttgacctaatgagtgggtctgtcttatgcaccaacatcatgayattatttgggagtaggggttagctctcatatgcaccaacatcatgacatt

SEQ ID NO: 670 - DNA, 101 bp, Phoenix dactylifera
atcatgacattatttgggagtaggggttagctctcattagaatggttagakgcaactaaagttcacttggtattggaatccatgtaaatattatgctcata

SEQ ID NO: 671 - DNA, 101 bp, Phoenix dactylifera
ctagtctaagagcttgatcatcaattagattgcatatggatctcacaaaaytgtatccaacccagttggactgtatagacccatgtccaacccaacaacaa

SEQ ID NO: 672 - DNA, 101 bp, Phoenix dactylifera
gcctagaagtggctcatttccactcactctgggctcaccaaacagatattkcaagagaagtagcagcagaaacgtttccagcttcgtacagtgaatatggt

SEQ ID NO: 673 - DNA, 101 bp, Phoenix dactylifera
ggatgacaagataaaaatcttaagcaataattaatatcgtatcaagccaayatatcacatatcagtttcaatcaagcagggcaacaagaactcataaactg

SEQ ID NO: 674 - DNA, 101 bp, Phoenix dactylifera
tttgggtagttgtacatatggttttactgtgtataacaaatttctggcagrgacgaaaactgttacaacgagtaggtaattaacatgtacatgtaatctaa

SEQ ID NO: 675 - DNA, 101 bp, Phoenix dactylifera
acacagccaatttgacctctttcagaaataataagctcaaagaacttgagstacccaaccacgttcacaaggaaagcgaagtccttacactatactgctgt

SEQ ID NO: 676 - DNA, 101 bp, Phoenix dactylifera
tgagaagttgacatacgccctcttccacggggctggtatcattttctgttkgttctttcttcatcggatttcctgccctgacttaagggtgtgagtccaca

SEQ ID NO: 677 - DNA, 101 bp, Phoenix dactylifera
tgcgggaccatctgtgcgacccatatttgatgatcgaaatgctgaagggaratgccaaggagaataagagaagtgagtaggaaaaagacacaaggcattat

SEQ ID NO: 678 - DNA, 101 bp, Phoenix dactylifera
ggattagggtcatgttcttcctaggattggtttaaacttttgataataagsccatggactwatttcaatgtcaaaaatagattggtcartgcttatacaac

SEQ ID NO: 679 - DNA, 101 bp, Phoenix dactylifera
catgttcttcctaggattggtttaaacttttgataataagsccatggactwatttcaatgtcaaaaatagattggtcartgcttatacaacaacttgcaaa

SEQ ID NO: 680 - DNA, 101 bp, Phoenix dactylifera
tttgataataagsccatggactwatttcaatgtcaaaaatagattggtcartgcttatacaacaacttgcaaagtcaactcgcgcgatgccttactaattt

SEQ ID NO: 681 - DNA, 101 bp, Phoenix dactylifera
aatgtcatacacatgagattctctccattagaaaaatatggaaagttgttycctattaaacaaacttctctgagatcgggattctaaaaatatagagagaa

SEQ ID NO: 682 - DNA, 101 bp, Phoenix dactylifera
tttaacgttgttttagttatatgagaaatgttattaaactctacttatttygttacacaatatgaactttattttcatgtagatggcttgtccatacaatt

SEQ ID NO: 683 - DNA, 101 bp, Phoenix dactylifera
ttattcatgactgattgtttttttcttttcttatttatcttttaagttggwggcagatgagaagatgatgcaacagctagctaagtgaaagcatgtctaga

SEQ ID NO: 684 - DNA, 101 bp, Phoenix dactylifera
tactggaagtgtcaagaccagtcagctatggctgttatatcatataacgtyggtggattttgaatattggatcaggtgatggcagctcaaatatcagcttg

SEQ ID NO: 685 - DNA, 101 bp, Phoenix dactylifera
tattggatcaggtgatggcagctcaaatatcagcttgatgatgattgcatrgtagagaggacttgggttgtagaaacaaacatttagttctcyccaactcc

SEQ ID NO: 686 - DNA, 101 bp, Phoenix dactylifera
gattgcatrgtagagaggacttgggttgtagaaacaaacatttagttctcyccaactccaattaggattgcttgttaaatgatccaggagaaytattagag

SEQ ID NO: 687 - DNA, 101 bp, Phoenix dactylifera
tagttctcyccaactccaattaggattgcttgttaaatgatccaggagaaytattagagttgatgtggaatactttgactactattaggaaaactaaatag

SEQ ID NO: 688 - DNA, 101 bp, Phoenix dactylifera
gtacattagaaagaaatcgagtactaaacttctggaggccattacttatckctacaacatccaattgaggtgctctattttctaaaatcaggtgacatttt

SEQ ID NO: 689 - DNA, 101 bp, Phoenix dactylifera
ccaattgaggtgctctattttctaaaatcaggtgacattttggagttctaygatgtaacatccctcctaaaaggtgtggcacactggaaattaaggccaaa

SEQ ID NO: 690 - DNA, 101 bp, Phoenix dactylifera
gacttgaactcgcatgttatgcatgatgatggatccgaatggtgaatcaartacaatcagaaggcatggatgatgaattttgattctyggtacctttcaga

SEQ ID NO: 691 - DNA, 101 bp, Phoenix dactylifera
aatggtgaatcaartacaatcagaaggcatggatgatgaattttgattctyggtacctttcagattcctgcactacaactacaattaccaaaccttawaat

SEQ ID NO: 692 - DNA, 101 bp, Phoenix dactylifera
tctyggtacctttcagattcctgcactacaactacaattaccaaaccttawaatgatcataaaagtcatgcaaatcttttgtcgaatccattaggtcttaa

SEQ ID NO: 693 - DNA, 101 bp, Phoenix dactylifera
atcctgtattttattagaggatgttccctcaactcaacaaagaaaacactytttagatgactgcaaaatatgcactgatggatctatccagaactattaga

SEQ ID NO: 694 - DNA, 101 bp, Phoenix dactylifera
agaaattagaacttcatgattttttgctctgcatctgataggaaactcgtrttactcaccttagaaggagcttttggtcatsgtaccatctggttcacata

SEQ ID NO: 695 - DNA, 101 bp, Phoenix dactylifera
catctgataggaaactcgtrttactcaccttagaaggagcttttggtcatsgtaccatctggttcacatagacctagaactttcacaagtgacacacagcc

SEQ ID NO: 696 - DNA, 101 bp, Phoenix dactylifera
ctaggaaatatcagcatgcaaaacctttatgtgacaaaaaaagaaaagacrgagagtcatcaataatcactggcaattaactttcttttcactgaatatga

SEQ ID NO: 697 - DNA, 101 bp, Phoenix dactylifera
attttcaatggaataacaggacaatctctgtttaactgataattcagtgarcaattcaygcatcaaagatcaggagcagtcatccaaatctctttaataat

SEQ ID NO: 698 - DNA, 101 bp, Phoenix dactylifera
tggaataacaggacaatctctgtttaactgataattcagtgarcaattcaygcatcaaagatcaggagcagtcatccaaatctctttaataatttatgaag

SEQ ID NO: 699 - DNA, 101 bp, Phoenix dactylifera
ataacaagtaaaaatttatgagatgcacacaaggttgcatatatttctatracactctaatgatcactaacaggtatcagtacctgaaaataggcttttcc

SEQ ID NO: 700 - DNA, 101 bp, Phoenix dactylifera
accctgtgcatagcgtgtgctcagttcttgtcacagccaacttcaaatgawtttctcctaacatttagactccgtttgctttggaatccaagcagaataaa

SEQ ID NO: 701 - DNA, 101 bp, Phoenix dactylifera
tattattacggcccaccatttccagacgatgtcgaccgtcggatcaaagamcatacgaggcaaggggtaawcaagatctactcaatattaccgcagggaag

SEQ ID NO: 702 - DNA, 101 bp, Phoenix dactylifera
tccagacgatgtcgaccgtcggatcaaagamcatacgaggcaaggggtaawcaagatctactcaatattaccgcagggaagggcatttctggaataacggt

SEQ ID NO: 703 - DNA, 101 bp, Phoenix dactylifera
aacgaggaaatagtatcatatgaaattaaagagctggaagagcaaaaatarttccgagcaactggaaactagcagtcccgatgagaattgtcaacaaaact

SEQ ID NO: 704 - DNA, 101 bp, Phoenix dactylifera
ttcacgtgtcgctctcctattcgctgaaccctcgtcgggacccactgtttrcactatgtaggtttttgatatccaccgtctttttatctccataattcata

SEQ ID NO: 705 - DNA, 101 bp, Phoenix dactylifera
tggattcctccgctggcctggccgtcccttctagttggacttgggtcctgyccactgatcggcacttgggtatccccccatagtcaagggttgtttgggtg

SEQ ID NO: 706 - DNA, 101 bp, Phoenix dactylifera
aggaaccatgcaggccataacctttgcaacacctaacatagttatggactrgtgttgaaaggaatatgggctgtttggtcatgggaaatgcagaagatagc

SEQ ID NO: 707 - DNA, 101 bp, Phoenix dactylifera
aaaaataatttgagactcctttctatttgtagcagagctctttgcttcatwagatgcagactacaattataaatttggtaaaagtttgttgcaaaagattt

SEQ ID NO: 708 - DNA, 101 bp, Phoenix dactylifera
cgatcgtcctcgcgattccgtgtctaccttcttcgtctctccgtttgtgtytgtgtcggtggttcccatgggatcgaagtgggctctgatccggcccttct

SEQ ID NO: 709 - DNA, 101 bp, Phoenix dactylifera
ttttttctttttgtgatgggagagaccaaaactcagcagcatcatgcatgygaataaaattaggtgataggttgatcattactagaacatttgtataggat

SEQ ID NO: 710 - DNA, 101 bp, Phoenix dactylifera
taattcaagtgcatttaaagcaattaattgccattaaaggcacaatcacartcccaccaattgaggggcggttacagctcatcccaagcggatcgaccatt

SEQ ID NO: 711 - DNA, 101 bp, Phoenix dactylifera
gcctttgatcctattagagaaattgaggaagtttgccatgacaaagtcacrgagagctcaagaagcaccaattactagtcagcaggaggcgtagtcagatg

SEQ ID NO: 712 - DNA, 101 bp, Phoenix dactylifera
ctcaagaagcaccaattactagtcagcaggaggcgtagtcagatgcctctmatcgaagcctgcagagagcacagcttccccaaagctcttagctaggagtt

SEQ ID NO: 713 - DNA, 101 bp, Phoenix dactylifera
acaaagtcaccactcttgttcgtggagctcgggacgagataggagatgccratctctaagcctgcattccaaggactccactccaggccgctcctcccctc

SEQ ID NO: 714 - DNA, 101 bp, Phoenix dactylifera
tggaaatctgcgacttgaatcactctattgtgatgattgcaccgaagagcrgattgagaccttcttggtttctcttttctttagactttgtcgtgcttctt

SEQ ID NO: 715 - DNA, 101 bp, Phoenix dactylifera
accttcttggtttctcttttctttagactttgtcgtgcttcttgcttgagycaagaagtatgttaatgttgagaaggcgatggtagctcagygagagttag

SEQ ID NO: 716 - DNA, 101 bp, Phoenix dactylifera
ttgcttgagycaagaagtatgttaatgttgagaaggcgatggtagctcagygagagttagcaggggtaagcatggcaagaagaaaaatgaaagaaaccaag

SEQ ID NO: 717 - DNA, 101 bp, Phoenix dactylifera
gaaaaagaagagccgaagaggaggcctaggagaaatgaaggaagctctcgycgctcgaggagcccaccttggaagtatgagaggtacactcctctcaacac

SEQ ID NO: 718 - DNA, 101 bp, Phoenix dactylifera
gacttacacctaggaagccctagcagaagatttttttcctgaagagacagmggaccaaggaggttatctccttcttggatgatgatttaaagcaagatacg

SEQ ID NO: 719 - DNA, 101 bp, Phoenix dactylifera
caatgcgagctgtggtatcgacctatcatcttgtagtgtagttttctaccragcatggagttggagaagttcggggtgatcatgcaatagctcgatagtgt

SEQ ID NO: 720 - DNA, 101 bp, Phoenix dactylifera
ccttccttgttggcgacgaagggataggttgaattgtttatgtcagctccyaactagatgcagcaacctgagctcagctcatcatctttctttgtgccaac

SEQ ID NO: 721 - DNA, 101 bp, Phoenix dactylifera
tcatctttctttgtgccaacgtaggcatctttgcttggttagcctcagatrtgccaggtatagattctagcattattatgcataggttgaatatggaatcg

SEQ ID NO: 722 - DNA, 101 bp, Phoenix dactylifera
gacctatgcggtagaaaaagagaagtctcaaagcgccaataggcgatggaygaggaggtggacaaactcctaaatgtcggcttcatctgtgaagtcaacta

SEQ ID NO: 723 - DNA, 101 bp, Phoenix dactylifera
attgtgcaagtgcttgttagagaacaagggtactgagtgtgaccaaaacargtagtcacttggatgtctatccactcgtcagtgatttacttcatgctgca

SEQ ID NO: 724 - DNA, 101 bp, Phoenix dactylifera
ccgctgcgattgtcgcaacgcgtgcggaggtaacccgacaattgactggtygacattgtcttggtgaagaaggctaataagaaatggagtgtttgcatcga

SEQ ID NO: 725 - DNA, 101 bp, Phoenix dactylifera
ccctgcggatgagaagacctttttcatcattgacgatggtctttgttgctrcagggttatgcctttggtctaaagaacaccggtgtagcaaattagatatt

SEQ ID NO: 726 - DNA, 101 bp, Phoenix dactylifera
ttagatattggtcaatcgtatattccaaagagtagatcggccgtaacatgraagtctatgttaatgacatattattaaagagtaaagaagccgagcagcat

SEQ ID NO: 727 - DNA, 101 bp, Phoenix dactylifera
cgggatcgaggccaaccctgagaagatccaagctatgctggagatgtcacyacmgagaacaataaaggaggtgccatgctttataggttggattgcgacgc

SEQ ID NO: 728 - DNA, 101 bp, Phoenix dactylifera
gatcgaggccaaccctgagaagatccaagctatgctggagatgtcacyacmgagaacaataaaggaggtgccatgctttataggttggattgcgacgccga

SEQ ID NO: 729 - DNA, 101 bp, Phoenix dactylifera
caccacctttccttaccaaactgagcttgggagagatgctgtacctttacstcgctatttctccttgagccttgagttcgatcctggtccgaggagttcag

SEQ ID NO: 730 - DNA, 101 bp, Phoenix dactylifera
atcctggtccgaggagttcagaagctgatctactacacaagcaaagttctycgagatgctgagacgaggtcactaaggtggaaaaagatggtctatgctct

SEQ ID NO: 731 - DNA, 101 bp, Phoenix dactylifera
taaccaaccaatagatgatgcaaatattgcagaagctagacacatcggggytgttgatcaaatgggcagtcaagctcggggagttcgacrtccatttttga

SEQ ID NO: 732 - DNA, 101 bp, Phoenix dactylifera
acacatcggggytgttgatcaaatgggcagtcaagctcggggagttcgacrtccatttttgaccttggctcgccatcaaggctcaagtgttagcagatttt

SEQ ID NO: 733 - DNA, 101 bp, Phoenix dactylifera
agtttatgaaggcatctatggtaaccacctaggaggcataatgctagcttrcaaagttatgaggtaaggttattactggctgaccttgcagaatgatgtag

SEQ ID NO: 734 - DNA, 101 bp, Phoenix dactylifera
aacatcagccaacggtgtaattaacactgttcagcgccccttggccatttrcccagtggggaatggatattctcaratcttttcctccatcagctgagtag

SEQ ID NO: 735 - DNA, 101 bp, Phoenix dactylifera
actgttcagcgccccttggccatttrcccagtggggaatggatattctcaratcttttcctccatcagctgagtagagaaaatttttaatcatagccattg

SEQ ID NO: 736 - DNA, 101 bp, Phoenix dactylifera
taaaaacccaaccaattaaagatcggaggacatccaaattgacctcttcargtccgaaacactgaggtaacactcaatgagtggggaaaaaaaagcctagc

SEQ ID NO: 737 - DNA, 101 bp, Phoenix dactylifera
acaattcccaacatcaacatgctagtgttttggcttttaggtgttcgaatktcaaattccaggcatccwggcatttgargcttagaatgaagagttttgat

SEQ ID NO: 738 - DNA, 101 bp, Phoenix dactylifera
atgctagtgttttggcttttaggtgttcgaatktcaaattccaggcatccwggcatttgargcttagaatgaagagttttgatgttctcattccttggctc

SEQ ID NO: 739 - DNA, 101 bp, Phoenix dactylifera
tttggcttttaggtgttcgaatktcaaattccaggcatccwggcatttgargcttagaatgaagagttttgatgttctcattccttggctctgatcaagtt

SEQ ID NO: 740 - DNA, 101 bp, Phoenix dactylifera
atgtatatcttattcacacctcccatgcaagcattagaataggacatttcrtctacctataatattttttaaaaagggacaaagtgtgtcttatgttctat

SEQ ID NO: 741 - DNA, 101 bp, Phoenix dactylifera
ctgtgctgagcatgctttgactcaaggagaaataacccacatcgatgactmctgcaattgacatakgcatcttgacaatcaacaattgactggcaatcaca

SEQ ID NO: 742 - DNA, 101 bp, Phoenix dactylifera
tttgactcaaggagaaataacccacatcgatgactmctgcaattgacatakgcatcttgacaatcaacaattgactggcaatcacacattttagtagagta

SEQ ID NO: 743 - DNA, 101 bp, Phoenix dactylifera
ggcacactcgaaatttgctcatgggaatagttataccattctaaactttcwcatctttctcgcgtaagcatatatatgtagcttagtaacttttatttgtc

SEQ ID NO: 744 - DNA, 101 bp, Phoenix dactylifera
cccatacccttcttcctagatttactaactttgtaagcacccaccctactmctaaggaccctattgaaagtgggatatactctaaaagcyctctgcatatt

SEQ ID NO: 745 - DNA, 101 bp, Phoenix dactylifera
cccaccctactmctaaggaccctattgaaagtgggatatactctaaaagcyctctgcatatttaatatactcgagaaattatgtctatctcctcttatagt

SEQ ID NO: 746 - DNA, 101 bp, Phoenix dactylifera
cagaacaggataccttactatattcaaagaccagtaaaattacaaccaaayttatgttttgactatggcatcttgaatatacccttgatagatgccaatgg

SEQ ID NO: 747 - DNA, 101 bp, Phoenix dactylifera
aggcacttcaaaagcatggagatagcactgaagccgtatgatatgtggatkaaaattgcacacyaagataaataatatatgctatttgatgacacaaaact

SEQ ID NO: 748 - DNA, 101 bp, Phoenix dactylifera
gcatggagatagcactgaagccgtatgatatgtggatkaaaattgcacacyaagataaataatatatgctatttgatgacacaaaactcacaaggaccatc

SEQ ID NO: 749 - DNA, 101 bp, Phoenix dactylifera
tcttggtttaataagatgttaaacttttaccatatatacaggcaaggcagyggacccgctgtttaggttgcaagtattggytcatcaggaaaaatatgtaa

SEQ ID NO: 750 - DNA, 101 bp, Phoenix dactylifera
catatatacaggcaaggcagyggacccgctgtttaggttgcaagtattggytcatcaggaaaaatatgtaaacattaaaatagttttgctcctaaaatttt

SEQ ID NO: 751 - DNA, 101 bp, Phoenix dactylifera
aaaggatcaatcacctgtgctcgagggaaaaaagttaaaaaagaactcctwatctcaagatgaaaacactcccaacaattataggatactttagtgttaca

SEQ ID NO: 752 - DNA, 101 bp, Phoenix dactylifera
agaatcactatagcaacaaccaacaagagcaggcacatacatgaacaaccwccttttgtacgcttgttaagaatcgccagcctcctttgtactctctgcat

SEQ ID NO: 753 - DNA, 101 bp, Phoenix dactylifera
cacacactagatggacatctaatggctgagatgaaccacagccttttggasagagatcataagcaccggccctggctctcctaaaaacaactccacttctc

SEQ ID NO: 754 - DNA, 101 bp, Phoenix dactylifera
gaacttggcatagccaactagtgatgagcgcaagttgagctcaagtcagcytcacagaataccgagccgagctcaagcagatctgaactagctcaagatca

SEQ ID NO: 755 - DNA, 101 bp, Phoenix dactylifera
atgatggtccaagttatcctgtaccagcgcatgaaaagttatagtagtaargatgctgtcagacatgttgaaaagttatcctgtgtcatggygatacatac

SEQ ID NO: 756 - DNA, 101 bp, Phoenix dactylifera
tagtagtaargatgctgtcagacatgttgaaaagttatcctgtgtcatggygatacatacaatcaatcttgtatgcagatccagttcctcgttaactgtta

SEQ ID NO: 757 - DNA, 101 bp, Phoenix dactylifera
aaatttgagagcatgtcctggcatttatgcaactccttatcagatctgcawaaggcagtaggtaacataagttaaaggaaaaatrgtattcatgtctaaca

SEQ ID NO: 758 - DNA, 101 bp, Phoenix dactylifera
ccttatcagatctgcawaaggcagtaggtaacataagttaaaggaaaaatrgtattcatgtctaacaaattatagaaggaacctacagactatatcatgct

SEQ ID NO: 759 - DNA, 101 bp, Phoenix dactylifera
caaacaaaaagaaatatatattttccagactgatctgttccaaggtattgkacatcaaaaatgtaaagcaagttggtgcactcaacttcwatcaaaatctt

SEQ ID NO: 760 - DNA, 101 bp, Phoenix dactylifera
ccaaggtattgkacatcaaaaatgtaaagcaagttggtgcactcaacttcwatcaaaatcttttgtatcaactaaaytgctaaaaatacaaatgmtcattc

SEQ ID NO: 761 - DNA, 101 bp, Phoenix dactylifera
aagcaagttggtgcactcaacttcwatcaaaatcttttgtatcaactaaaytgctaaaaatacaaatgmtcattctaaatacctcttggcttacccactac

SEQ ID NO: 762 - DNA, 101 bp, Phoenix dactylifera
aacttcwatcaaaatcttttgtatcaactaaaytgctaaaaatacaaatgmtcattctaaatacctcttggcttacccactactgcatcggcattagacct

SEQ ID NO: 763 - DNA, 101 bp, Phoenix dactylifera
cctgactaatttcaattatatctttggctgagcccagcccacctgtggaayaggtctaataggcattatcaatttttcaattctatatgcaagtaaaagaa

SEQ ID NO: 764 - DNA, 101 bp, Phoenix dactylifera
ccagcatcctggaggcctatattcctgcaacaatttctaggataaagaccyggtcatggaccatttatttctttaagtagacaggtgaaaaaaaccttttt

SEQ ID NO: 765 - DNA, 101 bp, Phoenix dactylifera
atcataattgagcaaaggaaaaacgaaagtgaacatatgaaagaaatgaarggcccttgaygagatagaaacccaaggaatgccggctaaaagtaatatga

SEQ ID NO: 766 - DNA, 101 bp, Phoenix dactylifera
agcaaaggaaaaacgaaagtgaacatatgaaagaaatgaarggcccttgaygagatagaaacccaaggaatgccggctaaaagtaatatgaatattctttt

SEQ ID NO: 767 - DNA, 101 bp, Phoenix dactylifera
caccacaacaaaaaaatagctaattttaataatcaatactccatgtagatmtaggatttgtcataattcatccatataacacagacataggcccatgccta

SEQ ID NO: 768 - DNA, 101 bp, Phoenix dactylifera
aacaacacaaggcagcacagacaaatacttgccaatatggacaggtacatygtatgcaccaatgttctatgcaaacatccttgtagcttggcacttagaaa

SEQ ID NO: 769 - DNA, 101 bp, Phoenix dactylifera
tatatactacccttttagactcataagttagtttttggctgaaaatctaaytggtcctagtttcggttggattatggaaacatttcagtttaagggttttg

SEQ ID NO: 770 - DNA, 101 bp, Phoenix dactylifera
gtttaagggttttgggtgaaattcaaatcatagcctttgtactttactgtmttctcccatggcagccttcttcctatgatcagcctcyatatagcaaaatt

SEQ ID NO: 771 - DNA, 101 bp, Phoenix dactylifera
tgtactttactgtmttctcccatggcagccttcttcctatgatcagcctcyatatagcaaaatttgcgatacaccgctcgtgtttaattccgaacatctta

SEQ ID NO: 772 - DNA, 101 bp, Phoenix dactylifera
ttgtcagtatcttcccctccttagttcttctccctcttcccccttttatcsttttgaacaatagccattgcggcaaggtgatagtagttttagcatccatt

SEQ ID NO: 773 - DNA, 101 bp, Phoenix dactylifera
tgccaggagttggggaccaagtgggttgggactaatgaatagcaaattagragatggttgatgaaagggtgaagaggaagcctcaacctgactttgagact

SEQ ID NO: 774 - DNA, 101 bp, Phoenix dactylifera
atcggaaagatgacgatcgtcggaagcagagtgcatggaaatcttctcgtwgcgcttgttggagaagattaaggtgcagaygcaacggggaagagctctca

SEQ ID NO: 775 - DNA, 101 bp, Phoenix dactylifera
gtgcatggaaatcttctcgtwgcgcttgttggagaagattaaggtgcagaygcaacggggaagagctctcaacygcctcagactcaaatgagaggagaarg

SEQ ID NO: 776 - DNA, 101 bp, Phoenix dactylifera
gcttgttggagaagattaaggtgcagaygcaacggggaagagctctcaacygcctcagactcaaatgagaggagaargggtaaaccctttatataaaggga

SEQ ID NO: 777 - DNA, 101 bp, Phoenix dactylifera
aygcaacggggaagagctctcaacygcctcagactcaaatgagaggagaargggtaaaccctttatataaagggatggacgacctagatcaaagcgactat

SEQ ID NO: 778 - DNA, 101 bp, Phoenix dactylifera
attgccacttcaaatcctcttgaagagtctaattgtcatctagggaatccygccaaaatgatgatgcttygaggatgtcaatagatgctgccaagcattta

SEQ ID NO: 779 - DNA, 101 bp, Phoenix dactylifera
ttgaagagtctaattgtcatctagggaatccygccaaaatgatgatgcttygaggatgtcaatagatgctgccaagcatttagtgctacctcccaaacctt

SEQ ID NO: 780 - DNA, 101 bp, Phoenix dactylifera
tgactcagatgtggggggcaattgatatggcatgtattcatcaacttgacyttggcaattgaccgaagtgatgatctcgacatcaacgaagctgctagtct

SEQ ID NO: 781 - DNA, 101 bp, Phoenix dactylifera
ccgaccaacggtaataagccacgtgtctccaattcaagtatatttaaagcmattaacaaccattaaaggaacaatcacaatcccgtaattgaggggcggtt

SEQ ID NO: 782 - DNA, 101 bp, Phoenix dactylifera
tgatacaaaagttggagcaatataatagtatcttggaggtaaattttcttraaattcaactatttaaatattaattgcagtaaaagcactcttgcctagct

SEQ ID NO: 783 - DNA, 101 bp, Phoenix dactylifera
aattcaactatttaaatattaattgcagtaaaagcactcttgcctagcttmtttttcttgctttgacttagaagcaaattatgttgccactagggaagaat

SEQ ID NO: 784 - DNA, 101 bp, Phoenix dactylifera
ttttggacctagtcaaacaactgatgtgtgttcattgaactaattgaagcratcacttaaataatgaaacaaaacaacctttaataatcgaagtgcagcta

SEQ ID NO: 785 - DNA, 101 bp, Phoenix dactylifera
ctccagtgaggtgctcttatatgagcacttatttcctaggctttagggctytataatgctaaatttttcttatttctcatgcttatgtttgtgcttgtggg

SEQ ID NO: 786 - DNA, 101 bp, Phoenix dactylifera
ataaatatatgactttcacgtaagttatatcttctggatgcaatatgtatkttccctatgtggactgataatataaattgacaacctactcgaagtcttct

SEQ ID NO: 787 - DNA, 101 bp, Phoenix dactylifera
tctcataaaaaatagagaatttcttccatcctccctgtttgtattataagygtgtgaatccttcaaacagacaaacatagacacatgcacatattcacaca

SEQ ID NO: 788 - DNA, 101 bp, Phoenix dactylifera
aattgctggcaggtaatgtttatgcccacttgtttatgctaggataacatyattctcagtgtaaatgggaggaacagggctacgtaattgaatgaaaatat

SEQ ID NO: 789 - DNA, 101 bp, Phoenix dactylifera
agtacagggagaggagggtgttatgggaggaagccacctcacttcttcaaytggggcatccggtattgattgcgggggatttcaactgtatcgatggtcca

SEQ ID NO: 790 - DNA, 101 bp, Phoenix dactylifera
tgctatatattattagtttggtgatgtttctatggccattaggattcaatkaaaagttgcctttaatactaaattacttatctggtggtatgtcatgcatt

SEQ ID NO: 791 - DNA, 101 bp, Phoenix dactylifera
attattatggtctttttatcttgcaggtgaaaagatacattgatctctatkctgcaacatcagttattggattaccttcagaagacttgcaccatgtagca

SEQ ID NO: 792 - DNA, 101 bp, Phoenix dactylifera
gctcatctcagtccattgggtgtcagaaagcaaattaatcgctatgtccayggtgattcaagatattggaaggaatttggcaccycgaatagtttwtgctg

SEQ ID NO: 793 - DNA, 101 bp, Phoenix dactylifera
ttaatcgctatgtccayggtgattcaagatattggaaggaatttggcaccycgaatagtttwtgctgttggattagggctcctagtatcatcattgctttc

SEQ ID NO: 794 - DNA, 101 bp, Phoenix dactylifera
gtccayggtgattcaagatattggaaggaatttggcaccycgaatagtttwtgctgttggattagggctcctagtatcatcattgctttctcggattcttg

SEQ ID NO: 795 - DNA, 101 bp, Phoenix dactylifera
ttgttgtgctctctctctctcttccagtgtttaggcacatgcatgcagatrtacacagtaactcataatcggagttattggtgtcttacatgattgttgac

SEQ ID NO: 796 - DNA, 101 bp, Phoenix dactylifera
aatgcaccttttctcctgaagacatttactagcatcattgttgttgtttgygcaacaaatgcgctgcaaaagttgatggtgcctttgcatctgtatacatg

SEQ ID NO: 797 - DNA, 101 bp, Phoenix dactylifera
aaaaatggtgttatgttcatctttagaagactgacacctgagttcatgctycacgtattgcattgttttctgtttaatacatatggaatgggttttctaca

SEQ ID NO: 798 - DNA, 101 bp, Phoenix dactylifera
gcatgtatatattctagtatgatatgttgtccatgtgctgagaattgagcytacatatgaaaatatagaggggcaatttttcaagaagagatatgttgcat

SEQ ID NO: 799 - DNA, 101 bp, Phoenix dactylifera
cttatgaccctccaaaaataaaatttcctaattttcaaataaaataaaatwttctaaattctatatttgcgcctaacataagtgcgcccagatttcttatt

SEQ ID NO: 800 - DNA, 101 bp, Phoenix dactylifera
cctttccatcggataagataacaatggaaaccatcattaaaagaagtaacwatctcattttagtctcmtttggttgctgagaaaaatgtggcgccttagga

SEQ ID NO: 801 - DNA, 101 bp, Phoenix dactylifera
ataacaatggaaaccatcattaaaagaagtaacwatctcattttagtctcmtttggttgctgagaaaaatgtggcgccttaggaattgaagtatctacaag

SEQ ID NO: 802 - DNA, 101 bp, Phoenix dactylifera
cattttggatatatgtgaaccatgacaatattctgggttatagtgtgataygatcaagtatagggacaatgaaagtatgcagatgtagacttaaaattgca

SEQ ID NO: 803 - DNA, 101 bp, Phoenix dactylifera
gactggatgctgattggttgttggcacaacgactgaatgctgacgtgacartattgactggaggggataaaagccaaatgggttggatttggattagattt

SEQ ID NO: 804 - DNA, 101 bp, Phoenix dactylifera
tgcgaggcctcaaatctgaagccctagtgtgtgtggccatgtgtgaggccrtctagtgattgaggggagaacgatttgtgggaggtttgagagttggagac

SEQ ID NO: 805 - DNA, 101 bp, Phoenix dactylifera
atttgcacgtaacacaagtgcgcctagattccttattggtggatggtcgtytgtcactactttcgaatgataaatgacgttcattcacataaacactgtag

SEQ ID NO: 806 - DNA, 101 bp, Phoenix dactylifera
tgataaatgacgttcattcacataaacactgtagtatgcacatatataagwcgtgtctaatttatgtctctggttcattcttatttacatgcatattcttt

SEQ ID NO: 807 - DNA, 101 bp, Phoenix dactylifera
cttctaacagatgtcgtaatttgcttggcatctctctactattattgacarggcagyaacctgtgtcaagaaagctgccaaaagtcaggtgcagcattttc

SEQ ID NO: 808 - DNA, 101 bp, Phoenix dactylifera
acagatgtcgtaatttgcttggcatctctctactattattgacarggcagyaacctgtgtcaagaaagctgccaaaagtcaggtgcagcattttctttgtt

SEQ ID NO: 809 - DNA, 101 bp, Phoenix dactylifera
catttgaatctatgcaagaactccatcaggatccctcattttccttttttyctccttttttgacttttgtctatactctgcatatacatgtttcaggctat

SEQ ID NO: 810 - DNA, 101 bp, Phoenix dactylifera
agtaaccaaaaaattggcaagcgagattaggggtttgaatgataagagcgygaagtttttgcaggtggaaacttgcagaaagaagaaawtgggtatattgt

SEQ ID NO: 811 - DNA, 101 bp, Phoenix dactylifera
atgataagagcgygaagtttttgcaggtggaaacttgcagaaagaagaaawtgggtatattgtttgatcatgctccaacctaaaattacatgccgagtgac

SEQ ID NO: 812 - DNA, 101 bp, Phoenix dactylifera
ttttcatgttaaattagcatgaataaggtggatgagaacatttacacacaytaactgttgtggttcaaatctggcccgagggtgaccatgccggaggagtc

SEQ ID NO: 813 - DNA, 101 bp, Phoenix dactylifera
agggttgttgccattgctctcgtcatcacgtgccgacgatgtatccacgtrttttgagcaatccatttttctcccaacactaacattttggcacatgaaca

SEQ ID NO: 814 - DNA, 101 bp, Phoenix dactylifera
tctatatgcaatgacatagtttaggtgtcttgccttatgatgatttattaygcttaatgatgttctatgcaatgtaagaaatgactaaatgcatgcccaaa

SEQ ID NO: 815 - DNA, 101 bp, Phoenix dactylifera
aacacactactagttttggatttagtaacatgaaaaaatatcttgttggakaaggggattactcagagatgttcttggacatagtattagaaatttgacag

SEQ ID NO: 816 - DNA, 101 bp, Phoenix dactylifera
tagtaaaggttcgtagtctgaaagcagagatagaagagataaggcagagcyatcagacagagatacaatctttgaaagctcagcttggccagattgcattc

SEQ ID NO: 817 - DNA, 101 bp, Phoenix dactylifera
taggttcttgatcctttcagtagttgtagagctgatgatgatgatgccatrgacccctgatagcgcttagactatttttttaagagttttatgtatatatt

SEQ ID NO: 818 - DNA, 101 bp, Phoenix dactylifera
acgatgcgaaccaaaagcatcgtaaaattattttttcatgacaacattacygatgcttttgtgattttttagaaggcatcagaaatctgactcacatcgct

SEQ ID NO: 819 - DNA, 101 bp, Phoenix dactylifera
atgttgtgctgaaagctaataatgccatgccatatcgtatcaagcatgacrtatactgacaatagactaatggcaccatagcatctcatgccaactaaagc

SEQ ID NO: 820 - DNA, 101 bp, Phoenix dactylifera
ggtgaacgccgggaggctgcaacaactgatttgctcggttgcttcttcttygctgatttgttcctcttggtatccattaatgatcagatcacccacttcga

SEQ ID NO: 821 - DNA, 101 bp, Phoenix dactylifera
cttttatttttgcaggtggcacttgctaaaggccaactgaagcaaggctaktttttatagttttcatgaacattggataaaatgattttgtttacttcagt

SEQ ID NO: 822 - DNA, 101 bp, Phoenix dactylifera
aggtaatatataaataaaatagccattatagatgattttcccaacacttaygagtgccggtaagaagcacgatattctacaaatgctggaaaagatccatt

SEQ ID NO: 823 - DNA, 101 bp, Phoenix dactylifera
tttgtgagggagttgtgtttcaatcttggcttgtaattttttaataacaayttttctcctctttactaaattttttcttcraacatctcttccatattctt

SEQ ID NO: 824 - DNA, 101 bp, Phoenix dactylifera
ttgtaattttttaataacaayttttctcctctttactaaattttttcttcraacatctcttccatattctttgttttttttcttagtattttctagatcct

SEQ ID NO: 825 - DNA, 101 bp, Phoenix dactylifera
cggcggccatacaatagtttttcagtacaacagtgatggcagtagattggyagaggactttcatcattttgataccgaagtggcaggacgccaaggagccg

SEQ ID NO: 826 - DNA, 101 bp, Phoenix dactylifera
gaggatcctcgccgttaggttgagagatgttcttcctaggcttattagccyagagcagggggcttttttggagggatggagtatatctgataatgtgctta

SEQ ID NO: 827 - DNA, 101 bp, Phoenix dactylifera
gagggcctatgacaagatgagataggattttttgcagcagtccctacaggrgttcgaattttatgagacatggttagatgggttatgtgcattcgtgctcc

SEQ ID NO: 828 - DNA, 101 bp, Phoenix dactylifera
acatggttagatgggttatgtgcattcgtgctcctttctttgctattttgrtgaatggcacaccgtcccgtttctttgtgtcgtcggagggttgcactagg

SEQ ID NO: 829 - DNA, 101 bp, Phoenix dactylifera
aggtcgtcttcgaggatggtggtagacagagcgacgctatacgcgaggaargttattgcggctaccatcggattttcttctaggatagctagggacatctg

SEQ ID NO: 830 - DNA, 101 bp, Phoenix dactylifera
gatggtcatcccctcgttcgggagactcgcatgatggctcagttgatcggyaatgttcaggtggtacacgtgtttagggaaacgaatagggcagcagactg

SEQ ID NO: 831 - DNA, 101 bp, Phoenix dactylifera
tgacatggtgagagatttaggcttgcggagttttcttggaaaatgctctgstttggattgttatcggaattctggccatattcttaggcatggaatttatt

SEQ ID NO: 832 - DNA, 101 bp, Phoenix dactylifera
tcctattcatctagaacttcactgcatgcaatttttttttcctttgtttckgcatttttgttggtttacgaatccatctaagcacctaaaaaaaaatggac

SEQ ID NO: 833 - DNA, 101 bp, Phoenix dactylifera
cttatgaatcttatacttgttgcttcttgtacagcctcttttgcctcttgygaatgatcagtatgtatgaagctattttgttttggacatttatctactgg

SEQ ID NO: 834 - DNA, 101 bp, Phoenix dactylifera
gtaaaatttcccctgctgttttcatcactattgttattgctcggtatcgaktattctgatgcttggcatcaccagttaggacattcaaccttgtgcaaact

SEQ ID NO: 835 - DNA, 101 bp, Phoenix dactylifera
agcagacaatttctaaacagactcctcatttattagataaagaagatcgtyaagatttcgtgatccgctgccgaacatattccaattctaacagctcggac

SEQ ID NO: 836 - DNA, 101 bp, Phoenix dactylifera
cataaaacacactgagcgtgacgaaggcccgcccaagcggccctcagctcygcacccgagaccgatatgtcaaagagttgactgccccttgyagccacgat

SEQ ID NO: 837 - DNA, 101 bp, Phoenix dactylifera
cctcagctcygcacccgagaccgatatgtcaaagagttgactgccccttgyagccacgatcctggcagatgggttccgaataacaaaacccgcgccgcccc

SEQ ID NO: 838 - DNA, 101 bp, Phoenix dactylifera
gaactgtcaaggtcccttcgataggtatgatgtgactaagctccgctgcckacacacaggcactctccaccacaaatctcggcgacatactgcgttcgcca

SEQ ID NO: 839 - DNA, 101 bp, Phoenix dactylifera
gtgctaccctcaggtggagtctcaagcgccagatccaggtgcagtctgccyaggctcgtactgccgctggagaatgaggaagagatcgcctaccctaacac

SEQ ID NO: 840 - DNA, 101 bp, Phoenix dactylifera
agaagctttgcttgcttgcaattgtctctgctcgtaccaatacttatgctycttgagcattgttggatgaggccttgtgaagctwgtcatagtcctaagat

SEQ ID NO: 841 - DNA, 101 bp, Phoenix dactylifera
taccaatacttatgctycttgagcattgttggatgaggccttgtgaagctwgtcatagtcctaagatccaccttcctcctaatggaagatttttcgtgact

SEQ ID NO: 842 - DNA, 101 bp, Phoenix dactylifera
catagtcctaagatccaccttcctcctaatggaagatttttcgtgacttargcagtatcatgctcttgaacccttcgaaacctctagtcctcttaagaaat

SEQ ID NO: 843 - DNA, 101 bp, Phoenix dactylifera
acctctagtcctcttaagaaattccatagagttctcattagctccacctaygattgatccctttaatgcaacaaagtccacgacttctgctacctctactt

SEQ ID NO: 844 - DNA, 101 bp, Phoenix dactylifera
tttaatgatgcatactagttgtattgaggcattagcaccatagtaaattgyctraagaacttcattcctgccaccacatcgaagtcatcaacgagcattat

SEQ ID NO: 845 - DNA, 101 bp, Phoenix dactylifera
aatgatgcatactagttgtattgaggcattagcaccatagtaaattgyctraagaacttcattcctgccaccacatcgaagtcatcaacgagcattatctt

SEQ ID NO: 846 - DNA, 101 bp, Phoenix dactylifera
tcgaagtcatcaacgagcattatcttgaaatcaaccttacaactccaattkccwaggtgcaactctactccatgagctacatcattaagggcctgggcttg

SEQ ID NO: 847 - DNA, 101 bp, Phoenix dactylifera
aagtcatcaacgagcattatcttgaaatcaaccttacaactccaattkccwaggtgcaactctactccatgagctacatcattaagggcctgggcttgagc

SEQ ID NO: 848 - DNA, 101 bp, Phoenix dactylifera
ttaagggcctgggcttgagcattcactgtcttcatctagcccttctccttkatcacctcaaccccaagccattttgcctcattcagcttgataaagttgtr

SEQ ID NO: 849 - DNA, 101 bp, Phoenix dactylifera
katcacctcaaccccaagccattttgcctcattcagcttgataaagttgtrtgttgcacttatgttgaccatcacatgagtagactttccattgatttggg

SEQ ID NO: 850 - DNA, 101 bp, Phoenix dactylifera
aggcatttaagtgtgaagacgactcatttgggtagcatcctagttctctcrctcctctattatagtaytgagtgcactcygattgggacaacccttgccat

SEQ ID NO: 851 - DNA, 101 bp, Phoenix dactylifera
gacgactcatttgggtagcatcctagttctctcrctcctctattatagtaytgagtgcactcygattgggacaacccttgccatatgtggactatcatata

SEQ ID NO: 852 - DNA, 101 bp, Phoenix dactylifera
gggtagcatcctagttctctcrctcctctattatagtaytgagtgcactcygattgggacaacccttgccatatgtggactatcatatagraagcggttac

SEQ ID NO: 853 - DNA, 101 bp, Phoenix dactylifera
gagtgcactcygattgggacaacccttgccatatgtggactatcatatagraagcggttacctcaagggttcttttttctatccaaccttttatcttttgg

SEQ ID NO: 854 - DNA, 101 bp, Phoenix dactylifera
cattggcatgatcaccctttaaaatccttagtctggatgactcggactttygaactacaagatttttrctactgtgagagtgattgagatgtccttcacac

SEQ ID NO: 855 - DNA, 101 bp, Phoenix dactylifera
tttaaaatccttagtctggatgactcggactttygaactacaagatttttrctactgtgagagtgattgagatgtccttcacaccataccttcgtaggtcg

SEQ ID NO: 856 - DNA, 101 bp, Phoenix dactylifera
gagttaaggagtcgatcttccttacccattctaagaatttggagaataagraaagagaatttcttcacacaattctagatcgagtttatatgcttcaggtc

SEQ ID NO: 857 - DNA, 101 bp, Phoenix dactylifera
ctagatcgagtttatatgcttcaggtccttcattttcttccatacctcatragtcacattcttggggtagaactggttctttaattacctttttagcttat

SEQ ID NO: 858 - DNA, 101 bp, Phoenix dactylifera
agcaaacaactttctccatctcagcatgcttcctatgccacacatagcaayaagattaataagaaagagggtagccattctcaccttgagcttctcatcct

SEQ ID NO: 859 - DNA, 101 bp, Phoenix dactylifera
accttgagcttctcatcctccatatatgttgaatgcctcaaaatatctctygatgttcataagaagttgtcagtctcctaggatccctygcaccatcatac

SEQ ID NO: 860 - DNA, 101 bp, Phoenix dactylifera
caaaatatctctygatgttcataagaagttgtcagtctcctaggatccctygcaccatcatactctwgggaaaaagcttcaatccttggagtgaaaattgc

SEQ ID NO: 861 - DNA, 101 bp, Phoenix dactylifera
gttcataagaagttgtcagtctcctaggatccctygcaccatcatactctwgggaaaaagcttcaatccttggagtgaaaattgctgcaagagtagcaacc

SEQ ID NO: 862 - DNA, 101 bp, Phoenix dactylifera
aatttaaagattggtcaaagccttcaatatttaggcttggaatatgagatwctcttgacattgagcttccttcgtagagttgatgatgcattatrtattct

SEQ ID NO: 863 - DNA, 101 bp, Phoenix dactylifera
tgagatwctcttgacattgagcttccttcgtagagttgatgatgcattatrtattctcatkgagctctttaacttgcctctcaaagttttcatgaygctcc

SEQ ID NO: 864 - DNA, 101 bp, Phoenix dactylifera
ttgacattgagcttccttcgtagagttgatgatgcattatrtattctcatkgagctctttaacttgcctctcaaagttttcatgaygctcctccatgcctt

SEQ ID NO: 865 - DNA, 101 bp, Phoenix dactylifera
attatrtattctcatkgagctctttaacttgcctctcaaagttttcatgaygctcctccatgccttgccccaactcttcaaaccaatttttgatatcagcc

SEQ ID NO: 866 - DNA, 101 bp, Phoenix dactylifera
cagaaacctccatcttggcggcctttgcctctatggatctaacgccatcargagacttgttttagcttcttttgagctttttccttacattccaattttgc

SEQ ID NO: 867 - DNA, 101 bp, Phoenix dactylifera
ccaattttgcttggccacctcttggtgcacattgacaacttttgaaggcayagtattccttcccayaagtaagctctgataccacttgtcaaaagcttggc

SEQ ID NO: 868 - DNA, 101 bp, Phoenix dactylifera
cacctcttggtgcacattgacaacttttgaaggcayagtattccttcccayaagtaagctctgataccacttgtcaaaagcttggcatttcgtcaagcaac

SEQ ID NO: 869 - DNA, 101 bp, Phoenix dactylifera
cctatcactctagcccctcaagctagaactctcaataagggaggagaaaarcacacacaaaagagaaaargagtaggagaatgacgagtagaattccacta

SEQ ID NO: 870 - DNA, 101 bp, Phoenix dactylifera
aagctagaactctcaataagggaggagaaaarcacacacaaaagagaaaargagtaggagaatgacgagtagaattccactagagaactcttaaaactctt

SEQ ID NO: 871 - DNA, 101 bp, Phoenix dactylifera
agtaggagaatgacgagtagaattccactagagaactcttaaaactctttrcttaggtggtttggtccakaagacctcccctctttttataggctctacct

SEQ ID NO: 872 - DNA, 101 bp, Phoenix dactylifera
gaattccactagagaactcttaaaactctttrcttaggtggtttggtccakaagacctcccctctttttataggctctacctagcacaatgaatggtcagg

SEQ ID NO: 873 - DNA, 101 bp, Phoenix dactylifera
tggttaaagctgcctattataaaatgatacaatcatgtaaatgagtgctakcttaayctagtggccatcactaaaatgtcgatacrtcccttygggttcgg

SEQ ID NO: 874 - DNA, 101 bp, Phoenix dactylifera
aagctgcctattataaaatgatacaatcatgtaaatgagtgctakcttaayctagtggccatcactaaaatgtcgatacrtcccttygggttcggctacat

SEQ ID NO: 875 - DNA, 101 bp, Phoenix dactylifera
tgtaaatgagtgctakcttaayctagtggccatcactaaaatgtcgatacrtcccttygggttcggctacatacttagtactaataagtcttttatttttc

SEQ ID NO: 876 - DNA, 101 bp, Phoenix dactylifera
gagtgctakcttaayctagtggccatcactaaaatgtcgatacrtcccttygggttcggctacatacttagtactaataagtcttttatttttcaagttaa

SEQ ID NO: 877 - DNA, 101 bp, Phoenix dactylifera
cggctacatacttagtactaataagtcttttatttttcaagttaagatttrtagtaatgcagagaaaccactagtctttyaatacactcttagcacaacct

SEQ ID NO: 878 - DNA, 101 bp, Phoenix dactylifera
ttatttttcaagttaagatttrtagtaatgcagagaaaccactagtctttyaatacactcttagcacaacctagtagagtgcataaatacatcagtgacca

SEQ ID NO: 879 - DNA, 101 bp, Phoenix dactylifera
ataaatacatcagtgaccaaatgtagcccgatggtccattaatacatacgrcaacttgactaaagtttcgagcaaaggctcaagtagcctatactygagga

SEQ ID NO: 880 - DNA, 101 bp, Phoenix dactylifera
atacgrcaacttgactaaagtttcgagcaaaggctcaagtagcctatactygaggattgcattcgccrctaaactattgaagccacctattatcaagtgat

SEQ ID NO: 881 - DNA, 101 bp, Phoenix dactylifera
aagtttcgagcaaaggctcaagtagcctatactygaggattgcattcgccrctaaactattgaagccacctattatcaagtgatgtgatcaagtacatgwg

SEQ ID NO: 882 - DNA, 101 bp, Phoenix dactylifera
crctaaactattgaagccacctattatcaagtgatgtgatcaagtacatgwgccaagcacaacccaatggccctcactaaatgttaacaagcctattatcc

SEQ ID NO: 883 - DNA, 101 bp, Phoenix dactylifera
gcacaacccaatggccctcactaaatgttaacaagcctattatccctcaartcaagctctatgtcgatgcatacaaataactagtccttcatagcrctcct

SEQ ID NO: 884 - DNA, 101 bp, Phoenix dactylifera
ctcaartcaagctctatgtcgatgcatacaaataactagtccttcatagcrctcctagtgcaatccaatagagtgcacacatcctactttgaccaaagaca

SEQ ID NO: 885 - DNA, 101 bp, Phoenix dactylifera
gagtgcacacatcctactttgaccaaagacaatttggcggtaacatattayatacaactatgattcaacatgcatttgcatttacrccatgctaccatcat

SEQ ID NO: 886 - DNA, 101 bp, Phoenix dactylifera
ggcggtaacatattayatacaactatgattcaacatgcatttgcatttacrccatgctaccatcatttcccaaaacatgataatagaggcaacccttttta

SEQ ID NO: 887 - DNA, 101 bp, Phoenix dactylifera
catcatttcccaaaacatgataatagaggcaaccctttttaaagtcaccgytgctcttttcacctcttcaattgggggctatcrtccttwgctttgacact

SEQ ID NO: 888 - DNA, 101 bp, Phoenix dactylifera
cctttttaaagtcaccgytgctcttttcacctcttcaattgggggctatcrtccttwgctttgacactctcccttttcatagcctttaaaagtgacataat

SEQ ID NO: 889 - DNA, 101 bp, Phoenix dactylifera
taaagtcaccgytgctcttttcacctcttcaattgggggctatcrtccttwgctttgacactctcccttttcatagcctttaaaagtgacataatgtagac

SEQ ID NO: 890 - DNA, 101 bp, Phoenix dactylifera
tttgacactctcccttttcatagcctttaaaagtgacataatgtagacacsytagggtgagttatttatttatttatttttgcttggttgcatctaaccaa

SEQ ID NO: 891 - DNA, 101 bp, Phoenix dactylifera
ttgacactctcccttttcatagcctttaaaagtgacataatgtagacacsytagggtgagttatttatttatttatttttgcttggttgcatctaaccaat

SEQ ID NO: 892 - DNA, 101 bp, Phoenix dactylifera
tggttgcatctaaccaatctcacgtatcctagttgattcaacccaatctcrcctattccaagctaaggtcacactagatctatctgattagaccttacttg

SEQ ID NO: 893 - DNA, 101 bp, Phoenix dactylifera
ttagaccttacttgactttatccaacctaaccctagccatctgatcacacycgatcctacctagtccaacctaatcatcgctatcccatcacacctactct

SEQ ID NO: 894 - DNA, 101 bp, Phoenix dactylifera
gatcctactcaatctcacatcttacttaatctaacctatacttagccatcmgaccataccaaattcaacccaatcatactgaatgttatccaatcttgcct

SEQ ID NO: 895 - DNA, 101 bp, Phoenix dactylifera
tgttatccaatcttgcctaatctcacataatccaacctaaaccgagtcacrtgatcttacttgatsatactcaataatactcgatcacatctgrtcatacg

SEQ ID NO: 896 - DNA, 101 bp, Phoenix dactylifera
cctaatctcacataatccaacctaaaccgagtcacrtgatcttacttgatsatactcaataatactcgatcacatctgrtcatacgtaatctaatcctacc

SEQ ID NO: 897 - DNA, 101 bp, Phoenix dactylifera
gagtcacrtgatcttacttgatsatactcaataatactcgatcacatctgrtcatacgtaatctaatcctaccttggtcctccttatcctagcatagtttg

SEQ ID NO: 898 - DNA, 101 bp, Phoenix dactylifera
gtccacccctttagtataatgcatgctatgaataatattctatcgtatagkatgtgtatcttgcaaaaattattttcatttttaacctccaccatccttct

SEQ ID NO: 899 - DNA, 101 bp, Phoenix dactylifera
attattttcatttttaacctccaccatccttctcttggaacaactctactmttartgagacaaggggcacaacaacaaaagctctatcttatttttgtcat

SEQ ID NO: 900 - DNA, 101 bp, Phoenix dactylifera
ttttcatttttaacctccaccatccttctcttggaacaactctactmttartgagacaaggggcacaacaacaaaagctctatcttatttttgtcatgatt

SEQ ID NO: 901 - DNA, 101 bp, Phoenix dactylifera
acaaggggcacaacaacaaaagctctatcttatttttgtcatgattccttmtaactaactcaatccctcactctccctccttatactatgtgtctcttcaa

SEQ ID NO: 902 - DNA, 101 bp, Phoenix dactylifera
ttcaaacatcatttctctatgctaccatccttgtatgaaatcccaaccctrcaagaagataaaagtgagatattatctataacaatgcayttccccccaac

SEQ ID NO: 903 - DNA, 101 bp, Phoenix dactylifera
atcccaaccctrcaagaagataaaagtgagatattatctataacaatgcayttccccccaacttgatattatttttctgaatrcattggtcaatctcatct

SEQ ID NO: 904 - DNA, 101 bp, Phoenix dactylifera
attatctataacaatgcayttccccccaacttgatattatttttctgaatrcattggtcaatctcatcttccatctagcatccctctttatccttagatta

SEQ ID NO: 905 - DNA, 101 bp, Phoenix dactylifera
ctagatggaaactctcatacccataaggtagcctacattgagattggtggraggaaaagtggttcatgtgtcattggaaaattggtgtcaaagatagcgat

SEQ ID NO: 906 - DNA, 101 bp, Phoenix dactylifera
atgtgtcattggaaaattggtgtcaaagatagcgatagatatgtcaacttrgacgatgatggcacagacgaggagattatgtacmacactgaagcaatgtg

SEQ ID NO: 907 - DNA, 101 bp, Phoenix dactylifera
atagatatgtcaacttrgacgatgatggcacagacgaggagattatgtacmacactgaagcaatgtgtaaacwcgagattgaaggaggggactgagggaya

SEQ ID NO: 908 - DNA, 101 bp, Phoenix dactylifera
tgatggcacagacgaggagattatgtacmacactgaagcaatgtgtaaacwcgagattgaaggaggggactgagggayagctgtctatstcaagatctctt

SEQ ID NO: 909 - DNA, 101 bp, Phoenix dactylifera
cmacactgaagcaatgtgtaaacwcgagattgaaggaggggactgagggayagctgtctatstcaagatctcttctttactttcattgatatctctttagc

SEQ ID NO: 910 - DNA, 101 bp, Phoenix dactylifera
caatgtgtaaacwcgagattgaaggaggggactgagggayagctgtctatstcaagatctcttctttactttcattgatatctctttagcactccattttg

SEQ ID NO: 911 - DNA, 101 bp, Phoenix dactylifera
tctcttctttactttcattgatatctctttagcactccattttgaagtatyctattatctctcaactagatcyatatatctatggctctctccgccgctct

SEQ ID NO: 912 - DNA, 101 bp, Phoenix dactylifera
atctctttagcactccattttgaagtatyctattatctctcaactagatcyatatatctatggctctctccgccgctctcaccctctaccttctatttctc

SEQ ID NO: 913 - DNA, 101 bp, Phoenix dactylifera
ggtacgagagttctacaacaatgctagatttgaacaaggttagatctcatycaaagttaaagagacttctctcctaatctcagaagaagtacttaaggatg

SEQ ID NO: 914 - DNA, 101 bp, Phoenix dactylifera
aaccttagaaagaaaacaacaaggaagagagaaacattcaatcaagaaaamgatgtttccacaaagaaaggagaagaaaaaagcaaaaagttcatcatyct

SEQ ID NO: 915 - DNA, 101 bp, Phoenix dactylifera
aamgatgtttccacaaagaaaggagaagaaaaaagcaaaaagttcatcatyctcaccggtattcctctgaaacttgctcctgtgaggaaatcagtcaaagc

SEQ ID NO: 916 - DNA, 101 bp, Phoenix dactylifera
agctcgaaaaaacaaacggtgatatattttctgttggttgcctggaaaaaycgactgggtttctgattttgcatccggaaaacaatcaggctataatttgc

SEQ ID NO: 917 - DNA, 101 bp, Phoenix dactylifera
ctaaatgcaaattaaacaagagcaagtagacaagtaaaagataaagagaastctgcaaaataagtacacaacaaacacaagtattttgtaaaggttcggaa

SEQ ID NO: 918 - DNA, 101 bp, Phoenix dactylifera
cctggttgcatcgtagaaatcacttgttttgtctctactctatgaccactytgaaattggctctttcgagactagagtcggctcctagcttgtactagtta

SEQ ID NO: 919 - DNA, 101 bp, Phoenix dactylifera
atgacatatttcttttttcaaaacatataaaacctatagtatcatattcaratattttatatcatcaaaatcaattcttagggtcaacataagatatcccg

SEQ ID NO: 920 - DNA, 101 bp, Phoenix dactylifera
tttcgatcttaactcttcttaatggatgtgtgtgtgtgatttagttagtgycaagtgtatatgtgaaattgaaaacacaaacaagacaatacaaatacaca

SEQ ID NO: 921 - DNA, 101 bp, Phoenix dactylifera
atagtggttcggtgcaccccacggcaccaacgtccactcttcaagcgtccycttgagaattcactataattcctcggactacaactagattgttttccagg

SEQ ID NO: 922 - DNA, 101 bp, Phoenix dactylifera
ttttcgggctcaccaaaaaccgatgttagttttccggactcaccaacaacytattccgttggttttacgggctcaccaacaaaccttacaacaatattaag

SEQ ID NO: 923 - DNA, 101 bp, Phoenix dactylifera
tggtggagctcttgatttctctttgaatatgctcaaccaccttcttttgaytttagaatgaagcaaattgatgaaacacttgaagactaggatttttactw

SEQ ID NO: 924 - DNA, 101 bp, Phoenix dactylifera
ytttagaatgaagcaaattgatgaaacacttgaagactaggatttttactwggatgctcttcttaattgtaaaagattctttccactgatgaatagtaccc

SEQ ID NO: 925 - DNA, 101 bp, Phoenix dactylifera
ctgatgaatagtaccccttaaatacttctccaacctctaatagtacctctytaactgtttgaatggagaaactagccgtttctatctgttggagactcaaa

SEQ ID NO: 926 - DNA, 101 bp, Phoenix dactylifera
aaggagtgctagggtgactctattacttctggggtcgactccaattctatrccaagtgttccaaggttgtgccatatcgtgccaggatgtgccaaatgtat

SEQ ID NO: 927 - DNA, 101 bp, Phoenix dactylifera
gtgttccaaggttgtgccatatcgtgccaggatgtgccaaatgtatcaggrtcgactccaaactacttggggtcgactctaatcctatttttttgcacttg

SEQ ID NO: 928 - DNA, 101 bp, Phoenix dactylifera
aaaattacattactaggataggggtgataaatttttgttttgatgcaaaaytacactactaggataggatgactcacataacaaaaggtacgaattgcaca

SEQ ID NO: 929 - DNA, 101 bp, Phoenix dactylifera
tgcttgtaaataagaaatttgaaatttttgataacatatgttagatgttcrcatttacatgtcaaaagaccrtgccatagaagctgattagattctactat

SEQ ID NO: 930 - DNA, 101 bp, Phoenix dactylifera
aaatttttgataacatatgttagatgttcrcatttacatgtcaaaagaccrtgccatagaagctgattagattctactatatctcccagactacatttctt

SEQ ID NO: 931 - DNA, 101 bp, Phoenix dactylifera
cataattctatgttttattagtggacaataaagtttgtttctggaaaggaytkcaatccactcctctccaatccttatgttcataatcacgagatgaccgg

SEQ ID NO: 932 - DNA, 101 bp, Phoenix dactylifera
taattctatgttttattagtggacaataaagtttgtttctggaaaggaytkcaatccactcctctccaatccttatgttcataatcacgagatgaccggtc

SEQ ID NO: 933 - DNA, 101 bp, Phoenix dactylifera
gaagtaattaataaattagaaagcagaattacgatgatgcgaagcatctaktcatggtctgttcatttctacaattgattcaatcacagcgatatattacg

SEQ ID NO: 934 - DNA, 101 bp, Phoenix dactylifera
aacttcatcatgtgcattaaattatataggagaagttgagaaaactgacartctcggtgaattaaaaagacttgagttattaactggaccaargtatcagt

SEQ ID NO: 935 - DNA, 101 bp, Phoenix dactylifera
aactgacartctcggtgaattaaaaagacttgagttattaactggaccaargtatcagttgaaggtacatggaaaactaatagttctgataactatcagac

SEQ ID NO: 936 - DNA, 101 bp, Phoenix dactylifera
cagaaatccagagaagatgcaatataactggaaatcccagaattaccttcrtgatgaccagcaaatgcagaagccacagagaaggtcccatctttggactc

SEQ ID NO: 937 - DNA, 101 bp, Phoenix dactylifera
ttcattgaacaataatgcatatatctccatcaaaaagggcaagctagagamtatgcaggtgtcacaagatgagattccttcataattgcaaaccacttagc

SEQ ID NO: 938 - DNA, 101 bp, Phoenix dactylifera
agggagaaggccaaagagaaggttaaggaagggtatgatgccgcccaagcwaaggccggggagactctggatgccgccaaggagatccttgcctccaacta

SEQ ID NO: 939 - DNA, 101 bp, Phoenix dactylifera
gacgagaagatgagctttaagagtttcttcttttcccgttagatgagactytggccgccatgatatctttgttcgttttcttatgtaatatgcaaactgga

SEQ ID NO: 940 - DNA, 101 bp, Phoenix dactylifera
gccgccatgatatctttgttcgttttcttatgtaatatgcaaactggaccscgtgtatacaattttcttatttcataatggaggattgtgagtttgttttt

SEQ ID NO: 941 - DNA, 101 bp, Phoenix dactylifera
ggtagggctgaaattgggaagggttggatgcctatctgaacaatttcgaamtgaagattggtcaaagtttaagatttctttycggtacctrtacttgagat

SEQ ID NO: 942 - DNA, 101 bp, Phoenix dactylifera
ctatctgaacaatttcgaamtgaagattggtcaaagtttaagatttctttycggtacctrtacttgagatagacccgaaaccctagtgcattagcctagaa

SEQ ID NO: 943 - DNA, 101 bp, Phoenix dactylifera
caatttcgaamtgaagattggtcaaagtttaagatttctttycggtacctrtacttgagatagacccgaaaccctagtgcattagcctagaaccgagtttc

SEQ ID NO: 944 - DNA, 101 bp, Phoenix dactylifera
ctagaccccatgcatttgcggagccaatgttgctggtcctgttgctgcccrcaaaagaccacccccaagatgacagggatggcgaaaaggtgccaggttct

SEQ ID NO: 945 - DNA, 101 bp, Phoenix dactylifera
aaaaggtgccaggttctgaaatcgaagaggatgctgcaccagaccagatcraagaaggacaagatgaaacaaatgactcactgccaatatctgatcgtctg

SEQ ID NO: 946 - DNA, 101 bp, Phoenix dactylifera
ttgactgctggtgttcctgaaaccggaaacccacaattgttatatatggamaatcagcctgccgtgaaagagttaataccttaaccttaggcacytcccaa

SEQ ID NO: 947 - DNA, 101 bp, Phoenix dactylifera
tatggamaatcagcctgccgtgaaagagttaataccttaaccttaggcacytcccaagtaaacaaaaaagcagcacaccgggcttttctttacagcaaaac

SEQ ID NO: 948 - DNA, 101 bp, Phoenix dactylifera
agtaaacaaaaaagcagcacaccgggcttttctttacagcaaaacaagttmctcaactttcagcaaagaatattttaaagagtcattagatatttattagg

SEQ ID NO: 949 - DNA, 101 bp, Phoenix dactylifera
cccggcgaagaagcttctccaaaagcaaggcatgagctcaagggcttcccygcccgtcgctatggarctgaggtcatgaggattttgtcctcttgtctgtg

SEQ ID NO: 950 - DNA, 101 bp, Phoenix dactylifera
ctccaaaagcaaggcatgagctcaagggcttcccygcccgtcgctatggarctgaggtcatgaggattttgtcctcttgtctgtgcgtcggtgaacgtagc

SEQ ID NO: 951 - DNA, 101 bp, Phoenix dactylifera
acaaaaaaaaatgaaatgggtatgtataatatgaagctcgtcaaatcagtytcaatgatacttcaaaaatagaatcgtgctgcaagacaattaaggagaga

SEQ ID NO: 952 - DNA, 101 bp, Phoenix dactylifera
aaatagaatcgtgctgcaagacaattaaggagagataaccaacatcatgargaggcagatcaaagaaaatcttatgataagtgaaggccaatagcatatga

SEQ ID NO: 953 - DNA, 101 bp, Phoenix dactylifera
aaatcttatgataagtgaaggccaatagcatatgagtatcggtctaacagwtggtgccaagtagccatcctaatggtcatgtagtgrtggaaagttaacya

SEQ ID NO: 954 - DNA, 101 bp, Phoenix dactylifera
tatcggtctaacagwtggtgccaagtagccatcctaatggtcatgtagtgrtggaaagttaacyagcatcagtcaccagaaccaccagaagtggaggccag

SEQ ID NO: 955 - DNA, 101 bp, Phoenix dactylifera
gwtggtgccaagtagccatcctaatggtcatgtagtgrtggaaagttaacyagcatcagtcaccagaaccaccagaagtggaggccagatatgaaaccgtt

SEQ ID NO: 956 - DNA, 101 bp, Phoenix dactylifera
agaggagatctccttcactttctgcacatacttcactattgtaggatctcragcttcatactctcttygaacttgattaatgactagytggaaaatgttgt

SEQ ID NO: 957 - DNA, 101 bp, Phoenix dactylifera
ctttctgcacatacttcactattgtaggatctcragcttcatactctcttygaacttgattaatgactagytggaaaatgttgtaggtcctgaattgggtt

SEQ ID NO: 958 - DNA, 101 bp, Phoenix dactylifera
attgtaggatctcragcttcatactctcttygaacttgattaatgactagytggaaaatgttgtaggtcctgaattgggttacytggacaacttgggtaat

SEQ ID NO: 959 - DNA, 101 bp, Phoenix dactylifera
acttgattaatgactagytggaaaatgttgtaggtcctgaattgggttacytggacaacttgggtaatcttaatccaataatcactgcatcatactctgtg

SEQ ID NO: 960 - DNA, 101 bp, Phoenix dactylifera
cacttgagtatatttcatattatactaataaattcaaatgagtgtttcagraaaatattgctraatgtgctaaaatgacttaaaaaattggttcaaggatt

SEQ ID NO: 961 - DNA, 101 bp, Phoenix dactylifera
tttcatattatactaataaattcaaatgagtgtttcagraaaatattgctraatgtgctaaaatgacttaaaaaattggttcaaggattttaaactcaacc

SEQ ID NO: 962 - DNA, 101 bp, Phoenix dactylifera
taaaaaattggttcaaggattttaaactcaacctttactatgtttggaatyatttatcttcgccctatactcgagtcaactccgaagattctcaagtcgac

SEQ ID NO: 963 - DNA, 101 bp, Phoenix dactylifera
ccaagtgagatatcaagtttttaaagcttcttataccaagtcgactccaaytrtggacgagtcaactccaacaagaaggacagaaagtcagtttctgatat

SEQ ID NO: 964 - DNA, 101 bp, Phoenix dactylifera
aagtgagatatcaagtttttaaagcttcttataccaagtcgactccaaytrtggacgagtcaactccaacaagaaggacagaaagtcagtttctgatatac

SEQ ID NO: 965 - DNA, 101 bp, Phoenix dactylifera
gactccaatctcaacggtcatattgtcaagctgcacagtaatatcagaacrgctctcttactctctctaacggctatttttttaaaaaaaagagccttgaa

SEQ ID NO: 966 - DNA, 101 bp, Phoenix dactylifera
gattatctgattctcaaccaccggcgcattcaagagaggattcagaaaagragaagctacatmttcctttagcataaaaatctgtttgagagcttctaccc

SEQ ID NO: 967 - DNA, 101 bp, Phoenix dactylifera
ctcaaccaccggcgcattcaagagaggattcagaaaagragaagctacatmttcctttagcataaaaatctgtttgagagcttctaccctttttcatattg

SEQ ID NO: 968 - DNA, 101 bp, Phoenix dactylifera
tcatattgtttatattgtgttgtaactcatatattctgattttgtttcttrattcaatcccctaattgaatcaaggggattaaggtttgttggtgaaccag

SEQ ID NO: 969 - DNA, 101 bp, Phoenix dactylifera
accactataaaattctatgtgtttgtgtcgtagttctttgagcatcttttytatattgcttttttaataggttgatttatcttcttagattaaaatcaaga

SEQ ID NO: 970 - DNA, 101 bp, Phoenix dactylifera
tagattactcaaaaaagtaccaaagtcttattagtacccaattcacccccyttcttgggttgtctactgggcaacactatgcttcatgttggaggtcttga

SEQ ID NO: 971 - DNA, 101 bp, Phoenix dactylifera
cactatgcttcatgttggaggtcttgaagttaaacctttgagcatactcgrccatgatgtcctcagggctggtcaggatcaatccagatttgcatctttct

SEQ ID NO: 972 - DNA, 101 bp, Phoenix dactylifera
ccatgatgtcctcagggctggtcaggatcaatccagatttgcatctttctrcactagaagacccataaacatggagtgtccagaaaggttgtgcctctgaa

SEQ ID NO: 973 - DNA, 24 bp, Artificial
  Primer
gagttaatatctccttgccatcct

SEQ ID NO: 974 - DNA, 22 bp, Artificial
  Primer
gtcaagggatctccctattgta

SEQ ID NO: 975 - DNA, 45 bp, Artificial
  Primer
tatgtacacargtaagkctcctttttcacttgsagacatatagag

SEQ ID NO: 976 - DNA, 36 bp, Artificial
  Primer
attcctgccaccacatcgaagtcatcaacgagcatt

SEQ ID NO: 977 - DNA, 11 bp, Artificial
  Primer
ccttkatcacc

SEQ ID NO: 978 - DNA, 31 bp, Artificial
  Primer
ctgattgggacaacccttgccatatgtggac

SEQ ID NO: 979 - DNA, 31 bp, Artificial
  Primer
cagccatgctcaatgcccttagtcgcctagt

SEQ ID NO: 980 - DNA, 11 bp, Artificial
  Primer
caccrgggtca

SEQ ID NO: 981 - DNA, 11 bp, Artificial
  Primer
caagccccggc

SEQ ID NO: 982 - DNA, 11 bp, Artificial
  Primer
tgcttccggtg

SEQ ID NO: 983 - DNA, 37 bp, Artificial
  Primer
ttcccttgactttatcgaagggttaccgttgtccaaa

SEQ ID NO: 984 - DNA, 41 bp, Artificial
  Primer
tcctaggaagaggtacttctcaaagaatctgtagaaatgca

SEQ ID NO: 985 - DNA, 11 bp, Artificial
  Primer
catatgtaygc

SEQ ID NO: 986 - DNA, 39 bp, Artificial
  Primer
atatgttcaacattttgggtcaggtctaggtttattgaa

SEQ ID NO: 987 - DNA, 41 bp, Artificial
  Primer
ttgtattgaggcattagcaccatagtaaattgtctgaagaa

SEQ ID NO: 988 - DNA, 41 bp, Artificial
  Primer
ttgtattgcggcaatagcaccatagtaaattgcctaaagaa

SEQ ID NO: 989 - DNA, 36 bp, Artificial
  Primer
attatagtattgagtgcactctgattgggacaaccc

SEQ ID NO: 990 - DNA, 36 bp, Artificial
  Primer
attatagtactgagtgcactccgattgggacaaccc

SEQ ID NO: 991 - DNA, 31 bp, Artificial
  Primer
catagtaaattgtctgaagaacttcattcct

SEQ ID NO: 992 - DNA, 31 bp, Artificial
  Primer
catagtaaattgcctaaagaacttcattcct

SEQ ID NO: 993 - DNA, 31 bp, Artificial
  Primer
ttttggagatccgcacaccgagttagccctc

SEQ ID NO: 994 - DNA, 31 bp, Artificial
  Primer
ttttagagatccgtgcaccaagttagccctc